SEQUENCE LISTING (513 sequences)

SEQ ID NO: 1 - DNA, 75 bp, Artificial Sequence
  Synthetic oligonucleotide BRCA1ex18NNNNNN5_10selPCR (machine-mixing)
gagtgtttttcattctgcagatgcnnnnnntgtgtggatatccacactgaaatattttctaggaattgcgggagg

SEQ ID NO: 2 - DNA, 20 bp, Artificial Sequence
  Synthetic oligonucleotide BRCA1ex18_Hexamers_F
gagtgtttttcattctgcag

SEQ ID NO: 3 - DNA, 20 bp, Artificial Sequence
  Synthetic oligonucleotide BRCA1ex18_Hexamers_R
cctcccgcaattcctagaaa

SEQ ID NO: 4 - DNA, 35 bp, Artificial Sequence
  Synthetic oligonucleotide pUC_gBRCA1_INF_5′
cggtacccggggatcgaactcccgacatcaggtga

SEQ ID NO: 5 - DNA, 35 bp, Artificial Sequence
  Synthetic oligonucleotide pUC_gBRCA1_INF_3′
cgactctagaggatcaaggattctgtgagggagca

SEQ ID NO: 6 - DNA, 20 bp, Artificial Sequence
  Synthetic oligonucleotide Lin_pUC_BRCA1ex18_5′
ctgcagaatgaaaaacactc

SEQ ID NO: 7 - DNA, 20 bp, Artificial Sequence
  Synthetic oligonucleotide Lin_pUC_BRCA1ex18_3′
tttctaggaattgcgggagg

SEQ ID NO: 8 - DNA, 100 bp, Artificial Sequence
  Synthetic oligonucleotide BRCA1ex18_3%_mut Template (blue = 97wt/1/1/1 bases; hand-mixing)
attctgcagatgctgagtttgtgtgtgaacggacactgaaatattttctaggaattgcgggaggaaaatgggtagttagctatttctgtaagtataatac

SEQ ID NO: 9 - DNA, 20 bp, Artificial Sequence
  Synthetic oligonucleotide BRCA1ex18_51_Amp
gagtgtttttcattctgcag

SEQ ID NO: 10 - DNA, 47 bp, Artificial Sequence
  Synthetic oligonucleotide 5′2_Syn_Amp
ttctgcagatgctgagtttgtctgcgagagaacactgaaatattttc

SEQ ID NO: 11 - DNA, 47 bp, Artificial Sequence
  Synthetic oligonucleotide 5′2_Eco_Amp
ttctgcagatgctgagtttgtgtggatatccacactgaaatattttc

SEQ ID NO: 12 - DNA, 22 bp, Artificial Sequence
  Synthetic oligonucleotide BRCA1ex18_ 31_Amp
ggagaaatagtattatacttac

SEQ ID NO: 13 - DNA, 49 bp, Artificial Sequence
  Synthetic oligonucleotide  32_Syn_Amp
ttatacttacagaaatagcttacaacccacttaccacctgcaattccta

SEQ ID NO: 14 - DNA, 44 bp, Artificial Sequence
  Synthetic oligonucleotide  3′2 _Eco_Amp
ttatacttacagaaatagctaaggatatcttttcctcccgcaat

SEQ ID NO: 15 - DNA, 20 bp, Artificial Sequence
  Synthetic oligonucleotide  In-Fusion_Lin_5′1
ctgcagaatgaaaaacactc

SEQ ID NO: 16 - DNA, 22 bp, Artificial Sequence
  Synthetic oligonucleotide  In-Fusion_Lin_3′1
gtaagtataatactatttctcc

SEQ ID NO: 17 - DNA, 20 bp, Artificial Sequence
  Synthetic oligonucleotide  In-Fusion_Lin_5′2
caaactcagcatctgcagaa

SEQ ID NO: 18 - DNA, 20 bp, Artificial Sequence
  Synthetic oligonucleotide  In-Fusion_Lin_3′2
agctatttctgtaagtataa

SEQ ID NO: 19 - DNA, 25 bp, Artificial Sequence
  Synthetic oligonucleotide  sgRNA_BRCA1ex18_F
caccgatgctgagtttgtgtgtgaa

SEQ ID NO: 20 - DNA, 25 bp, Artificial Sequence
  Synthetic oligonucleotide  sgRNA_BRCA1ex18_R
aaacttcacacacaaactcagcatc

SEQ ID NO: 21 - DNA, 25 bp, Artificial Sequence
  Synthetic oligonucleotide  BRCA1ex19_RT
tttctttctttaatagactgggtca

SEQ ID NO: 22 - DNA, 18 bp, Artificial Sequence
  Synthetic oligonucleotide  BRCA1ex21_RT
gtgggcatgttggtgaag

SEQ ID NO: 23 - DNA, 22 bp, Artificial Sequence
  Synthetic oligonucleotide external_BRCA1ex18_F
cggcttttgtagcagttaaaca

SEQ ID NO: 24 - DNA, 27 bp, Artificial Sequence
  Synthetic oligonucleotide BRCA1ex18SelPCR_Hex_R
cctagaaaatatttcagtgtggatatc

SEQ ID NO: 25 - DNA, 19 bp, Artificial Sequence
  Synthetic oligonucleotide  BRCA1ex16_cDNA1_F
ggagaagccagaattgaca

SEQ ID NO: 26 - DNA, 45 bp, Artificial Sequence
  Synthetic oligonucleotide PU1L_BRCA1intron17_F
ctaaatggctgtgagagagctcagtggtgttttcagcctctgatt

SEQ ID NO: 27 - DNA, 51 bp, Artificial Sequence
  Synthetic oligonucleotide PU1R_BRCA1ex18SelPCR_Hex_R
actttatcaatctcgctccaaacccctagaaaatatttcagtgtggatatc

SEQ ID NO: 28 - DNA, 43 bp, Artificial Sequence
  Synthetic oligonucleotide PU1L_BRCA1ex16_cDNA2_F
ctaaatggctgtgagagagctcaggcttcaacagaaagggtca

SEQ ID NO: 29 - DNA, 46 bp, Artificial Sequence
  Synthetic oligonucleotide PU1R_BRCA1ex18_ 31_Amp
actttatcaatctcgctccaaaccggagaaatagtattatacttac

SEQ ID NO: 30 - DNA, 61 bp, Artificial Sequence
  Synthetic oligonucleotide  FC_PU1L
aatgatacggcgaccaccgagatctacacacgtaggcctaaatggctgtgagagagctcag

SEQ ID NO: 31 - DNA, 67 bp, Artificial Sequence
  Synthetic oligonucleotide  FC_index_PU1R_142
caagcagaagacggcatacgagataagcgttcagaccgtcggcactttatcaatctcgctccaaacc

SEQ ID NO: 32 - DNA, 67 bp, Artificial Sequence
  Synthetic oligonucleotide  FC_index_PU1R_143
caagcagaagacggcatacgagatcgcaagcgtgaccgtcggcactttatcaatctcgctccaaacc

SEQ ID NO: 33 - DNA, 67 bp, Artificial Sequence
  Synthetic oligonucleotide  FC_index_PU1R_144
caagcagaagacggcatacgagatgcagcgcgagaccgtcggcactttatcaatctcgctccaaacc

SEQ ID NO: 34 - DNA, 67 bp, Artificial Sequence
  Synthetic oligonucleotide  FC_index_PU1R_145
caagcagaagacggcatacgagatcgcgcagctgaccgtcggcactttatcaatctcgctccaaacc

SEQ ID NO: 35 - DNA, 67 bp, Artificial Sequence
  Synthetic oligonucleotide  FC_index_PU1R_146
caagcagaagacggcatacgagattcaagcgcagaccgtcggcactttatcaatctcgctccaaacc

SEQ ID NO: 36 - DNA, 67 bp, Artificial Sequence
  Synthetic oligonucleotide  FC_index_PU1R_147
caagcagaagacggcatacgagatcagtcgcaggaccgtcggcactttatcaatctcgctccaaacc

SEQ ID NO: 37 - DNA, 67 bp, Artificial Sequence
  Synthetic oligonucleotide  FC_index_PU1R_148
caagcagaagacggcatacgagatgcgtcagttgaccgtcggcactttatcaatctcgctccaaacc

SEQ ID NO: 38 - DNA, 67 bp, Artificial Sequence
  Synthetic oligonucleotide  FC_index_PU1R_149
caagcagaagacggcatacgagatagtcgcgcagaccgtcggcactttatcaatctcgctccaaacc

SEQ ID NO: 39 - DNA, 20 bp, Artificial Sequence
  Synthetic oligonucleotide  BRCA1ex20_R
ctttctgtcctgggattctc

SEQ ID NO: 40 - DNA, 44 bp, Artificial Sequence
  Synthetic oligonucleotide  PU1R_BRCA1ex20
actttatcaatctcgctccaaaccctttctgtcctgggattctc

SEQ ID NO: 41 - DNA, 19 bp, Artificial Sequence
  Synthetic oligonucleotide  BRCA1ex16_cDNA2_F
gcttcaacagaaagggtca

SEQ ID NO: 42 - DNA, 43 bp, Artificial Sequence; identical to SEQ ID NO: 28
  Synthetic oligonucleotide PU1L_BRCA1ex16_cDNA2_F

SEQ ID NO: 43 - DNA, 44 bp, Artificial Sequence
  Synthetic oligonucleotide PU1L_BRCA1intron17_2
ctaaatggctgtgagagagctcagccagattgatcttgggagtg

SEQ ID NO: 44 - DNA, 44 bp, Artificial Sequence
  Synthetic oligonucleotide  PU1R_BRCA1intron18
actttatcaatctcgctccaaaccggtaactcagactcagcatc

SEQ ID NO: 45 - DNA, 19 bp, Artificial Sequence
  Synthetic oligonucleotide  BRCA1ex18_sel5Syn_F
tgagtttgtctgcgagaga

SEQ ID NO: 46 - DNA, 43 bp, Artificial Sequence
  Synthetic oligonucleotide PU1L_BRCA1ex18_sel5syn
ctaaatggctgtgagagagctcagtgagtttgtctgcgagaga

SEQ ID NO: 47 - DNA, 21 bp, Artificial Sequence
  Synthetic oligonucleotide  BRCA1ex18_sel5Eco_F
gctgagtttgtgtggatatcc

SEQ ID NO: 48 - DNA, 45 bp, Artificial Sequence
  Synthetic oligonucleotide PU1L_BRCA1ex18_sel5Eco
ctaaatggctgtgagagagctcaggctgagtttgtgtggatatcc

SEQ ID NO: 49 - DNA, 19 bp, Artificial Sequence
  Synthetic oligonucleotide  BRCA1ex18_sel3Syn_R
tacaacccacttaccacct

SEQ ID NO: 50 - DNA, 43 bp, Artificial Sequence
  Synthetic oligonucleotide PU1R_BRCA1ex18_sel3Syn
actttatcaatctcgctccaaacctacaacccacttaccacct

SEQ ID NO: 51 - DNA, 25 bp, Artificial Sequence
  Synthetic oligonucleotide  BRCA1ex18_sel3Eco_R
acttacagaaatagctaaggatatc

SEQ ID NO: 52 - DNA, 49 bp, Artificial Sequence
  Synthetic oligonucleotide PU1R_BRCA1ex18_sel3Eco
actttatcaatctcgctccaaaccacttacagaaatagctaaggatatc

SEQ ID NO: 53 - DNA, 124 bp, Artificial Sequence
  Synthetic oligonucleotide CustomArray DBR1 oligonucleotide pool
ccagttctcacgctcttcatcggcggaaaccatgaagcctcaaatcatttgcaagagttaccctatggtggctgggtggcaccaaacatttattatttaggtatgtgtgatactttgtggatac

SEQ ID NO: 54 - DNA, 20 bp, Artificial Sequence
  Synthetic oligonucleotide  Dbr1X2D_AMP_F
tctcacgctcttcatcggcg

SEQ ID NO: 55 - DNA, 19 bp, Artificial Sequence
  Synthetic oligonucleotide  Dbr1X2D_AMP_R
tccacaaagtatcacacat

SEQ ID NO: 56 - DNA, 35 bp, Artificial Sequence
  Synthetic oligonucleotide  pUC19_Dbr1_Inf_F
cggtacccggggatcgaggcgggaaatgtattcag

SEQ ID NO: 57 - DNA, 35 bp, Artificial Sequence
  Synthetic oligonucleotide  pUC19_Dbr1_Inf_R
cgactctagaggatccaggaaatgctgcaagacaa

SEQ ID NO: 58 - DNA, 25 bp, Artificial Sequence
  Synthetic oligonucleotide  Lin_pUC19-Dbr1X2_5
cgccgatgaagagcgtgagaactgg

SEQ ID NO: 59 - DNA, 36 bp, Artificial Sequence
  Synthetic oligonucleotide  Lin_pUC19-Dbr1X2_3
atgtgtgatactttgtggatacttttttttcctggg

SEQ ID NO: 60 - DNA, 20 bp, Artificial Sequence
  Synthetic oligonucleotide  external_Dbr1_F
gcctgtttcccttagatcct

SEQ ID NO: 61 - DNA, 21 bp, Artificial Sequence
  Synthetic oligonucleotide  Dbr1Int2_selAMP_R
agtatccacaaagtatcacac

SEQ ID NO: 62 - DNA, 20 bp, Artificial Sequence
  Synthetic oligonucleotide  Dbr1_Int2_R2
cctgataagctcttccatcc

SEQ ID NO: 63 - DNA, 44 bp, Artificial Sequence
  Synthetic oligonucleotide  PU1L_Dbr1Int1_AMP
ctaaatggctgtgagagagctcaggcctaattgtggtaactgac

SEQ ID NO: 64 - DNA, 45 bp, Artificial Sequence
  Synthetic oligonucleotide PU1R_Dbr1Int2_selAMP_R
actttatcaatctcgctccaaaccagtatccacaaagtatcacac

SEQ ID NO: 65 - DNA, 44 bp, Artificial Sequence
  Synthetic oligonucleotide  PU1R_Dbr1_Int2_R2
actttatcaatctcgctccaaacccctgataagctcttccatcc

SEQ ID NO: 66 - DNA, 25 bp, Artificial Sequence
  Synthetic oligonucleotide  sgRNA_Dbr1X2_F
caccgcagttctcacgctcttcatt

SEQ ID NO: 67 - DNA, 25 bp, Artificial Sequence
  Synthetic oligonucleotide  sgRNA_Dbr1X2_R
aaacaatgaagagcgtgagaactgc

SEQ ID NO: 68 - DNA, 117 bp, Artificial Sequence
  Synthetic oligonucleotide  WT BRCA1ex18 (PAM , protospacer underlined; introns gray)
gagtgtttttcattctgcagatgctgagtttgtgtgtgaacggacactgaaatattttctaggaattgcgggaggaaaatgggtagttagctatttctgtaagtataatactatttc

SEQ ID NO: 69 - DNA, 117 bp, Artificial Sequence
  Synthetic oligonucleotide HDRL Random Hexamer BRCA1ex18
gagtgtttttcattctgcagatgcnnnnnntgtgtggatatccacactgaaatattttctaggaattgcgggaggaaaatgggtagttagctatttctgtaagtataatactatttc

SEQ ID NO: 70 - DNA, 117 bp, Artificial Sequence
  Synthetic oligonucleotide HDRL 3% mut 5 SYN BRCA1ex18
gagtgtttttcattctgcagatgctgagtttgtctgcgagagaacactgaaatattttctaggaattgcgggaggaaaatgggtagttagctatttctgtaagtataatactatttc

SEQ ID NO: 71 - DNA, 117 bp, Artificial Sequence
  Synthetic oligonucleotide HDRL 3% mut 5 NONSYN BRCA1ex18
gagtgtttttcattctgcagatgctgagtttgtgtggatatccacactgaaatattttctaggaattgcgggaggaaaatgggtagttagctatttctgtaagtataatactatttc

SEQ ID NO: 72 - DNA, 117 bp, Artificial Sequence
  Synthetic oligonucleotide HDRL 3% mut 3 NONSYN BRCA1ex18
gagtgtttttcattctgcagatgctgagtttgtgtgtgaacggacactgaaatattttctaggaattgcgggaggaaaagatatccttagctatttctgtaagtataatactatttc

SEQ ID NO: 73 - DNA, 124 bp, Artificial Sequence
  Synthetic oligonucleotide  WT DBR1ex2
ccagttctcacgctcttcattgggggaaaccatgaagcctcaaatcatttgcaagagttaccctatggtggctgggtggcaccaaacatttattatttaggtatgtgattgtgtttgtggatac

SEQ ID NO: 74 - DNA, 124 bp, Artificial Sequence; identical to SEQ ID NO: 53
  Synthetic oligonucleotide  DBR1ex2 HDRL

SEQ ID NO: 75 - DNA, 75 bp, Artificial Sequence
  Synthetic oligonucleotide
aaacatgaagcctcaaatcatttgcaagagttaccctatggtggctgggtggcaccaaacatttattatttaggt

SEQ ID NO: 76 - DNA, 75 bp, Artificial Sequence
  Synthetic oligonucleotide
aacaaagaagcctcaaatcatttgcaagagttaccctatggtggctgggtggcaccaaacatttattatttaggt

SEQ ID NO: 77 - DNA, 75 bp, Artificial Sequence
  Synthetic oligonucleotide
aacaacgaagcctcaaatcatttgcaagagttaccctatggtggctgggtggcaccaaacatttattatttaggt

SEQ ID NO: 78 - DNA, 75 bp, Artificial Sequence
  Synthetic oligonucleotide
aacaaggaagcctcaaatcatttgcaagagttaccctatggtggctgggtggcaccaaacatttattatttaggt

SEQ ID NO: 79 - DNA, 75 bp, Artificial Sequence
  Synthetic oligonucleotide
aacaatgaagcctcaaatcatttgcaagagttaccctatggtggctgggtggcaccaaacatttattatttaggt

SEQ ID NO: 80 - DNA, 75 bp, Artificial Sequence
  Synthetic oligonucleotide
aacacagaagcctcaaatcatttgcaagagttaccctatggtggctgggtggcaccaaacatttattatttaggt

SEQ ID NO: 81 - DNA, 75 bp, Artificial Sequence
  Synthetic oligonucleotide
aacaccgaagcctcaaatcatttgcaagagttaccctatggtggctgggtggcaccaaacatttattatttaggt

SEQ ID NO: 82 - DNA, 75 bp, Artificial Sequence
  Synthetic oligonucleotide
aacacggaagcctcaaatcatttgcaagagttaccctatggtggctgggtggcaccaaacatttattatttaggt

SEQ ID NO: 83 - DNA, 75 bp, Artificial Sequence
  Synthetic oligonucleotide
aacactgaagcctcaaatcatttgcaagagttaccctatggtggctgggtggcaccaaacatttattatttaggt

SEQ ID NO: 84 - DNA, 75 bp, Artificial Sequence
  Synthetic oligonucleotide
aacagagaagcctcaaatcatttgcaagagttaccctatggtggctgggtggcaccaaacatttattatttaggt

SEQ ID NO: 85 - DNA, 75 bp, Artificial Sequence
  Synthetic oligonucleotide
aacagcgaagcctcaaatcatttgcaagagttaccctatggtggctgggtggcaccaaacatttattatttaggt

SEQ ID NO: 86 - DNA, 75 bp, Artificial Sequence
  Synthetic oligonucleotide
aacagggaagcctcaaatcatttgcaagagttaccctatggtggctgggtggcaccaaacatttattatttaggt

SEQ ID NO: 87 - DNA, 75 bp, Artificial Sequence
  Synthetic oligonucleotide
aacagtgaagcctcaaatcatttgcaagagttaccctatggtggctgggtggcaccaaacatttattatttaggt

SEQ ID NO: 88 - DNA, 75 bp, Artificial Sequence
  Synthetic oligonucleotide
aacatagaagcctcaaatcatttgcaagagttaccctatggtggctgggtggcaccaaacatttattatttaggt

SEQ ID NO: 89 - DNA, 75 bp, Artificial Sequence
  Synthetic oligonucleotide
aacatcgaagcctcaaatcatttgcaagagttaccctatggtggctgggtggcaccaaacatttattatttaggt

SEQ ID NO: 90 - DNA, 74 bp, Artificial Sequence
  Synthetic oligonucleotide
aacatgaagcctcaaatcatttgcaagagttaccctatggtggctgggtggcaccaaacatttattatttaggt

SEQ ID NO: 91 - DNA, 75 bp, Artificial Sequence
  Synthetic oligonucleotide
aacatggaagcctcaaatcatttgcaagagttaccctatggtggctgggtggcaccaaacatttattatttaggt

SEQ ID NO: 92 - DNA, 75 bp, Artificial Sequence
  Synthetic oligonucleotide
aacattgaagcctcaaatcatttgcaagagttaccctatggtggctgggtggcaccaaacatttattatttaggt

SEQ ID NO: 93 - DNA, 75 bp, Artificial Sequence
  Synthetic oligonucleotide
aaccaagaagcctcaaatcatttgcaagagttaccctatggtggctgggtggcaccaaacatttattatttaggt

SEQ ID NO: 94 - DNA, 75 bp, Artificial Sequence
  Synthetic oligonucleotide
aaccacgaagcctcaaatcatttgcaagagttaccctatggtggctgggtggcaccaaacatttattatttaggt

SEQ ID NO: 95 - DNA, 74 bp, Artificial Sequence
  Synthetic oligonucleotide
aaccagaagcctcaaatcatttgcaagagttaccctatggtggctgggtggcaccaaacatttattatttaggt

SEQ ID NO: 96 - DNA, 75 bp, Artificial Sequence
  Synthetic oligonucleotide
aaccaggaagcctcaaatcatttgcaagagttaccctatggtggctgggtggcaccaaacatttattatttaggt

SEQ ID NO: 97 - DNA, 75 bp, Artificial Sequence
  Synthetic oligonucleotide
aaccataaagcctcaaatcatttgcaagagttaccctatggtggctgggtggcaccaaacatttattatttaggt

SEQ ID NO: 98 - DNA, 75 bp, Artificial Sequence
  Synthetic oligonucleotide
aaccataacgcctcaaatcatttgcaagagttaccctatggtggctgggtggcaccaaacatttattatttaggt

SEQ ID NO: 99 - DNA, 74 bp, Artificial Sequence
  Synthetic oligonucleotide
aaccataagcctcaaatcatttgcaagagttaccctatggtggctgggtggcaccaaacatttattatttaggt

SEQ ID NO: 100 - DNA, 75 bp, Artificial Sequence
  Synthetic oligonucleotide
aaccataaggcctcaaatcatttgcaagagttaccctatggtggctgggtggcaccaaacatttattatttaggt

SEQ ID NO: 101 - DNA, 75 bp, Artificial Sequence
  Synthetic oligonucleotide
aaccataatgcctcaaatcatttgcaagagttaccctatggtggctgggtggcaccaaacatttattatttaggt

SEQ ID NO: 102 - DNA, 75 bp, Artificial Sequence
  Synthetic oligonucleotide
aaccatacagcctcaaatcatttgcaagagttaccctatggtggctgggtggcaccaaacatttattatttaggt

SEQ ID NO: 103 - DNA, 75 bp, Artificial Sequence
  Synthetic oligonucleotide
aaccataccgcctcaaatcatttgcaagagttaccctatggtggctgggtggcaccaaacatttattatttaggt

SEQ ID NO: 104 - DNA, 75 bp, Artificial Sequence
  Synthetic oligonucleotide
aaccatacggcctcaaatcatttgcaagagttaccctatggtggctgggtggcaccaaacatttattatttaggt

SEQ ID NO: 105 - DNA, 75 bp, Artificial Sequence
  Synthetic oligonucleotide
aaccatactgcctcaaatcatttgcaagagttaccctatggtggctgggtggcaccaaacatttattatttaggt

SEQ ID NO: 106 - DNA, 75 bp, Artificial Sequence
  Synthetic oligonucleotide
aaccatagagcctcaaatcatttgcaagagttaccctatggtggctgggtggcaccaaacatttattatttaggt

SEQ ID NO: 107 - DNA, 75 bp, Artificial Sequence
  Synthetic oligonucleotide
aaccatagcgcctcaaatcatttgcaagagttaccctatggtggctgggtggcaccaaacatttattatttaggt

SEQ ID NO: 108 - DNA, 75 bp, Artificial Sequence
  Synthetic oligonucleotide
aaccatagggcctcaaatcatttgcaagagttaccctatggtggctgggtggcaccaaacatttattatttaggt

SEQ ID NO: 109 - DNA, 75 bp, Artificial Sequence
  Synthetic oligonucleotide
aaccatagtgcctcaaatcatttgcaagagttaccctatggtggctgggtggcaccaaacatttattatttaggt

SEQ ID NO: 110 - DNA, 75 bp, Artificial Sequence
  Synthetic oligonucleotide
aaccatatagcctcaaatcatttgcaagagttaccctatggtggctgggtggcaccaaacatttattatttaggt

SEQ ID NO: 111 - DNA, 75 bp, Artificial Sequence
  Synthetic oligonucleotide
aaccatatcgcctcaaatcatttgcaagagttaccctatggtggctgggtggcaccaaacatttattatttaggt

SEQ ID NO: 112 - DNA, 75 bp, Artificial Sequence
  Synthetic oligonucleotide
aaccatatggcctcaaatcatttgcaagagttaccctatggtggctgggtggcaccaaacatttattatttaggt

SEQ ID NO: 113 - DNA, 75 bp, Artificial Sequence
  Synthetic oligonucleotide
aaccatattgcctcaaatcatttgcaagagttaccctatggtggctgggtggcaccaaacatttattatttaggt

SEQ ID NO: 114 - DNA, 75 bp, Artificial Sequence
  Synthetic oligonucleotide
aaccatcaagcctcaaatcatttgcaagagttaccctatggtggctgggtggcaccaaacatttattatttaggt

SEQ ID NO: 115 - DNA, 75 bp, Artificial Sequence
  Synthetic oligonucleotide
aaccatcacgcctcaaatcatttgcaagagttaccctatggtggctgggtggcaccaaacatttattatttaggt

SEQ ID NO: 116 - DNA, 75 bp, Artificial Sequence
  Synthetic oligonucleotide
aaccatcaggcctcaaatcatttgcaagagttaccctatggtggctgggtggcaccaaacatttattatttaggt

SEQ ID NO: 117 - DNA, 75 bp, Artificial Sequence
  Synthetic oligonucleotide
aaccatcatgcctcaaatcatttgcaagagttaccctatggtggctgggtggcaccaaacatttattatttaggt

SEQ ID NO: 118 - DNA, 75 bp, Artificial Sequence
  Synthetic oligonucleotide
aaccatccagcctcaaatcatttgcaagagttaccctatggtggctgggtggcaccaaacatttattatttaggt

SEQ ID NO: 119 - DNA, 75 bp, Artificial Sequence
  Synthetic oligonucleotide
aaccatcccgcctcaaatcatttgcaagagttaccctatggtggctgggtggcaccaaacatttattatttaggt

SEQ ID NO: 120 - DNA, 75 bp, Artificial Sequence
  Synthetic oligonucleotide
aaccatccggcctcaaatcatttgcaagagttaccctatggtggctgggtggcaccaaacatttattatttaggt

SEQ ID NO: 121 - DNA, 75 bp, Artificial Sequence
  Synthetic oligonucleotide
aaccatcctgcctcaaatcatttgcaagagttaccctatggtggctgggtggcaccaaacatttattatttaggt

SEQ ID NO: 122 - DNA, 75 bp, Artificial Sequence
  Synthetic oligonucleotide
aaccatcgagcctcaaatcatttgcaagagttaccctatggtggctgggtggcaccaaacatttattatttaggt

SEQ ID NO: 123 - DNA, 75 bp, Artificial Sequence
  Synthetic oligonucleotide
aaccatcgcgcctcaaatcatttgcaagagttaccctatggtggctgggtggcaccaaacatttattatttaggt

SEQ ID NO: 124 - DNA, 75 bp, Artificial Sequence
  Synthetic oligonucleotide
aaccatcgggcctcaaatcatttgcaagagttaccctatggtggctgggtggcaccaaacatttattatttaggt

SEQ ID NO: 125 - DNA, 75 bp, Artificial Sequence
  Synthetic oligonucleotide
aaccatcgtgcctcaaatcatttgcaagagttaccctatggtggctgggtggcaccaaacatttattatttaggt

SEQ ID NO: 126 - DNA, 75 bp, Artificial Sequence
  Synthetic oligonucleotide
aaccatctagcctcaaatcatttgcaagagttaccctatggtggctgggtggcaccaaacatttattatttaggt

SEQ ID NO: 127 - DNA, 75 bp, Artificial Sequence
  Synthetic oligonucleotide
aaccatctcgcctcaaatcatttgcaagagttaccctatggtggctgggtggcaccaaacatttattatttaggt

SEQ ID NO: 128 - DNA, 75 bp, Artificial Sequence
  Synthetic oligonucleotide
aaccatctggcctcaaatcatttgcaagagttaccctatggtggctgggtggcaccaaacatttattatttaggt

SEQ ID NO: 129 - DNA, 75 bp, Artificial Sequence
  Synthetic oligonucleotide
aaccatcttgcctcaaatcatttgcaagagttaccctatggtggctgggtggcaccaaacatttattatttaggt

SEQ ID NO: 130 - DNA, 75 bp, Artificial Sequence
  Synthetic oligonucleotide
aaccatgaaacctcaaatcatttgcaagagttaccctatggtggctgggtggcaccaaacatttattatttaggt

SEQ ID NO: 131 - DNA, 75 bp, Artificial Sequence
  Synthetic oligonucleotide
aaccatgaaccctcaaatcatttgcaagagttaccctatggtggctgggtggcaccaaacatttattatttaggt

SEQ ID NO: 132 - DNA, 74 bp, Artificial Sequence
  Synthetic oligonucleotide
aaccatgaacctcaaatcatttgcaagagttaccctatggtggctgggtggcaccaaacatttattatttaggt

SEQ ID NO: 133 - DNA, 75 bp, Artificial Sequence
  Synthetic oligonucleotide
aaccatgaagactcaaatcatttgcaagagttaccctatggtggctgggtggcaccaaacatttattatttaggt

SEQ ID NO: 134 - DNA, 75 bp, Artificial Sequence
  Synthetic oligonucleotide
aaccatgaagcatcaaatcatttgcaagagttaccctatggtggctgggtggcaccaaacatttattatttaggt

SEQ ID NO: 135 - DNA, 75 bp, Artificial Sequence
  Synthetic oligonucleotide
aaccatgaagccacaaatcatttgcaagagttaccctatggtggctgggtggcaccaaacatttattatttaggt

SEQ ID NO: 136 - DNA, 74 bp, Artificial Sequence
  Synthetic oligonucleotide
aaccatgaagcccaaatcatttgcaagagttaccctatggtggctgggtggcaccaaacatttattatttaggt

SEQ ID NO: 137 - DNA, 75 bp, Artificial Sequence
  Synthetic oligonucleotide
aaccatgaagccccaaatcatttgcaagagttaccctatggtggctgggtggcaccaaacatttattatttaggt

SEQ ID NO: 138 - DNA, 75 bp, Artificial Sequence
  Synthetic oligonucleotide
aaccatgaagccgcaaatcatttgcaagagttaccctatggtggctgggtggcaccaaacatttattatttaggt

SEQ ID NO: 139 - DNA, 75 bp, Artificial Sequence
  Synthetic oligonucleotide
aaccatgaagcctaaaatcatttgcaagagttaccctatggtggctgggtggcaccaaacatttattatttaggt

SEQ ID NO: 140 - DNA, 74 bp, Artificial Sequence
  Synthetic oligonucleotide
aaccatgaagcctaaatcatttgcaagagttaccctatggtggctgggtggcaccaaacatttattatttaggt

SEQ ID NO: 141 - DNA, 75 bp, Artificial Sequence
  Synthetic oligonucleotide
aaccatgaagcctcaaaacatttgcaagagttaccctatggtggctgggtggcaccaaacatttattatttaggt

SEQ ID NO: 142 - DNA, 74 bp, Artificial Sequence
  Synthetic oligonucleotide
aaccatgaagcctcaaacatttgcaagagttaccctatggtggctgggtggcaccaaacatttattatttaggt

SEQ ID NO: 143 - DNA, 75 bp, Artificial Sequence
  Synthetic oligonucleotide
aaccatgaagcctcaaaccatttgcaagagttaccctatggtggctgggtggcaccaaacatttattatttaggt

SEQ ID NO: 144 - DNA, 75 bp, Artificial Sequence
  Synthetic oligonucleotide
aaccatgaagcctcaaagcatttgcaagagttaccctatggtggctgggtggcaccaaacatttattatttaggt

SEQ ID NO: 145 - DNA, 75 bp, Artificial Sequence
  Synthetic oligonucleotide
aaccatgaagcctcaaataatttgcaagagttaccctatggtggctgggtggcaccaaacatttattatttaggt

SEQ ID NO: 146 - DNA, 74 bp, Artificial Sequence
  Synthetic oligonucleotide
aaccatgaagcctcaaatatttgcaagagttaccctatggtggctgggtggcaccaaacatttattatttaggt

SEQ ID NO: 147 - DNA, 75 bp, Artificial Sequence
  Synthetic oligonucleotide
aaccatgaagcctcaaatcaattgcaagagttaccctatggtggctgggtggcaccaaacatttattatttaggt

SEQ ID NO: 148 - DNA, 75 bp, Artificial Sequence
  Synthetic oligonucleotide
aaccatgaagcctcaaatcacttgcaagagttaccctatggtggctgggtggcaccaaacatttattatttaggt

SEQ ID NO: 149 - DNA, 75 bp, Artificial Sequence
  Synthetic oligonucleotide
aaccatgaagcctcaaatcagttgcaagagttaccctatggtggctgggtggcaccaaacatttattatttaggt

SEQ ID NO: 150 - DNA, 75 bp, Artificial Sequence
  Synthetic oligonucleotide
aaccatgaagcctcaaatcatatgcaagagttaccctatggtggctgggtggcaccaaacatttattatttaggt

SEQ ID NO: 151 - DNA, 75 bp, Artificial Sequence
  Synthetic oligonucleotide
aaccatgaagcctcaaatcatctgcaagagttaccctatggtggctgggtggcaccaaacatttattatttaggt

SEQ ID NO: 152 - DNA, 75 bp, Artificial Sequence
  Synthetic oligonucleotide
aaccatgaagcctcaaatcatgtgcaagagttaccctatggtggctgggtggcaccaaacatttattatttaggt

SEQ ID NO: 153 - DNA, 75 bp, Artificial Sequence
  Synthetic oligonucleotide
aaccatgaagcctcaaatcattagcaagagttaccctatggtggctgggtggcaccaaacatttattatttaggt

SEQ ID NO: 154 - DNA, 75 bp, Artificial Sequence
  Synthetic oligonucleotide
aaccatgaagcctcaaatcattcgcaagagttaccctatggtggctgggtggcaccaaacatttattatttaggt

SEQ ID NO: 155 - DNA, 74 bp, Artificial Sequence
  Synthetic oligonucleotide
aaccatgaagcctcaaatcattgcaagagttaccctatggtggctgggtggcaccaaacatttattatttaggt

SEQ ID NO: 156 - DNA, 75 bp, Artificial Sequence
  Synthetic oligonucleotide
aaccatgaagcctcaaatcattggcaagagttaccctatggtggctgggtggcaccaaacatttattatttaggt

SEQ ID NO: 157 - DNA, 75 bp, Artificial Sequence
  Synthetic oligonucleotide
aaccatgaagcctcaaatcatttacaagagttaccctatggtggctgggtggcaccaaacatttattatttaggt

SEQ ID NO: 158 - DNA, 74 bp, Artificial Sequence
  Synthetic oligonucleotide
aaccatgaagcctcaaatcatttcaagagttaccctatggtggctgggtggcaccaaacatttattatttaggt

SEQ ID NO: 159 - DNA, 75 bp, Artificial Sequence
  Synthetic oligonucleotide
aaccatgaagcctcaaatcatttccaagagttaccctatggtggctgggtggcaccaaacatttattatttaggt

SEQ ID NO: 160 - DNA, 75 bp, Artificial Sequence
  Synthetic oligonucleotide
aaccatgaagcctcaaatcatttgaaagagttaccctatggtggctgggtggcaccaaacatttattatttaggt

SEQ ID NO: 161 - DNA, 74 bp, Artificial Sequence
  Synthetic oligonucleotide
aaccatgaagcctcaaatcatttgaagagttaccctatggtggctgggtggcaccaaacatttattatttaggt

SEQ ID NO: 162 - DNA, 75 bp, Artificial Sequence
  Synthetic oligonucleotide
aaccatgaagcctcaaatcatttgcaaaagttaccctatggtggctgggtggcaccaaacatttattatttaggt

SEQ ID NO: 163 - DNA, 74 bp, Artificial Sequence
  Synthetic oligonucleotide
aaccatgaagcctcaaatcatttgcaaagttaccctatggtggctgggtggcaccaaacatttattatttaggt

SEQ ID NO: 164 - DNA, 75 bp, Artificial Sequence
  Synthetic oligonucleotide
aaccatgaagcctcaaatcatttgcaacagttaccctatggtggctgggtggcaccaaacatttattatttaggt

SEQ ID NO: 165 - DNA, 75 bp, Artificial Sequence
  Synthetic oligonucleotide
aaccatgaagcctcaaatcatttgcaagaattaccctatggtggctgggtggcaccaaacatttattatttaggt

SEQ ID NO: 166 - DNA, 75 bp, Artificial Sequence
  Synthetic oligonucleotide
aaccatgaagcctcaaatcatttgcaagacttaccctatggtggctgggtggcaccaaacatttattatttaggt

SEQ ID NO: 167 - DNA, 75 bp, Artificial Sequence
  Synthetic oligonucleotide
aaccatgaagcctcaaatcatttgcaagagataccctatggtggctgggtggcaccaaacatttattatttaggt

SEQ ID NO: 168 - DNA, 75 bp, Artificial Sequence
  Synthetic oligonucleotide
aaccatgaagcctcaaatcatttgcaagagctaccctatggtggctgggtggcaccaaacatttattatttaggt

SEQ ID NO: 169 - DNA, 75 bp, Artificial Sequence
  Synthetic oligonucleotide
aaccatgaagcctcaaatcatttgcaagaggtaccctatggtggctgggtggcaccaaacatttattatttaggt

SEQ ID NO: 170 - DNA, 75 bp, Artificial Sequence
  Synthetic oligonucleotide
aaccatgaagcctcaaatcatttgcaagagtaaccctatggtggctgggtggcaccaaacatttattatttaggt

SEQ ID NO: 171 - DNA, 74 bp, Artificial Sequence
  Synthetic oligonucleotide
aaccatgaagcctcaaatcatttgcaagagtaccctatggtggctgggtggcaccaaacatttattatttaggt

SEQ ID NO: 172 - DNA, 75 bp, Artificial Sequence
  Synthetic oligonucleotide
aaccatgaagcctcaaatcatttgcaagagtcaccctatggtggctgggtggcaccaaacatttattatttaggt

SEQ ID NO: 173 - DNA, 75 bp, Artificial Sequence
  Synthetic oligonucleotide
aaccatgaagcctcaaatcatttgcaagagtgaccctatggtggctgggtggcaccaaacatttattatttaggt

SEQ ID NO: 174 - DNA, 75 bp, Artificial Sequence
  Synthetic oligonucleotide
aaccatgaagcctcaaatcatttgcaagagttaacctatggtggctgggtggcaccaaacatttattatttaggt

SEQ ID NO: 175 - DNA, 75 bp, Artificial Sequence
  Synthetic oligonucleotide
aaccatgaagcctcaaatcatttgcaagagttacactatggtggctgggtggcaccaaacatttattatttaggt

SEQ ID NO: 176 - DNA, 75 bp, Artificial Sequence
  Synthetic oligonucleotide
aaccatgaagcctcaaatcatttgcaagagttaccatatggtggctgggtggcaccaaacatttattatttaggt

SEQ ID NO: 177 - DNA, 75 bp, Artificial Sequence
  Synthetic oligonucleotide
aaccatgaagcctcaaatcatttgcaagagttacccaatggtggctgggtggcaccaaacatttattatttaggt

SEQ ID NO: 178 - DNA, 74 bp, Artificial Sequence
  Synthetic oligonucleotide
aaccatgaagcctcaaatcatttgcaagagttacccatggtggctgggtggcaccaaacatttattatttaggt

SEQ ID NO: 179 - DNA, 75 bp, Artificial Sequence
  Synthetic oligonucleotide
aaccatgaagcctcaaatcatttgcaagagttaccccatggtggctgggtggcaccaaacatttattatttaggt

SEQ ID NO: 180 - DNA, 75 bp, Artificial Sequence
  Synthetic oligonucleotide
aaccatgaagcctcaaatcatttgcaagagttacccgatggtggctgggtggcaccaaacatttattatttaggt

SEQ ID NO: 181 - DNA, 75 bp, Artificial Sequence
  Synthetic oligonucleotide
aaccatgaagcctcaaatcatttgcaagagttaccctaaggtggctgggtggcaccaaacatttattatttaggt

SEQ ID NO: 182 - DNA, 75 bp, Artificial Sequence
  Synthetic oligonucleotide
aaccatgaagcctcaaatcatttgcaagagttaccctacggtggctgggtggcaccaaacatttattatttaggt

SEQ ID NO: 183 - DNA, 75 bp, Artificial Sequence
  Synthetic oligonucleotide
aaccatgaagcctcaaatcatttgcaagagttaccctagggtggctgggtggcaccaaacatttattatttaggt

SEQ ID NO: 184 - DNA, 74 bp, Artificial Sequence
  Synthetic oligonucleotide
aaccatgaagcctcaaatcatttgcaagagttaccctaggtggctgggtggcaccaaacatttattatttaggt

SEQ ID NO: 185 - DNA, 75 bp, Artificial Sequence
  Synthetic oligonucleotide
aaccatgaagcctcaaatcatttgcaagagttaccctatagtggctgggtggcaccaaacatttattatttaggt

SEQ ID NO: 186 - DNA, 75 bp, Artificial Sequence
  Synthetic oligonucleotide
aaccatgaagcctcaaatcatttgcaagagttaccctatcgtggctgggtggcaccaaacatttattatttaggt

SEQ ID NO: 187 - DNA, 75 bp, Artificial Sequence
  Synthetic oligonucleotide
aaccatgaagcctcaaatcatttgcaagagttaccctatgatggctgggtggcaccaaacatttattatttaggt

SEQ ID NO: 188 - DNA, 75 bp, Artificial Sequence
  Synthetic oligonucleotide
aaccatgaagcctcaaatcatttgcaagagttaccctatgctggctgggtggcaccaaacatttattatttaggt

SEQ ID NO: 189 - DNA, 75 bp, Artificial Sequence
  Synthetic oligonucleotide
aaccatgaagcctcaaatcatttgcaagagttaccctatggaggctgggtggcaccaaacatttattatttaggt

SEQ ID NO: 190 - DNA, 75 bp, Artificial Sequence
  Synthetic oligonucleotide
aaccatgaagcctcaaatcatttgcaagagttaccctatggcggctgggtggcaccaaacatttattatttaggt

SEQ ID NO: 191 - DNA, 74 bp, Artificial Sequence
  Synthetic oligonucleotide
aaccatgaagcctcaaatcatttgcaagagttaccctatggggctgggtggcaccaaacatttattatttaggt

SEQ ID NO: 192 - DNA, 75 bp, Artificial Sequence
  Synthetic oligonucleotide
aaccatgaagcctcaaatcatttgcaagagttaccctatgggggctgggtggcaccaaacatttattatttaggt

SEQ ID NO: 193 - DNA, 75 bp, Artificial Sequence
  Synthetic oligonucleotide
aaccatgaagcctcaaatcatttgcaagagttaccctatggtagctgggtggcaccaaacatttattatttaggt

SEQ ID NO: 194 - DNA, 75 bp, Artificial Sequence
  Synthetic oligonucleotide
aaccatgaagcctcaaatcatttgcaagagttaccctatggtcgctgggtggcaccaaacatttattatttaggt

SEQ ID NO: 195 - DNA, 75 bp, Artificial Sequence
  Synthetic oligonucleotide
aaccatgaagcctcaaatcatttgcaagagttaccctatggtgactgggtggcaccaaacatttattatttaggt

SEQ ID NO: 196 - DNA, 75 bp, Artificial Sequence
  Synthetic oligonucleotide
aaccatgaagcctcaaatcatttgcaagagttaccctatggtgcctgggtggcaccaaacatttattatttaggt

SEQ ID NO: 197 - DNA, 74 bp, Artificial Sequence
  Synthetic oligonucleotide
aaccatgaagcctcaaatcatttgcaagagttaccctatggtgctgggtggcaccaaacatttattatttaggt

SEQ ID NO: 198 - DNA, 75 bp, Artificial Sequence
  Synthetic oligonucleotide
aaccatgaagcctcaaatcatttgcaagagttaccctatggtggatgggtggcaccaaacatttattatttaggt

SEQ ID NO: 199 - DNA, 75 bp, Artificial Sequence
  Synthetic oligonucleotide
aaccatgaagcctcaaatcatttgcaagagttaccctatggtggcagggtggcaccaaacatttattatttaggt

SEQ ID NO: 200 - DNA, 75 bp, Artificial Sequence
  Synthetic oligonucleotide
aaccatgaagcctcaaatcatttgcaagagttaccctatggtggccgggtggcaccaaacatttattatttaggt

SEQ ID NO: 201 - DNA, 75 bp, Artificial Sequence
  Synthetic oligonucleotide
aaccatgaagcctcaaatcatttgcaagagttaccctatggtggcggggtggcaccaaacatttattatttaggt

SEQ ID NO: 202 - DNA, 74 bp, Artificial Sequence
  Synthetic oligonucleotide
aaccatgaagcctcaaatcatttgcaagagttaccctatggtggcgggtggcaccaaacatttattatttaggt

SEQ ID NO: 203 - DNA, 75 bp, Artificial Sequence
  Synthetic oligonucleotide
aaccatgaagcctcaaatcatttgcaagagttaccctatggtggctaggtggcaccaaacatttattatttaggt

SEQ ID NO: 204 - DNA, 75 bp, Artificial Sequence
  Synthetic oligonucleotide
aaccatgaagcctcaaatcatttgcaagagttaccctatggtggctcggtggcaccaaacatttattatttaggt

SEQ ID NO: 205 - DNA, 75 bp, Artificial Sequence
  Synthetic oligonucleotide
aaccatgaagcctcaaatcatttgcaagagttaccctatggtggctgagtggcaccaaacatttattatttaggt

SEQ ID NO: 206 - DNA, 75 bp, Artificial Sequence
  Synthetic oligonucleotide
aaccatgaagcctcaaatcatttgcaagagttaccctatggtggctgcgtggcaccaaacatttattatttaggt

SEQ ID NO: 207 - DNA, 75 bp, Artificial Sequence
  Synthetic oligonucleotide
aaccatgaagcctcaaatcatttgcaagagttaccctatggtggctggatggcaccaaacatttattatttaggt

SEQ ID NO: 208 - DNA, 75 bp, Artificial Sequence
  Synthetic oligonucleotide
aaccatgaagcctcaaatcatttgcaagagttaccctatggtggctggctggcaccaaacatttattatttaggt

SEQ ID NO: 209 - DNA, 75 bp, Artificial Sequence
  Synthetic oligonucleotide
aaccatgaagcctcaaatcatttgcaagagttaccctatggtggctgggaggcaccaaacatttattatttaggt

SEQ ID NO: 210 - DNA, 75 bp, Artificial Sequence
  Synthetic oligonucleotide
aaccatgaagcctcaaatcatttgcaagagttaccctatggtggctgggcggcaccaaacatttattatttaggt

SEQ ID NO: 211 - DNA, 74 bp, Artificial Sequence
  Synthetic oligonucleotide
aaccatgaagcctcaaatcatttgcaagagttaccctatggtggctgggggcaccaaacatttattatttaggt

SEQ ID NO: 212 - DNA, 75 bp, Artificial Sequence
  Synthetic oligonucleotide
aaccatgaagcctcaaatcatttgcaagagttaccctatggtggctggggggcaccaaacatttattatttaggt

SEQ ID NO: 213 - DNA, 75 bp, Artificial Sequence
  Synthetic oligonucleotide
aaccatgaagcctcaaatcatttgcaagagttaccctatggtggctgggtagcaccaaacatttattatttaggt

SEQ ID NO: 214 - DNA, 75 bp, Artificial Sequence
  Synthetic oligonucleotide
aaccatgaagcctcaaatcatttgcaagagttaccctatggtggctgggtcgcaccaaacatttattatttaggt

SEQ ID NO: 215 - DNA, 75 bp, Artificial Sequence
  Synthetic oligonucleotide
aaccatgaagcctcaaatcatttgcaagagttaccctatggtggctgggtgacaccaaacatttattatttaggt

SEQ ID NO: 216 - DNA, 74 bp, Artificial Sequence
  Synthetic oligonucleotide
aaccatgaagcctcaaatcatttgcaagagttaccctatggtggctgggtgcaccaaacatttattatttaggt

SEQ ID NO: 217 - DNA, 75 bp, Artificial Sequence
  Synthetic oligonucleotide
aaccatgaagcctcaaatcatttgcaagagttaccctatggtggctgggtgccaccaaacatttattatttaggt

SEQ ID NO: 218 - DNA, 75 bp, Artificial Sequence
  Synthetic oligonucleotide
aaccatgaagcctcaaatcatttgcaagagttaccctatggtggctgggtggaaccaaacatttattatttaggt

SEQ ID NO: 219 - DNA, 74 bp, Artificial Sequence
  Synthetic oligonucleotide
aaccatgaagcctcaaatcatttgcaagagttaccctatggtggctgggtggaccaaacatttattatttaggt

SEQ ID NO: 220 - DNA, 75 bp, Artificial Sequence
  Synthetic oligonucleotide
aaccatgaagcctcaaatcatttgcaagagttaccctatggtggctgggtggcaacaaacatttattatttaggt

SEQ ID NO: 221 - DNA, 75 bp, Artificial Sequence
  Synthetic oligonucleotide
aaccatgaagcctcaaatcatttgcaagagttaccctatggtggctgggtggcacaaaacatttattatttaggt

SEQ ID NO: 222 - DNA, 74 bp, Artificial Sequence
  Synthetic oligonucleotide
aaccatgaagcctcaaatcatttgcaagagttaccctatggtggctgggtggcacaaacatttattatttaggt

SEQ ID NO: 223 - DNA, 75 bp, Artificial Sequence
  Synthetic oligonucleotide
aaccatgaagcctcaaatcatttgcaagagttaccctatggtggctgggtggcaccaaaaatttattatttaggt

SEQ ID NO: 224 - DNA, 74 bp, Artificial Sequence
  Synthetic oligonucleotide
aaccatgaagcctcaaatcatttgcaagagttaccctatggtggctgggtggcaccaaaatttattatttaggt

SEQ ID NO: 225 - DNA, 75 bp, Artificial Sequence
  Synthetic oligonucleotide
aaccatgaagcctcaaatcatttgcaagagttaccctatggtggctgggtggcaccaaacaattattatttaggt

SEQ ID NO: 226 - DNA, 75 bp, Artificial Sequence
  Synthetic oligonucleotide
aaccatgaagcctcaaatcatttgcaagagttaccctatggtggctgggtggcaccaaacacttattatttaggt

SEQ ID NO: 227 - DNA, 75 bp, Artificial Sequence
  Synthetic oligonucleotide
aaccatgaagcctcaaatcatttgcaagagttaccctatggtggctgggtggcaccaaacagttattatttaggt

SEQ ID NO: 228 - DNA, 75 bp, Artificial Sequence
  Synthetic oligonucleotide
aaccatgaagcctcaaatcatttgcaagagttaccctatggtggctgggtggcaccaaacatatattatttaggt

SEQ ID NO: 229 - DNA, 75 bp, Artificial Sequence
  Synthetic oligonucleotide
aaccatgaagcctcaaatcatttgcaagagttaccctatggtggctgggtggcaccaaacatctattatttaggt

SEQ ID NO: 230 - DNA, 75 bp, Artificial Sequence
  Synthetic oligonucleotide
aaccatgaagcctcaaatcatttgcaagagttaccctatggtggctgggtggcaccaaacatgtattatttaggt

SEQ ID NO: 231 - DNA, 75 bp, Artificial Sequence
  Synthetic oligonucleotide
aaccatgaagcctcaaatcatttgcaagagttaccctatggtggctgggtggcaccaaacattaattatttaggt

SEQ ID NO: 232 - DNA, 74 bp, Artificial Sequence
  Synthetic oligonucleotide
aaccatgaagcctcaaatcatttgcaagagttaccctatggtggctgggtggcaccaaacattattatttaggt

SEQ ID NO: 233 - DNA, 75 bp, Artificial Sequence
  Synthetic oligonucleotide
aaccatgaagcctcaaatcatttgcaagagttaccctatggtggctgggtggcaccaaacattcattatttaggt

SEQ ID NO: 234 - DNA, 75 bp, Artificial Sequence
  Synthetic oligonucleotide
aaccatgaagcctcaaatcatttgcaagagttaccctatggtggctgggtggcaccaaacattgattatttaggt

SEQ ID NO: 235 - DNA, 75 bp, Artificial Sequence
  Synthetic oligonucleotide
aaccatgaagcctcaaatcatttgcaagagttaccctatggtggctgggtggcaccaaacatttaatatttaggt

SEQ ID NO: 236 - DNA, 75 bp, Artificial Sequence
  Synthetic oligonucleotide
aaccatgaagcctcaaatcatttgcaagagttaccctatggtggctgggtggcaccaaacatttactatttaggt

SEQ ID NO: 237 - DNA, 75 bp, Artificial Sequence
  Synthetic oligonucleotide
aaccatgaagcctcaaatcatttgcaagagttaccctatggtggctgggtggcaccaaacatttagtatttaggt

SEQ ID NO: 238 - DNA, 75 bp, Artificial Sequence
  Synthetic oligonucleotide
aaccatgaagcctcaaatcatttgcaagagttaccctatggtggctgggtggcaccaaacatttataatttaggt

SEQ ID NO: 239 - DNA, 74 bp, Artificial Sequence
  Synthetic oligonucleotide
aaccatgaagcctcaaatcatttgcaagagttaccctatggtggctgggtggcaccaaacatttatatttaggt

SEQ ID NO: 240 - DNA, 75 bp, Artificial Sequence
  Synthetic oligonucleotide
aaccatgaagcctcaaatcatttgcaagagttaccctatggtggctgggtggcaccaaacatttatcatttaggt

SEQ ID NO: 241 - DNA, 75 bp, Artificial Sequence
  Synthetic oligonucleotide
aaccatgaagcctcaaatcatttgcaagagttaccctatggtggctgggtggcaccaaacatttatgatttaggt

SEQ ID NO: 242 - DNA, 75 bp, Artificial Sequence
  Synthetic oligonucleotide
aaccatgaagcctcaaatcatttgcaagagttaccctatggtggctgggtggcaccaaacatttattaattaggt

SEQ ID NO: 243 - DNA, 75 bp, Artificial Sequence
  Synthetic oligonucleotide
aaccatgaagcctcaaatcatttgcaagagttaccctatggtggctgggtggcaccaaacatttattacttaggt

SEQ ID NO: 244 - DNA, 75 bp, Artificial Sequence
  Synthetic oligonucleotide
aaccatgaagcctcaaatcatttgcaagagttaccctatggtggctgggtggcaccaaacatttattagttaggt

SEQ ID NO: 245 - DNA, 75 bp, Artificial Sequence
  Synthetic oligonucleotide
aaccatgaagcctcaaatcatttgcaagagttaccctatggtggctgggtggcaccaaacatttattatataggt

SEQ ID NO: 246 - DNA, 75 bp, Artificial Sequence
  Synthetic oligonucleotide
aaccatgaagcctcaaatcatttgcaagagttaccctatggtggctgggtggcaccaaacatttattatctaggt

SEQ ID NO: 247 - DNA, 75 bp, Artificial Sequence
  Synthetic oligonucleotide
aaccatgaagcctcaaatcatttgcaagagttaccctatggtggctgggtggcaccaaacatttattatgtaggt

SEQ ID NO: 248 - DNA, 75 bp, Artificial Sequence
  Synthetic oligonucleotide
aaccatgaagcctcaaatcatttgcaagagttaccctatggtggctgggtggcaccaaacatttattattaaggt

SEQ ID NO: 249 - DNA, 74 bp, Artificial Sequence
  Synthetic oligonucleotide
aaccatgaagcctcaaatcatttgcaagagttaccctatggtggctgggtggcaccaaacatttattattaggt

SEQ ID NO: 250 - DNA, 75 bp, Artificial Sequence
  Synthetic oligonucleotide
aaccatgaagcctcaaatcatttgcaagagttaccctatggtggctgggtggcaccaaacatttattattcaggt

SEQ ID NO: 251 - DNA, 75 bp, Artificial Sequence
  Synthetic oligonucleotide
aaccatgaagcctcaaatcatttgcaagagttaccctatggtggctgggtggcaccaaacatttattattgaggt

SEQ ID NO: 252 - DNA, 75 bp, Artificial Sequence
  Synthetic oligonucleotide
aaccatgaagcctcaaatcatttgcaagagttaccctatggtggctgggtggcaccaaacatttattatttaagt

SEQ ID NO: 253 - DNA, 75 bp, Artificial Sequence
  Synthetic oligonucleotide
aaccatgaagcctcaaatcatttgcaagagttaccctatggtggctgggtggcaccaaacatttattatttacgt

SEQ ID NO: 254 - DNA, 75 bp, Artificial Sequence
  Synthetic oligonucleotide
aaccatgaagcctcaaatcatttgcaagagttaccctatggtggctgggtggcaccaaacatttattatttagat

SEQ ID NO: 255 - DNA, 75 bp, Artificial Sequence
  Synthetic oligonucleotide
aaccatgaagcctcaaatcatttgcaagagttaccctatggtggctgggtggcaccaaacatttattatttagct

SEQ ID NO: 256 - DNA, 74 bp, Artificial Sequence
  Synthetic oligonucleotide
aaccatgaagcctcaaatcatttgcaagagttaccctatggtggctgggtggcaccaaacatttattatttagg

SEQ ID NO: 257 - DNA, 75 bp, Artificial Sequence
  Synthetic oligonucleotide
aaccatgaagcctcaaatcatttgcaagagttaccctatggtggctgggtggcaccaaacatttattatttagga

SEQ ID NO: 258 - DNA, 75 bp, Artificial Sequence
  Synthetic oligonucleotide
aaccatgaagcctcaaatcatttgcaagagttaccctatggtggctgggtggcaccaaacatttattatttaggc

SEQ ID NO: 259 - DNA, 75 bp, Artificial Sequence
  Synthetic oligonucleotide
aaccatgaagcctcaaatcatttgcaagagttaccctatggtggctgggtggcaccaaacatttattatttaggg

SEQ ID NO: 260 - DNA, 75 bp, Artificial Sequence
  Synthetic oligonucleotide
aaccatgaagcctcaaatcatttgcaagagttaccctatggtggctgggtggcaccaaacatttattatttaggt

SEQ ID NO: 261 - DNA, 74 bp, Artificial Sequence
  Synthetic oligonucleotide
aaccatgaagcctcaaatcatttgcaagagttaccctatggtggctgggtggcaccaaacatttattatttagt

SEQ ID NO: 262 - DNA, 75 bp, Artificial Sequence
  Synthetic oligonucleotide
aaccatgaagcctcaaatcatttgcaagagttaccctatggtggctgggtggcaccaaacatttattatttagtt

SEQ ID NO: 263 - DNA, 75 bp, Artificial Sequence
  Synthetic oligonucleotide
aaccatgaagcctcaaatcatttgcaagagttaccctatggtggctgggtggcaccaaacatttattatttatgt

SEQ ID NO: 264 - DNA, 75 bp, Artificial Sequence
  Synthetic oligonucleotide
aaccatgaagcctcaaatcatttgcaagagttaccctatggtggctgggtggcaccaaacatttattatttcggt

SEQ ID NO: 265 - DNA, 75 bp, Artificial Sequence
  Synthetic oligonucleotide
aaccatgaagcctcaaatcatttgcaagagttaccctatggtggctgggtggcaccaaacatttattatttgggt

SEQ ID NO: 266 - DNA, 74 bp, Artificial Sequence
  Synthetic oligonucleotide
aaccatgaagcctcaaatcatttgcaagagttaccctatggtggctgggtggcaccaaacatttattatttggt

SEQ ID NO: 267 - DNA, 75 bp, Artificial Sequence
  Synthetic oligonucleotide
aaccatgaagcctcaaatcatttgcaagagttaccctatggtggctgggtggcaccaaacatttattattttggt

SEQ ID NO: 268 - DNA, 75 bp, Artificial Sequence
  Synthetic oligonucleotide
aaccatgaagcctcaaatcatttgcaagagttaccctatggtggctgggtggcaccaaacatttattctttaggt

SEQ ID NO: 269 - DNA, 75 bp, Artificial Sequence
  Synthetic oligonucleotide
aaccatgaagcctcaaatcatttgcaagagttaccctatggtggctgggtggcaccaaacatttattgtttaggt

SEQ ID NO: 270 - DNA, 74 bp, Artificial Sequence
  Synthetic oligonucleotide
aaccatgaagcctcaaatcatttgcaagagttaccctatggtggctgggtggcaccaaacatttatttttaggt

SEQ ID NO: 271 - DNA, 75 bp, Artificial Sequence
  Synthetic oligonucleotide
aaccatgaagcctcaaatcatttgcaagagttaccctatggtggctgggtggcaccaaacatttattttttaggt

SEQ ID NO: 272 - DNA, 75 bp, Artificial Sequence
  Synthetic oligonucleotide
aaccatgaagcctcaaatcatttgcaagagttaccctatggtggctgggtggcaccaaacatttcttatttaggt

SEQ ID NO: 273 - DNA, 75 bp, Artificial Sequence
  Synthetic oligonucleotide
aaccatgaagcctcaaatcatttgcaagagttaccctatggtggctgggtggcaccaaacatttgttatttaggt

SEQ ID NO: 274 - DNA, 74 bp, Artificial Sequence
  Synthetic oligonucleotide
aaccatgaagcctcaaatcatttgcaagagttaccctatggtggctgggtggcaccaaacatttttatttaggt

SEQ ID NO: 275 - DNA, 75 bp, Artificial Sequence
  Synthetic oligonucleotide
aaccatgaagcctcaaatcatttgcaagagttaccctatggtggctgggtggcaccaaacattttttatttaggt

SEQ ID NO: 276 - DNA, 75 bp, Artificial Sequence
  Synthetic oligonucleotide
aaccatgaagcctcaaatcatttgcaagagttaccctatggtggctgggtggcaccaaacctttattatttaggt

SEQ ID NO: 277 - DNA, 75 bp, Artificial Sequence
  Synthetic oligonucleotide
aaccatgaagcctcaaatcatttgcaagagttaccctatggtggctgggtggcaccaaacgtttattatttaggt

SEQ ID NO: 278 - DNA, 74 bp, Artificial Sequence
  Synthetic oligonucleotide
aaccatgaagcctcaaatcatttgcaagagttaccctatggtggctgggtggcaccaaactttattatttaggt

SEQ ID NO: 279 - DNA, 75 bp, Artificial Sequence
  Synthetic oligonucleotide
aaccatgaagcctcaaatcatttgcaagagttaccctatggtggctgggtggcaccaaacttttattatttaggt

SEQ ID NO: 280 - DNA, 75 bp, Artificial Sequence
  Synthetic oligonucleotide
aaccatgaagcctcaaatcatttgcaagagttaccctatggtggctgggtggcaccaaagatttattatttaggt

SEQ ID NO: 281 - DNA, 75 bp, Artificial Sequence
  Synthetic oligonucleotide
aaccatgaagcctcaaatcatttgcaagagttaccctatggtggctgggtggcaccaaatatttattatttaggt

SEQ ID NO: 282 - DNA, 74 bp, Artificial Sequence
  Synthetic oligonucleotide
aaccatgaagcctcaaatcatttgcaagagttaccctatggtggctgggtggcaccaacatttattatttaggt

SEQ ID NO: 283 - DNA, 75 bp, Artificial Sequence
  Synthetic oligonucleotide
aaccatgaagcctcaaatcatttgcaagagttaccctatggtggctgggtggcaccaaccatttattatttaggt

SEQ ID NO: 284 - DNA, 75 bp, Artificial Sequence
  Synthetic oligonucleotide
aaccatgaagcctcaaatcatttgcaagagttaccctatggtggctgggtggcaccaagcatttattatttaggt

SEQ ID NO: 285 - DNA, 75 bp, Artificial Sequence
  Synthetic oligonucleotide
aaccatgaagcctcaaatcatttgcaagagttaccctatggtggctgggtggcaccaatcatttattatttaggt

SEQ ID NO: 286 - DNA, 75 bp, Artificial Sequence
  Synthetic oligonucleotide
aaccatgaagcctcaaatcatttgcaagagttaccctatggtggctgggtggcaccacacatttattatttaggt

SEQ ID NO: 287 - DNA, 75 bp, Artificial Sequence
  Synthetic oligonucleotide
aaccatgaagcctcaaatcatttgcaagagttaccctatggtggctgggtggcaccagacatttattatttaggt

SEQ ID NO: 288 - DNA, 75 bp, Artificial Sequence
  Synthetic oligonucleotide
aaccatgaagcctcaaatcatttgcaagagttaccctatggtggctgggtggcaccatacatttattatttaggt

SEQ ID NO: 289 - DNA, 75 bp, Artificial Sequence
  Synthetic oligonucleotide
aaccatgaagcctcaaatcatttgcaagagttaccctatggtggctgggtggcacccaacatttattatttaggt

SEQ ID NO: 290 - DNA, 75 bp, Artificial Sequence
  Synthetic oligonucleotide
aaccatgaagcctcaaatcatttgcaagagttaccctatggtggctgggtggcaccgaacatttattatttaggt

SEQ ID NO: 291 - DNA, 75 bp, Artificial Sequence
  Synthetic oligonucleotide
aaccatgaagcctcaaatcatttgcaagagttaccctatggtggctgggtggcacctaacatttattatttaggt

SEQ ID NO: 292 - DNA, 75 bp, Artificial Sequence
  Synthetic oligonucleotide
aaccatgaagcctcaaatcatttgcaagagttaccctatggtggctgggtggcacgaaacatttattatttaggt

SEQ ID NO: 293 - DNA, 75 bp, Artificial Sequence
  Synthetic oligonucleotide
aaccatgaagcctcaaatcatttgcaagagttaccctatggtggctgggtggcactaaacatttattatttaggt

SEQ ID NO: 294 - DNA, 75 bp, Artificial Sequence
  Synthetic oligonucleotide
aaccatgaagcctcaaatcatttgcaagagttaccctatggtggctgggtggcagcaaacatttattatttaggt

SEQ ID NO: 295 - DNA, 75 bp, Artificial Sequence
  Synthetic oligonucleotide
aaccatgaagcctcaaatcatttgcaagagttaccctatggtggctgggtggcatcaaacatttattatttaggt

SEQ ID NO: 296 - DNA, 74 bp, Artificial Sequence
  Synthetic oligonucleotide
aaccatgaagcctcaaatcatttgcaagagttaccctatggtggctgggtggcccaaacatttattatttaggt

SEQ ID NO: 297 - DNA, 75 bp, Artificial Sequence
  Synthetic oligonucleotide
aaccatgaagcctcaaatcatttgcaagagttaccctatggtggctgggtggccccaaacatttattatttaggt

SEQ ID NO: 298 - DNA, 75 bp, Artificial Sequence
  Synthetic oligonucleotide
aaccatgaagcctcaaatcatttgcaagagttaccctatggtggctgggtggcgccaaacatttattatttaggt

SEQ ID NO: 299 - DNA, 75 bp, Artificial Sequence
  Synthetic oligonucleotide
aaccatgaagcctcaaatcatttgcaagagttaccctatggtggctgggtggctccaaacatttattatttaggt

SEQ ID NO: 300 - DNA, 75 bp, Artificial Sequence
  Synthetic oligonucleotide
aaccatgaagcctcaaatcatttgcaagagttaccctatggtggctgggtgggaccaaacatttattatttaggt

SEQ ID NO: 301 - DNA, 75 bp, Artificial Sequence
  Synthetic oligonucleotide
aaccatgaagcctcaaatcatttgcaagagttaccctatggtggctgggtggtaccaaacatttattatttaggt

SEQ ID NO: 302 - DNA, 75 bp, Artificial Sequence
  Synthetic oligonucleotide
aaccatgaagcctcaaatcatttgcaagagttaccctatggtggctgggtgtcaccaaacatttattatttaggt

SEQ ID NO: 303 - DNA, 75 bp, Artificial Sequence
  Synthetic oligonucleotide
aaccatgaagcctcaaatcatttgcaagagttaccctatggtggctgggttgcaccaaacatttattatttaggt

SEQ ID NO: 304 - DNA, 74 bp, Artificial Sequence
  Synthetic oligonucleotide
aaccatgaagcctcaaatcatttgcaagagttaccctatggtggctggtggcaccaaacatttattatttaggt

SEQ ID NO: 305 - DNA, 75 bp, Artificial Sequence
  Synthetic oligonucleotide
aaccatgaagcctcaaatcatttgcaagagttaccctatggtggctggttggcaccaaacatttattatttaggt

SEQ ID NO: 306 - DNA, 75 bp, Artificial Sequence
  Synthetic oligonucleotide
aaccatgaagcctcaaatcatttgcaagagttaccctatggtggctgtgtggcaccaaacatttattatttaggt

SEQ ID NO: 307 - DNA, 75 bp, Artificial Sequence
  Synthetic oligonucleotide
aaccatgaagcctcaaatcatttgcaagagttaccctatggtggcttggtggcaccaaacatttattatttaggt

SEQ ID NO: 308 - DNA, 75 bp, Artificial Sequence
  Synthetic oligonucleotide
aaccatgaagcctcaaatcatttgcaagagttaccctatggtgggtgggtggcaccaaacatttattatttaggt

SEQ ID NO: 309 - DNA, 74 bp, Artificial Sequence
  Synthetic oligonucleotide
aaccatgaagcctcaaatcatttgcaagagttaccctatggtggtgggtggcaccaaacatttattatttaggt

SEQ ID NO: 310 - DNA, 75 bp, Artificial Sequence
  Synthetic oligonucleotide
aaccatgaagcctcaaatcatttgcaagagttaccctatggtggttgggtggcaccaaacatttattatttaggt

SEQ ID NO: 311 - DNA, 75 bp, Artificial Sequence
  Synthetic oligonucleotide
aaccatgaagcctcaaatcatttgcaagagttaccctatggtgtctgggtggcaccaaacatttattatttaggt

SEQ ID NO: 312 - DNA, 75 bp, Artificial Sequence
  Synthetic oligonucleotide
aaccatgaagcctcaaatcatttgcaagagttaccctatggttgctgggtggcaccaaacatttattatttaggt

SEQ ID NO: 313 - DNA, 74 bp, Artificial Sequence
  Synthetic oligonucleotide
aaccatgaagcctcaaatcatttgcaagagttaccctatgtggctgggtggcaccaaacatttattatttaggt

SEQ ID NO: 314 - DNA, 75 bp, Artificial Sequence
  Synthetic oligonucleotide
aaccatgaagcctcaaatcatttgcaagagttaccctatgttggctgggtggcaccaaacatttattatttaggt

SEQ ID NO: 315 - DNA, 75 bp, Artificial Sequence
  Synthetic oligonucleotide
aaccatgaagcctcaaatcatttgcaagagttaccctattgtggctgggtggcaccaaacatttattatttaggt

SEQ ID NO: 316 - DNA, 75 bp, Artificial Sequence
  Synthetic oligonucleotide
aaccatgaagcctcaaatcatttgcaagagttaccctctggtggctgggtggcaccaaacatttattatttaggt

SEQ ID NO: 317 - DNA, 75 bp, Artificial Sequence
  Synthetic oligonucleotide
aaccatgaagcctcaaatcatttgcaagagttaccctgtggtggctgggtggcaccaaacatttattatttaggt

SEQ ID NO: 318 - DNA, 74 bp, Artificial Sequence
  Synthetic oligonucleotide
aaccatgaagcctcaaatcatttgcaagagttacccttggtggctgggtggcaccaaacatttattatttaggt

SEQ ID NO: 319 - DNA, 75 bp, Artificial Sequence
  Synthetic oligonucleotide
aaccatgaagcctcaaatcatttgcaagagttaccctttggtggctgggtggcaccaaacatttattatttaggt

SEQ ID NO: 320 - DNA, 75 bp, Artificial Sequence
  Synthetic oligonucleotide
aaccatgaagcctcaaatcatttgcaagagttaccgtatggtggctgggtggcaccaaacatttattatttaggt

SEQ ID NO: 321 - DNA, 74 bp, Artificial Sequence
  Synthetic oligonucleotide
aaccatgaagcctcaaatcatttgcaagagttacctatggtggctgggtggcaccaaacatttattatttaggt

SEQ ID NO: 322 - DNA, 75 bp, Artificial Sequence
  Synthetic oligonucleotide
aaccatgaagcctcaaatcatttgcaagagttaccttatggtggctgggtggcaccaaacatttattatttaggt

SEQ ID NO: 323 - DNA, 75 bp, Artificial Sequence
  Synthetic oligonucleotide
aaccatgaagcctcaaatcatttgcaagagttacgctatggtggctgggtggcaccaaacatttattatttaggt

SEQ ID NO: 324 - DNA, 75 bp, Artificial Sequence
  Synthetic oligonucleotide
aaccatgaagcctcaaatcatttgcaagagttactctatggtggctgggtggcaccaaacatttattatttaggt

SEQ ID NO: 325 - DNA, 75 bp, Artificial Sequence
  Synthetic oligonucleotide
aaccatgaagcctcaaatcatttgcaagagttagcctatggtggctgggtggcaccaaacatttattatttaggt

SEQ ID NO: 326 - DNA, 75 bp, Artificial Sequence
  Synthetic oligonucleotide
aaccatgaagcctcaaatcatttgcaagagttatcctatggtggctgggtggcaccaaacatttattatttaggt

SEQ ID NO: 327 - DNA, 75 bp, Artificial Sequence
  Synthetic oligonucleotide
aaccatgaagcctcaaatcatttgcaagagttcccctatggtggctgggtggcaccaaacatttattatttaggt

SEQ ID NO: 328 - DNA, 74 bp, Artificial Sequence
  Synthetic oligonucleotide
aaccatgaagcctcaaatcatttgcaagagttccctatggtggctgggtggcaccaaacatttattatttaggt

SEQ ID NO: 329 - DNA, 75 bp, Artificial Sequence
  Synthetic oligonucleotide
aaccatgaagcctcaaatcatttgcaagagttgccctatggtggctgggtggcaccaaacatttattatttaggt

SEQ ID NO: 330 - DNA, 75 bp, Artificial Sequence
  Synthetic oligonucleotide
aaccatgaagcctcaaatcatttgcaagagtttccctatggtggctgggtggcaccaaacatttattatttaggt

SEQ ID NO: 331 - DNA, 74 bp, Artificial Sequence
  Synthetic oligonucleotide
aaccatgaagcctcaaatcatttgcaagattaccctatggtggctgggtggcaccaaacatttattatttaggt

SEQ ID NO: 332 - DNA, 75 bp, Artificial Sequence
  Synthetic oligonucleotide
aaccatgaagcctcaaatcatttgcaagatttaccctatggtggctgggtggcaccaaacatttattatttaggt

SEQ ID NO: 333 - DNA, 75 bp, Artificial Sequence
  Synthetic oligonucleotide
aaccatgaagcctcaaatcatttgcaagcgttaccctatggtggctgggtggcaccaaacatttattatttaggt

SEQ ID NO: 334 - DNA, 75 bp, Artificial Sequence
  Synthetic oligonucleotide
aaccatgaagcctcaaatcatttgcaagggttaccctatggtggctgggtggcaccaaacatttattatttaggt

SEQ ID NO: 335 - DNA, 74 bp, Artificial Sequence
  Synthetic oligonucleotide
aaccatgaagcctcaaatcatttgcaaggttaccctatggtggctgggtggcaccaaacatttattatttaggt

SEQ ID NO: 336 - DNA, 75 bp, Artificial Sequence
  Synthetic oligonucleotide
aaccatgaagcctcaaatcatttgcaagtgttaccctatggtggctgggtggcaccaaacatttattatttaggt

SEQ ID NO: 337 - DNA, 75 bp, Artificial Sequence
  Synthetic oligonucleotide
aaccatgaagcctcaaatcatttgcaatagttaccctatggtggctgggtggcaccaaacatttattatttaggt

SEQ ID NO: 338 - DNA, 75 bp, Artificial Sequence
  Synthetic oligonucleotide
aaccatgaagcctcaaatcatttgcacgagttaccctatggtggctgggtggcaccaaacatttattatttaggt

SEQ ID NO: 339 - DNA, 74 bp, Artificial Sequence
  Synthetic oligonucleotide
aaccatgaagcctcaaatcatttgcagagttaccctatggtggctgggtggcaccaaacatttattatttaggt

SEQ ID NO: 340 - DNA, 75 bp, Artificial Sequence
  Synthetic oligonucleotide
aaccatgaagcctcaaatcatttgcaggagttaccctatggtggctgggtggcaccaaacatttattatttaggt

SEQ ID NO: 341 - DNA, 75 bp, Artificial Sequence
  Synthetic oligonucleotide
aaccatgaagcctcaaatcatttgcatgagttaccctatggtggctgggtggcaccaaacatttattatttaggt

SEQ ID NO: 342 - DNA, 75 bp, Artificial Sequence
  Synthetic oligonucleotide
aaccatgaagcctcaaatcatttgccagagttaccctatggtggctgggtggcaccaaacatttattatttaggt

SEQ ID NO: 343 - DNA, 75 bp, Artificial Sequence
  Synthetic oligonucleotide
aaccatgaagcctcaaatcatttgcgagagttaccctatggtggctgggtggcaccaaacatttattatttaggt

SEQ ID NO: 344 - DNA, 75 bp, Artificial Sequence
  Synthetic oligonucleotide
aaccatgaagcctcaaatcatttgctagagttaccctatggtggctgggtggcaccaaacatttattatttaggt

SEQ ID NO: 345 - DNA, 75 bp, Artificial Sequence
  Synthetic oligonucleotide
aaccatgaagcctcaaatcatttggaagagttaccctatggtggctgggtggcaccaaacatttattatttaggt

SEQ ID NO: 346 - DNA, 75 bp, Artificial Sequence
  Synthetic oligonucleotide
aaccatgaagcctcaaatcatttgtaagagttaccctatggtggctgggtggcaccaaacatttattatttaggt

SEQ ID NO: 347 - DNA, 75 bp, Artificial Sequence
  Synthetic oligonucleotide
aaccatgaagcctcaaatcattttcaagagttaccctatggtggctgggtggcaccaaacatttattatttaggt

SEQ ID NO: 348 - DNA, 75 bp, Artificial Sequence
  Synthetic oligonucleotide
aaccatgaagcctcaaatcctttgcaagagttaccctatggtggctgggtggcaccaaacatttattatttaggt

SEQ ID NO: 349 - DNA, 75 bp, Artificial Sequence
  Synthetic oligonucleotide
aaccatgaagcctcaaatcgtttgcaagagttaccctatggtggctgggtggcaccaaacatttattatttaggt

SEQ ID NO: 350 - DNA, 74 bp, Artificial Sequence
  Synthetic oligonucleotide
aaccatgaagcctcaaatctttgcaagagttaccctatggtggctgggtggcaccaaacatttattatttaggt

SEQ ID NO: 351 - DNA, 75 bp, Artificial Sequence
  Synthetic oligonucleotide
aaccatgaagcctcaaatcttttgcaagagttaccctatggtggctgggtggcaccaaacatttattatttaggt

SEQ ID NO: 352 - DNA, 75 bp, Artificial Sequence
  Synthetic oligonucleotide
aaccatgaagcctcaaatgatttgcaagagttaccctatggtggctgggtggcaccaaacatttattatttaggt

SEQ ID NO: 353 - DNA, 75 bp, Artificial Sequence
  Synthetic oligonucleotide
aaccatgaagcctcaaattatttgcaagagttaccctatggtggctgggtggcaccaaacatttattatttaggt

SEQ ID NO: 354 - DNA, 75 bp, Artificial Sequence
  Synthetic oligonucleotide
aaccatgaagcctcaactcatttgcaagagttaccctatggtggctgggtggcaccaaacatttattatttaggt

SEQ ID NO: 355 - DNA, 75 bp, Artificial Sequence
  Synthetic oligonucleotide
aaccatgaagcctcaagtcatttgcaagagttaccctatggtggctgggtggcaccaaacatttattatttaggt

SEQ ID NO: 356 - DNA, 74 bp, Artificial Sequence
  Synthetic oligonucleotide
aaccatgaagcctcaatcatttgcaagagttaccctatggtggctgggtggcaccaaacatttattatttaggt

SEQ ID NO: 357 - DNA, 75 bp, Artificial Sequence
  Synthetic oligonucleotide
aaccatgaagcctcaattcatttgcaagagttaccctatggtggctgggtggcaccaaacatttattatttaggt

SEQ ID NO: 358 - DNA, 75 bp, Artificial Sequence
  Synthetic oligonucleotide
aaccatgaagcctcacatcatttgcaagagttaccctatggtggctgggtggcaccaaacatttattatttaggt

SEQ ID NO: 359 - DNA, 75 bp, Artificial Sequence
  Synthetic oligonucleotide
aaccatgaagcctcagatcatttgcaagagttaccctatggtggctgggtggcaccaaacatttattatttaggt

SEQ ID NO: 360 - DNA, 75 bp, Artificial Sequence
  Synthetic oligonucleotide
aaccatgaagcctcatatcatttgcaagagttaccctatggtggctgggtggcaccaaacatttattatttaggt

SEQ ID NO: 361 - DNA, 75 bp, Artificial Sequence
  Synthetic oligonucleotide
aaccatgaagcctccaatcatttgcaagagttaccctatggtggctgggtggcaccaaacatttattatttaggt

SEQ ID NO: 362 - DNA, 75 bp, Artificial Sequence
  Synthetic oligonucleotide
aaccatgaagcctcgaatcatttgcaagagttaccctatggtggctgggtggcaccaaacatttattatttaggt

SEQ ID NO: 363 - DNA, 75 bp, Artificial Sequence
  Synthetic oligonucleotide
aaccatgaagcctctaatcatttgcaagagttaccctatggtggctgggtggcaccaaacatttattatttaggt

SEQ ID NO: 364 - DNA, 75 bp, Artificial Sequence
  Synthetic oligonucleotide
aaccatgaagcctgaaatcatttgcaagagttaccctatggtggctgggtggcaccaaacatttattatttaggt

SEQ ID NO: 365 - DNA, 75 bp, Artificial Sequence
  Synthetic oligonucleotide
aaccatgaagccttaaatcatttgcaagagttaccctatggtggctgggtggcaccaaacatttattatttaggt

SEQ ID NO: 366 - DNA, 75 bp, Artificial Sequence
  Synthetic oligonucleotide
aaccatgaagcgtcaaatcatttgcaagagttaccctatggtggctgggtggcaccaaacatttattatttaggt

SEQ ID NO: 367 - DNA, 74 bp, Artificial Sequence
  Synthetic oligonucleotide
aaccatgaagctcaaatcatttgcaagagttaccctatggtggctgggtggcaccaaacatttattatttaggt

SEQ ID NO: 368 - DNA, 75 bp, Artificial Sequence
  Synthetic oligonucleotide
aaccatgaagcttcaaatcatttgcaagagttaccctatggtggctgggtggcaccaaacatttattatttaggt

SEQ ID NO: 369 - DNA, 75 bp, Artificial Sequence
  Synthetic oligonucleotide
aaccatgaaggctcaaatcatttgcaagagttaccctatggtggctgggtggcaccaaacatttattatttaggt

SEQ ID NO: 370 - DNA, 75 bp, Artificial Sequence
  Synthetic oligonucleotide
aaccatgaagtctcaaatcatttgcaagagttaccctatggtggctgggtggcaccaaacatttattatttaggt

SEQ ID NO: 371 - DNA, 75 bp, Artificial Sequence
  Synthetic oligonucleotide
aaccatgaatcctcaaatcatttgcaagagttaccctatggtggctgggtggcaccaaacatttattatttaggt

SEQ ID NO: 372 - DNA, 75 bp, Artificial Sequence
  Synthetic oligonucleotide
aaccatgacgcctcaaatcatttgcaagagttaccctatggtggctgggtggcaccaaacatttattatttaggt

SEQ ID NO: 373 - DNA, 74 bp, Artificial Sequence
  Synthetic oligonucleotide
aaccatgagcctcaaatcatttgcaagagttaccctatggtggctgggtggcaccaaacatttattatttaggt

SEQ ID NO: 374 - DNA, 75 bp, Artificial Sequence
  Synthetic oligonucleotide
aaccatgaggcctcaaatcatttgcaagagttaccctatggtggctgggtggcaccaaacatttattatttaggt

SEQ ID NO: 375 - DNA, 75 bp, Artificial Sequence
  Synthetic oligonucleotide
aaccatgatgcctcaaatcatttgcaagagttaccctatggtggctgggtggcaccaaacatttattatttaggt

SEQ ID NO: 376 - DNA, 75 bp, Artificial Sequence
  Synthetic oligonucleotide
aaccatgcagcctcaaatcatttgcaagagttaccctatggtggctgggtggcaccaaacatttattatttaggt

SEQ ID NO: 377 - DNA, 75 bp, Artificial Sequence
  Synthetic oligonucleotide
aaccatgccgcctcaaatcatttgcaagagttaccctatggtggctgggtggcaccaaacatttattatttaggt

SEQ ID NO: 378 - DNA, 75 bp, Artificial Sequence
  Synthetic oligonucleotide
aaccatgcggcctcaaatcatttgcaagagttaccctatggtggctgggtggcaccaaacatttattatttaggt

SEQ ID NO: 379 - DNA, 75 bp, Artificial Sequence
  Synthetic oligonucleotide
aaccatgctgcctcaaatcatttgcaagagttaccctatggtggctgggtggcaccaaacatttattatttaggt

SEQ ID NO: 380 - DNA, 75 bp, Artificial Sequence
  Synthetic oligonucleotide
aaccatggagcctcaaatcatttgcaagagttaccctatggtggctgggtggcaccaaacatttattatttaggt

SEQ ID NO: 381 - DNA, 75 bp, Artificial Sequence
  Synthetic oligonucleotide
aaccatggcgcctcaaatcatttgcaagagttaccctatggtggctgggtggcaccaaacatttattatttaggt

SEQ ID NO: 382 - DNA, 75 bp, Artificial Sequence
  Synthetic oligonucleotide
aaccatggggcctcaaatcatttgcaagagttaccctatggtggctgggtggcaccaaacatttattatttaggt

SEQ ID NO: 383 - DNA, 75 bp, Artificial Sequence
  Synthetic oligonucleotide
aaccatggtgcctcaaatcatttgcaagagttaccctatggtggctgggtggcaccaaacatttattatttaggt

SEQ ID NO: 384 - DNA, 75 bp, Artificial Sequence
  Synthetic oligonucleotide
aaccatgtagcctcaaatcatttgcaagagttaccctatggtggctgggtggcaccaaacatttattatttaggt

SEQ ID NO: 385 - DNA, 75 bp, Artificial Sequence
  Synthetic oligonucleotide
aaccatgtcgcctcaaatcatttgcaagagttaccctatggtggctgggtggcaccaaacatttattatttaggt

SEQ ID NO: 386 - DNA, 75 bp, Artificial Sequence
  Synthetic oligonucleotide
aaccatgtggcctcaaatcatttgcaagagttaccctatggtggctgggtggcaccaaacatttattatttaggt

SEQ ID NO: 387 - DNA, 75 bp, Artificial Sequence
  Synthetic oligonucleotide
aaccatgttgcctcaaatcatttgcaagagttaccctatggtggctgggtggcaccaaacatttattatttaggt

SEQ ID NO: 388 - DNA, 75 bp, Artificial Sequence
  Synthetic oligonucleotide
aaccattaagcctcaaatcatttgcaagagttaccctatggtggctgggtggcaccaaacatttattatttaggt

SEQ ID NO: 389 - DNA, 75 bp, Artificial Sequence
  Synthetic oligonucleotide
aaccattacgcctcaaatcatttgcaagagttaccctatggtggctgggtggcaccaaacatttattatttaggt

SEQ ID NO: 390 - DNA, 75 bp, Artificial Sequence
  Synthetic oligonucleotide
aaccattaggcctcaaatcatttgcaagagttaccctatggtggctgggtggcaccaaacatttattatttaggt

SEQ ID NO: 391 - DNA, 75 bp, Artificial Sequence
  Synthetic oligonucleotide
aaccattatgcctcaaatcatttgcaagagttaccctatggtggctgggtggcaccaaacatttattatttaggt

SEQ ID NO: 392 - DNA, 75 bp, Artificial Sequence
  Synthetic oligonucleotide
aaccattcagcctcaaatcatttgcaagagttaccctatggtggctgggtggcaccaaacatttattatttaggt

SEQ ID NO: 393 - DNA, 75 bp, Artificial Sequence
  Synthetic oligonucleotide
aaccattccgcctcaaatcatttgcaagagttaccctatggtggctgggtggcaccaaacatttattatttaggt

SEQ ID NO: 394 - DNA, 75 bp, Artificial Sequence
  Synthetic oligonucleotide
aaccattcggcctcaaatcatttgcaagagttaccctatggtggctgggtggcaccaaacatttattatttaggt

SEQ ID NO: 395 - DNA, 75 bp, Artificial Sequence
  Synthetic oligonucleotide
aaccattctgcctcaaatcatttgcaagagttaccctatggtggctgggtggcaccaaacatttattatttaggt

SEQ ID NO: 396 - DNA, 75 bp, Artificial Sequence
  Synthetic oligonucleotide
aaccattgagcctcaaatcatttgcaagagttaccctatggtggctgggtggcaccaaacatttattatttaggt

SEQ ID NO: 397 - DNA, 75 bp, Artificial Sequence
  Synthetic oligonucleotide
aaccattgcgcctcaaatcatttgcaagagttaccctatggtggctgggtggcaccaaacatttattatttaggt

SEQ ID NO: 398 - DNA, 75 bp, Artificial Sequence
  Synthetic oligonucleotide
aaccattgggcctcaaatcatttgcaagagttaccctatggtggctgggtggcaccaaacatttattatttaggt

SEQ ID NO: 399 - DNA, 75 bp, Artificial Sequence
  Synthetic oligonucleotide
aaccattgtgcctcaaatcatttgcaagagttaccctatggtggctgggtggcaccaaacatttattatttaggt

SEQ ID NO: 400 - DNA, 75 bp, Artificial Sequence
  Synthetic oligonucleotide
aaccatttagcctcaaatcatttgcaagagttaccctatggtggctgggtggcaccaaacatttattatttaggt

SEQ ID NO: 401 - DNA, 75 bp, Artificial Sequence
  Synthetic oligonucleotide
aaccatttcgcctcaaatcatttgcaagagttaccctatggtggctgggtggcaccaaacatttattatttaggt

SEQ ID NO: 402 - DNA, 75 bp, Artificial Sequence
  Synthetic oligonucleotide
aaccatttggcctcaaatcatttgcaagagttaccctatggtggctgggtggcaccaaacatttattatttaggt

SEQ ID NO: 403 - DNA, 75 bp, Artificial Sequence
  Synthetic oligonucleotide
aaccattttgcctcaaatcatttgcaagagttaccctatggtggctgggtggcaccaaacatttattatttaggt

SEQ ID NO: 404 - DNA, 75 bp, Artificial Sequence
  Synthetic oligonucleotide
aacccagaagcctcaaatcatttgcaagagttaccctatggtggctgggtggcaccaaacatttattatttaggt

SEQ ID NO: 405 - DNA, 75 bp, Artificial Sequence
  Synthetic oligonucleotide
aaccccgaagcctcaaatcatttgcaagagttaccctatggtggctgggtggcaccaaacatttattatttaggt

SEQ ID NO: 406 - DNA, 75 bp, Artificial Sequence
  Synthetic oligonucleotide
aacccggaagcctcaaatcatttgcaagagttaccctatggtggctgggtggcaccaaacatttattatttaggt

SEQ ID NO: 407 - DNA, 75 bp, Artificial Sequence
  Synthetic oligonucleotide
aaccctgaagcctcaaatcatttgcaagagttaccctatggtggctgggtggcaccaaacatttattatttaggt

SEQ ID NO: 408 - DNA, 75 bp, Artificial Sequence
  Synthetic oligonucleotide
aaccgagaagcctcaaatcatttgcaagagttaccctatggtggctgggtggcaccaaacatttattatttaggt

SEQ ID NO: 409 - DNA, 75 bp, Artificial Sequence
  Synthetic oligonucleotide
aaccgcgaagcctcaaatcatttgcaagagttaccctatggtggctgggtggcaccaaacatttattatttaggt

SEQ ID NO: 410 - DNA, 75 bp, Artificial Sequence
  Synthetic oligonucleotide
aaccgggaagcctcaaatcatttgcaagagttaccctatggtggctgggtggcaccaaacatttattatttaggt

SEQ ID NO: 411 - DNA, 75 bp, Artificial Sequence
  Synthetic oligonucleotide
aaccgtgaagcctcaaatcatttgcaagagttaccctatggtggctgggtggcaccaaacatttattatttaggt

SEQ ID NO: 412 - DNA, 75 bp, Artificial Sequence
  Synthetic oligonucleotide
aacctagaagcctcaaatcatttgcaagagttaccctatggtggctgggtggcaccaaacatttattatttaggt

SEQ ID NO: 413 - DNA, 75 bp, Artificial Sequence
  Synthetic oligonucleotide
aacctcgaagcctcaaatcatttgcaagagttaccctatggtggctgggtggcaccaaacatttattatttaggt

SEQ ID NO: 414 - DNA, 74 bp, Artificial Sequence
  Synthetic oligonucleotide
aacctgaagcctcaaatcatttgcaagagttaccctatggtggctgggtggcaccaaacatttattatttaggt

SEQ ID NO: 415 - DNA, 75 bp, Artificial Sequence
  Synthetic oligonucleotide
aacctggaagcctcaaatcatttgcaagagttaccctatggtggctgggtggcaccaaacatttattatttaggt

SEQ ID NO: 416 - DNA, 75 bp, Artificial Sequence
  Synthetic oligonucleotide
aaccttgaagcctcaaatcatttgcaagagttaccctatggtggctgggtggcaccaaacatttattatttaggt

SEQ ID NO: 417 - DNA, 75 bp, Artificial Sequence
  Synthetic oligonucleotide
aacgaagaagcctcaaatcatttgcaagagttaccctatggtggctgggtggcaccaaacatttattatttaggt

SEQ ID NO: 418 - DNA, 75 bp, Artificial Sequence
  Synthetic oligonucleotide
aacgacgaagcctcaaatcatttgcaagagttaccctatggtggctgggtggcaccaaacatttattatttaggt

SEQ ID NO: 419 - DNA, 75 bp, Artificial Sequence
  Synthetic oligonucleotide
aacgaggaagcctcaaatcatttgcaagagttaccctatggtggctgggtggcaccaaacatttattatttaggt

SEQ ID NO: 420 - DNA, 75 bp, Artificial Sequence
  Synthetic oligonucleotide
aacgatgaagcctcaaatcatttgcaagagttaccctatggtggctgggtggcaccaaacatttattatttaggt

SEQ ID NO: 421 - DNA, 75 bp, Artificial Sequence
  Synthetic oligonucleotide
aacgcagaagcctcaaatcatttgcaagagttaccctatggtggctgggtggcaccaaacatttattatttaggt

SEQ ID NO: 422 - DNA, 75 bp, Artificial Sequence
  Synthetic oligonucleotide
aacgccgaagcctcaaatcatttgcaagagttaccctatggtggctgggtggcaccaaacatttattatttaggt

SEQ ID NO: 423 - DNA, 75 bp, Artificial Sequence
  Synthetic oligonucleotide
aacgcggaagcctcaaatcatttgcaagagttaccctatggtggctgggtggcaccaaacatttattatttaggt

SEQ ID NO: 424 - DNA, 75 bp, Artificial Sequence
  Synthetic oligonucleotide
aacgctgaagcctcaaatcatttgcaagagttaccctatggtggctgggtggcaccaaacatttattatttaggt

SEQ ID NO: 425 - DNA, 75 bp, Artificial Sequence
  Synthetic oligonucleotide
aacggagaagcctcaaatcatttgcaagagttaccctatggtggctgggtggcaccaaacatttattatttaggt

SEQ ID NO: 426 - DNA, 75 bp, Artificial Sequence
  Synthetic oligonucleotide
aacggcgaagcctcaaatcatttgcaagagttaccctatggtggctgggtggcaccaaacatttattatttaggt

SEQ ID NO: 427 - DNA, 75 bp, Artificial Sequence
  Synthetic oligonucleotide
aacggggaagcctcaaatcatttgcaagagttaccctatggtggctgggtggcaccaaacatttattatttaggt

SEQ ID NO: 428 - DNA, 75 bp, Artificial Sequence
  Synthetic oligonucleotide
aacggtgaagcctcaaatcatttgcaagagttaccctatggtggctgggtggcaccaaacatttattatttaggt

SEQ ID NO: 429 - DNA, 75 bp, Artificial Sequence
  Synthetic oligonucleotide
aacgtagaagcctcaaatcatttgcaagagttaccctatggtggctgggtggcaccaaacatttattatttaggt

SEQ ID NO: 430 - DNA, 75 bp, Artificial Sequence
  Synthetic oligonucleotide
aacgtcgaagcctcaaatcatttgcaagagttaccctatggtggctgggtggcaccaaacatttattatttaggt

SEQ ID NO: 431 - DNA, 75 bp, Artificial Sequence
  Synthetic oligonucleotide
aacgtggaagcctcaaatcatttgcaagagttaccctatggtggctgggtggcaccaaacatttattatttaggt

SEQ ID NO: 432 - DNA, 75 bp, Artificial Sequence
  Synthetic oligonucleotide
aacgttgaagcctcaaatcatttgcaagagttaccctatggtggctgggtggcaccaaacatttattatttaggt

SEQ ID NO: 433 - DNA, 75 bp, Artificial Sequence
  Synthetic oligonucleotide
aactaagaagcctcaaatcatttgcaagagttaccctatggtggctgggtggcaccaaacatttattatttaggt

SEQ ID NO: 434 - DNA, 75 bp, Artificial Sequence
  Synthetic oligonucleotide
aactacgaagcctcaaatcatttgcaagagttaccctatggtggctgggtggcaccaaacatttattatttaggt

SEQ ID NO: 435 - DNA, 75 bp, Artificial Sequence
  Synthetic oligonucleotide
aactaggaagcctcaaatcatttgcaagagttaccctatggtggctgggtggcaccaaacatttattatttaggt

SEQ ID NO: 436 - DNA, 75 bp, Artificial Sequence
  Synthetic oligonucleotide
aactatgaagcctcaaatcatttgcaagagttaccctatggtggctgggtggcaccaaacatttattatttaggt

SEQ ID NO: 437 - DNA, 75 bp, Artificial Sequence
  Synthetic oligonucleotide
aactcagaagcctcaaatcatttgcaagagttaccctatggtggctgggtggcaccaaacatttattatttaggt

SEQ ID NO: 438 - DNA, 75 bp, Artificial Sequence
  Synthetic oligonucleotide
aactccgaagcctcaaatcatttgcaagagttaccctatggtggctgggtggcaccaaacatttattatttaggt

SEQ ID NO: 439 - DNA, 75 bp, Artificial Sequence
  Synthetic oligonucleotide
aactcggaagcctcaaatcatttgcaagagttaccctatggtggctgggtggcaccaaacatttattatttaggt

SEQ ID NO: 440 - DNA, 75 bp, Artificial Sequence
  Synthetic oligonucleotide
aactctgaagcctcaaatcatttgcaagagttaccctatggtggctgggtggcaccaaacatttattatttaggt

SEQ ID NO: 441 - DNA, 75 bp, Artificial Sequence
  Synthetic oligonucleotide
aactgagaagcctcaaatcatttgcaagagttaccctatggtggctgggtggcaccaaacatttattatttaggt

SEQ ID NO: 442 - DNA, 75 bp, Artificial Sequence
  Synthetic oligonucleotide
aactgcgaagcctcaaatcatttgcaagagttaccctatggtggctgggtggcaccaaacatttattatttaggt

SEQ ID NO: 443 - DNA, 75 bp, Artificial Sequence
  Synthetic oligonucleotide
aactgggaagcctcaaatcatttgcaagagttaccctatggtggctgggtggcaccaaacatttattatttaggt

SEQ ID NO: 444 - DNA, 75 bp, Artificial Sequence
  Synthetic oligonucleotide
aactgtgaagcctcaaatcatttgcaagagttaccctatggtggctgggtggcaccaaacatttattatttaggt

SEQ ID NO: 445 - DNA, 75 bp, Artificial Sequence
  Synthetic oligonucleotide
aacttagaagcctcaaatcatttgcaagagttaccctatggtggctgggtggcaccaaacatttattatttaggt

SEQ ID NO: 446 - DNA, 75 bp, Artificial Sequence
  Synthetic oligonucleotide
aacttcgaagcctcaaatcatttgcaagagttaccctatggtggctgggtggcaccaaacatttattatttaggt

SEQ ID NO: 447 - DNA, 75 bp, Artificial Sequence
  Synthetic oligonucleotide
aacttggaagcctcaaatcatttgcaagagttaccctatggtggctgggtggcaccaaacatttattatttaggt

SEQ ID NO: 448 - DNA, 75 bp, Artificial Sequence
  Synthetic oligonucleotide
aactttgaagcctcaaatcatttgcaagagttaccctatggtggctgggtggcaccaaacatttattatttaggt

SEQ ID NO: 449 - DNA, 75 bp, Artificial Sequence
  Synthetic oligonucleotide
aagcatgaagcctcaaatcatttgcaagagttaccctatggtggctgggtggcaccaaacatttattatttaggt

SEQ ID NO: 450 - DNA, 75 bp, Artificial Sequence
  Synthetic oligonucleotide
aatcatgaagcctcaaatcatttgcaagagttaccctatggtggctgggtggcaccaaacatttattatttaggt

SEQ ID NO: 451 - DNA, 75 bp, Artificial Sequence
  Synthetic oligonucleotide
acacatgaagcctcaaatcatttgcaagagttaccctatggtggctgggtggcaccaaacatttattatttaggt

SEQ ID NO: 452 - DNA, 74 bp, Artificial Sequence
  Synthetic oligonucleotide
accatgaagcctcaaatcatttgcaagagttaccctatggtggctgggtggcaccaaacatttattatttaggt

SEQ ID NO: 453 - DNA, 75 bp, Artificial Sequence
  Synthetic oligonucleotide
acccatgaagcctcaaatcatttgcaagagttaccctatggtggctgggtggcaccaaacatttattatttaggt

SEQ ID NO: 454 - DNA, 75 bp, Artificial Sequence
  Synthetic oligonucleotide
acgcatgaagcctcaaatcatttgcaagagttaccctatggtggctgggtggcaccaaacatttattatttaggt

SEQ ID NO: 455 - DNA, 75 bp, Artificial Sequence
  Synthetic oligonucleotide
actcatgaagcctcaaatcatttgcaagagttaccctatggtggctgggtggcaccaaacatttattatttaggt

SEQ ID NO: 456 - DNA, 75 bp, Artificial Sequence
  Synthetic oligonucleotide
agacatgaagcctcaaatcatttgcaagagttaccctatggtggctgggtggcaccaaacatttattatttaggt

SEQ ID NO: 457 - DNA, 75 bp, Artificial Sequence
  Synthetic oligonucleotide
agccatgaagcctcaaatcatttgcaagagttaccctatggtggctgggtggcaccaaacatttattatttaggt

SEQ ID NO: 458 - DNA, 75 bp, Artificial Sequence
  Synthetic oligonucleotide
aggcatgaagcctcaaatcatttgcaagagttaccctatggtggctgggtggcaccaaacatttattatttaggt

SEQ ID NO: 459 - DNA, 75 bp, Artificial Sequence
  Synthetic oligonucleotide
agtcatgaagcctcaaatcatttgcaagagttaccctatggtggctgggtggcaccaaacatttattatttaggt

SEQ ID NO: 460 - DNA, 75 bp, Artificial Sequence
  Synthetic oligonucleotide
atacatgaagcctcaaatcatttgcaagagttaccctatggtggctgggtggcaccaaacatttattatttaggt

SEQ ID NO: 461 - DNA, 75 bp, Artificial Sequence
  Synthetic oligonucleotide
atccatgaagcctcaaatcatttgcaagagttaccctatggtggctgggtggcaccaaacatttattatttaggt

SEQ ID NO: 462 - DNA, 75 bp, Artificial Sequence
  Synthetic oligonucleotide
atgcatgaagcctcaaatcatttgcaagagttaccctatggtggctgggtggcaccaaacatttattatttaggt

SEQ ID NO: 463 - DNA, 75 bp, Artificial Sequence
  Synthetic oligonucleotide
attcatgaagcctcaaatcatttgcaagagttaccctatggtggctgggtggcaccaaacatttattatttaggt

SEQ ID NO: 464 - DNA, 75 bp, Artificial Sequence
  Synthetic oligonucleotide
caacatgaagcctcaaatcatttgcaagagttaccctatggtggctgggtggcaccaaacatttattatttaggt

SEQ ID NO: 465 - DNA, 75 bp, Artificial Sequence
  Synthetic oligonucleotide
caccatgaagcctcaaatcatttgcaagagttaccctatggtggctgggtggcaccaaacatttattatttaggt

SEQ ID NO: 466 - DNA, 75 bp, Artificial Sequence
  Synthetic oligonucleotide
cagcatgaagcctcaaatcatttgcaagagttaccctatggtggctgggtggcaccaaacatttattatttaggt

SEQ ID NO: 467 - DNA, 75 bp, Artificial Sequence
  Synthetic oligonucleotide
catcatgaagcctcaaatcatttgcaagagttaccctatggtggctgggtggcaccaaacatttattatttaggt

SEQ ID NO: 468 - DNA, 75 bp, Artificial Sequence
  Synthetic oligonucleotide
ccacatgaagcctcaaatcatttgcaagagttaccctatggtggctgggtggcaccaaacatttattatttaggt

SEQ ID NO: 469 - DNA, 75 bp, Artificial Sequence
  Synthetic oligonucleotide
ccccatgaagcctcaaatcatttgcaagagttaccctatggtggctgggtggcaccaaacatttattatttaggt

SEQ ID NO: 470 - DNA, 75 bp, Artificial Sequence
  Synthetic oligonucleotide
ccgcatgaagcctcaaatcatttgcaagagttaccctatggtggctgggtggcaccaaacatttattatttaggt

SEQ ID NO: 471 - DNA, 75 bp, Artificial Sequence
  Synthetic oligonucleotide
cctcatgaagcctcaaatcatttgcaagagttaccctatggtggctgggtggcaccaaacatttattatttaggt

SEQ ID NO: 472 - DNA, 75 bp, Artificial Sequence
  Synthetic oligonucleotide
cgacatgaagcctcaaatcatttgcaagagttaccctatggtggctgggtggcaccaaacatttattatttaggt

SEQ ID NO: 473 - DNA, 75 bp, Artificial Sequence
  Synthetic oligonucleotide
cgccatgaagcctcaaatcatttgcaagagttaccctatggtggctgggtggcaccaaacatttattatttaggt

SEQ ID NO: 474 - DNA, 75 bp, Artificial Sequence
  Synthetic oligonucleotide
cggcatgaagcctcaaatcatttgcaagagttaccctatggtggctgggtggcaccaaacatttattatttaggt

SEQ ID NO: 475 - DNA, 75 bp, Artificial Sequence
  Synthetic oligonucleotide
cgtcatgaagcctcaaatcatttgcaagagttaccctatggtggctgggtggcaccaaacatttattatttaggt

SEQ ID NO: 476 - DNA, 75 bp, Artificial Sequence
  Synthetic oligonucleotide
ctacatgaagcctcaaatcatttgcaagagttaccctatggtggctgggtggcaccaaacatttattatttaggt

SEQ ID NO: 477 - DNA, 75 bp, Artificial Sequence
  Synthetic oligonucleotide
ctccatgaagcctcaaatcatttgcaagagttaccctatggtggctgggtggcaccaaacatttattatttaggt

SEQ ID NO: 478 - DNA, 75 bp, Artificial Sequence
  Synthetic oligonucleotide
ctgcatgaagcctcaaatcatttgcaagagttaccctatggtggctgggtggcaccaaacatttattatttaggt

SEQ ID NO: 479 - DNA, 75 bp, Artificial Sequence
  Synthetic oligonucleotide
cttcatgaagcctcaaatcatttgcaagagttaccctatggtggctgggtggcaccaaacatttattatttaggt

SEQ ID NO: 480 - DNA, 75 bp, Artificial Sequence
  Synthetic oligonucleotide
gaacatgaagcctcaaatcatttgcaagagttaccctatggtggctgggtggcaccaaacatttattatttaggt

SEQ ID NO: 481 - DNA, 75 bp, Artificial Sequence
  Synthetic oligonucleotide
gaccatgaagcctcaaatcatttgcaagagttaccctatggtggctgggtggcaccaaacatttattatttaggt

SEQ ID NO: 482 - DNA, 75 bp, Artificial Sequence
  Synthetic oligonucleotide
gagcatgaagcctcaaatcatttgcaagagttaccctatggtggctgggtggcaccaaacatttattatttaggt

SEQ ID NO: 483 - DNA, 75 bp, Artificial Sequence
  Synthetic oligonucleotide
gatcatgaagcctcaaatcatttgcaagagttaccctatggtggctgggtggcaccaaacatttattatttaggt

SEQ ID NO: 484 - DNA, 75 bp, Artificial Sequence
  Synthetic oligonucleotide
gcacatgaagcctcaaatcatttgcaagagttaccctatggtggctgggtggcaccaaacatttattatttaggt

SEQ ID NO: 485 - DNA, 75 bp, Artificial Sequence
  Synthetic oligonucleotide
gcccatgaagcctcaaatcatttgcaagagttaccctatggtggctgggtggcaccaaacatttattatttaggt

SEQ ID NO: 486 - DNA, 75 bp, Artificial Sequence
  Synthetic oligonucleotide
gcgcatgaagcctcaaatcatttgcaagagttaccctatggtggctgggtggcaccaaacatttattatttaggt

SEQ ID NO: 487 - DNA, 75 bp, Artificial Sequence
  Synthetic oligonucleotide
gctcatgaagcctcaaatcatttgcaagagttaccctatggtggctgggtggcaccaaacatttattatttaggt

SEQ ID NO: 488 - DNA, 75 bp, Artificial Sequence
  Synthetic oligonucleotide
ggacatgaagcctcaaatcatttgcaagagttaccctatggtggctgggtggcaccaaacatttattatttaggt

SEQ ID NO: 489 - DNA, 75 bp, Artificial Sequence
  Synthetic oligonucleotide
ggccatgaagcctcaaatcatttgcaagagttaccctatggtggctgggtggcaccaaacatttattatttaggt

SEQ ID NO: 490 - DNA, 75 bp, Artificial Sequence
  Synthetic oligonucleotide
gggcatgaagcctcaaatcatttgcaagagttaccctatggtggctgggtggcaccaaacatttattatttaggt

SEQ ID NO: 491 - DNA, 75 bp, Artificial Sequence
  Synthetic oligonucleotide
ggtcatgaagcctcaaatcatttgcaagagttaccctatggtggctgggtggcaccaaacatttattatttaggt

SEQ ID NO: 492 - DNA, 75 bp, Artificial Sequence
  Synthetic oligonucleotide
gtacatgaagcctcaaatcatttgcaagagttaccctatggtggctgggtggcaccaaacatttattatttaggt

SEQ ID NO: 493 - DNA, 75 bp, Artificial Sequence
  Synthetic oligonucleotide
gtccatgaagcctcaaatcatttgcaagagttaccctatggtggctgggtggcaccaaacatttattatttaggt

SEQ ID NO: 494 - DNA, 75 bp, Artificial Sequence
  Synthetic oligonucleotide
gtgcatgaagcctcaaatcatttgcaagagttaccctatggtggctgggtggcaccaaacatttattatttaggt

SEQ ID NO: 495 - DNA, 75 bp, Artificial Sequence
  Synthetic oligonucleotide
gttcatgaagcctcaaatcatttgcaagagttaccctatggtggctgggtggcaccaaacatttattatttaggt

SEQ ID NO: 496 - DNA, 75 bp, Artificial Sequence
  Synthetic oligonucleotide
taacatgaagcctcaaatcatttgcaagagttaccctatggtggctgggtggcaccaaacatttattatttaggt

SEQ ID NO: 497 - DNA, 75 bp, Artificial Sequence
  Synthetic oligonucleotide
taccatgaagcctcaaatcatttgcaagagttaccctatggtggctgggtggcaccaaacatttattatttaggt

SEQ ID NO: 498 - DNA, 75 bp, Artificial Sequence
  Synthetic oligonucleotide
tagcatgaagcctcaaatcatttgcaagagttaccctatggtggctgggtggcaccaaacatttattatttaggt

SEQ ID NO: 499 - DNA, 75 bp, Artificial Sequence
  Synthetic oligonucleotide
tatcatgaagcctcaaatcatttgcaagagttaccctatggtggctgggtggcaccaaacatttattatttaggt

SEQ ID NO: 500 - DNA, 75 bp, Artificial Sequence
  Synthetic oligonucleotide
tcacatgaagcctcaaatcatttgcaagagttaccctatggtggctgggtggcaccaaacatttattatttaggt

SEQ ID NO: 501 - DNA, 75 bp, Artificial Sequence
  Synthetic oligonucleotide
tcccatgaagcctcaaatcatttgcaagagttaccctatggtggctgggtggcaccaaacatttattatttaggt

SEQ ID NO: 502 - DNA, 75 bp, Artificial Sequence
  Synthetic oligonucleotide
tcgcatgaagcctcaaatcatttgcaagagttaccctatggtggctgggtggcaccaaacatttattatttaggt

SEQ ID NO: 503 - DNA, 75 bp, Artificial Sequence
  Synthetic oligonucleotide
tctcatgaagcctcaaatcatttgcaagagttaccctatggtggctgggtggcaccaaacatttattatttaggt

SEQ ID NO: 504 - DNA, 75 bp, Artificial Sequence
  Synthetic oligonucleotide
tgacatgaagcctcaaatcatttgcaagagttaccctatggtggctgggtggcaccaaacatttattatttaggt

SEQ ID NO: 505 - DNA, 75 bp, Artificial Sequence
  Synthetic oligonucleotide
tgccatgaagcctcaaatcatttgcaagagttaccctatggtggctgggtggcaccaaacatttattatttaggt

SEQ ID NO: 506 - DNA, 75 bp, Artificial Sequence
  Synthetic oligonucleotide
tggcatgaagcctcaaatcatttgcaagagttaccctatggtggctgggtggcaccaaacatttattatttaggt

SEQ ID NO: 507 - DNA, 75 bp, Artificial Sequence
  Synthetic oligonucleotide
tgtcatgaagcctcaaatcatttgcaagagttaccctatggtggctgggtggcaccaaacatttattatttaggt

SEQ ID NO: 508 - DNA, 75 bp, Artificial Sequence
  Synthetic oligonucleotide
ttacatgaagcctcaaatcatttgcaagagttaccctatggtggctgggtggcaccaaacatttattatttaggt

SEQ ID NO: 509 - DNA, 75 bp, Artificial Sequence
  Synthetic oligonucleotide
ttccatgaagcctcaaatcatttgcaagagttaccctatggtggctgggtggcaccaaacatttattatttaggt

SEQ ID NO: 510 - DNA, 75 bp, Artificial Sequence
  Synthetic oligonucleotide
ttgcatgaagcctcaaatcatttgcaagagttaccctatggtggctgggtggcaccaaacatttattatttaggt

SEQ ID NO: 511 - DNA, 75 bp, Artificial Sequence
  Synthetic oligonucleotide
tttcatgaagcctcaaatcatttgcaagagttaccctatggtggctgggtggcaccaaacatttattatttaggt

SEQ ID NO: 512 - DNA, 25 bp, Artificial Sequence
  Synthetic oligonucleotide
atgctgagtttgtgtgtgaacggac

SEQ ID NO: 513 - DNA, 25 bp, Artificial Sequence
  Synthetic oligonucleotide
atgcnnnnnntgtgtggatatccac

(19) United States
(12) Patent Application Publication
(10) Pub. No.: US 2016/0076093 A1
(43) Pub. Date: Mar. 17, 2016

(54) MULTIPLEX HOMOLOGY-DIRECTED REPAIR

(71) Applicant: UNIVERSITY OF WASHINGTON, Seattle, WA (US)

(72) Inventors: Jay Shendure, Seattle, WA (US); Evan Boyle, Seattle, WA (US); Gregory Findlay, Seattle, WA (US)

(21) Appl. No.: 14/818,217

(22) Filed: Aug. 4, 2015

Related U.S. Application Data
(60) Provisional application No. 62/032,734, filed on Aug. 4, 2014.
(60) Provisional application No. 62/046,074, filed on Sep. 4, 2014.

(51) Int. Cl. C12Q 1/68

(52) U.S. Cl. CPC C12Q 1/6874; C12Q 1/6806

(57) ABSTRACT

Disclosed is a method for introducing a plurality of programmed nucleotide modifications into a single locus of a desired genomic DNA sequence in a single experiment. The method entails synthesizing a homology-directed repair (HDR) library comprising a plurality of oligonucleotides, wherein each oligonucleotide comprises a programmed nucleotide modification in the locus of the desired genome, and co-transfecting a population of cells with (i) an expression system capable of expressing Cas9 and a single guide RNA (sgRNA) and (ii) introducing a plurality of programmed nucleotide modifications to the locus of the desired genomic DNA sequence in one or more cells of the population. Also disclosed are methods for analyzing the functional consequence of a genomic mutation and for genomic screening.

### PRIORITY CLAIM

This application claims priority to U.S. Provisional Patent Application No. 62/032,734, filed Aug. 4, 2014, and U.S. Provisional Patent Application No. 62/046,074, filed Sep. 4, 2014, the subject matters of which are hereby incorporated by reference in their entireties as if fully set forth herein.

### STATEMENT REGARDING FEDERALLY SPONSORED RESEARCH OR DEVELOPMENT

This invention was made with government support under Grant No. DP1HG007811, awarded by the National Institutes of Health. The government has certain rights in the invention.

### BACKGROUND

Saturation mutagenesis [1, 2]—coupled to an appropriate biological assay—represents a fundamental means of achieving a high-resolution understanding of regulatory [3] and protein-coding [4] nucleic acid sequences of interest. However, mutagenized sequences introduced in trans on episomes or via random or “safe-harbour” integration fail to capture the native context of the endogenous chromosomal locus [5]. This shortcoming markedly limits the interpretability of the resulting measurements of mutational impact.

Functional consequences of genetic variants are studied by manipulating the endogenous locus, which provides the native chromosomal context with respect to DNA sequence and epigenetic milieu, and for proteins, endogenous levels and patterns of expression [6]. Programmable endonucleases, e.g. zinc-finger nucleases (ZFNs), transcription activator-like effector nucleases (TALENs) or clustered regularly interspaced short palindromic repeat (CRISPR)/Cas-based RNA-guided DNA endonucleases, enable direct genome editing with increasing practicality [7]. However, genome editing has primarily been applied to introduce single changes to one or a few genomic loci [8], rather than many programmed changes to a single genomic locus. There remains a need for a genomic editing method for introducing multiple programmed changes into a single genomic locus in a single experiment.

### SUMMARY

In one aspect, this application relates to a method for introducing a plurality of programmed nucleotide modifications into a single locus of a desired genomic DNA sequence. The method entails the steps of: (a) synthesizing a homology-directed repair (HDR) library comprising a plurality of oligonucleotides, and (b) co-transfecting a population of cells with (i) an expression system capable of expressing Cas9 and a single guide RNA (sgRNA) and (ii) the HDR library, wherein the expression system is capable of introducing the plurality of oligonucleotides having the programmed nucleotide modifications to the locus of the desired genomic DNA sequence in one or more cells of the population. This method is carried out in a single experiment, i.e., in a single culture dish during a series of reactions carried out at the same time or within a single experimental protocol. In step (a), each oligonucleotide includes a programmed nucleotide modification in the locus of the desired genome.

In some embodiments, each programmed nucleotide modification is a single nucleotide variant. In some embodiments, the HDR library is constructed using an oligonucleotide including a degenerate sequence and optionally, a selective PCR site. For example, the degenerate sequence is between 1 and 100 nucleotides in length. In some embodiments, the HDR library contains a set of oligonucleotides having at least 100 unique programmed nucleotide modifications, at least 200 unique programmed nucleotide modifications, at least 300 unique programmed nucleotide modifications, at least 400 unique programmed nucleotide modifications, at least 500 unique programmed nucleotide modifications, at least 600 unique programmed nucleotide modifications, at least 700 unique programmed nucleotide modifications, at least 800 unique programmed nucleotide modifications, at least 900 unique programmed nucleotide modifications, at least 1,000 unique programmed nucleotide modifications, at least 3,000 unique programmed nucleotide modifications, at least 4,000 unique programmed nucleotide modifications, at least 5,000 unique programmed nucleotide modifications, at least 6,000 unique programmed nucleotide modifications, at least 7,000 unique programmed nucleotide modifications, at least 8,000 unique programmed nucleotide modifications, at least 9,000 unique programmed nucleotide modifications, at least 10,000 unique programmed nucleotide modifications, at least 12,000 unique programmed nucleotide modifications, at least 14,000 unique programmed nucleotide modifications, at least 16,000 unique programmed nucleotide modifications, at least 18,000 unique programmed nucleotide modifications, at least 20,000 unique programmed nucleotide modifications, at least 25,000 unique programmed nucleotide modifications, at least 30,000 unique programmed nucleotide modifications, at least 40,000 unique programmed nucleotide modifications, or at least 50,000 unique programmed nucleotide modifications.

In some embodiments, the plurality of programmed nucleotide modifications that are introduced to the locus of the desired genomic DNA sequence results in a saturating set of programmed nucleotide modifications. In some embodiments, the plurality of oligonucleotides are synthesized on a microarray. In other embodiments, the plurality of oligonucleotides are synthesized in column-based synthesis.

In some embodiments, the plurality of oligonucleotides that are synthesized as described above are used directly without any additional amplification or cloning steps. Alternatively, the plurality of oligonucleotides are amplified or cloned cloned to an HDR library before being used to introduce programmed nucleotide modifications.

In some embodiments, the expression system includes a Cas9 expression cassette that includes a nucleotide sequence which encodes a Cas9 nuclease, an sgRNA expression cassette, and a species-specific promoter that is specific to the population of cells.

In certain embodiments, each oligonucleotide of the HDR library includes a pair of homology arms.

In some embodiments, the method for introducing a plurality of programmed nucleotide modifications into a single locus of a desired genomic DNA sequence further entails the steps of: (c) harvesting the population of cells, (d) selectively amplifying a genomic DNA and RNA sample, wherein the edited sequences are amplified and the non-edited sequence are not amplified, and (e) sequencing the genomic DNA and RNA sample that has been selectively amplified, resulting in a set of genomic transcripts which include the plurality of programmed nucleotide modifications. Optionally, the method further entails functionally analyzing the set of genomic transcripts using a functional assay. For example, the functional assay is selected from the group consisting of targeted RNA sequencing to measure transcript abundance, targeted DNA sequencing to measure reduced cellular fitness, targeted chromatin immunoprecipitation-sequencing (CHiP-seq) of co-activators to assay enhancers, increased cellular growth rate to assay cancer drivers or drug resistance, and FACS-based phenotypic sorting for cellular assays.

In another aspect, this application relates to a method for analyzing the functional consequence of a genomic mutation. The method entails the steps of: (a) synthesizing a homology-directed repair (HDR) library including a plurality of oligonucleotides, wherein each oligonucleotide contains a programmed nucleotide modification in the locus of the desired genome, (b) co-transfecting a population of cells with (i) an expression system capable of expressing Cas9 and a single guide RNA (sgRNA) and (ii) the HDR library, wherein the expression system is capable of introducing the plurality of oligonucleotides having the programmed nucleotide modifications to the locus of the desired genomic DNA sequence in one or more cells of the population, (c) harvesting the population of cells, (d) selectively amplifying a genomic DNA and RNA sample, wherein the edited sequences are amplified and the non-edited sequence are not amplified, (e) sequencing the genomic DNA and RNA sample that has been selectively amplified, resulting in a set of genomic transcripts which include the plurality of programmed nucleotide modifications, and (f) functionally analyzing the set of genomic transcripts using a functional assay. This method is carried out in a single experiment, i.e., during a series of reactions carried out at the same time or within a single experimental protocol.

In some embodiments, the HDR library is constructed using an oligonucleotide containing a degenerate sequence and optionally, a selective PCR site. The degenerate sequence is between 1 and 100 nucleotides in length. In some embodiments, the plurality of oligonucleotides are synthesized on a microarray. In other embodiments, the plurality of oligonucleotides are synthesized in column-based synthesis. In some embodiments, the plurality of oligonucleotides are used directly without any cloning step to introduce programmed nucleotide modifications. Alternatively, the plurality of oligonucleotides are cloned to an HDR library before being used to introduce programmed nucleotide modifications.

In some embodiments, the expression system includes a Cas9 expression cassette having a nucleotide sequence which encodes a Cas9 nuclease, an sgRNA expression cassette, and a species promoter that is specific to the population of cells

In some embodiments, each oligonucleotide of the HDR library comprises a pair of homology arms.

In some embodiments, the functional assay is selected from the group consisting of targeted RNA sequencing to measure transcript abundance, targeted DNA sequencing to measure reduced cellular fitness, targeted chromatin immunoprecipitation-sequencing (CHiP-seq) of co-activators to assay enhancers, increased cellular growth rate to assay cancer drivers or drug resistance, and FACS-based phenotypic sorting for cellular assays.

In another aspect, this application relates to a method for genomic screening. The method entails the steps of: (a) introducing a plurality of programmed nucleotide modifications to a single genomic locus, wherein the plurality of programmed nucleotide modifications are introduced in a single experiment, i.e., during a series of reactions carried out at the same time or within a single experimental protocol, (b) sequencing the genomic DNA or cDNA of the edited locus, and (c) quantifying the transcript abundance of each mutation.

In some embodiments, step (a) includes (1) synthesizing a homology-directed repair (HDR) library comprising a plurality of oligonucleotides, wherein each oligonucleotide includes a programmed nucleotide modification in the locus of the desired genome, and (2) co-transfecting a population of cells with (i) an expression system capable of expressing Cas9 and a guide RNA (sgRNA) and (ii) the HDR library, wherein the expression system is capable of introducing the plurality of oligonucleotides having the programmed nucleotide modifications to the locus of the desired genomic DNA sequence in one or more cells of the population. This step is carried out in a single experiment, i.e., during a series of reactions carried out at the same time or within a single experimental protocol. In some embodiments, step (c) includes calculating an enrichment score for each mutation.

### BRIEF DESCRIPTION OF THE DRAWINGS

FIGS. 1a-1c show saturation genome editing and multiplex functional analysis of a hexamer region influencing BRCA1 splicing. FIG. 1a is an experimental schematic. Cultured cells were co-transfected with a single Cas9-sgRNA construct (CRISPR) and a complex homology-directed repair (HDR) library containing an edited exon that harbors a random hexamer (blue, green, orange) and a fixed selective PCR site (red). CRISPR-induced cutting stimulated homologous recombination with the HDR library, inserting mutant exons into the genomes of many cells. At five days post-transfection, cells were harvested for gDNA and RNA. After reverse transcription, selective PCR was performed followed by sequencing of gDNA and cDNA derived amplicons. Hexamer enrichment scores were calculated by dividing cDNA counts normalized by gDNA counts. FIG. 1b shows correlation of enrichment scores between biological replicates for hexamers observed in each experiment with positions of previously identified [9] exonic splicing enhancers (ESEs), exonic splicing silencers (ESSs) and stop codons indicated. FIG. 1c shows rank-ordered plot of enrichment scores with positions of ESEs, ESSs, and stop codons indicated.

FIGS. 2a-2c show that multiplex homology-directed repair reveals effects of single nucleotide variants on transcript abundance. Three separate HDR libraries (R, R2, and L) containing a 3% mutation rate (97% wt, 1% each non-wt base) in either half of BRCA1 exon 18 were introduced to the genome via co-transfection with pCas9-sgBRCA1x18. Enrichment scores were calculated for each haplotype observed at least 10 times in the gDNA, and effect sizes of SNVs were determined by weighted linear regression modeling. ‘Sense’ includes both missense and synonymous SNVs. FIG. 2a shows effect sizes calculated from replicate transfections of HDR library R, consisting of a 3% per-nucleotide mutation rate in the 3′-most 39 bases and the same selective PCR site used in FIG. 1, were highly correlated (R=0.846). FIG. 2b shows that Library R2 harbored a selective PCR site composed of 5 synonymous changes, none of which are present in Library R. When effect sizes derived from experiments with library R2 were plotted against those from library R, there was a strong correlation (R=0.847), indicating reproducibility and demonstrating that differences between selective PCR sites did not strongly influence scores. FIG. 2c shows effect sizes for SNVs across the exon are displayed. Datasets from libraries R and L were combined to span the entire exon. Dashed lines represent SNVs that introduce nonsense codons.

FIGS. 3a-3c show saturation genome editing and multiplex functional analysis at an essential gene, DBR1, in Hap1 cells. An HDR library targeting a highly conserved region of DBR1 exon 2 was used with pCas9-EGFP-sgDbr1x2 to introduce point mutations across 75 base pair (bp) and all possible codon substitutions at three residues believed to participate at the enzyme's active site. FIG. 3a shows that sequencing of gDNA from the HDR library and populations of edited cells at D5, D8, and D11 reveals selection for synonymous mutations, and depletion of frameshift, nonsense, and missense variants. FIG. 3b shows that mean D11 enrichment scores are plotted as line segments for SNVs in the 3′-most 73 bases of exon 2 and two bases of intron 2. Above the enrichment scores in ascending order are the wt nucleotide at each position, each one by genome edit, the wild-type amino acid (AA), and the AA derived from each genome edit (asterisk indicates a stop codon). Segment color indicates mutation type, faded segments indicate discordant effects between replicates, and AAs are colored according to the Lesk color scheme (small nonpolar—orange, hydrophobic—green, polar—magenta, negatively-charged—red, and positively charged—blue). The first nine bases shown correspond to the active site residues. FIG. 3c shows that D8 and D11 amino acid level enrichment scores were calculated for active site residues N84, H85, E86 after excluding discordant observations between replicates (FIG. 13c). On both D8 and D11 strong selective effects and tolerance of only synonymous (green boxes) and a few missense variants were observed.

FIGS. 4a-4d show the distribution and pair-wise correlations of hexamer abundances. FIG. 4a shows the relative abundance of hexamers within the HDR library (top), gDNA (middle), cDNA data (bottom) are shown for a single experiment. The vertical black line represents our threshold of 10 gDNA reads. FIG. 4b-4d demonstrate that scatterplots from a single replicate show pair-wise correlations between sequencing counts for the HDR library, gDNA, and cDNA for hexamers with at least 10 observations in the gDNA library, excluding wild type and control hexamers (n=3,633). The HDR library and the gDNA data are most highly correlated (R 95% confidence interval (CI): 0.596-0.636), followed by the gDNA and cDNA (R 95% CI: 0.419-0.471) and the HDR library and cDNA (R 95% CI: 0.341-0.394).

FIGS. 5a-5d show correlations for hexamer genome editing efficiency and enrichment scores between replicates. FIG. 5a shows that gDNA counts for all hexamers with at least ten reads in each of two gDNA preps from separate transfections with the same HDR library (n=2,980) exhibited moderate correlation (R 95% CI: 0.355-0.416). FIG. 5b shows that hexamer editing rates, defined as gDNA counts normalized to HDR library counts, were substantially less correlated (R 95% CI: 0.084-0.155), consistent with a hexamer's HDR library abundance contributing more to its gDNA abundance than systematic differences in HDR efficiency secondary to the hexamer sequence itself. FIG. 5c shows that hexamer enrichment scores for two pools of cells from a single transfection split on D3 were well-correlated (R 95% CI: 0.643-0.681). FIG. 5d shows that pooling data from cells split on D3 replicates from a single transfection yielded an improved correlation between biological replicates (that is, independent transfections; R 95% CI: 0.690-0.722.

FIGS. 6a-6b show comparison of genome-based hexamer enrichment scores to plasmid-based hexamer scores. FIG. 6a shows that there was a modest correlation between ESS and ESE hexamers defined by a previous study [9] (x-axis) and the enrichment scores calculated here (y-axis; Spearman p=0.524). The previous study also interrogated hexamers positioned +5 to +10 nucleotides relative to a splice junction, but was plasmid-based rather than genome-based and in the context of different exons. FIG. 6B shows that to reveal effects of GC content on hexamer abundance, histograms display the distribution of enrichment scores for each possible G+C level (0-6). Hexamers containing two or fewer G+C base pairs exhibited broadly lower enrichment scores than hexamers containing three or more G+C base pairs.

FIG. 7 shows experimental schematic for genome editing and functional analysis of BRCA1 exon 18. Cultured cells were co-transfected with a single Cas9-sgRNA construct (CRISPR) and an HDR library. Each HDR library was generated from cloning of an oligonucleotide synthesized with 3% nucleotide degeneracy (97WT:1:1:1) for approximately half of the exon and a selective PCR site introduced to the other (fixed) half of the exon (red). CRISPR-induced HDR integrates mutant exons into the genome. Cells were cultured for five days post-transfection, and then harvested for gDNA and total RNA. After reverse transcription, selective PCR was performed before sequencing the edited pools of gDNA and cDNA. Each exon haplotype's enrichment score was measured by dividing cDNA reads by gDNA reads, and effect sizes for each SNV were calculated via weighted linear regression.

FIGS. 8a-8c show positional SNV editing rates and replication of effect sizes. FIG. 8a shows that editing rates for each SNV in BRCA1 exon 18 were calculated by dividing each SNV's gDNA sequencing abundance by its HDR library abundance. Editing rates were then plotted across the exon for each library (red=L, blue=R, green=R2) with locations of their selective PCR sites and the CRISPR-targeted PAM illustrated below. For HDR libraries R and R2, there was a subtle decrease in editing rate with increasing distance from the Cas9 cleavage site (rho^{R}=−0.264, p^{R}=4.1×10⁻³; rho^{R2}=−0.361, p^{R2}=4.8×10⁻⁵). For library L, which allowed re-cutting by not destroying the PAM, there was a sharp peak of editing centered on the Cas9 cleavage site, and a rapid decline in efficiencies in the 5′ direction (further from the 3′ selective PCR handle). FIGS. 8b-8c shows that SNV effect sizes were concordant across biological replicates for libraries R2 (FIG. 8b) and L (FIG. 8c) (library R shown in FIG. 2). Variants of high effect size scored similarly across independent transfections.

FIG. 9 shows biological replicate effect size reproducibility for all libraries. Three separate HDR libraries (R, R2, and L) containing 3% nucleotide degeneracy in either half of BRCA1 exon 18 were introduced to the genome via co-transfection with pCas9-sgBRCA1x18. Enrichment scores were calculated for each haplotype observed at least ten times in the gDNA, and effect sizes of SNVs were determined by weighted linear regression. Effect sizes of individual variants for libraries R2 (left), R (middle), and L (right) were well correlated between biological replicates. Dashed lines represent SNVs that introduce nonsense codons.

FIGS. 10a-10c show correlation between effect sizes and predicted disruption of splicing motifs and indel effects. FIG. 10a shows that MutPred Splice [18] was used to predict the functional impact of all 234 single nucleotide substitutions on splicing in BRCA1 exon 18 (x-axis), and these scores were compared to absolute values of the empirically measured effect sizes (y-axis; p=0.322). Although nonsense variants contributed to this trend, the sense variants with the largest effect sizes generally had high MutPred Splice scores. FIG. 10b shows that for indels observed in gDNA from library 2 (virtually all of which occur at the Cas9 cleavage site), size frequencies are plotted. Indel size=0 includes all haplotypes with wild type length. FIG. 10c shows that for each indel size, enrichment scores were calculated and normalized to that of the average full length exon. As predicted by nonsense-mediated decay, indels that shift the coding frame were associated with low transcript abundance.

FIG. 11 shows experimental schematic for saturation genome editing and multiplex functional analysis of DBR1 exon 2. Hap1 cells were co-transfected with a single Cas9-2A-EGFP-sgRNA construct (CRISPR) and an HDR library cloned from array-synthesized oligonucleotides containing programmed SNVs (orange, blue) and active site codon substitutions (green). The HDR library exon haplotypes also included two synonymous mutations (red) to disrupt PAM and protospacer sequences to prevent Cas9 re-cutting, and a 6 bp selective PCR site (light blue) substituted in the downstream intron. Successfully transfected cells (EGFP+) were selected on D2 by FACS, and cultured. On D5, D8, and D11, samples of cells were taken and selective PCR was performed before targeted sequencing of gDNA. Each haplotype's enrichment score, a measure of the haplotype's fitness in cell culture, was calculated by dividing D8 or D11 abundance by D5 abundance.

FIGS. 12a-12b show DBR1 editing rates by position and comparison of haplotype abundances between D5 and the HDR library, D8, and D11. FIG. 12a shows that editing rates for programmed SNVs represented in the DBR1 gDNA library above threshold (n=216) were calculated by normalizing each SNV's gDNA abundance by its HDR library abundance. Rates are plotted by position, with the locations of the targeted PAM (orange) and selective PCR site (purple) indicated below. The editing rate did not significantly change with position (P>0.05), consistent with positional effects being negated by eliminating re-cutting and performing selective PCR from a distal site. FIG. 12b shows that scatterplots display the frequencies at which each haplotype was observed in the D5 sample vs the HDR library, D8, and D11 samples. To account for bottlenecking from editing of a limited number of cells in this representative experiment, analysis of individual haplotypes was restricted to those present at frequencies above 5×10⁻⁵ in the D5 sample (n=377; represented by the vertical line). Selection was evident by the depletion of many haplotypes in D8 and D11 samples.

FIGS. 13a-13c show performance of computational predictions of deleterious DBR1 mutations and reproducibility between biological replicates. FIG. 13a shows that D11 enrichment scores from a single experiment were used to empirically define deleterious mutations as those with scores fourfold below wild type (vertical line). FIG. 13b shows that three in silico metrics of functional impairment were tested for their ability to anticipate the deleteriousness of these mutations as indicated by the area under the receiver operating characteristic curve (AUC): BLOSUM62 [20] (AUC=0.672, 214 SNVs), PolyPhen-2 [21] (AUC=0.671, 155 non-synonymous SNVs), and CADD [22] (AUC=0.701, 214 SNVs). Despite the different approaches of these algorithms, all three exhibited comparably moderate predictive power. FIG. 13c shows that a biological replicate of the DBR1 experiment was performed and D11 enrichment scores for amino acid substitutions were well correlated (grey lines on scatterplot indicate the ‘deleteriousness’ threshold of fourfold depletion). The distribution of amino acid level enrichment scores for each experiment is displayed along each axis, reflecting bimodality. Unexpected effects (that is, nonsense mutations scoring as tolerated) were among the relatively small percentage of effects not consistent between replicates.

### DETAILED DESCRIPTION

Methods for introducing multiple programmed nucleotide modifications into a single locus of a desired genomic DNA sequence are provided herein. The methods described herein are carried out in a single experiment, i.e., during a series of reactions carried out concurrently within a single experimental protocol in a single culture dish. Such methods may be used to analyze the functional consequence of a genomic mutation or for genomic screening.

To overcome the limitations of previously used methods, a method was developed to generate and functionally analyze hundreds to thousands of programmed genome edits at a single locus in a single experiment. The method allows a more accurate and scalable measurement of the functional consequences of genetic variations. Measurement of the functional consequences of large numbers of mutations with saturation genome editing potentially facilitates high-resolution functional dissection of both cis-regulatory elements and trans-acting factors, as well as the interpretation of variants of uncertain significance observed in clinical sequencing.

According to the embodiments described herein, this application relates to a method for introducing a plurality of programmed nucleotide modifications into a single locus of a desired genomic DNA sequence. Saturation editing of genomic regions may be achieved by coupling CRISPR/Cas9 RNA-guided cleavage [10] with multiplex homology-directed repair (HDR) using a complex library of donor templates. “Saturation editing” as used herein means that for a particular sequence, each nucleotide position of that sequence is systematically modified with each of all four traditional bases, A, T, G and C. For example, a hexamer substituted at each position would have 4,096 possible single nucleotide variants (four possible substitutions at each of the six nucleotide positions of the hexamer, or 4⁶=4,096).

The multiple programmed nucleotide modifications may be introduced during a single experiment. The phrase “a single experiment” means that multiple programmed edits are introduced to a region of a particular size within a single culture dish, during the course of one experiment or a series of reactions within a single experimental protocol. In other words, the programmed edits are introduced concurrently in a single culture dish. In certain embodiments, the single experimental protocol includes one or more concurrent reactions, i.e., the multiple programmed edits are introduced at approximately the same time. This is in contrast to programmed edits being introduced one-by-one or several at a time in physically separated reactions or experiments carried out using one or more multi-well or otherwise separated culture dishes. The region to be introduced with the programmed edits may have an optimal size that allows efficient multiplex editing in one experiment, for example, the region is about between 1 and 100 base pairs in size. The window associated with HDR mechanisms in mammalian cells [11] may affect the size of the region that can be subjected to multiple editing in one experiment. Therefore, it is within the purview of one skilled in the art to determine the size of the region for multiplex editing. By the same token, saturation genome editing of a full gene—e.g. to measure functional consequences of all possible variants of uncertain significance—will likely require multiple experiments tiling along its exons.

The terms “programmed modifications,” “programmed gene edits,” and “programmed edits” as used herein are interchangeable, meaning that for a particular oligonucleotide, one or more nucleotides at a particular position is changed, for example, from A to T, G, or C. In some embodiments, each programmed nucleotide modification or edit is a single nucleotide variant (SNV). The programmed changes may result in a deletion, substitution, insertion, or other type of mutation to the gene.

The method includes a step of synthesizing a homology-directed repair (HDR) library comprising a plurality of oligonucleotides, each of which includes a programmed nucleotide modification. The plurality of oligonucleotides may be synthesized on a microarray or in column-based synthesis. In some embodiments, the HDR library is constructed using an oligonucleotide having a degenerate sequence and optionally, a selective PCR site.

The degenerate sequence may be between 1 and 100 nucleotides in length, and the constructed library using the degenerate sequence may have a set of oligonucleotides having at least 100 unique programmed nucleotide modifications, at least 200 unique programmed nucleotide modifications, at least 300 unique programmed nucleotide modifications, at least 400 unique programmed nucleotide modifications, at least 500 unique programmed nucleotide modifications, at least 600 unique programmed nucleotide modifications, at least 700 unique programmed nucleotide modifications, at least 800 unique programmed nucleotide modifications, at least 900 unique programmed nucleotide modifications, at least 1,000 unique programmed nucleotide modifications, at least 3,000 unique programmed nucleotide modifications, at least 4,000 unique programmed nucleotide modifications, at least 5,000 unique programmed nucleotide modifications, at least 6,000 unique programmed nucleotide modifications, at least 7,000 unique programmed nucleotide modifications, at least 8,000 unique programmed nucleotide modifications, at least 9,000 unique programmed nucleotide modifications, at least 10,000 unique programmed nucleotide modifications, at least 12,000 unique programmed nucleotide modifications, at least 14,000 unique programmed nucleotide modifications, at least 16,000 unique programmed nucleotide modifications, at least 18,000 unique programmed nucleotide modifications, at least 20,000 unique programmed nucleotide modifications, at least 25,000 unique programmed nucleotide modifications, at least 30,000 unique programmed nucleotide modifications, at least 40,000 unique programmed nucleotide modifications, or at least 50,000 unique programmed nucleotide modifications.

The method further includes a step of co-transfecting a population of cells with (i) an expression system capable of expressing Cas9 and a guide RNA (sgRNA) and (ii) the HDR library. In such embodiments, the expression system acts to introduce a plurality of oligonucleotides (each of which includes a programmed nucleotide modification) to the locus of the desired genomic DNA sequence in one or more cells of the population. In certain embodiments, the expression system includes a plasmid which includes a Cas9 expression cassette that includes a nucleotide sequence which encodes a Cas9 nuclease, an sgRNA expression cassette, and a species-specific promoter that is specific to the population of cells. In certain aspects, each oligonucleotide member of the HDR library includes a pair of homology arms in order to target the desired genomic DNA sequence.

The method described herein may further include one or more steps of harvesting the population of cells after culturing the transfected cells, selectively amplifying a genomic DNA and RNA sample wherein the edited sequences are amplified and the non-edited sequence are not amplified, and sequencing the genomic DNA and RNA sample that has been selectively amplified, resulting in a set of genomic transcripts which include the plurality of programmed nucleotide modifications. In some embodiments, the method includes functionally analyzing the set of genomic transcripts using a functional assay, such as targeted RNA sequencing to measure transcript abundance, targeted DNA sequencing to measure reduced cellular fitness, targeted chromatin immunoprecipitation-sequencing (CHiP-seq) of co-activators to assay enhancers, increased cellular growth rate to assay cancer drivers or drug resistance, and FACS-based phenotypic sorting for cellular assays.

In one aspect, this application also relates to analyzing the functional consequence of a genomic mutation by carrying out the above steps, including: (a) synthesizing a homology-directed repair (HDR) library comprising a plurality of oligonucleotides, wherein each oligonucleotide comprises a programmed nucleotide modification in the locus of the desired genome; (b) co-transfecting a population of cells with (i) an expression system capable of expressing Cas9 and a guide RNA (sgRNA) and (ii) the HDR library, wherein the expression system is capable of introducing the plurality of oligonucleotides having the programmed nucleotide modifications to the locus of the desired genomic DNA sequence in one or more cells of the population; (c) harvesting the population of cells; (d) selectively amplifying a genomic DNA and RNA sample, wherein the edited sequences are amplified and the non-edited sequence are not amplified; (e) sequencing the genomic DNA and RNA sample that has been selectively amplified, resulting in a set of genomic transcripts which include the plurality of programmed nucleotide modifications; and (f) functionally analyzing the set of genomic transcripts using a functional assay.

In some embodiments, the functional assay is biologically relevant and technically viable. In some embodiments, the functional assay directly links genotype to phenotype. For example, the functional assay is a targeted RNA sequencing to measure transcript abundance or targeted DNA sequencing to measure reduced cellular fitness. In other embodiments, the functional assay is targeted ChIP-seq of co-activators to assay enhancers, increased cellular growth rate to assay cancer drivers or drug resistance [31], or FACS-based phenotypic sorting for cellular assays [32].

Also described herein is a method for genomic screening. The method includes a first step of introducing a plurality of programmed nucleotide modifications to a single genomic locus, for example, as described above. The genomic screening method further includes sequencing the genomic DNA or cDNA of the edited locus, and quantifying the transcript abundance of each mutation, e.g., by calculating an enrichment score for each mutation.

For illustration purposes, the saturation genome edits were introduced to exon 18 of BRCA1 and to a well-conserved coding region of an essential gene, DBR1, respectively. By no means the scope of this application is limited to these particular genes. It is within the purview of one skilled in the art to introduce genome edits including saturation genome edits to any gene of interest by carrying out the methods disclosed herein.

In exon 18 of BRCA1, a six base-pair (bp) genomic region was replaced with all possible hexamers, or the full exon was replaced with all possible single nucleotide variants (SNVs), and the effects on transcript abundance attributable to nonsense-mediated decay and exonic splicing elements were measured. Saturation genome edits were introduced to DBR1 in a similar fashion and the relative effects on growth that correlate with functional impact were measured.

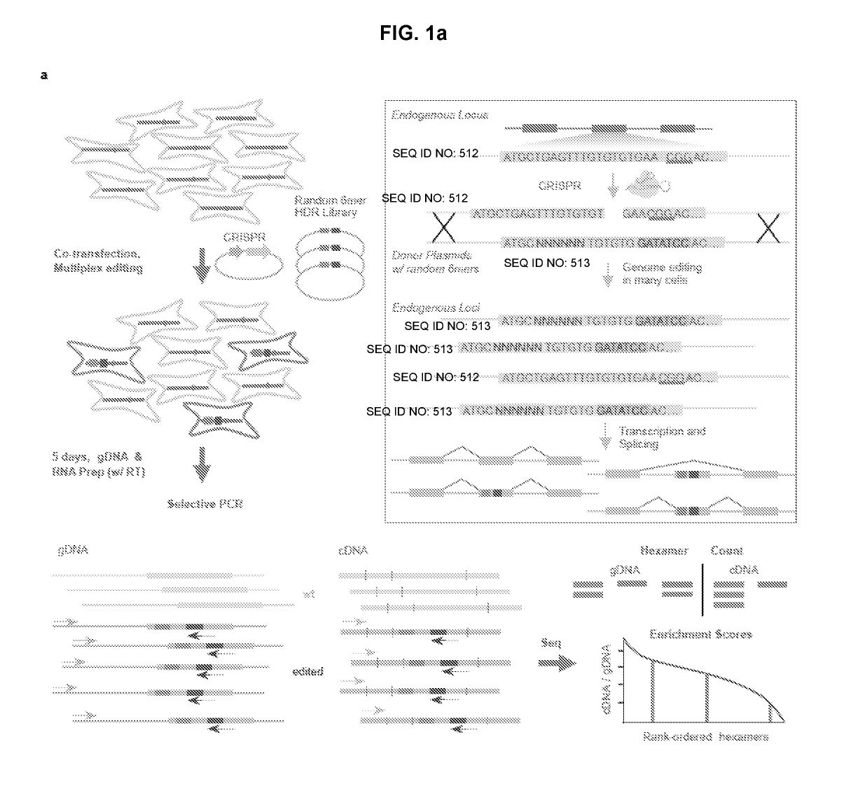

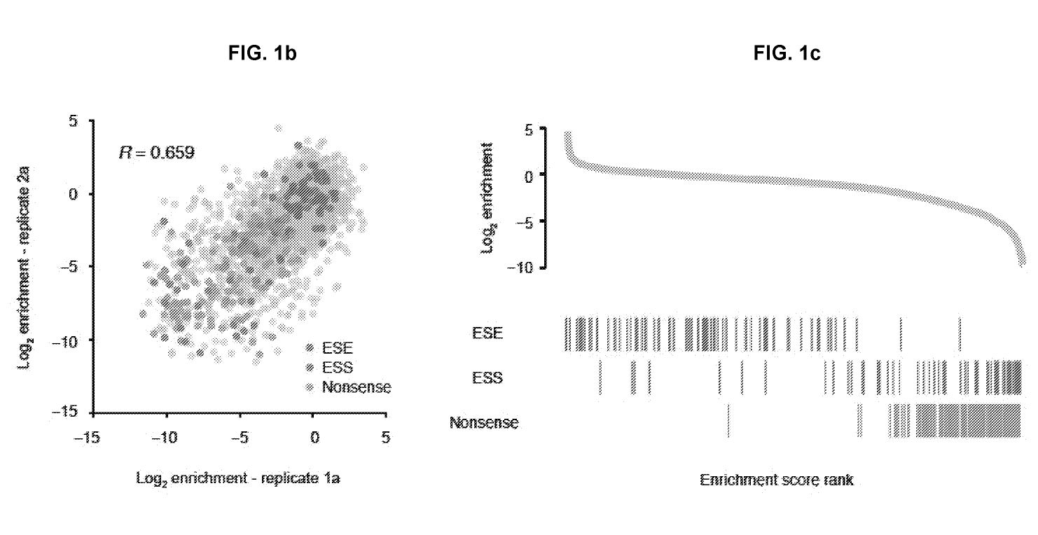

In one embodiment, the methods described herein are exemplified by leveraging CRISPR/Cas9 [10, 12, 13] to introduce saturating sets of programmed edits to a specific locus via multiplex HDR. As illustrated in FIG. 1a, six bases of a BRCA1 exon are targeted [14] by cloning an HDR library containing random hexamers substituted at positions +5 to +10 of BRCA1 exon 18 and fixing nonsynonymous changes at positions +17 to +23 (as a ‘handle’ for selective PCR and to prevent re-cutting [15] by destroying the protospacer adjacent motif (PAM)). The oligonucleotide sequences used are shown in Table 1.

pCas9-sgBRCA1x18 and the HDR library were co-transfected into ˜800,000 HEK293T cells, achieving 3.33% HDR efficiency. Two independent transfections were performed with the same HDR library (biological replicates' 1, 2), and cells were split on day 3 (‘D3 replicates’ a, b).

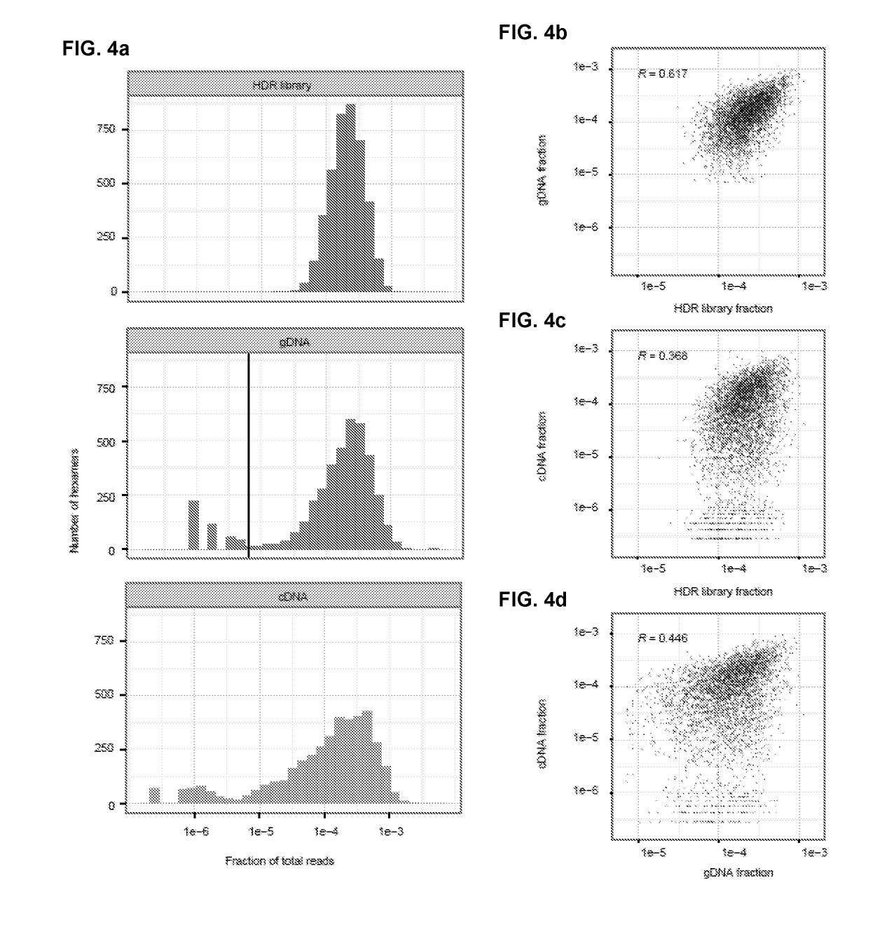

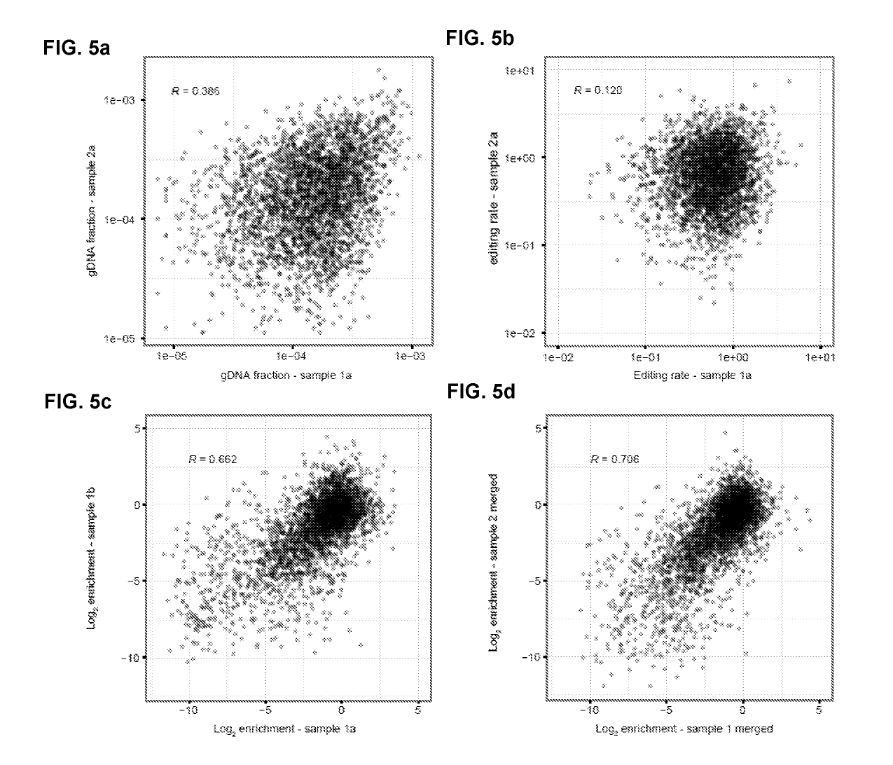

Genomic DNA (gDNA) and cDNA from bulk cells on D5 were prepared. PCR reactions were primed on the ‘handle’ uniquely present within successfully edited genomes. Amplification was observed in HDR library/pCas9-sgBRCA1x18 transfected samples, but not in HDR library-only controls. Amplicons derived from gDNA and cDNA were deeply sequenced (FIG. 1a). The relative abundances of hexamers within replicates and the correlation between the HDR library and edited gDNA were consistent with limited ‘bottlenecking’ during transfection and minimal influence of hexamer identity on HDR efficiency (FIG. 4 and FIG. 5).

The effect of introducing each hexamer to these genomic coordinates on transcript abundance was estimated by calculating enrichment scores (cDNA divided by gDNA counts, calibrated to wild-type). These enrichment scores were well correlated between biological replicates (FIG. 1b, 1a vs. 2a: R=0.659) and between D3 replicates (FIG. 5c; 1a vs. 1b: R=0.662). Correlation between biological replicates improved when read counts were pooled from D3 replicates (FIG. 5d; 1 vs. 2: R=0.706).

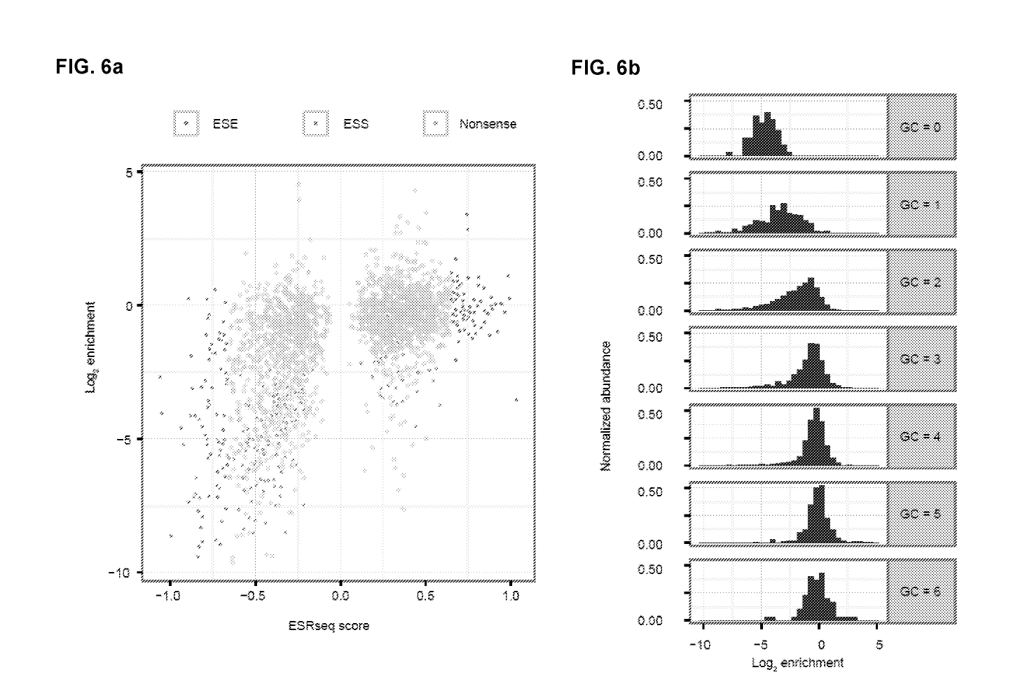

To maximize precision, data across all four replicates for 4,048 hexamers were merged (FIG. 1c; Table 2). Several results support the biological validity of the resulting enrichment scores. First, as predicated by nonsense-mediated decay (NMD), hexamers introducing stop codons were associated with markedly reduced mRNA levels (FIG. 1c; Wilcoxon rank sum test (WRST) P=9.7×10-84; median for nonsense hexamers 12-fold below overall median). Second, previous studies measured hexamer influence on splicing at analogous coordinates of different exons via a plasmid minigene assay [9]. Despite these contextual differences, the strongest exonic splicing silencers (ESSs) (bottom 2% in ref [9]) scored 9-fold below median (FIG. 1c; WRST P=2.0×10-24), the strongest exonic splicing enhancers (ESEs) (top 2% in ref 14) scored 1.5-fold above median (FIG. 1c; WRST P=2.4×10-11), and the complete datasets correlated reasonably well (FIG. 6a; p=0.524). Correlation between GC content and enrichment scores was also observed (FIG. 6b), strongest for bases most proximal to the splice junction, consistent with a posited role for GC content in the stability of splicing structures [16] (although reverse transcription bias is a potential confounder).

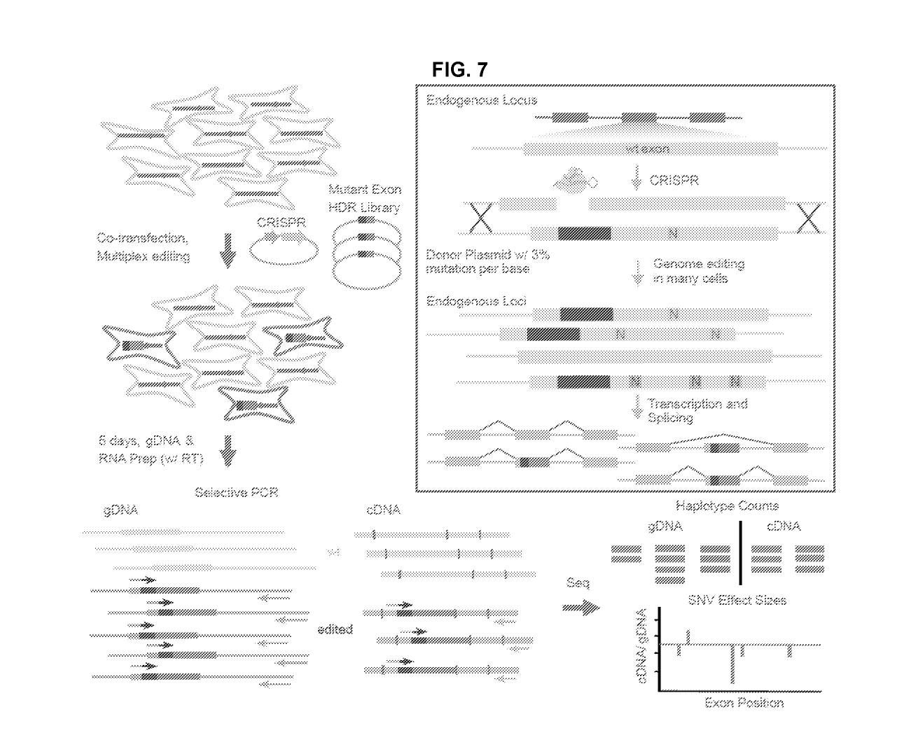

In some embodiments, FIG. 7 illustrates the assay of the effects of SNVs across the full 78 bp BRCA1 exon 18. Three HDR libraries were cloned with selective PCR sites in either the 5′ or 3′ region and 3% doping [17] (97 (wt):1:1:1) in the other half of the exon (L: 5′ degeneracy, 3′ nonsynonymous selective PCR site; R: 3′ degeneracy, 5′ nonsynonymous selective PCR site; R2: 3′ degeneracy, 5′ synonymous selective PCR site) (Table 1). Five days post-transfection with pCas9-sgBRCA1x18 (1.02-1.29% HDR efficiency), gDNA and cDNA were selectively amplified and deeply sequenced.

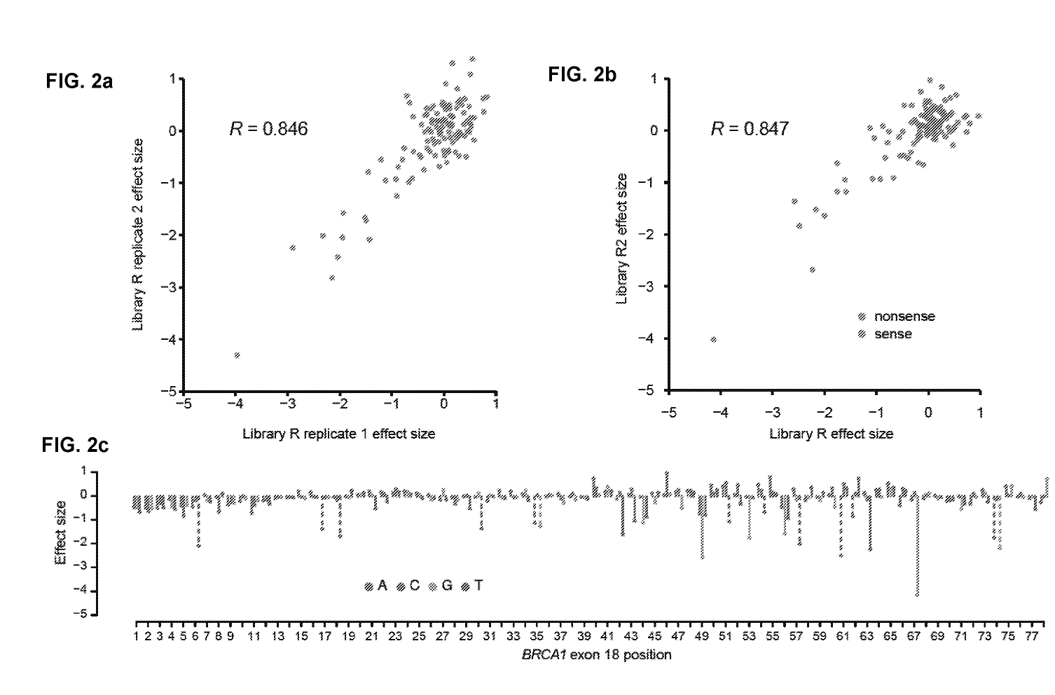

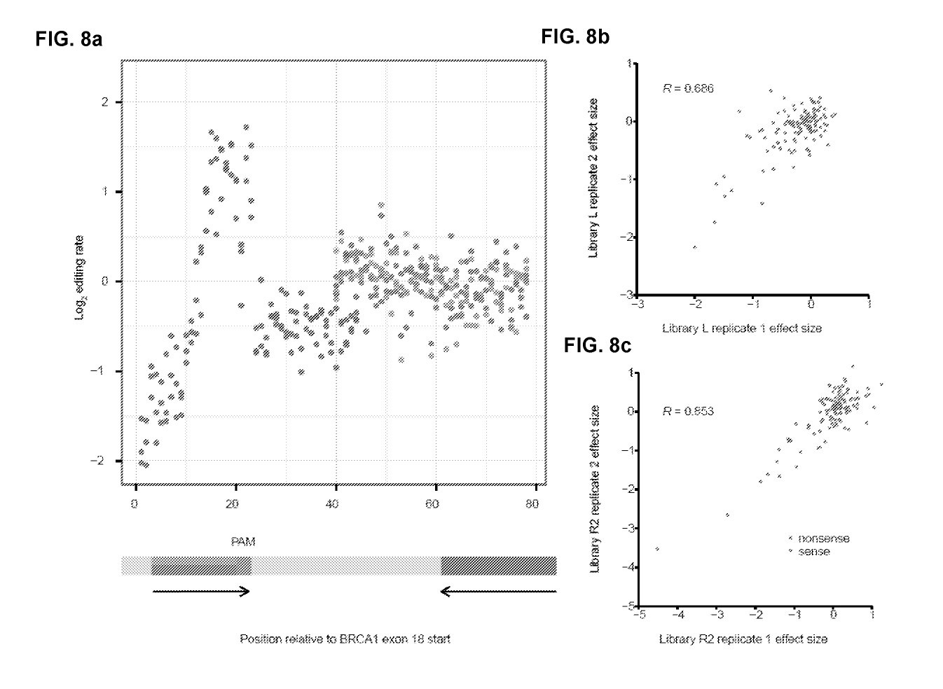

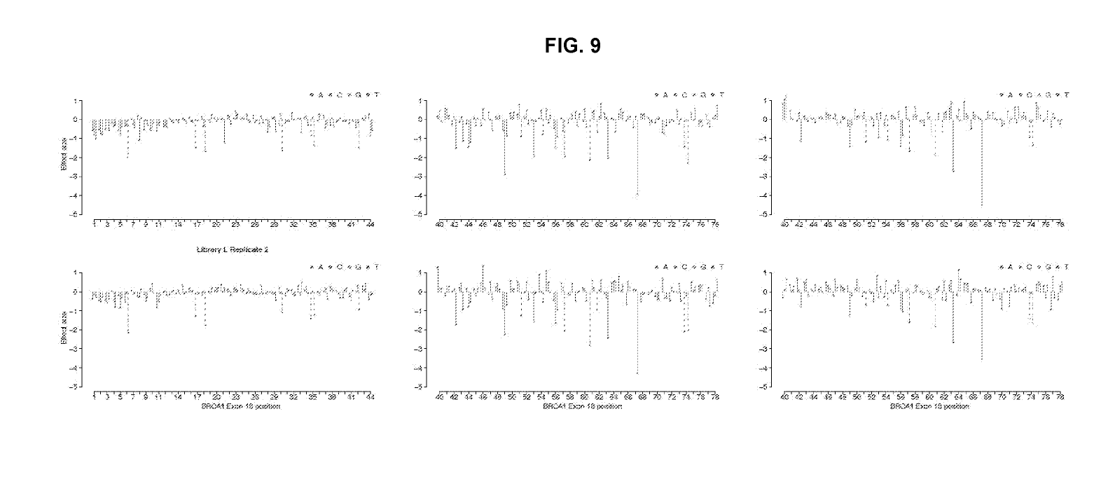

Using data from all edited exons with ≧1 mutation and ≧10 gDNA counts, effect sizes (beta values) of all possible SNVs were estimated using a weighted linear model. Estimated effect sizes were reproducible (R=0.846 (R), 0.853 (R2), and 0.686 (L); FIG. 2a, FIGS. 8-9, Table 3). Effect sizes for the same SNVs interrogated with different selective PCR strategies (R vs. R2) were also well correlated (R=0.847; FIG. 2b).

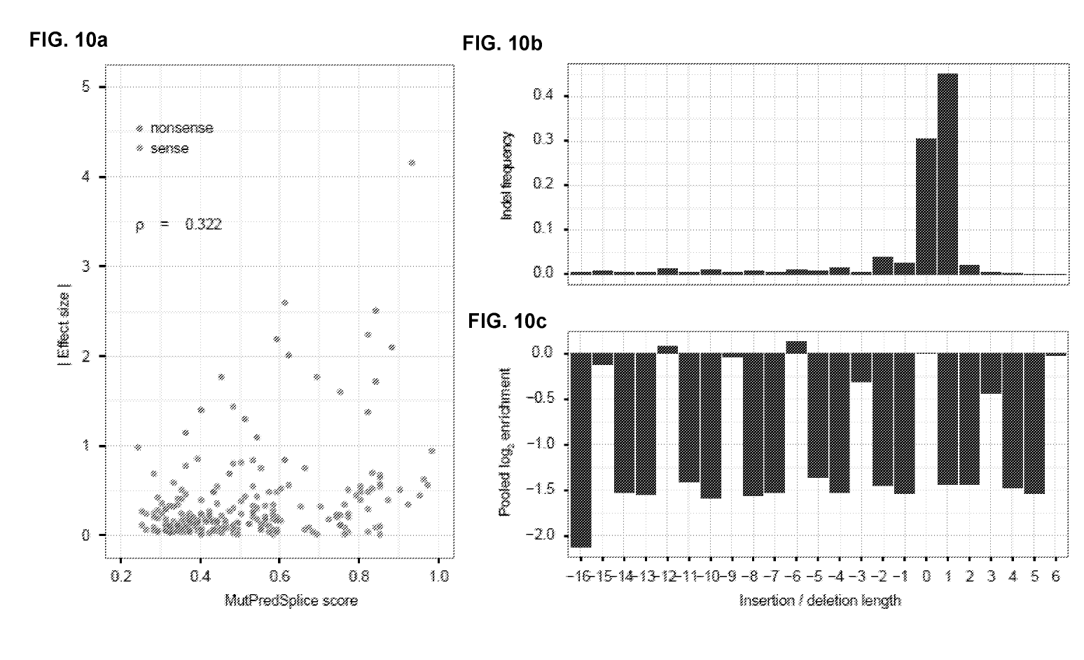

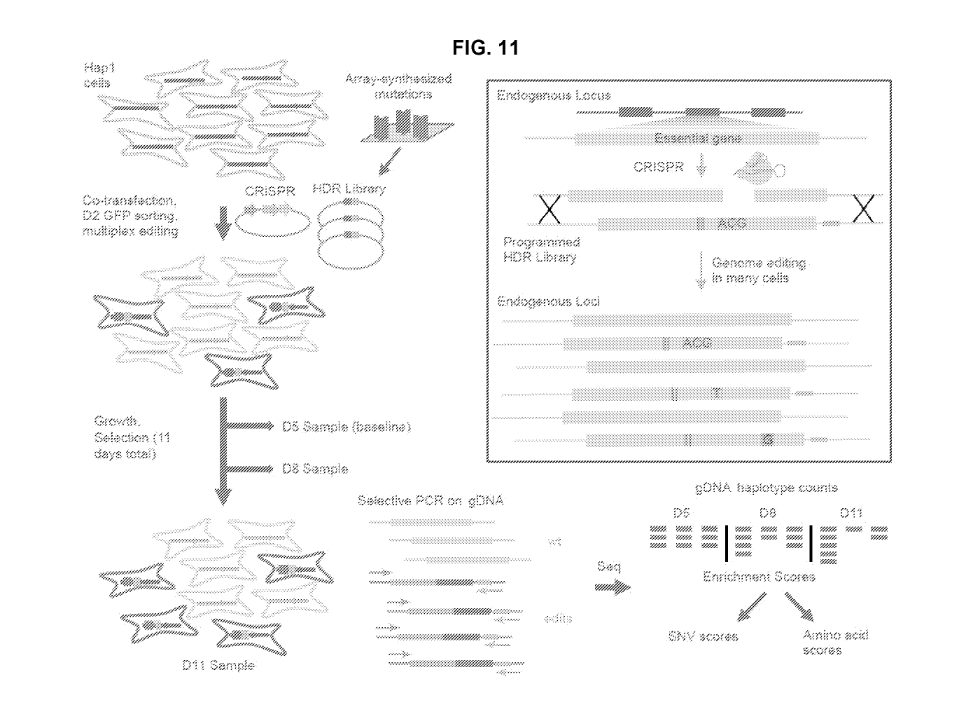

The estimated effect sizes reflect empirically measured changes in transcript abundance resulting from programmed edits (FIG. 2c). As predicted with NMD, nonsense mutations reduced transcript abundance (WRST P=1.4×10−203; 5.6−fold below median). Additionally, several missense and synonymous SNVs reproducibly resulted in large reductions in transcript abundance, and SNV effect sizes correlated with a predictive model for exonic variants that disrupt splicing [18] (p=0.322; FIG. 10a). Because library L does not destroy the PAM, enrichment scores for indels were calculated from non-homologous end-joining (NHEJ). As predicted with NMD, only frameshifting indels were associated with large depletions (FIGS. 10b, 10c).

In another embodiment, FIG. 11 illustrates targeting a well-conserved region of DBR1, the RNA lariat debranching enzyme, which scored highly in a genome-wide screen for essentiality [19]. Array-synthesized oligonucleotides were used to program a DBR1 HDR library to include the wild-type sequence and every possible SNV across 75 bp (73 3′-most bases of exon 2 and first two bases of intron 2), and also all 63 possible codon substitutions at three residues (388 genome edits were programmed; single base deletions were abundant from synthesis errors). The HDR library also introduced two fixed synonymous changes (to disrupt the PAM and prevent re-cutting [15]) and a selective PCR site in intron 2.

An optimized single guide RNA (sgRNA) sequence [23, 24] was cloned into a bicistronic sgRNA/Cas9-2A-EGFP vector (pCas9-EGFP-sgDbr1x2). Five million haploid human cells [25] (Hap1) were co-transfected with the DBR1 HDR library and pCas9-EGFP-sgDbr1x2. On D2, ˜250,000 EGFP+ cells were FACS sorted and further cultured, taking samples on D5, D8 and D11 (1.14% HDR efficiency, estimated on D8). Following gDNA isolation and selective PCR, deep sequencing was performed to quantify the relative abundance of edited haplotypes in each sample.

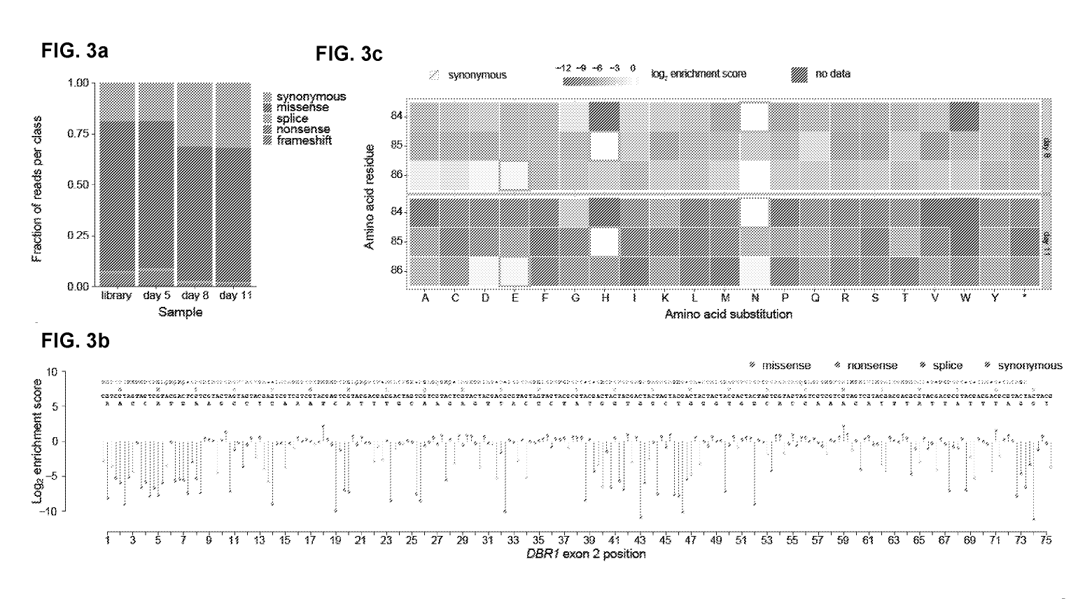

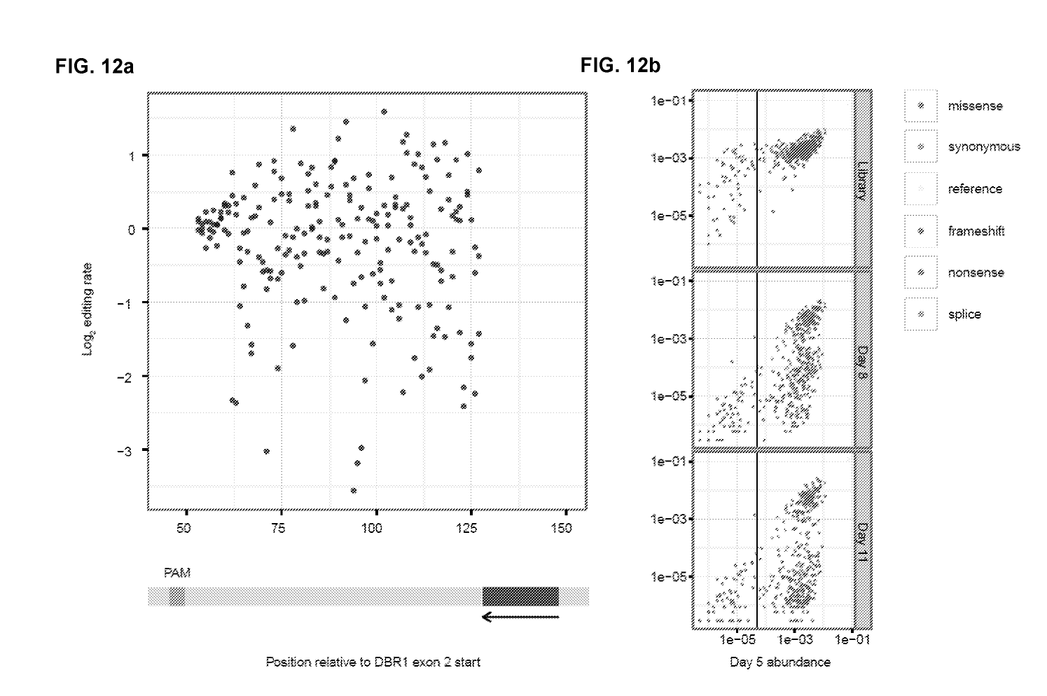

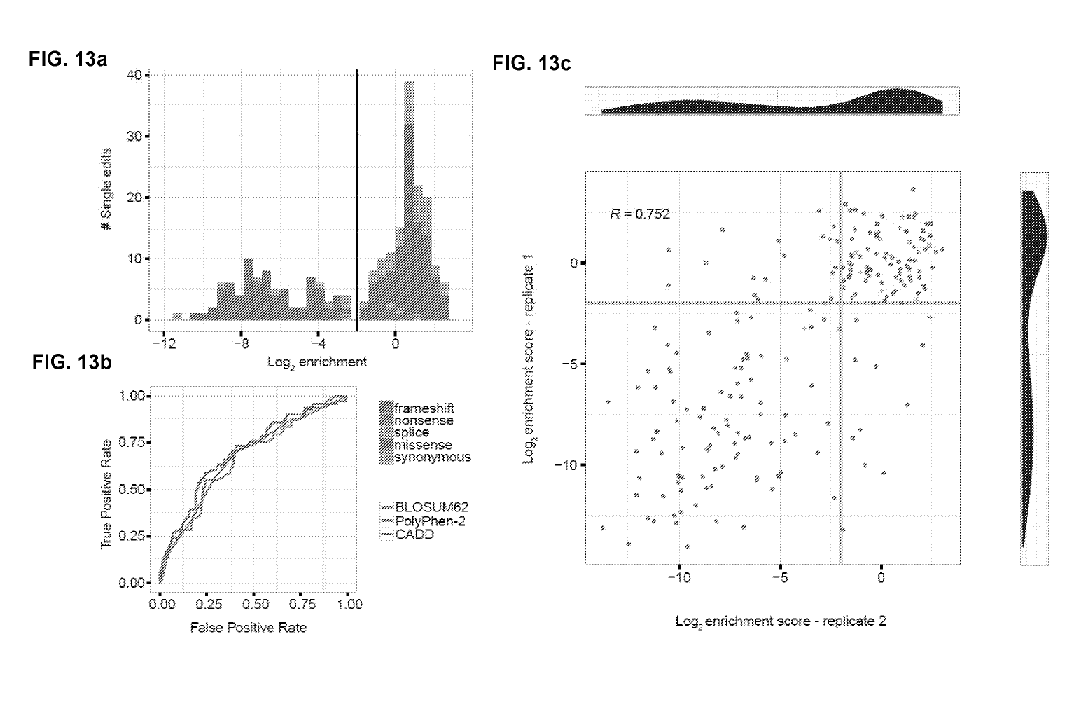

The relative proportions of mutation classes at each time point were first examined (FIG. 3a). The strong enrichment of synonymous mutations and depletion of nonsense and frameshifting mutations over time indicated that selection was acting on edited cells in culture, consistent with DBR1 essentiality. The enrichment scores (D8 or D11 counts divided by D5 counts) for 365 of the 388 (94%) programmed edits and 12 single base deletions (the subset with relative abundance >5×10-5 on D5) were calculated (FIG. 3b; FIG. 12; Table 4). Enrichment scores strongly correlated with functional consequence. The median enrichment score for synonymous edits was nearly identical to wild-type (1.006-fold lower), but 73-fold lower for missense edits (P=1.7×10-8; WRST against synonymous edits), 207-fold lower for nonsense edits (P=1.9×10-9), and 211-fold lower for frameshifting single base deletion edits (P=1.5×10-8). Furthermore, enrichment scores for SNVs were inversely correlated with metrics of predicted deleteriousness like CADD [22] (p=−0.295; P=1.2×10-5; FIGS. 13a, 13b). Residues N84, H85 and E86 of DBR1 were edited to all 63 possible non-wild-type codons. Consistent with their predicted role in the active site of an essential enzyme [26], only synonymous mutations and a few missense substitutions were tolerated (FIG. 3c).

Amino-acid level enrichment scores were well correlated between D11 biological replicates (R=0.752; P=2.6×10-40; FIG. 13c), and were bimodally distributed in each replicate, allowing broad classification of changes as tolerated or deleterious. The small proportion of discordantly classified variants might be explained by Hap1 reversion to diploidy or off-target effects, highlighting the importance of biological replicates for this experimental design. There were no reproducibly tolerated nonsense or frameshifting edits. These data indicate that the empirically derived enrichment scores reflect true biological effects of specific genomic point mutations within DBR1.

Genome editing efficiency may be affected by factors such as bottlenecking complexity, limiting reproducibility and in some cases, necessitating the optional selective PCR sites. However, selective PCR sites are not necessarily required in all cases. In some embodiments, a variety of techniques, e.g. transient hypothermia [27] or oligonucleotide-based HDR [28], can be used to improve editing efficiency. In some embodiments, ZFNs and TALENs may improve efficiencies up to 50% [29, 30].

In some embodiments, haploid cells for DBR1 mutagenesis can be used to improve editing efficiency. In other embodiments, mutagenesis can be performed in diploid cells by knocking out one allele via NHEJ and then knocking in the HDR library to the other allele.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entireties, as if fully set forth herein.

### Examples

### BRCA1 Experimental Design

An exon in a clinically relevant gene in which known mutations cause aberrant splicing was chosen to be targeted. Previous molecular studies of a G to T nonsense mutation occurring naturally in cancer patients at chr17:41,215,963 suggested exon skipping [14] was secondary to the creation of an exonic splicing silencer site [33]. It is hypothesized that saturation genome editing of this exon could result in a wide range of splicing outcomes.

When performing parallel functional analysis of complex allelic series, how to associate each of many mutations with the biological effects they produce should be considered. It is more difficult when attempting such approaches at the endogenous genomic locus, and with limited editing efficiencies. By performing these experiments in an exon and focusing on the effects of mutations on transcript abundance, genotype and phenotype are directly linked by observing the frequency of each genome edit in the transcript pool, relative to its frequency in genomic DNA. This design is advantageous because it requires no specialized (i.e. gene-specific) functional assay, thus making it amenable to interrogation of transcribed variants' effects on splicing/transcript abundance in any gene.

Inclusion of Selective PCR Sites

Given the modest proportion of HDR-edited loci in a given experiment and the high number of variants to be interrogated (i.e. hundreds to thousands), it would require a large amount of sequencing to sufficiently sample every variant in gDNA and cDNA pools from a population of cells that are predominantly unedited or harboring products of NHEJ. Furthermore, at such efficiencies, the rate of error in high-throughput sequencing is high enough to obscure signal from single nucleotide variants (SNVs) (unpublished observations). Therefore, in certain embodiments, until better methods are developed, techniques to selectively sequence molecules derived from edited cells are likely to be advantageous to isolate populations of cells that have been successfully edited with HDR techniques. In some embodiments, selective PCR sites are present regardless how the HDR libraries are generated, for example, by degenerate oligonucleotide or by programmed edits via microarray-based synthesis. In other embodiments, selective PCR sites are not used.

The HDR libraries were designed to include short, fixed edits to serve as unique priming sites in genomes that successfully undergo HDR. PCR reactions primed at this site, therefore, should only amplify material from edited cells, thus reducing both the noise associated with error from sequencing unedited material and the cost of sequencing in each experiment. Additionally, selective PCR sites that mutate the PAM and protospacer sequences could prevent Cas9 from re-cutting HDR-edited genomes. This should have the effect of increasing the proportion of cells bearing experimentally informative edits, and given the bottleneck imposed by limitations on how many successfully edited cells can be sampled, should result in more robust experimental signal.

DBR1 Experimental Design

To demonstrate that saturation genome editing can be used to explore effects of mutations on protein function and cellular fitness, DBR1, a well-conserved gene that scored highly in a human haploid cell genome-wide loss-of-function screen for essentiality [19] was targeted. Using haploid cells prevents gene compensation from an unedited copy [25]. Without knowing how sensitive the cells would be to mutations, it was chosen to target a region of exon 2 that was highly conserved, included in all transcript annotations on the UCSC Genome Browser, and coded for at least 2 residues (N84, H85) predicted to participate at the enzyme's active site [26]. Selection against edited cells in culture allows phenotype to be linked to genotype from sequencing of the gDNA pool over a series of time points. During HDR library construction, a selective PCR site in a downstream intron was designed to minimize any effect on gene function, and two synonymous mutations to abrogate Cas9 re-cutting were used.

Given the lower transfection efficiency of Hap1 cells (˜4% for the plasmids used here), a DBR1-targeting CRISPR construct that expressed EGFP with Cas9 was cloned and FACS was used to sort a population of successfully transfected cells. The sgRNA was designed using the Zhang Lab tool [described at http://crispr.mit.edu/], and selected to minimize off-target effects that could potentially impair cellular fitness [23].

HDR Library and Cas9-sgRNA Cloning

A homology-directed repair (HDR) library containing all possible 4,096 DNA hexamers substituted at positions +5 to +10 of BRCA1 exon 18 (chr17:41,215,962-41,215,967; CCDS11453.1) was constructed using a partially degenerate oligonucleotide (IDT DNA; “BRCA1ex18NNNNNN5_{—}10 selPCR”) containing a 7 bp selective PCR site/EcoRV restriction digest site at position +17 to +23 (FIG. 1a, Table 1). The oligonucleotide was PCR amplified and cloned via the In-Fusion reaction (Clontech) into a PCR-linearized pUC19-BRCA1ex18 vector containing a pre-inserted 1,573 bp fragment amplified from the surrounding BRCA1ex18 locus in HEK293T cells (chr17:41,215,127-41,216,699) to serve as homologous arms. Additional libraries from a second degenerate oligonucleotide that was synthesized with a 3% mutation rate (97% wt, 1% each non-wt base) across the 78 bp exon were cloned similarly, such that one end of the exon would be fixed and contain either missense (as above) or synonymous mutations for selective PCR. Complete oligonucleotide and HDR library exon sequences are listed in Table 1. All PCR reactions were performed with the KAPA HiFi HotStart ReadyMix PCR Kit.

The DBR1 HDR library was cloned as above except with the following differences. HDR library variants were derived from 388 oligonucleotides synthesized on a microarray (CustomArray) to include all possible single base pair changes in a 75 bp region comprising part of DBR1 exon 2 (chr3:137,892,342-137,892,416), all codon variants at the first three residues of the 75 bp region (chr3:137,892,408-137,892,416), and the reference 75 bp sequence. All DBR1 HDR library sequences also included two synonymous mutations designed to prevent re-cutting of edited genomes by disrupting PAM and protospacer sequences (chr3:137,892,424 and chr3:137,892,421), and a 6 bp selective PCR site in intron 2 of DBR1 (chr3:137,892,331-137,892,336). The library was cloned into a pUC19-DBR1ex2 backbone, a vector containing the surrounding DBR1 sequence cloned from Hap1 gDNA (chr3:137,891,573-137,893,293).

A bicistronic Cas9-sgRNA vector designed to cleave within BRCA1 exon 18 (“pCas9-sgBRCA1x18”) was cloned according to a published protocol[24] by ligating annealed oligonucleotides into a human codon-optimized S. pyogenous Cas9-sgRNA vector from the lab of Feng Zhang (pX330-U6-Chimeric_BB-CBh-hSpCas9; Addgene plasmid #42230). The same protocol was followed to create pCas9-EGFP-sgDbr1x2 from a similar Zhang lab vector that allows for fluorescent identification of Cas9-expressing cells (pSpCas9(BB)-2A-GFP (pX458); Addgene plasmid #48138).

Cell Culture and Transfection

For BRCA1 experiments, HEK293T cells were cultured in Dulbecco's Modified Eagle Medium (Life Technologies) supplemented with 10% FBS (AATC) and 100 U/ml penicillin+100 ug/ml streptomycin (Life Technologies). One day prior to transfection, cells were split to ˜40% confluency in 12-well plates with antibiotic-free media. The next day, 0.5-1.0 ug of each library was co-transfected (Lipofectamine 2000, Invitrogen) with an equivalent amount of pCas9-sgBRCA1x18. Cells were expanded to 6-well plates, then split 1:4 on day 3 into two pools, and DNA and RNA were harvested on D5 (AllPrep DNA/RNA Mini Kit, Qiagen). Biological replicates of each transfection and negative control transfections of each library without pCas9-sgBRCA1x18 were also performed.

For the DBR1 experiment, Hap1 cells (Haplogen) were cultured in Iscove's Modified Dulbecco's Medium supplemented with 10% FBS and 100 U/ml penicillin+100 ug/ml streptomycin. ˜3×10⁶ Hap1 cells were passaged to a 60 mm dish in antibiotic-free media one day prior to co-transfection with 3 ug each of pCas9-EGFP-sgDbr1x2 and the DBR1 HDR library via Turbofectin 8.0 (OriGene) according to protocol. On D2, FACS was performed (BD FACSAria III) to isolate ˜250,000 EGFP+ cells, which were then expanded in culture with samples taken of ˜1×10⁶ cells on D5, and 4-8×10⁶ on D8 and D11. gDNA was isolated according to protocol with the QiaAmp Kit (Qiagen). A biological replicate was performed, as well as negative controls in which the HDR library was transfected with the empty pSpCas9(BB)-2A-GFP construct (to enable FACS of transfected cells without editing).

RT, Selective PCR and Sequencing

For BRCA1 experiments, reverse transcription (RT) was performed using SuperScriptIII (Invitrogen) with a gene-specific primer located in either BRCA1 exon 19 (hexamer experiments) or exon 21 (whole exon experiments). Initial rounds of PCR were performed on large quantities of sample gDNA (8-12 ug gDNA, 100-150 ng/reaction) and cDNA (25 ug total RNA reverse transcribed and split into 45-47 reactions) using the KAPA HiFi HotStart ReadyMix PCR kit. In the first gDNA PCR, a primer external to the HDR library was used to prevent amplification of plasmid DNA. cDNA reactions were either primed from exons 16 and 18 (hexamer experiment; Library L) or exons 18 and 20 (Libraries R, R2). After the initial gDNA and cDNA reactions, all PCR products from a single sample were pooled and purified using the QIAquick PCR Purification Kit (Qiagen).

For both cDNA and gDNA reactions, a primer designed to selectively amplify edited molecules bearing the selective PCR site was used either in the first or second reaction. Optimal annealing temperatures for each primer pair were determined via gradient PCR, and negative control reactions were performed using input from HDR library-only transfections to ensure products were derived from edited genomes as opposed to the HDR library. Negative controls failed to amplify for all experiments. Two subsequent PCRs were performed to add sequencing adaptors (“PU1L” and “PU1R”), sample indices, and flow cell adaptors.

For the DBR1 experiment, 30 cycles of selective PCR were performed on gDNA (300 ng per reaction) from D5 (3 ug), D8 and D11 (27 ug each). Wells from each sample were pooled, PCR purified, and then re-amplified for 15 additional cycles. The 1,055 bp product was gel-purified (QIAquick Gel Extraction Kit, Qiagen), and two subsequent PCRs were performed to incorporate sequencing and flow cell adaptors prior to sequencing as above.

After final reactions were purified (AMPure XP beads, Agencourt), paired-end sequencing was performed on all samples with the Illumina MiSeq to quantify gDNA and/or cDNA abundances for each edited haplotype. All primer sequences for RT, selective PCR, and sequencing library preparation are provided in Table 1.

HDR efficiencies were estimated for all experiments via deep sequencing of target loci by performing PCR on 150-300 ng of gDNA using primers external to the region of editing and the selective PCR site. Reported HDR efficiencies were conservatively calculated as the fraction of sequencing reads containing the selective PCR site and bearing at least one variant represented in the HDR library.

Analysis of Sequencing Data

For quality control, fully overlapping paired-end reads were merged with PEAR [34] (Paired-End reAd mergeR) and discordant pairs were eliminated. By design, the mutagenized region is covered by both the forward and reverse reads on the Illumina platform, resulting in high-confidence calls per site.

For BRCA1 hexamer reads to be included, the six bases on either side of the hexamer were required to match the reference sequence, and every base call in the hexamer required a quality score of at least Q30. For BRCA1 whole-exon mutagenesis, the full read was required to be the correct length and match the library consensus sequence outside of the mutagenized region, every base quality score inside the mutagenized region was required to be at least Q30, and no indels were tolerated in alignment with BWA-MEM [35]. cDNA reads not matching any gDNA haplotype with at least 10 reads were eliminated. After normalizing for sequencing coverage, enrichment scores were calculated as cDNA read counts incremented by one pseudocount divided by gDNA reads, calibrated to the wild-type hexamer.

For DBR1 mutagenesis, reads were subjected to the same requirements of the sequence outside the mutagenized bases matching the consensus and every quality score in the mutagenized region exceeding Q30. Only reads matching programmed haplotypes were analyzed, and haplotypes below a D5 relative abundance of 5E-5 were excluded from analysis. After incrementing all read counts by one pseudocount and dividing by the total number of reads, the abundance of each haplotype on D8 or D11 was divided by the corresponding abundance on D5, and the fold change relative to the wild type sequence was taken to calculate an enrichment score. Based on the bimodal distribution observed in each replicate, mutations with log₂-transformed enrichment scores less than −2 were considered “deleterious”; otherwise, mutations were considered “tolerated”. Discordant effects between replicates were defined as mutations “tolerated” in one replicate but “deleterious” in the other. Amino acid level enrichment scores were calculated as the median of SNV enrichment scores for programmed edits resulting in the same change (or lack of change, for synonymous edits).

SNV Effect Size Linear Modeling and Replicate Pooling

To determine effects of SNVs in the BRCA1 whole-exon experiments, cDNA and gDNA read counts were converted into percentages (number of reads for a given haplotype divided by the total number of reads for a given replicate) after discarding haplotypes with fewer than 10 gDNA reads. Because we had variance in the number of reads for each haplotype, the null expectation of equal variance (σ2) for each cDNA/gDNA ratio was violated. Because each effect size (yij) was the average of nij observations (reads), then var(yij)=var εij=σ2/nij, suggesting that the weight for each variable should be nij. To predict single nucleotide effect size across exon 18 of BRCA1, we then fit the weighted linear model:

yᵢⱼ=β₀+wᵢⱼβᵢⱼXᵢⱼ

where yᵢⱼ is the log₂ enrichment score for a given haplotype, is the number of gDNA reads for a given haplotype, βᵢⱼ is the effect of nucleotide i at position j relative to the wild-type allele, and Xᵢⱼ is a dummy variable indicating the presence or absence of a particular nucleotide change i at position j relative to the wild type allele. Regression analyses were performed in R 3.0.0 using the lm( ) function. The resulting coefficients of the model adjusted for the model intercepts (β₀+βᵢⱼ) were taken as effect sizes of the individual SNVs on exon splicing/stability. To merge data across replicates, effect sizes were averaged (including across overlapping bases between libraries L and R in BRCA1 exon).

Comparisons to Other Metrics of Functional Impact

For comparison to plasmid studies, ESR-seq scores were taken from Ke et al. (2011) [9]. Hexamers with positive ESR-seq scores are deemed exonic splicing enhancers, whereas negative ESR-seq scores denote exonic splicing silencers. For comparison of BRCA1 exon 18's SNV effect sizes to an in silico method, all SNVs were queried on MutPredSplice's web server (http://mutdb.org/mutpredsplice/submit.htm). MutPredSplice reports a single score estimating the likelihood that a variant will disrupt splicing at any genomic locus. Absolute values of BRCA1 exon 18 splicing effect sizes were then correlated with MutPredSplice scores to determine concordance between our data and predicted effects on splicing.

For DBR1, calculated enrichment scores were compared to BLOSUM62 substitution scores [20] (obtained from NCBI), PolyPhen-2 [21], and CADD [22] (PolyPhen-2 and CADD scores obtained from querying genomic coordinates from CADD's precomputed genomic annotations (http://cadd.gs.washington.edu/download). Whereas BLOSUM62 is derived from evolutionary conservation and PolyPhen-2 predicts changes in protein function, CADD is an integrated measure of deleteriousness that incorporates many functional annotations (including PolyPhen-2).

### Discussion

Reproducibility of Saturation Genome Editing Experiments

The correlations between replicates for each of the experiments suggest that while this technique reproducibly measures effects of many concurrent programmed genome edits, there are also sources of noise.

The noise observed may relate to the fact that modest editing efficiencies lead to relatively few cells in each experiment harboring each specific edit. In the BRCA1 hexamer experiment and the DBR1 experiment, a bimodal distribution of gDNA read counts is observed (FIGS. 4a, 12b). This is consistent with a bottleneck resulting in not all HDR library edits being present in post-editing gDNA. If some edits are not present at all, some edits may be only made once or a few times, and may be represented by very few cells when measurements are made, possibly as few as one.

Consistent with lowly sampled edits being more prone to noise, hexamers that are more highly represented in gDNA counts are more reproducible. For example, whereas R=0.659 between two biological replicates overall, hexamers falling into the top third with respect to gDNA count correlated much more highly (R=0.857). Furthermore, considering the two BRCA1 experiments, because there were far fewer possible SNVs (n=234; experiment in FIG. 2) than hexamer substitutions (n=4,095; experiment in FIG. 1), each individual edit is expected to be created independently many more times during editing, given a generally similar experimental setup with respect to number of cells, functional assay, etc. This may give rise to the higher reproducibility of the SNV effect sizes as compared with the hexamer enrichment scores (i.e. FIG. 2a vs. FIG. 1b).

Whether the noise represents biological variability (for instance, two cells with the same edit producing transcripts at different rates) or technical variability (stochastic effects inherent to sample prep) it is reasoned that by pooling or averaging replicates, the number of successfully edited cells sampled is effectively increased, and therefore noise attributable to low sampling is reduced. Consistent with this, pooling read counts from D3 replicates in the BRCA1 hexamer experiment improved correlation between biological replicates.

For the DBR1 experiment, the overall reproducibility of D11 enrichment scores is reasonable (R=0.752; FIG. 13c). In each biological replicate, a bimodal distribution of enrichment scores (i.e. corresponding to tolerated vs. deleterious) is observed. While there are some observations, e.g. nonsense mutations that are tolerated in one replicate, not all of the observations replicated. While two synonymous changes score as reproducibly deleterious, there are no nonsense or frameshift mutations that are reproducibly tolerated.

This experiment, subject to bottlenecking at the editing step, generates clonal populations possibly expanded from a single edited cell. Falsely tolerated edits (i.e. nonsense mutations not selected against) in a given replicate could be explained by Hap1 cells' reversion to diploidy prior to editing occurring, as noted by Haplogen (the cell line's source). Falsely deleterious edits in a given replicate could be observed due to off-target CRISPR cutting in other essential regions, or random dropout when half the sample is split on D5.

These findings suggest that while the technique is sensitive enough to measure effects from very few edited cells, noise associated with sampling such small populations mandates the necessity of replicating data sets to improve confidence in the measurements associated with individual genome edits. The data also suggest that increased reproducibility may be achievable by a) transfecting and analyzing a higher number of cells, b) limiting complexity of HDR libraries, or c) improving HDR efficiency to allow for sampling of more edited cells.

Potential Applications of Saturation Genome Editing

In the experiments disclosed herein, genotype is directly linked to phenotype to assay pools of multiplex HDR-derived variants. Targeted RNA and DNA sequencing of the edits themselves via selective PCR are well suited to catalog variants' effects on splicing and cellular fitness, respectively. However, with relatively simple adaptations of the method, complex pools of genome edits can be subjected to many additional assays that measure diverse aspects of biology.

First, the approach illustrated in the BRCA1 experiments is broadly applicable to study how genomic variation within virtually any transcribed element affects its own RNA abundance. Specifically, this approach could readily be adopted to study how other transcribed elements contribute to expression levels (e.g. the influence of 5′- and 3′ UTRs sequence on RNA stability, etc.). In this context, enhancers are transcribed at low levels (eRNA), suggesting an approach for studying enhancer activity, as well.

Additionally, assays such as targeted ChIP-Seq could be performed to characterize how libraries of genomic edits affect epigenetic states in coding or non-coding regions. By taking large quantities of DNA from expanded populations of edited cells and functionally separating edits based on biochemical interactions (i.e. transcription factor binding, associated histone modification, nucleosome positioning, etc.), genotype-phenotype associations would be preserved.

Apart from the molecular assays described above, the DBR1 experiment is just one example of a cell-based assay that can be read out with high-throughput sequencing. In addition to essentiality (in haploid cells or diploid cells made functionally haploid through previous gene disruption), gain-of-function (such as drug resistance or growth gain), haploid insufficiency and dominant negative effects could be measured with appropriate selection assays. In fact, any well-customized assay that allows functionally-based separation of cell populations (e.g., with FACS) is amenable to downstream sequencing of edited populations of assayed cells as a readout. For instance, reporter cell lines engineered to express fluorescently tagged genes of interest could be used to assay multiplex HDR-edited transcription factors or enhancers.

Given the relative ease of targeted nuclease production and mutagenesis library cloning, the methods disclosed herein are readily scalable. Exons could be tiled to functionally assess each coding SNV across entire genes. Therefore, the methods disclosed herein provide a valuable approach for determining functional effects of large numbers of programmed genomic mutations in many biological contexts.

### REFERENCES

The references, patents and published patent applications listed below, and all references cited in the specification above are hereby incorporated by reference in their entirety, as if fully set forth herein.
1. Myers, R. M., Tilly, K. & Maniatis, T. Fine structure genetic analysis of a beta-globin promoter. Science 232, 613-618 (1986).
2. Cunningham, B. C. & Wells, J. A. High-resolution epitope mapping of hGH-receptor interactions by alanine-scanning mutagenesis. Science 244, 1081-1085 (1989).
3. Patwardhan, R. P. et al. High-resolution analysis of DNA regulatory elements by synthetic saturation mutagenesis. Nature Biotechnol. 27, 1173-1175 (2009).
4. Fowler, D. M. et al. High-resolution mapping of protein sequence-function relationships. Nature Methods 7, 741-746 (2010).
5. Botstein, D. & Shortle, D. Strategies and applications of in vitro mutagenesis. Science 229, 1193-1201 (1985).
6. Gibson, T. J., Seiler, M. & Veitia, R. A. The transience of transient overexpression. Nature Methods 10, 715-721 (2013).
7. Gaj, T., Gersbach, C. A. & Barbas, C. F., III ZFN, TALEN, and CRISPR/Cas-based methods for genome engineering. Trends Biotechnol. 31, 397-405 (2013).
8. Wang, H. et al. One-step generation of mice carrying mutations in multiple genes by CRISPR/Cas-mediated genome engineering. Cell 153, 910-918 (2013).
9. Ke, S. et al. Quantitative evaluation of all hexamers as exonic splicing elements. Genome Res. 21, 1360-1374 (2011).
10. Jinek, M. et al. A programmable dual-RNA-guided DNA endonuclease in adaptive bacterial immunity. Science 337, 816-821 (2012).
11. Elliott, B., Richardson, C., Winderbaum, J., Nickoloff, J. A. & Jasin, M. Gene conversion tracts from double-strand break repair in mammalian cells. Mol. Cell. Biol. 18, 93-101 (1998).
12. Mali, P. et al. RNA-guided human genome engineering via Cas9. Science 339, 823-826 (2013).
13. Cong, L. et al. Multiplex genome engineering using CRISPR/Cas systems. Science 339, 819-823 (2013).
14. Mazoyer, S. et al. A BRCA1 nonsense mutation causes exon skipping. Am. J. Hum. Genet. 62, 713-715 (1998).
15. Sander, J. D. & Joung, J. K. CRISPR-Cas systems for editing, regulating and targeting genomes. Nature Biotechnol. 32, 347-355 (2014).
16. Zhang, J., Kuo, C. C. & Chen, L. GC content around splice sites affects splicing through pre-mRNA secondary structures. BMC Genomics 12, 90 (2011).
17. Patwardhan, R. P. et al. Massively parallel functional dissection of mammalian enhancers in vivo. Nature Biotechnol. 30, 265-270 (2012).
18. Mort, M. et al. MutPred Splice: machine learning-based prediction of exonic variants that disrupt splicing. Genome Biol. 15, R19 (2014).
19. Wang, T., Wei, J. J., Sabatini, D. M. & Lander, E. S. Genetic screens in human cells using the CRISPR-Cas9 system. Science 343, 80-84 (2014).
20. Henikoff, S. & Henikoff, J. G. Amino acid substitution matrices from protein blocks. Proc. Natl Acad. Sci. USA 89, 10915-10919 (1992).
21. Adzhubei, I. A. et al. A method and server for predicting damaging missense mutations. Nature Methods 7, 248-249 (2010).
22. Kircher, M. et al. A general framework for estimating the relative pathogenicity of human genetic variants. Nature Genet. 46, 310-315 (2014).
23. Hsu, P. D. et al. DNA targeting specificity of RNA-guided Cas9 nucleases. Nature Biotechnol. 31, 827-832 (2013).
24. Ran, F. A. et al. Genome engineering using the CRISPR-Cas9 system. Nature Protocols 8, 2281-2308 (2013).
25. Carette, J. E. et al. Haploid genetic screens in human cells identify host factors used by pathogens. Science 326, 1231-1235 (2009).
26. Khalid, M. F., Damha, M. J., Shuman, S. & Schwer, B. Structure-function analysis of yeast RNA debranching enzyme (Dbr1), a manganese-dependent phosphodiesterase. Nucleic Acids Res. 33, 6349-6360 (2005).
27. Doyon, Y. et al. Transient cold shock enhances zinc-finger nuclease-mediated gene disruption. Nature Methods 7, 459-460 (2010).
28. Chen, F. et al. High-frequency genome editing using ssDNA oligonucleotides with zinc-finger nucleases. Nature Methods 8, 753-755 (2011).
29. Reyon, D. et al. FLASH assembly of TALENs for high-throughput genome editing. Nature Biotechnol. 30, 460-465 (2012).
30. Carroll, D. Genome engineering with targetable nucleases. Annu. Rev. Biochem. 83, 409-439 (2014).
31. Smurnyy, Y. et al. DNA sequencing and CRISPR-Cas9 gene editing for target validation in mammalian cells. Nature Chem. Biol. 10, 623-625 (2014).
32. Kinney, J. B., Murugan, A., Callan, C. G., Jr & Cox, E. C. Using deep sequencing to characterize the biophysical mechanism of a transcriptional regulatory sequence. Proc. Natl Acad. Sci. USA 107, 9158-9163 (2010).
33. Goina, E., Skoko, N. & Pagani, F. Binding of DAZAP1 and hnRNPA1/A2 to an exonic splicing silencer in a natural BRCA1 exon 18 mutant. Mol. Cell. Biol. 28, 3850-3860 (2008).
34. Zhang, J., Kobert, K., Flouri, T. & Stamatakis, A. PEAR: a fast and accurate Illumina Paired-End reAd mergeR. Bioinformatics 30, 614-620 (201).
35. Li, H. & Durbin, R. Fast and accurate short read alignment with Burrows-Wheeler transform. Bioinformatics 25, 1754-1760 (2009).

TABLE 1
List of all oligonucleotides used in saturation genome editing experiments

| BRCA1 Library Cloning | SEQ ID NO | |
|---|---|---|
| Template for random hexamer library | | |
| BRCA1ex18NNNNNN5_10selPCR (machine-mixing) | SEQ ID NO: 1 | GAGTGTTTTTCATTCTGCAGATGCNNNNNNTGTGTGGATATCCCACACTGAAATATTTTCTAGGAATTGCGGGAGG |
| To PCR-amplify hexamer library inserts | | |
| BRCA1ex18_Hexamers_F | SEQ ID NO: 2 | GAGTGTTTTTCATTCTGCAG |
| BRCA1ex18_Hexamers_R | SEQ ID NO: 3 | CCTCCCGCAATTCCTAGAAA |
| To PCR-amplify homologous arms from gDNA with pUC19 | | |
| adaptors | | |
| pUC_gBRCA1_INF_5′ | SEQ ID NO: 4 | CGGTACCCGGGGATCGAACTCCCGACATCAGGTGA |
| pUC_gBRCA1_INF_3′ | SEQ ID NO: 5 | CGACTCTAGAGGATCAAGGATTCTGTGAGGGAGCA |
| To PCR-linearize pUC19-BRCA1ex18 for random hexamer | | |
| insertion | | |
| Lin_pUC_BRCA1ex18_5′ | SEQ ID NO: 6 | CTGCAGAATGAAAAACACTC |
| Lin_pUC_BRCA1ex18_3′ | SEQ ID NO: 7 | TTTCTAGGAATTGCGGGAGG |
| BRCA1ex18_3%_mut Template (blue = 97 wt/1/1/1 bases; | SEQ ID NO: 8 | ATTCTGCAGATGCTGAGTTTGTGTGTGAACGGACACTGAAATATTTTCTAGGAATTGCGGGAGGA |
| hand-mixing) | | AAATGGGGAGGAAAATGGGTAGTTAGCTATTTCTGTAAGTATAATAC |
| To PCR-amplify BRCA1ex18 3% mutation inserts | | |
| BRCA1ex18_5′1_Amp | SEQ ID NO: 9 | GAGTGTTTTTCATTCTGCAG |
| 5′2_Syn_Amp | SEQ ID NO: 10 | TTCTGCAGATGCTGAGTTTGTCTGCGAGAGAACACTGAAATATTTTC |
| 5′2_Eco_Amp | SEQ ID NO: 11 | TTCTGCAGATGCTGAGTTTGTGTGGATATCCACACTGAAATATTTTC |
| BRCA1ex18_3′1_Amp | SEQ ID NO: 12 | GGAGAAATAGTATTATACTTAC |
| 3′2_Syn_Amp | SEQ ID NO: 13 | TTATACTTACAGAAATAGCTTACAACCCACTTACCACCTGCAATTCCTA |
| 3′2_Eco_Amp | SEQ ID NO: 14 | TTATACTTACAGAAATAGCTAAGGATATCTTTTCCTCCCGCAAT |
| To PCR-linearize pUC19_BRCA1ex18 for 3% mutation | | |
| inserts | | |
| In-Fusion_Lin_5′1 | SEQ ID NO: 15 | CTGCAGAATGAAAAACACTC |
| In-Fusion_Lin_3′1 | SEQ ID NO: 16 | GTAAGTATAATACTATTTCTCC |
| In-Fusion_Lin_5′2 | SEQ ID NO: 17 | CAAACTCAGCATCTGCAGAA |
| In-Fusion_Lin_3′2 | SEQ ID NO: 18 | AGCTATTTCTGTAAGTATAA |
| pCas9-sgBRCA1x18 cloning | | |
| sgRNA_BRCA1ex18_F | SEQ ID NO: 19 | CACCGATGCTGAGTTTGTGTGTGAA |
| sgRNA_BRCA1ex18_R | SEQ ID NO: 20 | AAACTTCACACACAAACTCAGCATC |
| BRCA1 Sample Processing for Sequencing | | |
| Reverse Transcription | | |
| BRCA1ex19_RT | SEQ ID NO: 21 | TTTCTTTCTTTAATAGACTGGGTCA |
| BRCA1ex21_RT | SEQ ID NO: 22 | GTGGGCATGTTGGTGAAG |
| Primer to PCR-amplify from gDNA external to HDR Library | | |
| external_BRCA1ex18_F | SEQ ID NO: 23 | CGGCTTTTGTAGCAGTTAAACA |
| gDNA/cDNA hexamer selective PCR primer | | |
| BRCA1ex18SelPCR_Hex_R | 24SEQ ID NO: | CCTAGAAAATATTTCAGTGTGGATATC |
| cDNA hexamer PCR primers | | |
| BRCA1ex16_cDNA1_F | SEQ ID NO: 25 | GGAGAAGCCAGAATTGACA |
| Hexamer sequencing adaptor primers | | |
| PU1L_BRCA1intron17_F | SEQ ID NO: 26 | CTAAATGGCTGTGAGAGAGCTCAGTGGTGTTTTCAGCCTCTGATT |
| PU1R_BRCA1ex18SelPCR_Hex_R | SEQ ID NO: 27 | ACTTTATCAATCTCGCTCCAAACCCCTAGAAAATATTTCAGTGTGGATATC |
| PU1L_BRCA1ex16_cDNA2_F | SEQ ID NO: 28 | CTAAATGGCTGTGAGAGAGCTCAGGCTTCAACAGAAAGGGTCA |
| PU1R_BRCA1ex18_3′1_Amp | SEQ ID NO: 29 | ACTTTATCAATCTCGCTCCAAACCGGAGAAATAGTATTATACTTAC |
| MiSeq Flowcell and Indexing Primers | | |
| FC_PU1L | SEQ ID NO: 30 | AATGATACGGCGACCACCGAGATCTACACACGTAGGCCTAAATGGCTGTGAGAGAGCTCAG |
| FC_index_PU1R_142 | SEQ ID NO: 31 | CAAGCAGAAGACGGCATACGAGATAAGCGTTCAGACCGTCGGCACTTTATCAATCTCGCTCCAAACC |
| FC_index_PU1R_143 | SEQ ID NO: 32 | CAAGCAGAAGACGGCATACGAGATCGCAAGCGTGACCGTCGGCACTTTATCAATCTCGCTCCAAACC |
| FC_index_PU1R_144 | SEQ ID NO: 33 | CAAGCAGAAGACGGCATACGAGATGCAGCGCGAGACCGTCGGCACTTTATCAATCTCGCTCCAAACC |
| FC_index_PU1R_145 | SEQ ID NO: 34 | CAAGCAGAAGACGGCATACGAGATCGCGCAGCTGACCGTCGGCACTTTATCAATCTCGCTCCAAACC |
| FC_index_PU1R_146 | SEQ ID NO: 35 | CAAGCAGAAGACGGCATACGAGATTCAAGCGCAGACCGTCGGCACTTTATCAATCTCGCTCCAAACC |
| FC_index_PU1R_147 | SEQ ID NO: 36 | CAAGCAGAAGACGGCATACGAGATCAGTCGCAGGACCGTCGGCACTTTATCAATCTCGCTCCAAACC |
| FC_index_PU1R_148 | SEQ ID NO: 37 | CAAGCAGAAGACGGCATACGAGATGCGTCAGTTGACCGTCGGCACTTTATCAATCTCGCTCCAAACC |
| FC_index_PU1R_149 | SEQ ID NO: 38 | CAAGCAGAAGACGGCATACGAGATAGTCGCGCAGACCGTCGGCACTTTATCAATCTCGCTCCAAACC |
| Selective PCR and seq prep for 3% mut experiments | | |
| BRCA1ex20_R | SEQ ID NO: 39 | CTTTCTGTCCTGGGATTCTC |
| PU1R_BRCA1ex20 | SEQ ID NO: 40 | ACTTTATCAATCTCGCTCCAAACCCTTTCTGTCCTGGGATTCTC |
| BRCA1ex16_cDNA2_F | SEQ ID NO: 41 | GCTTCAACAGAAAGGGTCA |
| PU1L_BRCA1ex16_cDNA2_F | SEQ ID NO: 42 | CTAAATGGCTGTGAGAGAGCTCAGGCTTCAACAGAAAGGGTCA |
| PU1L_BRCA1intron17_2 | SEQ ID NO: 43 | CTAAATGGCTGTGAGAGAGCTCAGCCAGATTGATCTTGGGAGTG |
| PU1R_BRCA1intron18 | SEQ ID NO: 44 | ACTTTATCAATCTCGCTCCAAACCGGTAACTCAGACTCAGCATC |
| BRCA1ex18_sel5Syn_F | SEQ ID NO: 45 | TGAGTTTGTCTGCGAGAGA |
| PU1L_BRCA1ex18_sel5syn | SEQ ID NO: 46 | CTAAATGGCTGTGAGAGAGCTCAGTGAGTTTGTCTGCGAGAGA |
| BRCA1ex18_sel5Eco_F | SEQ ID NO: 47 | GCTGAGTTTGTGTGGATATCC |
| PU1L_BRCA1ex18_sel5Eco | SEQ ID NO: 48 | CTAAATGGCTGTGAGAGAGCTCAGGCTGAGTTTGTGTGGATATCC |
| BRCA1ex18_sel3Syn_R | SEQ ID NO: 49 | TACAACCCACTTACCACCT |
| PU1R_BRCA1ex18_sel3Syn | SEQ ID NO: 50 | ACTTTATCAATCTCGCTCCAAACCTACAACCCACTTACCACCT |
| BRCA1ex18_sel3Eco_R | SEQ ID NO: 51 | ACTTACAGAAATAGCTAAGGATATC |
| PU1R_BRCA1ex18_sel3Eco | SEQ ID NO: 52 | ACTTTATCAATCTCGCTCCAAACCACTTACAGAAATAGCTAAGGATATC |
| DBR1 HDR Library Cloning | | |
| CustomArray DBR1 oligonucleotide pool | SEQ ID NO: 53 | CCAGTTCTCACGCTCTTCATCGGCGGAAACCATGAAGCCTCAATCATTTGCAAGAGTTACCCTATGGTGGGTGGCA |
| (63x codon mutations bold; 225 1bp mutations blue; non- | | CCAAACATTTATTATTTAGgtatgtgtgatactttgtggatac |
| reference red; intron lower-case) | | |
| To PCR-amplify HDR library inserts (oligonucleotide pool) | | |
| Dbr1X2D_AMP_F | SEQ ID NO: 54 | TCTCACGCTCTTCATCGGCG |
| Dbr1X2D_AMP_R | SEQ ID NO: 55 | TCCACAAAGTATCACACAT |
| To PCR-amplify homologous arms from Hap1 gDNA with | | |
| pUC19 adaptors | | |
| pUC19_Dbr1_Inf_F | SEQ ID NO: 56 | CGGTACCCGGGGATCGAGGCGGGAAATGTATTCAG |
| pUC19_Dbr1_Inf_R | SEQ ID NO: 57 | CGACTCTAGAGGATCCAGGAAATGCTGCAAGACAA |
| To PCR-linearize pUC19-Dbr1ex2 for library insertion | | |
| Lin_pUC19-Dbr1X2_5 | SEQ ID NO: 58 | CGCCGATGAAGAGCGTGAGAACTGG |
| Lin_pUC19-Dbr1X2_3 | SEQ ID NO: 59 | ATGTGTGATACTTTGTGGATACTTTTTTTTCCTGGG |
| DBR1 Sample Processing for Sequencing | | |
| To amplify gDNA from outside plasmid homology | | |
| external_Dbr1_F | SEQ ID NO: 60 | GCCTGTTTCCCTTAGATCCT |
| Selective PCR from edited gDNA | | |
| Dbr1Int2_selAMP_R | SEQ ID NO: 61 | AGTATCCACAAAGTATCACAC |
| Non-selective PCR from edited gDNA | | |
| Dbr1_Int2_R2 | SEQ ID NO: 62 | CCTGATAAGCTCTTCCATCC |
| To add adaptors for sequencing | | |
| PU1L_Dbr1Int1_AMP | SEQ ID NO: 63 | CTAAATGGCTGTGAGAGAGCTCAGGCCTAATTGTGGTAACTGAC |
| PU1R_Dbr1Int2_selAMP_R | SEQ ID NO: 64 | ACTTTATCAATCTCGCTCCAAACCAGTATCCACAAAGTATCACAC |
| PU1R_Dbr1_Int2_R2 | SEQ ID NO: 65 | ACTTTATCAATCTCGCTCCAAACCCCTGATAAGCTCTTCCATCC |
| pCas9-EGFP-sgDbr1x2 Cloning | | |
| sgRNA_Dbr1X2_F | SEQ ID NO: 66 | CACCGCAGTTCTCACGCTCTTCATT |
| sgRNA_Dbr1X2_R | SEQ ID NO: 67 | AAACAATGAAGAGCGTGAGAACTGC |
| Exon sequences in this study | SEQ ID NO: 68 | .GAGTGTTTTTCATTCTGCAGATGCTGAGTTTGTGTGTGAACGGACACTGAAATATTTTCTA |
| WT BRCA1ex18 (PAM, protospacer underlined; introns gray) | | GGAATTGCGGGAGGAAAATGGGTAGTTAGCTATTTCTGTAAGTATAATACTATTTC |
| BRCA1 and DBR1 HDR library exons in pUC19 | | |
| homology donor plasmids | | |
| HDRL Random Hexamer BRCA1ex18 | SEQ ID NO: 69 | ...GAGTGTTTTTCATTCTGCAGATGCNNNNNNTGTGTGGATATCCACACTGAAATATTTTCT |
| | | AGGAATTGCGGGAGGAAAATGGGTAGTTAGCTATTTCTGTAAGTATAATACTATTTC... |
| HDRL 3% mut 5′ SYN BRCA1ex18 | SEQ ID NO: 70 | .GAGTGTTTTTCATTCTGCAGATGCTGAGTTTGTCTGCGAGAGAACACTGAAATATTTTCTAGG |
| | | AATTGCGGGAGGAAAATGGGTAGTTAGCTATTTCTGTAAGTATAATACTATTTC. |
| HDRL 3% mut 5′ NONSYN BRCA1ex18 | SEQ ID NO: 71 | ...GAGTGTTTTTCATTCTGCAGATGCTGAGTTTGTGTGGATATCCACACTGAAATATTTTCT |
| | | AGGAATTGCGGGAGGAAAATGGGTAGTTAGCTATTTCTGTAAGTATAATACTATTTC .. |
| HDRL 3% mut 3′ NONSYN BRCA1ex18 | SEQ ID NO: 72 | ...GAGTGTTTTTCATTCTGCAGATGCTGAGTTTGTGTGTGAACGGACACTGAAATATTTTC |
| (selective PCR sites red; mutated region blue) | | TAGGAATTGCGGGAGGAAAAGATATCCTTAGCTATTTCTGTAAGTATAATACTATTTC |
| WT DBR1ex2 | SEQ ID NO: 73 | ...CCAGTTCTCACGCTCTTCATTGGGGGAAACCATGAAGCCTCAAATCATTTGCAAGA |
| | | GTTACCCTATGGTGGCTGGGTGGCACCAAACATTTATTATTTAGGTATGTGATTGTGTTTGTGGATAC |
| DBR1ex2 HDRL | SEQ ID NO: 74 | ...CCAGTTCTCACGCTCTTCATCGGCGGAAACCATGAAGCCTCAAATCATTTGCAAG |
| | | AGTTACCCTATGGTGGCTGGGTGGCACCAAACATTATTATTTAGGTATGTGTGATACTTTGTGGATAC |

(63x codon substitutions bold, PAM/protospacer mutations green)

TABLE 2
Empirical measurement of impact of introducing 4048
hexamers as genome edits to BRCA1 exon 18 (+5 to +10) on
abundance of exon 18 containing transcripts

| | | | | hamming | Log2 |
|---|---|---|---|---|---|
| | % in input | Ke et al. 2011 | nonsense | distance to | enrichment |
| Hexamer | library | ESRseq score | hexamer? | WT | score |
| TGAGTT | 1.197% | NA | no | 0 | 0.000 |
| AGAGTT | 0.049% | NA | no | 1 | −2.087 |
| CGAGTT | 0.014% | NA | no | 1 | −0.095 |
| GGAGTT | 0.040% | NA | no | 1 | −0.135 |
| TAAGTT | 0.024% | −0.493 | no | 1 | −0.744 |
| TCAGTT | 0.019% | −0.311 | no | 1 | −2.260 |
| TGAATT | 0.031% | NA | no | 1 | 0.444 |
| TGACTT | 0.017% | NA | no | 1 | 0.436 |
| TGAGAT | 0.033% | NA | no | 1 | −0.034 |
| TGAGCT | 0.019% | NA | no | 1 | 0.674 |
| TGAGGT | 0.034% | NA | no | 1 | −0.310 |
| TGAGTA | 0.037% | NA | no | 1 | 0.478 |
| TGAGTC | 0.016% | NA | no | 1 | −0.153 |
| TGAGTG | 0.023% | NA | no | 1 | −0.227 |
| TGATTT | 0.019% | NA | no | 1 | −0.051 |
| TGCGTT | 0.016% | NA | no | 1 | 0.309 |
| TGGGTT | 0.021% | −0.521 | no | 1 | −0.685 |
| TGTGTT | 0.022% | −0.198 | no | 1 | −0.440 |
| TTAGTT | 0.017% | −0.831 | yes | 1 | −4.278 |
| AAAGTT | 0.050% | NA | no | 2 | −2.917 |
| ACAGTT | 0.036% | NA | no | 2 | −3.111 |
| AGAATT | 0.037% | NA | no | 2 | −5.548 |
| AGACTT | 0.032% | NA | no | 2 | −3.599 |
| AGAGAT | 0.053% | 0.367 | no | 2 | −2.453 |
| AGAGCT | 0.033% | NA | no | 2 | −0.866 |
| AGAGGT | 0.054% | NA | no | 2 | −2.970 |
| AGAGTA | 0.062% | NA | no | 2 | −4.019 |
| AGAGTC | 0.039% | 0.300 | no | 2 | −3.721 |
| AGAGTG | 0.059% | NA | no | 2 | −3.279 |
| AGATTT | 0.032% | NA | no | 2 | −3.706 |
| AGCGTT | 0.028% | NA | no | 2 | −0.376 |
| AGGGTT | 0.051% | −0.470 | no | 2 | −5.913 |
| AGTGTT | 0.035% | NA | no | 2 | −0.708 |
| ATAGTT | 0.030% | −0.554 | yes | 2 | −5.399 |
| CAAGTT | 0.020% | NA | no | 2 | −1.781 |
| CGAATT | 0.023% | NA | no | 2 | 0.094 |
| CGACTT | 0.010% | NA | no | 2 | −0.889 |
| CGAGAT | 0.023% | 0.348 | no | 2 | 0.637 |
| CGAGCT | 0.015% | NA | no | 2 | 1.281 |
| CGAGGT | 0.019% | NA | no | 2 | −0.099 |
| CGAGTA | 0.022% | NA | no | 2 | −1.055 |
| CGAGTC | 0.011% | 0.399 | no | 2 | −1.001 |
| CGAGTG | 0.018% | 0.165 | no | 2 | −0.742 |
| CGATTT | 0.008% | NA | no | 2 | −1.125 |
| CGCGTT | 0.008% | 0.316 | no | 2 | 0.643 |
| CGGGTT | 0.017% | NA | no | 2 | 0.523 |
| CGTGTT | 0.008% | NA | no | 2 | −0.268 |
| CTAGTT | 0.007% | −0.555 | yes | 2 | −3.727 |
| GAAGTT | 0.040% | 0.468 | no | 2 | −1.915 |
| GCAGTT | 0.027% | NA | no | 2 | −1.427 |
| GGAATT | 0.044% | 0.242 | no | 2 | −1.710 |
| GGACTT | 0.028% | 0.338 | no | 2 | 0.237 |
| GGAGAT | 0.058% | 0.336 | no | 2 | −0.559 |
| GGAGCT | 0.034% | NA | no | 2 | −0.362 |
| GGAGGT | 0.043% | NA | no | 2 | −0.966 |
| GGAGTA | 0.053% | NA | no | 2 | −0.989 |
| GGAGTC | 0.031% | 0.516 | no | 2 | 1.196 |
| GGAGTG | 0.051% | NA | no | 2 | 0.448 |
| GGATTT | 0.037% | NA | no | 2 | 0.511 |
| GGCGTT | 0.014% | 0.278 | no | 2 | 0.622 |
| GGGGTT | 0.038% | −0.543 | no | 2 | −1.880 |
| GGTGTT | 0.023% | NA | no | 2 | 0.407 |
| GTAGTT | 0.020% | −0.658 | yes | 2 | −5.724 |
| TAAATT | 0.026% | −0.632 | no | 2 | −3.111 |
| TAACTT | 0.021% | −0.457 | no | 2 | −3.257 |
| TAAGAT | 0.035% | NA | no | 2 | −4.842 |
| TAAGCT | 0.023% | −0.368 | no | 2 | −3.123 |
| TAAGGT | 0.041% | −0.468 | no | 2 | −5.926 |
| TAAGTA | 0.037% | −0.667 | no | 2 | −5.980 |
| TAAGTC | 0.018% | NA | no | 2 | −4.899 |
| TAAGTG | 0.029% | −0.461 | no | 2 | −2.670 |
| TAATTT | 0.019% | −0.389 | no | 2 | −2.975 |
| TACGTT | 0.016% | NA | no | 2 | −0.949 |
| TAGGTT | 0.020% | −0.796 | no | 2 | −4.864 |
| TATGTT | 0.022% | −0.301 | no | 2 | 0.809 |
| TCAATT | 0.018% | NA | no | 2 | −1.685 |
| TCACTT | 0.020% | NA | no | 2 | −2.880 |
| TCAGAT | 0.030% | NA | no | 2 | −2.559 |
| TCAGCT | 0.018% | NA | no | 2 | −1.810 |
| TCAGGT | 0.034% | −0.438 | no | 2 | −3.941 |
| TCAGTA | 0.027% | −0.407 | no | 2 | −4.288 |
| TCAGTC | 0.019% | NA | no | 2 | −3.280 |
| TCAGTG | 0.019% | −0.267 | no | 2 | −1.379 |
| TCATTT | 0.021% | NA | no | 2 | −1.268 |
| TCCGTT | 0.010% | NA | no | 2 | −1.495 |
| TCGGTT | 0.009% | NA | no | 2 | 0.288 |
| TCTGTT | 0.009% | NA | no | 2 | −3.347 |
| TGAAAT | 0.044% | 0.190 | no | 2 | 0.723 |
| TGAACT | 0.025% | 0.339 | no | 2 | 0.102 |
| TGAAGT | 0.040% | 0.384 | no | 2 | 0.269 |
| TGAATA | 0.039% | NA | no | 2 | −0.574 |
| TGAATC | 0.023% | 0.426 | no | 2 | 0.250 |
| TGAATG | 0.033% | 0.131 | no | 2 | −0.509 |
| TGACAT | 0.028% | 0.198 | no | 2 | −0.058 |
| TGACCT | 0.016% | 0.459 | no | 2 | 0.587 |
| TGACGT | 0.020% | 0.482 | no | 2 | −0.401 |
| TGACTA | 0.020% | NA | no | 2 | −0.011 |
| TGACTC | 0.014% | 0.281 | no | 2 | 0.808 |
| TGACTG | 0.025% | 0.319 | no | 2 | 0.017 |
| TGAGAA | 0.042% | 0.338 | no | 2 | −0.485 |
| TGAGAC | 0.023% | 0.379 | no | 2 | 0.426 |
| TGAGAG | 0.039% | NA | no | 2 | 0.032 |
| TGAGCA | 0.032% | NA | no | 2 | 0.825 |
| TGAGCC | 0.013% | NA | no | 2 | −0.385 |
| TGAGCG | 0.028% | 0.298 | no | 2 | −0.504 |
| TGAGGA | 0.045% | 0.459 | no | 2 | −0.578 |
| TGAGGC | 0.026% | NA | no | 2 | −0.340 |
| TGAGGG | 0.036% | −0.255 | no | 2 | −0.259 |
| TGATAT | 0.020% | NA | no | 2 | 0.376 |
| TGATCT | 0.014% | 0.250 | no | 2 | −0.824 |
| TGATGT | 0.023% | 0.187 | no | 2 | −0.087 |
| TGATTA | 0.025% | −0.400 | no | 2 | −1.266 |
| TGATTC | 0.015% | NA | no | 2 | 0.431 |
| TGATTG | 0.023% | NA | no | 2 | −0.434 |
| TGCATT | 0.015% | −0.326 | no | 2 | 0.675 |
| TGCCTT | 0.012% | −0.306 | no | 2 | −0.259 |
| TGCGAT | 0.021% | NA | no | 2 | −0.172 |
| TGCGCT | 0.014% | NA | no | 2 | 0.218 |
| TGCGGT | 0.025% | NA | no | 2 | 0.378 |
| TGCGTA | 0.027% | −0.382 | no | 2 | −0.825 |
| TGCGTC | 0.013% | 0.277 | no | 2 | 0.398 |
| TGCGTG | 0.018% | NA | no | 2 | −0.198 |
| TGCTTT | 0.016% | −0.394 | no | 2 | −0.653 |
| TGGATT | 0.017% | 0.323 | no | 2 | 1.166 |
| TGGCTT | 0.019% | NA | no | 2 | 0.058 |
| TGGGAT | 0.029% | NA | no | 2 | −0.353 |
| TGGGCT | 0.017% | −0.259 | no | 2 | 0.128 |
| TGGGGT | 0.023% | −0.511 | no | 2 | −0.953 |
| TGGGTA | 0.039% | −0.617 | no | 2 | −0.059 |
| TGGGTC | 0.013% | −0.279 | no | 2 | 0.302 |
| TGGGTG | 0.024% | −0.379 | no | 2 | 0.271 |
| TGGTTT | 0.020% | −0.337 | no | 2 | 0.517 |
| TGTATT | 0.019% | −0.319 | no | 2 | 0.378 |
| TGTCTT | 0.017% | NA | no | 2 | −0.008 |
| TGTGAT | 0.027% | 0.111 | no | 2 | 0.512 |
| TGTGCT | 0.018% | −0.251 | no | 2 | 0.576 |
| TGTGGT | 0.020% | NA | no | 2 | 0.449 |
| TGTGTA | 0.021% | −0.320 | no | 2 | 0.571 |
| TGTGTC | 0.016% | −0.091 | no | 2 | −0.114 |
| TGTGTG | 0.018% | −0.073 | no | 2 | −0.106 |
| TGTTTT | 0.017% | −0.323 | no | 2 | −1.622 |
| TTAATT | 0.021% | −0.507 | yes | 2 | −5.963 |
| TTACTT | 0.010% | NA | no | 2 | −1.858 |
| TTAGAT | 0.029% | −0.578 | yes | 2 | −6.001 |
| TTAGCT | 0.011% | −0.539 | yes | 2 | −6.151 |
| TTAGGT | 0.020% | −0.783 | yes | 2 | −5.014 |
| TTAGTA | 0.022% | −0.852 | yes | 2 | −7.070 |
| TTAGTC | 0.013% | −0.539 | yes | 2 | −3.757 |
| TTAGTG | 0.018% | −0.572 | yes | 2 | −4.719 |
| TTATTT | 0.018% | −0.387 | no | 2 | −4.650 |
| TTCGTT | 0.010% | 0.263 | no | 2 | 0.414 |
| TTGGTT | 0.016% | −0.279 | no | 2 | −2.771 |
| TTTGTT | 0.014% | −0.219 | no | 2 | −4.474 |
| AAAATT | 0.041% | −0.420 | no | 3 | −3.370 |
| AAACTT | 0.036% | NA | no | 3 | −3.803 |
| AAAGAT | 0.060% | NA | no | 3 | −2.175 |
| AAAGCT | 0.045% | NA | no | 3 | −1.456 |
| AAAGGT | 0.067% | NA | no | 3 | −3.660 |
| AAAGTA | 0.068% | NA | no | 3 | −3.718 |
| AAAGTC | 0.045% | NA | no | 3 | −3.758 |
| AAAGTG | 0.066% | NA | no | 3 | −0.446 |
| AAATTT | 0.032% | −0.321 | no | 3 | −5.043 |
| AACGTT | 0.027% | 0.344 | no | 3 | −1.254 |
| AAGGTT | 0.039% | NA | no | 3 | −3.706 |
| AATGTT | 0.032% | NA | no | 3 | −3.322 |
| ACAATT | 0.039% | NA | no | 3 | −2.812 |
| ACACTT | 0.024% | NA | no | 3 | −0.816 |
| ACAGAT | 0.043% | 0.254 | no | 3 | −1.121 |
| ACAGCT | 0.032% | NA | no | 3 | −1.664 |
| ACAGGT | 0.047% | −0.315 | no | 3 | −3.492 |
| ACAGTA | 0.045% | NA | no | 3 | −3.203 |
| ACAGTC | 0.029% | NA | no | 3 | −2.148 |
| ACAGTG | 0.044% | −0.161 | no | 3 | −1.459 |
| ACATTT | 0.033% | NA | no | 3 | −3.087 |
| ACCGTT | 0.017% | NA | no | 3 | −0.727 |
| ACGGTT | 0.033% | NA | no | 3 | −1.170 |
| ACTGTT | 0.023% | NA | no | 3 | −2.396 |
| AGAAAT | 0.069% | NA | no | 3 | −5.660 |
| AGAACT | 0.037% | 0.370 | no | 3 | −6.371 |
| AGAAGT | 0.057% | 0.423 | no | 3 | −5.238 |
| AGAATA | 0.052% | NA | no | 3 | −6.123 |
| AGAATC | 0.036% | 0.368 | no | 3 | −7.624 |
| AGAATG | 0.032% | 0.157 | no | 3 | −3.970 |
| AGACAT | 0.048% | NA | no | 3 | −2.164 |
| AGACCT | 0.024% | 0.540 | no | 3 | −0.409 |
| AGACGT | 0.032% | 0.542 | no | 3 | −0.938 |
| AGACTA | 0.037% | NA | no | 3 | −3.628 |
| AGACTC | 0.030% | NA | no | 3 | −2.106 |
| AGACTG | 0.027% | 0.372 | no | 3 | −0.327 |
| AGAGAA | 0.078% | 0.392 | no | 3 | −3.001 |
| AGAGAC | 0.048% | 0.535 | no | 3 | −1.922 |
| AGAGAG | 0.062% | NA | no | 3 | −0.588 |
| AGAGCA | 0.049% | NA | no | 3 | −2.611 |
| AGAGCC | 0.034% | NA | no | 3 | −1.864 |
| AGAGCG | 0.046% | NA | no | 3 | −0.458 |
| AGAGGA | 0.072% | 0.526 | no | 3 | −2.083 |
| AGAGGC | 0.053% | NA | no | 3 | −2.144 |
| AGAGGG | 0.067% | −0.155 | no | 3 | −1.469 |
| AGATAT | 0.044% | NA | no | 3 | −3.245 |
| AGATCT | 0.026% | 0.293 | no | 3 | −4.228 |
| AGATGT | 0.032% | NA | no | 3 | −2.907 |
| AGATTA | 0.048% | NA | no | 3 | −3.769 |
| AGATTC | 0.034% | NA | no | 3 | −3.977 |
| AGATTG | 0.039% | NA | no | 3 | −1.023 |
| AGCATT | 0.030% | NA | no | 3 | −0.977 |
| AGCCTT | 0.020% | −0.300 | no | 3 | −0.676 |
| AGCGAT | 0.034% | NA | no | 3 | 0.558 |
| AGCGGT | 0.034% | NA | no | 3 | −0.746 |
| AGCGTA | 0.039% | NA | no | 3 | 0.056 |
| AGCGTC | 0.024% | 0.515 | no | 3 | 0.682 |
| AGCGTG | 0.031% | NA | no | 3 | −0.212 |
| AGCTTT | 0.016% | −0.353 | no | 3 | 0.785 |
| AGGATT | 0.044% | NA | no | 3 | −4.617 |
| AGGCTT | 0.027% | NA | no | 3 | −3.483 |
| AGGGAT | 0.059% | NA | no | 3 | −4.510 |
| AGGGCT | 0.038% | NA | no | 3 | −4.903 |
| AGGGGT | 0.059% | −0.340 | no | 3 | −5.948 |
| AGGGTA | 0.063% | −0.535 | no | 3 | −8.531 |
| AGGGTC | 0.040% | NA | no | 3 | −8.002 |
| AGGGTG | 0.054% | −0.480 | no | 3 | −1.698 |
| AGGTTT | 0.033% | −0.434 | no | 3 | −4.722 |
| AGTATT | 0.038% | −0.387 | no | 3 | −1.655 |
| AGTCTT | 0.024% | NA | no | 3 | −1.285 |
| AGTGAT | 0.043% | NA | no | 3 | −0.797 |
| AGTGCT | 0.028% | −0.116 | no | 3 | −1.139 |
| AGTGGT | 0.037% | NA | no | 3 | −0.459 |
| AGTGTA | 0.049% | −0.354 | no | 3 | −1.193 |
| AGTGTC | 0.030% | NA | no | 3 | −1.323 |
| AGTGTG | 0.039% | −0.255 | no | 3 | −0.577 |
| AGTTTT | 0.022% | −0.106 | no | 3 | −1.689 |
| ATAATT | 0.042% | −0.326 | yes | 3 | −4.629 |
| ATACTT | 0.029% | −0.253 | no | 3 | −3.405 |
| ATAGAT | 0.041% | NA | yes | 3 | −7.247 |
| ATAGCT | 0.021% | −0.266 | yes | 3 | −3.911 |
| ATAGGT | 0.031% | −0.528 | yes | 3 | −6.841 |
| ATAGTA | 0.038% | −0.504 | yes | 3 | −8.063 |
| ATAGTC | 0.023% | NA | yes | 3 | −8.443 |
| ATAGTG | 0.041% | −0.337 | yes | 3 | −4.360 |
| ATATTT | 0.031% | −0.534 | no | 3 | −3.525 |
| ATCGTT | 0.022% | NA | no | 3 | 0.820 |
| ATGGTT | 0.032% | −0.289 | no | 3 | −5.190 |
| ATTGTT | 0.030% | NA | no | 3 | −3.539 |
| CAAATT | 0.021% | −0.223 | no | 3 | −1.563 |
| CAACTT | 0.017% | NA | no | 3 | 0.004 |
| CAAGAT | 0.035% | 0.638 | no | 3 | 0.017 |
| CAAGCT | 0.018% | 0.212 | no | 3 | −2.428 |
| CAAGGT | 0.026% | NA | no | 3 | −2.327 |
| CAAGTA | 0.028% | NA | no | 3 | −2.177 |
| CAAGTC | 0.016% | 0.334 | no | 3 | −1.227 |
| CAAGTG | 0.025% | NA | no | 3 | −0.641 |
| CAATTT | 0.014% | NA | no | 3 | −3.535 |
| CACGTT | 0.015% | NA | no | 3 | −1.326 |
| CAGGTT | 0.023% | −0.433 | no | 3 | −3.256 |
| CATGTT | 0.016% | NA | no | 3 | −1.058 |
| CCAATT | 0.010% | −0.476 | no | 3 | −2.859 |
| CCACTT | 0.013% | −0.386 | no | 3 | −2.052 |
| CCAGAT | 0.024% | NA | no | 3 | 0.033 |
| CCAGCT | 0.016% | −0.365 | no | 3 | 0.244 |
| CCAGGT | 0.017% | −0.325 | no | 3 | −0.936 |
| CCAGTA | 0.018% | −0.671 | no | 3 | −1.440 |
| CCAGTC | 0.012% | −0.219 | no | 3 | −0.429 |
| CCAGTG | 0.015% | −0.143 | no | 3 | −1.218 |
| CCATTT | 0.009% | −0.590 | no | 3 | −0.899 |
| CCCGTT | 0.007% | NA | no | 3 | −0.463 |
| CCGGTT | 0.011% | NA | no | 3 | 0.340 |
| CCTGTT | 0.009% | NA | no | 3 | 1.394 |
| CGAAAT | 0.026% | NA | no | 3 | −0.612 |
| CGAACT | 0.016% | 0.321 | no | 3 | −0.873 |
| CGAAGT | 0.020% | 0.353 | no | 3 | 0.893 |
| CGAATA | 0.027% | NA | no | 3 | −0.691 |
| CGAATC | 0.015% | 0.352 | no | 3 | 0.276 |
| CGAATG | 0.027% | 0.207 | no | 3 | −1.068 |
| CGACAT | 0.020% | 0.286 | no | 3 | −0.265 |
| CGACCT | 0.009% | 0.288 | no | 3 | −0.156 |
| CGACGT | 0.017% | 0.644 | no | 3 | 1.045 |
| CGACTA | 0.012% | NA | no | 3 | −1.372 |
| CGACTC | 0.010% | NA | no | 3 | 2.608 |
| CGACTG | 0.014% | 0.515 | no | 3 | −0.431 |
| CGAGAA | 0.025% | NA | no | 3 | 1.080 |
| CGAGAC | 0.013% | NA | no | 3 | 1.099 |
| CGAGAG | 0.020% | 0.390 | no | 3 | −1.104 |
| CGAGCA | 0.019% | NA | no | 3 | 0.394 |
| CGAGCC | 0.009% | 0.281 | no | 3 | −0.259 |
| CGAGCG | 0.015% | 0.531 | no | 3 | −0.071 |
| CGAGGA | 0.021% | 0.471 | no | 3 | 0.149 |
| CGAGGC | 0.016% | 0.427 | no | 3 | 0.046 |
| CGAGGG | 0.026% | 0.233 | no | 3 | 0.945 |
| CGATAT | 0.016% | NA | no | 3 | −1.021 |
| CGATCT | 0.008% | NA | no | 3 | −1.982 |
| CGATGT | 0.015% | NA | no | 3 | 0.447 |
| CGATTA | 0.013% | −0.393 | no | 3 | 0.253 |
| CGATTC | 0.010% | NA | no | 3 | 3.144 |
| CGATTG | 0.013% | 0.149 | no | 3 | 1.897 |
| CGCATT | 0.012% | NA | no | 3 | −0.884 |
| CGCCTT | 0.007% | NA | no | 3 | 0.871 |
| CGCGAT | 0.015% | 0.491 | no | 3 | −0.672 |
| CGCGCT | 0.006% | 0.393 | no | 3 | 3.124 |
| CGCGGT | 0.011% | 0.296 | no | 3 | 0.299 |
| CGCGTA | 0.014% | NA | no | 3 | −1.456 |
| CGCGTC | 0.009% | 0.675 | no | 3 | 1.729 |
| CGCGTG | 0.008% | 0.357 | no | 3 | 1.836 |
| CGCTTT | 0.009% | NA | no | 3 | 0.506 |
| CGGATT | 0.016% | 0.372 | no | 3 | −0.897 |
| CGGCTT | 0.007% | NA | no | 3 | 0.312 |
| CGGGAT | 0.026% | 0.420 | no | 3 | 0.223 |
| CGGGCT | 0.015% | NA | no | 3 | 0.553 |
| CGGGGT | 0.018% | NA | no | 3 | −1.640 |
| CGGGTA | 0.025% | NA | no | 3 | 0.462 |
| CGGGTC | 0.014% | NA | no | 3 | 0.464 |
| CGGGTG | 0.013% | NA | no | 3 | −0.247 |
| CGGTTT | 0.014% | NA | no | 3 | −1.467 |
| CGTATT | 0.009% | NA | no | 3 | 1.250 |
| CGTCTT | 0.010% | NA | no | 3 | −2.684 |
| CGTGAT | 0.015% | 0.311 | no | 3 | −1.066 |
| CGTGGT | 0.009% | 0.292 | no | 3 | −1.389 |
| CGTGTC | 0.010% | 0.418 | no | 3 | −0.261 |
| CGTGTG | 0.013% | NA | no | 3 | −0.237 |
| CGTTTT | 0.007% | NA | no | 3 | 1.457 |
| CTAATT | 0.011% | −0.682 | yes | 3 | −5.037 |
| CTACTT | 0.006% | NA | no | 3 | −0.406 |
| CTAGCT | 0.008% | −0.441 | yes | 3 | −8.166 |
| CTAGGT | 0.010% | −0.665 | yes | 3 | −2.775 |
| CTAGTA | 0.011% | −0.925 | yes | 3 | −5.226 |
| CTAGTC | 0.008% | −0.378 | yes | 3 | −5.593 |
| CTAGTG | 0.010% | −0.387 | yes | 3 | −6.779 |
| CTCGTT | 0.005% | NA | no | 3 | 0.338 |
| CTGGTT | 0.010% | NA | no | 3 | −0.543 |
| CTTGTT | 0.011% | −0.302 | no | 3 | −2.109 |
| GAAATT | 0.041% | NA | no | 3 | −2.356 |
| GAACTT | 0.029% | 0.297 | no | 3 | −2.602 |
| GAAGAT | 0.051% | 0.992 | no | 3 | 0.259 |
| GAAGCT | 0.034% | 0.476 | no | 3 | −0.995 |
| GAAGGT | 0.044% | NA | no | 3 | −0.347 |
| GAAGTA | 0.057% | NA | no | 3 | −1.588 |
| GAAGTC | 0.034% | 0.614 | no | 3 | −0.335 |
| GAAGTG | 0.044% | 0.247 | no | 3 | −0.013 |
| GAATTT | 0.036% | NA | no | 3 | −2.861 |
| GACGTT | 0.021% | 0.592 | no | 3 | 0.041 |
| GAGGTT | 0.032% | −0.250 | no | 3 | −3.208 |
| GATGTT | 0.025% | 0.289 | no | 3 | −1.072 |
| GCAATT | 0.020% | NA | no | 3 | −2.914 |
| GCACTT | 0.020% | −0.392 | no | 3 | −0.899 |
| GCAGAT | 0.035% | NA | no | 3 | −3.309 |
| GCAGCT | 0.023% | −0.113 | no | 3 | −1.761 |
| GCAGGT | 0.032% | −0.475 | no | 3 | −3.457 |
| GCAGTA | 0.036% | −0.297 | no | 3 | −0.890 |
| GCAGTC | 0.019% | NA | no | 3 | −2.739 |
| GCAGTG | 0.039% | NA | no | 3 | −1.098 |
| GCATTT | 0.017% | −0.179 | no | 3 | −0.876 |
| GCCGTT | 0.019% | NA | no | 3 | −0.043 |
| GCGGTT | 0.019% | NA | no | 3 | −2.216 |
| GCTGTT | 0.017% | NA | no | 3 | 0.130 |
| GGAAAT | 0.066% | 0.176 | no | 3 | −0.475 |
| GGAACT | 0.046% | 0.428 | no | 3 | 0.155 |
| GGAAGT | 0.057% | 0.249 | no | 3 | 0.112 |
| GGAATA | 0.066% | NA | no | 3 | −1.106 |
| GGAATC | 0.043% | 0.485 | no | 3 | 0.551 |
| GGAATG | 0.062% | 0.208 | no | 3 | −1.925 |
| GGACAT | 0.042% | 0.452 | no | 3 | 0.202 |
| GGACCT | 0.029% | 0.675 | no | 3 | −0.141 |
| GGACGT | 0.034% | 0.642 | no | 3 | 0.326 |
| GGACTA | 0.040% | 0.212 | no | 3 | 0.117 |
| GGACTC | 0.021% | 0.357 | no | 3 | 0.563 |
| GGACTG | 0.038% | 0.479 | no | 3 | −0.061 |
| GGAGAA | 0.075% | 0.346 | no | 3 | 0.683 |
| GGAGAC | 0.041% | 0.555 | no | 3 | 0.194 |
| GGAGAG | 0.057% | NA | no | 3 | −0.267 |
| GGAGCA | 0.056% | 0.193 | no | 3 | −0.902 |
| GGAGCC | 0.026% | 0.507 | no | 3 | 0.546 |
| GGAGCG | 0.039% | 0.339 | no | 3 | 0.798 |
| GGAGGA | 0.060% | 0.409 | no | 3 | 0.328 |
| GGAGGC | 0.033% | 0.361 | no | 3 | 0.855 |
| GGAGGG | 0.058% | −0.143 | no | 3 | −0.684 |
| GGATAT | 0.041% | NA | no | 3 | −0.148 |
| GGATCT | 0.032% | NA | no | 3 | 0.188 |
| GGATGT | 0.040% | 0.229 | no | 3 | −0.189 |
| GGATTA | 0.048% | NA | no | 3 | −0.693 |
| GGATTC | 0.030% | 0.464 | no | 3 | 0.476 |
| GGATTG | 0.036% | 0.244 | no | 3 | 0.652 |
| GGCATT | 0.022% | −0.082 | no | 3 | −0.580 |
| GGCCTT | 0.013% | NA | no | 3 | −2.576 |
| GGCGAT | 0.023% | 0.316 | no | 3 | −0.263 |
| GGCGCT | 0.014% | 0.188 | no | 3 | 1.147 |
| GGCGGT | 0.025% | NA | no | 3 | 1.035 |
| GGCGTA | 0.027% | NA | no | 3 | −0.527 |
| GGCGTC | 0.019% | 0.613 | no | 3 | 0.207 |
| GGCGTG | 0.018% | 0.188 | no | 3 | −0.668 |
| GGCTTT | 0.013% | −0.234 | no | 3 | −1.419 |
| GGGATT | 0.033% | NA | no | 3 | 0.234 |
| GGGCTT | 0.021% | −0.350 | no | 3 | −0.716 |
| GGGGAT | 0.049% | −0.238 | no | 3 | −0.226 |
| GGGGCT | 0.032% | −0.400 | no | 3 | −0.763 |
| GGGGGT | 0.040% | −0.654 | no | 3 | −1.990 |
| GGGGTA | 0.050% | −0.594 | no | 3 | −0.603 |
| GGGGTC | 0.027% | −0.349 | no | 3 | −1.000 |
| GGGGTG | 0.038% | −0.457 | no | 3 | 0.388 |
| GGGTTT | 0.028% | −0.581 | no | 3 | −1.389 |
| GGTATT | 0.025% | −0.474 | no | 3 | −0.435 |
| GGTCTT | 0.016% | −0.179 | no | 3 | −0.127 |
| GGTGAT | 0.035% | NA | no | 3 | 0.495 |
| GGTGCT | 0.019% | −0.189 | no | 3 | 1.239 |
| GGTGGT | 0.026% | −0.454 | no | 3 | 0.208 |
| GGTGTA | 0.035% | −0.244 | no | 3 | 0.050 |
| GGTGTC | 0.021% | NA | no | 3 | 0.167 |
| GGTGTG | 0.031% | −0.217 | no | 3 | −0.378 |
| GGTTTT | 0.022% | −0.371 | no | 3 | 0.528 |
| GTAATT | 0.024% | −0.589 | yes | 3 | −2.628 |
| GTACTT | 0.020% | −0.338 | no | 3 | −3.345 |
| GTAGAT | 0.042% | −0.455 | yes | 3 | −6.967 |
| GTAGCT | 0.021% | −0.471 | yes | 3 | −6.518 |
| GTAGGT | 0.033% | −0.709 | yes | 3 | −6.858 |
| GTAGTA | 0.034% | −0.666 | yes | 3 | −5.616 |
| GTAGTC | 0.019% | −0.348 | yes | 3 | −6.261 |
| GTAGTG | 0.026% | −0.390 | yes | 3 | −3.622 |
| GTATTT | 0.026% | −0.477 | no | 3 | −5.037 |
| GTCGTT | 0.017% | NA | no | 3 | −0.515 |
| GTGGTT | 0.020% | −0.158 | no | 3 | −1.517 |
| GTTGTT | 0.012% | NA | no | 3 | −2.903 |
| TAAAAT | 0.038% | −0.616 | no | 3 | −5.006 |
| TAAACT | 0.028% | −0.351 | no | 3 | −3.662 |
| TAAAGT | 0.043% | −0.299 | no | 3 | −4.139 |
| TAAATA | 0.030% | −0.601 | no | 3 | −3.979 |
| TAAATC | 0.022% | −0.298 | no | 3 | −7.739 |
| TAAATG | 0.031% | −0.486 | no | 3 | −1.724 |
| TAACAT | 0.035% | NA | no | 3 | −5.569 |
| TAACCT | 0.016% | NA | no | 3 | −4.175 |
| TAACGT | 0.027% | NA | no | 3 | −2.501 |
| TAACTA | 0.025% | −0.440 | no | 3 | −4.034 |
| TAACTC | 0.017% | NA | no | 3 | −3.670 |
| TAACTG | 0.022% | −0.235 | no | 3 | −0.752 |
| TAAGAA | 0.055% | NA | no | 3 | −3.674 |
| TAAGAC | 0.028% | NA | no | 3 | −4.744 |
| TAAGAG | 0.038% | −0.362 | no | 3 | −1.026 |
| TAAGCA | 0.037% | −0.402 | no | 3 | −5.206 |
| TAAGCC | 0.018% | −0.286 | no | 3 | −4.348 |
| TAAGCG | 0.030% | NA | no | 3 | −1.758 |
| TAAGGA | 0.036% | NA | no | 3 | −5.836 |
| TAAGGC | 0.023% | −0.372 | no | 3 | −5.147 |
| TAAGGG | 0.045% | −0.466 | no | 3 | −4.923 |
| TAATAT | 0.022% | −0.539 | no | 3 | −4.854 |
| TAATCT | 0.017% | NA | no | 3 | −6.017 |
| TAATGT | 0.022% | −0.340 | no | 3 | −5.765 |
| TAATTA | 0.031% | −0.642 | no | 3 | −5.341 |
| TAATTC | 0.020% | −0.483 | no | 3 | −7.396 |
| TAATTG | 0.023% | −0.563 | no | 3 | −1.442 |
| TACATT | 0.017% | −0.302 | no | 3 | −2.043 |
| TACCTT | 0.007% | NA | no | 3 | −2.904 |
| TACGAT | 0.018% | NA | no | 3 | 0.187 |
| TACGCT | 0.011% | NA | no | 3 | −1.509 |
| TACGGT | 0.018% | NA | no | 3 | −1.481 |
| TACGTA | 0.020% | NA | no | 3 | −0.495 |
| TACGTC | 0.013% | 0.501 | no | 3 | 3.451 |
| TACGTG | 0.014% | NA | no | 3 | 0.538 |
| TACTTT | 0.010% | −0.357 | no | 3 | −3.068 |
| TAGATT | 0.019% | −0.550 | no | 3 | −6.172 |
| TAGCTT | 0.013% | −0.691 | no | 3 | −6.108 |
| TAGGAT | 0.025% | −0.343 | no | 3 | −4.815 |
| TAGGCT | 0.014% | −0.609 | no | 3 | −8.651 |
| TAGGGT | 0.028% | −0.822 | no | 3 | −8.320 |
| TAGGTA | 0.031% | −0.997 | no | 3 | −8.642 |
| TAGGTC | 0.010% | −0.723 | no | 3 | −7.192 |
| TAGGTG | 0.028% | −0.594 | no | 3 | −4.297 |
| TAGTTT | 0.015% | −0.678 | no | 3 | −6.430 |
| TATATT | 0.029% | −0.716 | no | 3 | −5.131 |
| TATCTT | 0.011% | −0.287 | no | 3 | −6.037 |
| TATGAT | 0.028% | NA | no | 3 | −3.092 |
| TATGCT | 0.015% | −0.210 | no | 3 | −3.754 |
| TATGGT | 0.025% | −0.271 | no | 3 | −3.190 |
| TATGTA | 0.028% | −0.546 | no | 3 | −3.796 |
| TATGTC | 0.024% | NA | no | 3 | −4.029 |
| TATGTG | 0.025% | −0.253 | no | 3 | −2.263 |
| TATTTT | 0.019% | −0.564 | no | 3 | −6.436 |
| TCAAAT | 0.029% | NA | no | 3 | −2.787 |
| TCAACT | 0.016% | 0.275 | no | 3 | −0.972 |
| TCAAGT | 0.031% | NA | no | 3 | −0.725 |
| TCAATA | 0.027% | NA | no | 3 | −1.793 |
| TCAATC | 0.018% | NA | no | 3 | −4.515 |
| TCAATG | 0.023% | NA | no | 3 | −0.014 |
| TCACAT | 0.021% | NA | no | 3 | −2.039 |
| TCACCT | 0.012% | 0.270 | no | 3 | −1.313 |
| TCACGT | 0.014% | NA | no | 3 | −0.998 |
| TCACTA | 0.018% | NA | no | 3 | −0.889 |
| TCACTC | 0.014% | NA | no | 3 | −1.772 |
| TCACTG | 0.016% | NA | no | 3 | −0.806 |
| TCAGAA | 0.047% | NA | no | 3 | −4.515 |
| TCAGAC | 0.021% | NA | no | 3 | −2.038 |
| TCAGAG | 0.035% | NA | no | 3 | −0.865 |
| TCAGCA | 0.025% | NA | no | 3 | −3.365 |
| TCAGCC | 0.012% | −0.241 | no | 3 | −2.827 |
| TCAGCG | 0.022% | 0.343 | no | 3 | 0.375 |
| TCAGGA | 0.034% | NA | no | 3 | −0.819 |
| TCAGGC | 0.016% | NA | no | 3 | −3.842 |
| TCAGGG | 0.033% | −0.370 | no | 3 | −3.648 |
| TCATAT | 0.017% | NA | no | 3 | −2.724 |
| TCATCT | 0.014% | 0.329 | no | 3 | −0.379 |
| TCATGT | 0.021% | NA | no | 3 | −1.760 |
| TCATTA | 0.025% | NA | no | 3 | −4.234 |
| TCATTC | 0.017% | NA | no | 3 | −2.387 |
| TCATTG | 0.016% | NA | no | 3 | −0.997 |
| TCCATT | 0.009% | −0.265 | no | 3 | −2.492 |
| TCCCTT | 0.009% | −0.278 | no | 3 | −0.816 |
| TCCGAT | 0.011% | NA | no | 3 | −1.785 |
| TCCGCT | 0.010% | NA | no | 3 | −0.905 |
| TCCGGT | 0.010% | NA | no | 3 | 0.209 |
| TCCGTA | 0.012% | NA | no | 3 | 0.018 |
| TCCGTC | 0.008% | 0.514 | no | 3 | −0.799 |
| TCCGTG | 0.008% | 0.274 | no | 3 | 0.624 |
| TCGATT | 0.014% | NA | no | 3 | −2.198 |
| TCGCTT | 0.010% | NA | no | 3 | −5.211 |
| TCGGAT | 0.022% | 0.495 | no | 3 | −0.641 |
| TCGGCT | 0.016% | 0.287 | no | 3 | −1.098 |
| TCGGGT | 0.019% | NA | no | 3 | −0.018 |
| TCGGTA | 0.020% | NA | no | 3 | −1.295 |
| TCGGTC | 0.012% | 0.427 | no | 3 | −0.464 |
| TCGGTG | 0.020% | NA | no | 3 | −0.376 |
| TCGTTT | 0.010% | NA | no | 3 | −0.219 |
| TCTCTT | 0.010% | NA | no | 3 | −3.415 |
| TCTGAT | 0.015% | NA | no | 3 | −1.266 |
| TCTGCT | 0.011% | 0.232 | no | 3 | −1.122 |
| TCTGGT | 0.014% | NA | no | 3 | −2.332 |
| TCTGTA | 0.016% | NA | no | 3 | −1.150 |
| TCTGTC | 0.015% | NA | no | 3 | −3.655 |
| TCTGTG | 0.015% | NA | no | 3 | −1.585 |
| TCTTTT | 0.015% | −0.284 | no | 3 | −7.775 |
| TGAAAA | 0.045% | NA | no | 3 | −0.600 |
| TGAAAC | 0.028% | 0.263 | no | 3 | −0.135 |
| TGAAAG | 0.049% | NA | no | 3 | −0.400 |
| TGAACA | 0.036% | 0.267 | no | 3 | −0.244 |
| TGAACC | 0.022% | 0.252 | no | 3 | −1.184 |
| TGAACG | 0.036% | 0.515 | no | 3 | 0.040 |
| TGAAGA | 0.045% | 0.943 | no | 3 | 0.059 |
| TGAAGC | 0.029% | 0.521 | no | 3 | −0.184 |
| TGAAGG | 0.050% | 0.166 | no | 3 | −0.633 |
| TGACAA | 0.037% | 0.229 | no | 3 | −0.700 |
| TGACAC | 0.018% | NA | no | 3 | −0.663 |
| TGACAG | 0.035% | NA | no | 3 | −0.803 |
| TGACCA | 0.023% | 0.293 | no | 3 | −1.279 |
| TGACCC | 0.013% | NA | no | 3 | 0.342 |
| TGACCG | 0.018% | 0.547 | no | 3 | 1.260 |
| TGACGA | 0.025% | 0.717 | no | 3 | −0.063 |
| TGACGC | 0.015% | 0.539 | no | 3 | −0.065 |
| TGACGG | 0.032% | 0.511 | no | 3 | −0.330 |
| TGATAA | 0.034% | −0.373 | no | 3 | −1.068 |
| TGATAC | 0.021% | NA | no | 3 | 0.083 |
| TGATAG | 0.039% | −0.446 | no | 3 | −0.368 |
| TGATCA | 0.024% | NA | no | 3 | −0.254 |
| TGATCC | 0.010% | 0.172 | no | 3 | −0.561 |
| TGATCG | 0.021% | 0.536 | no | 3 | −0.187 |
| TGATGA | 0.024% | 0.451 | no | 3 | 0.825 |
| TGATGC | 0.019% | 0.140 | no | 3 | −0.150 |
| TGATGG | 0.033% | 0.215 | no | 3 | −0.654 |
| TGCAAT | 0.026% | −0.466 | no | 3 | −0.898 |
| TGCACT | 0.016% | −0.272 | no | 3 | 0.361 |
| TGCAGT | 0.020% | −0.227 | no | 3 | −1.736 |
| TGCATA | 0.022% | −0.622 | no | 3 | 0.019 |
| TGCATC | 0.016% | NA | no | 3 | −0.201 |
| TGCATG | 0.019% | NA | no | 3 | 1.544 |
| TGCCAT | 0.018% | −0.287 | no | 3 | 0.349 |
| TGCCCT | 0.010% | −0.267 | no | 3 | −0.272 |
| TGCCGT | 0.013% | NA | no | 3 | −1.273 |
| TGCCTA | 0.011% | −0.641 | no | 3 | −0.768 |
| TGCCTC | 0.012% | NA | no | 3 | −0.002 |
| TGCCTG | 0.010% | 0.154 | no | 3 | 0.076 |
| TGCGAA | 0.031% | NA | no | 3 | −0.027 |
| TGCGAC | 0.021% | 0.357 | no | 3 | 0.251 |
| TGCGAG | 0.022% | NA | no | 3 | −0.411 |
| TGCGCA | 0.018% | NA | no | 3 | −0.683 |
| TGCGCC | 0.015% | 0.309 | no | 3 | 0.544 |
| TGCGCG | 0.017% | 0.311 | no | 3 | 0.159 |
| TGCGGA | 0.029% | 0.458 | no | 3 | 0.733 |
| TGCGGC | 0.020% | NA | no | 3 | −0.896 |
| TGCGGG | 0.024% | 0.298 | no | 3 | 0.135 |
| TGCTAT | 0.015% | −0.323 | no | 3 | 0.629 |
| TGCTCT | 0.014% | −0.181 | no | 3 | −0.038 |
| TGCTGT | 0.018% | NA | no | 3 | 0.114 |
| TGCTTA | 0.019% | −0.584 | no | 3 | −0.135 |
| TGCTTC | 0.010% | NA | no | 3 | 0.307 |
| TGCTTG | 0.013% | −0.181 | no | 3 | −1.193 |
| TGGAAT | 0.039% | 0.326 | no | 3 | 0.467 |
| TGGACT | 0.017% | 0.449 | no | 3 | 1.333 |
| TGGAGT | 0.030% | 0.275 | no | 3 | −0.605 |
| TGGATA | 0.034% | 0.123 | no | 3 | −0.884 |
| TGGATC | 0.014% | 0.359 | no | 3 | 1.283 |
| TGGATG | 0.034% | 0.414 | no | 3 | 0.515 |
| TGGCAT | 0.029% | NA | no | 3 | 0.327 |
| TGGCCT | 0.013% | NA | no | 3 | −0.865 |
| TGGCGT | 0.018% | 0.321 | no | 3 | 0.781 |
| TGGCTA | 0.019% | −0.156 | no | 3 | −0.691 |
| TGGCTC | 0.009% | NA | no | 3 | −1.112 |
| TGGCTG | 0.022% | 0.169 | no | 3 | −0.952 |
| TGGGAA | 0.044% | NA | no | 3 | −0.213 |
| TGGGAC | 0.025% | 0.286 | no | 3 | −0.590 |
| TGGGAG | 0.032% | 0.077 | no | 3 | −0.855 |
| TGGGCA | 0.029% | −0.105 | no | 3 | 0.600 |
| TGGGCC | 0.014% | NA | no | 3 | −0.785 |
| TGGGCG | 0.018% | NA | no | 3 | 0.933 |
| TGGGGA | 0.037% | −0.113 | no | 3 | 0.433 |
| TGGGGC | 0.021% | −0.149 | no | 3 | −0.473 |
| TGGGGG | 0.032% | −0.413 | no | 3 | −0.787 |
| TGGTAT | 0.023% | −0.363 | no | 3 | −0.380 |
| TGGTCT | 0.016% | NA | no | 3 | −0.378 |
| TGGTGT | 0.023% | NA | no | 3 | −0.162 |
| TGGTTA | 0.021% | −0.457 | no | 3 | −0.466 |
| TGGTTC | 0.013% | NA | no | 3 | 0.216 |
| TGGTTG | 0.022% | −0.211 | no | 3 | −1.223 |
| TGTAAT | 0.029% | −0.389 | no | 3 | −0.246 |
| TGTACT | 0.014% | NA | no | 3 | −1.112 |
| TGTAGT | 0.024% | −0.492 | no | 3 | 0.273 |
| TGTATA | 0.034% | −0.526 | no | 3 | −0.677 |
| TGTATC | 0.018% | NA | no | 3 | 0.045 |
| TGTATG | 0.030% | −0.152 | no | 3 | 0.022 |
| TGTCAT | 0.015% | NA | no | 3 | 0.044 |
| TGTCCT | 0.011% | NA | no | 3 | 1.450 |
| TGTCGT | 0.013% | 0.280 | no | 3 | −0.371 |
| TGTCTA | 0.012% | −0.259 | no | 3 | −0.468 |
| TGTCTC | 0.015% | NA | no | 3 | 0.228 |
| TGTCTG | 0.017% | NA | no | 3 | −0.289 |
| TGTGAA | 0.036% | 0.381 | no | 3 | −0.270 |
| TGTGAC | 0.019% | 0.329 | no | 3 | −0.279 |
| TGTGAG | 0.028% | 0.171 | no | 3 | 0.107 |
| TGTGCA | 0.021% | −0.247 | no | 3 | −0.470 |
| TGTGCG | 0.019% | −0.081 | no | 3 | −0.277 |
| TGTGGA | 0.021% | 0.595 | no | 3 | −1.476 |
| TGTGGC | 0.019% | 0.063 | no | 3 | −0.805 |
| TGTGGG | 0.030% | −0.060 | no | 3 | −0.614 |
| TGTTAT | 0.023% | −0.263 | no | 3 | −0.909 |
| TGTTCT | 0.011% | NA | no | 3 | 0.257 |
| TGTTGT | 0.018% | NA | no | 3 | 0.259 |
| TGTTTA | 0.021% | −0.532 | no | 3 | 0.716 |
| TGTTTC | 0.012% | NA | no | 3 | −0.515 |
| TGTTTG | 0.015% | −0.161 | no | 3 | −0.273 |
| TTAAAT | 0.028% | −0.558 | yes | 3 | −5.460 |
| TTAACT | 0.025% | −0.216 | yes | 3 | −3.918 |
| TTAAGT | 0.028% | −0.496 | yes | 3 | −6.742 |
| TTAATA | 0.023% | −0.424 | yes | 3 | −6.554 |
| TTAATC | 0.025% | NA | yes | 3 | −8.686 |
| TTAATG | 0.029% | −0.404 | yes | 3 | −4.133 |
| TTACAT | 0.020% | NA | no | 3 | −3.758 |
| TTACCT | 0.010% | 0.221 | no | 3 | −1.043 |
| TTACGT | 0.010% | NA | no | 3 | −0.727 |
| TTACTA | 0.013% | NA | no | 3 | −2.319 |
| TTACTC | 0.016% | NA | no | 3 | −5.081 |
| TTACTG | 0.017% | NA | no | 3 | −0.218 |
| TTAGAA | 0.034% | −0.513 | yes | 3 | −8.530 |
| TTAGAC | 0.016% | −0.392 | yes | 3 | −6.524 |
| TTAGAG | 0.029% | −0.545 | yes | 3 | −4.639 |
| TTAGCA | 0.023% | −0.523 | yes | 3 | −7.346 |
| TTAGCC | 0.009% | −0.575 | yes | 3 | −6.467 |
| TTAGCG | 0.021% | −0.326 | yes | 3 | −5.679 |
| TTAGGA | 0.025% | −0.627 | yes | 3 | −5.762 |
| TTAGGC | 0.011% | −0.712 | yes | 3 | −5.624 |
| TTAGGG | 0.027% | −0.827 | yes | 3 | −7.215 |
| TTATAT | 0.021% | −0.461 | no | 3 | −2.689 |
| TTATCT | 0.009% | NA | no | 3 | −3.800 |
| TTATGT | 0.018% | −0.272 | no | 3 | −4.521 |
| TTATTA | 0.022% | −0.478 | no | 3 | −4.891 |
| TTATTC | 0.016% | NA | no | 3 | −5.148 |
| TTATTG | 0.023% | −0.343 | no | 3 | −0.759 |
| TTCATT | 0.014% | NA | no | 3 | −1.816 |
| TTCGAT | 0.016% | 0.344 | no | 3 | 0.678 |
| TTCGCT | 0.011% | 0.308 | no | 3 | −1.573 |
| TTCGGT | 0.014% | NA | no | 3 | 0.331 |
| TTCGTA | 0.013% | NA | no | 3 | −1.039 |
| TTCGTC | 0.010% | 0.789 | no | 3 | −0.765 |
| TTCGTG | 0.012% | 0.334 | no | 3 | −0.550 |
| TTCTTT | 0.010% | −0.207 | no | 3 | −3.591 |
| TTGATT | 0.017% | NA | yes | 3 | −2.576 |
| TTGCTT | 0.009% | NA | no | 3 | −2.608 |
| TTGGAT | 0.027% | 0.261 | no | 3 | −0.211 |
| TTGGCT | 0.015% | NA | no | 3 | −0.851 |
| TTGGGT | 0.025% | −0.453 | no | 3 | −5.418 |
| TTGGTA | 0.023% | −0.369 | no | 3 | −3.830 |
| TTGGTC | 0.011% | NA | no | 3 | −2.246 |
| TTGGTG | 0.015% | −0.173 | no | 3 | 0.241 |
| TTGTTT | 0.016% | −0.196 | no | 3 | −2.805 |
| TTTATT | 0.013% | −0.491 | no | 3 | −4.281 |
| TTTCTT | 0.011% | −0.214 | no | 3 | −6.475 |
| TTTGAT | 0.015% | NA | no | 3 | −3.027 |
| TTTGCT | 0.011% | −0.219 | no | 3 | −1.789 |
| TTTGGT | 0.021% | −0.323 | no | 3 | −4.850 |
| TTTGTA | 0.018% | −0.291 | no | 3 | −3.348 |
| TTTGTC | 0.015% | NA | no | 3 | −2.165 |
| TTTGTG | 0.019% | −0.222 | no | 3 | −0.369 |
| TTTTTT | 0.011% | −0.381 | no | 3 | −4.402 |
| AAAAAT | 0.088% | −0.343 | no | 4 | −3.516 |
| AAAACT | 0.051% | NA | no | 4 | −2.594 |
| AAAAGT | 0.074% | NA | no | 4 | −3.958 |
| AAAATA | 0.054% | NA | no | 4 | −3.325 |
| AAAATC | 0.048% | NA | no | 4 | −4.381 |
| AAAATG | 0.069% | −0.216 | no | 4 | −1.247 |
| AAACAT | 0.056% | NA | no | 4 | −3.092 |
| AAACCT | 0.029% | 0.310 | no | 4 | −2.728 |
| AAACGT | 0.047% | 0.294 | no | 4 | −1.513 |
| AAACTA | 0.047% | NA | no | 4 | −3.020 |
| AAACTC | 0.034% | NA | no | 4 | −2.516 |
| AAACTG | 0.046% | NA | no | 4 | −0.916 |
| AAAGAA | 0.103% | 0.355 | no | 4 | −1.592 |
| AAAGAC | 0.054% | 0.390 | no | 4 | −1.483 |
| AAAGAG | 0.080% | NA | no | 4 | 0.060 |
| AAAGCA | 0.066% | NA | no | 4 | −3.346 |
| AAAGCC | 0.036% | NA | no | 4 | −0.725 |
| AAAGCG | 0.049% | 0.309 | no | 4 | −0.741 |
| AAAGGA | 0.084% | 0.418 | no | 4 | −1.714 |
| AAAGGC | 0.054% | NA | no | 4 | −0.894 |
| AAAGGG | 0.082% | −0.263 | no | 4 | −2.890 |
| AAATAT | 0.036% | −0.413 | no | 4 | −4.350 |
| AAATCT | 0.032% | NA | no | 4 | −3.355 |
| AAATGT | 0.048% | NA | no | 4 | −3.537 |
| AAATTA | 0.045% | NA | no | 4 | −5.631 |
| AAATTC | 0.024% | NA | no | 4 | −5.160 |
| AAATTG | 0.045% | −0.228 | no | 4 | −1.111 |
| AACATT | 0.036% | NA | no | 4 | −1.558 |
| AACCTT | 0.022% | NA | no | 4 | −2.150 |
| AACGAT | 0.042% | 0.386 | no | 4 | 0.296 |
| AACGCT | 0.026% | 0.361 | no | 4 | −0.309 |
| AACGGT | 0.039% | NA | no | 4 | −0.159 |
| AACGTA | 0.040% | NA | no | 4 | −0.906 |
| AACGTC | 0.032% | 0.684 | no | 4 | −0.611 |
| AACGTG | 0.037% | 0.265 | no | 4 | −0.098 |
| AACTTT | 0.022% | NA | no | 4 | −1.717 |
| AAGATT | 0.044% | 0.448 | no | 4 | −0.536 |
| AAGCTT | 0.031% | NA | no | 4 | −1.965 |
| AAGGAT | 0.053% | 0.567 | no | 4 | −0.813 |
| AAGGCT | 0.029% | NA | no | 4 | −3.067 |
| AAGGGT | 0.049% | NA | no | 4 | −7.986 |
| AAGGTA | 0.057% | NA | no | 4 | −5.594 |
| AAGGTC | 0.026% | NA | no | 4 | −2.533 |
| AAGGTG | 0.054% | −0.162 | no | 4 | −0.085 |
| AAGTTT | 0.032% | NA | no | 4 | −2.517 |
| AATATT | 0.036% | −0.315 | no | 4 | −4.895 |
| AATCTT | 0.021% | NA | no | 4 | −3.098 |
| AATGAT | 0.046% | NA | no | 4 | −2.293 |
| AATGCT | 0.024% | NA | no | 4 | −1.456 |
| AATGGT | 0.034% | −0.241 | no | 4 | −2.975 |
| AATGTA | 0.037% | NA | no | 4 | −4.311 |
| AATGTC | 0.019% | NA | no | 4 | −5.201 |
| AATGTG | 0.036% | NA | no | 4 | −0.676 |
| AATTTT | 0.018% | NA | no | 4 | −5.472 |
| ACAAAT | 0.060% | NA | no | 4 | −2.498 |
| ACAACT | 0.034% | NA | no | 4 | −1.343 |
| ACAAGT | 0.043% | NA | no | 4 | −1.335 |
| ACAATA | 0.051% | NA | no | 4 | −2.992 |
| ACAATC | 0.035% | NA | no | 4 | −2.196 |
| ACAATG | 0.044% | NA | no | 4 | −0.777 |
| ACACAT | 0.039% | NA | no | 4 | −2.724 |
| ACACCT | 0.027% | 0.299 | no | 4 | −0.778 |
| ACACGT | 0.032% | NA | no | 4 | −1.097 |
| ACACTA | 0.025% | −0.421 | no | 4 | −1.741 |
| ACACTC | 0.024% | 0.299 | no | 4 | −2.868 |
| ACACTG | 0.034% | 0.160 | no | 4 | −0.472 |
| ACAGAA | 0.065% | NA | no | 4 | −1.632 |
| ACAGAC | 0.040% | 0.393 | no | 4 | −0.419 |
| ACAGAG | 0.050% | NA | no | 4 | −0.040 |
| ACAGCA | 0.040% | NA | no | 4 | −0.830 |
| ACAGCC | 0.029% | NA | no | 4 | −0.837 |
| ACAGCG | 0.039% | 0.332 | no | 4 | 0.693 |
| ACAGGA | 0.059% | NA | no | 4 | −0.977 |
| ACAGGC | 0.039% | NA | no | 4 | −2.302 |
| ACAGGG | 0.053% | −0.305 | no | 4 | −3.989 |
| ACATAT | 0.038% | −0.375 | no | 4 | −3.134 |
| ACATCT | 0.026% | 0.265 | no | 4 | −0.972 |
| ACATGT | 0.028% | NA | no | 4 | −1.629 |
| ACATTA | 0.038% | NA | no | 4 | −3.513 |
| ACATTC | 0.024% | NA | no | 4 | −3.662 |
| ACATTG | 0.029% | NA | no | 4 | −1.130 |
| ACCATT | 0.020% | NA | no | 4 | −0.629 |
| ACCCTT | 0.011% | −0.246 | no | 4 | 0.422 |
| ACCGAT | 0.022% | NA | no | 4 | −0.135 |
| ACCGCT | 0.016% | NA | no | 4 | −1.150 |
| ACCGGT | 0.021% | NA | no | 4 | −0.674 |
| ACCGTA | 0.024% | NA | no | 4 | −0.726 |
| ACCGTC | 0.016% | NA | no | 4 | −0.451 |
| ACCGTG | 0.023% | 0.196 | no | 4 | 0.271 |
| ACCTTT | 0.014% | NA | no | 4 | −2.132 |
| ACGATT | 0.031% | NA | no | 4 | −1.041 |
| ACGCTT | 0.020% | NA | no | 4 | −1.397 |
| ACGGAT | 0.042% | 0.588 | no | 4 | 0.238 |
| ACGGCT | 0.019% | NA | no | 4 | −0.593 |
| ACGGGT | 0.038% | NA | no | 4 | −0.240 |
| ACGGTA | 0.043% | NA | no | 4 | −1.121 |
| ACGGTC | 0.028% | 0.541 | no | 4 | −0.430 |
| ACGGTG | 0.022% | NA | no | 4 | −0.185 |
| ACGTTT | 0.025% | NA | no | 4 | −1.488 |
| ACTATT | 0.023% | −0.296 | no | 4 | −5.484 |
| ACTCTT | 0.017% | NA | no | 4 | −3.181 |
| ACTGAT | 0.025% | 0.268 | no | 4 | −1.437 |
| ACTGCT | 0.020% | 0.258 | no | 4 | −2.009 |
| ACTGGT | 0.025% | NA | no | 4 | −2.074 |
| ACTGTA | 0.032% | NA | no | 4 | −2.334 |
| ACTGTC | 0.018% | 0.305 | no | 4 | −1.646 |
| ACTGTG | 0.030% | NA | no | 4 | −0.724 |
| ACTTTT | 0.018% | −0.426 | no | 4 | −6.098 |
| AGAAAA | 0.098% | NA | no | 4 | −5.216 |
| AGAAAC | 0.053% | 0.315 | no | 4 | −5.765 |
| AGAAAG | 0.090% | NA | no | 4 | −1.012 |
| AGAACA | 0.065% | NA | no | 4 | −5.222 |
| AGAACC | 0.041% | NA | no | 4 | −4.164 |
| AGAACG | 0.045% | 0.447 | no | 4 | −2.160 |
| AGAAGA | 0.067% | 1.034 | no | 4 | −3.548 |
| AGAAGC | 0.040% | 0.505 | no | 4 | −4.989 |
| AGAAGG | 0.072% | NA | no | 4 | −3.572 |
| AGACAA | 0.067% | NA | no | 4 | −2.772 |
| AGACAC | 0.034% | NA | no | 4 | −1.755 |
| AGACAG | 0.048% | NA | no | 4 | −1.138 |
| AGACCA | 0.035% | NA | no | 4 | −1.356 |
| AGACCC | 0.020% | NA | no | 4 | −2.073 |
| AGACCG | 0.032% | 0.453 | no | 4 | 0.067 |
| AGACGA | 0.037% | 0.601 | no | 4 | −1.130 |
| AGACGC | 0.032% | 0.526 | no | 4 | −0.923 |
| AGACGG | 0.045% | 0.309 | no | 4 | −0.496 |
| AGATAA | 0.054% | NA | no | 4 | −3.790 |
| AGATAC | 0.035% | NA | no | 4 | −2.669 |
| AGATAG | 0.049% | −0.630 | no | 4 | −2.242 |
| AGATCA | 0.040% | NA | no | 4 | −3.429 |
| AGATCC | 0.023% | 0.192 | no | 4 | −2.733 |
| AGATCG | 0.034% | 0.331 | no | 4 | −0.645 |
| AGATGA | 0.041% | NA | no | 4 | −4.270 |
| AGATGC | 0.037% | 0.194 | no | 4 | −1.573 |
| AGATGG | 0.042% | NA | no | 4 | −0.703 |
| AGCAAT | 0.044% | NA | no | 4 | −0.655 |
| AGCACT | 0.026% | NA | no | 4 | 0.946 |
| AGCAGT | 0.037% | NA | no | 4 | −0.523 |
| AGCATA | 0.048% | NA | no | 4 | −1.072 |
| AGCATC | 0.034% | NA | no | 4 | −0.139 |
| AGCATG | 0.033% | NA | no | 4 | 0.113 |
| AGCCAT | 0.024% | NA | no | 4 | 0.515 |
| AGCCCT | 0.015% | NA | no | 4 | 0.461 |
| AGCCGT | 0.021% | NA | no | 4 | −0.579 |
| AGCCTA | 0.022% | −0.677 | no | 4 | 0.105 |
| AGCCTC | 0.019% | NA | no | 4 | −0.799 |
| AGCCTG | 0.020% | NA | no | 4 | 0.459 |
| AGCGAA | 0.042% | NA | no | 4 | −0.480 |
| AGCGAC | 0.033% | 0.462 | no | 4 | −0.260 |
| AGCGAG | 0.039% | NA | no | 4 | 0.894 |
| AGCGCA | 0.032% | NA | no | 4 | −0.334 |
| AGCGCC | 0.016% | NA | no | 4 | 0.702 |
| AGCGCG | 0.037% | 0.413 | no | 4 | −0.188 |
| AGCGGA | 0.047% | 0.440 | no | 4 | 0.284 |
| AGCGGC | 0.031% | NA | no | 4 | 0.786 |
| AGCGGG | 0.038% | 0.181 | no | 4 | −0.245 |
| AGCTAT | 0.029% | −0.338 | no | 4 | −0.014 |
| AGCTCT | 0.017% | NA | no | 4 | −0.602 |
| AGCTGT | 0.022% | NA | no | 4 | −0.424 |
| AGCTTA | 0.024% | −0.405 | no | 4 | −1.115 |
| AGCTTC | 0.018% | NA | no | 4 | 0.101 |
| AGCTTG | 0.026% | −0.330 | no | 4 | −0.058 |
| AGGAAT | 0.067% | NA | no | 4 | −2.991 |
| AGGACT | 0.035% | 0.392 | no | 4 | −3.459 |
| AGGAGT | 0.039% | NA | no | 4 | −1.870 |
| AGGATA | 0.062% | NA | no | 4 | −7.902 |
| AGGATC | 0.035% | 0.379 | no | 4 | −4.373 |
| AGGATG | 0.052% | 0.187 | no | 4 | −5.328 |
| AGGCAT | 0.044% | NA | no | 4 | −1.828 |
| AGGCCT | 0.023% | NA | no | 4 | −0.382 |
| AGGCGT | 0.034% | NA | no | 4 | −0.397 |
| AGGCTA | 0.049% | −0.404 | no | 4 | −3.324 |
| AGGCTC | 0.030% | NA | no | 4 | −1.454 |
| AGGCTG | 0.030% | NA | no | 4 | 0.107 |
| AGGGAA | 0.077% | NA | no | 4 | −5.306 |
| AGGGAC | 0.043% | NA | no | 4 | −6.309 |
| AGGGAG | 0.059% | −0.373 | no | 4 | −2.135 |
| AGGGCA | 0.057% | −0.267 | no | 4 | −6.921 |
| AGGGCC | 0.032% | −0.371 | no | 4 | −4.173 |
| AGGGCG | 0.043% | −0.249 | no | 4 | −1.496 |
| AGGGGA | 0.080% | −0.275 | no | 4 | −3.922 |
| AGGGGC | 0.038% | −0.225 | no | 4 | −5.261 |
| AGGGGG | 0.065% | −0.464 | no | 4 | −3.875 |
| AGGTAT | 0.042% | −0.401 | no | 4 | −4.452 |
| AGGTCT | 0.029% | NA | no | 4 | −4.472 |
| AGGTGT | 0.037% | −0.286 | no | 4 | −2.006 |
| AGGTTA | 0.047% | −0.602 | no | 4 | −3.642 |
| AGGTTC | 0.028% | NA | no | 4 | −4.493 |
| AGGTTG | 0.041% | −0.476 | no | 4 | −1.140 |
| AGTAAT | 0.049% | −0.394 | no | 4 | −1.198 |
| AGTACT | 0.034% | NA | no | 4 | −0.954 |
| AGTAGT | 0.035% | −0.373 | no | 4 | −3.359 |
| AGTATA | 0.055% | −0.425 | no | 4 | −1.458 |
| AGTATC | 0.031% | NA | no | 4 | −1.182 |
| AGTATG | 0.041% | −0.251 | no | 4 | −0.309 |
| AGTCAT | 0.039% | NA | no | 4 | −1.017 |
| AGTCCT | 0.017% | NA | no | 4 | −0.880 |
| AGTCGT | 0.024% | 0.257 | no | 4 | 0.145 |
| AGTCTA | 0.035% | −0.467 | no | 4 | −0.810 |
| AGTCTC | 0.020% | NA | no | 4 | 1.087 |
| AGTCTG | 0.025% | NA | no | 4 | −0.660 |
| AGTGAA | 0.053% | NA | no | 4 | −0.256 |
| AGTGAC | 0.025% | 0.323 | no | 4 | −0.127 |
| AGTGAG | 0.045% | −0.374 | no | 4 | −0.022 |
| AGTGCA | 0.043% | −0.169 | no | 4 | −0.841 |
| AGTGCC | 0.017% | NA | no | 4 | 0.818 |
| AGTGCG | 0.038% | NA | no | 4 | 0.561 |
| AGTGGA | 0.060% | NA | no | 4 | −0.600 |
| AGTGGC | 0.030% | NA | no | 4 | 0.516 |
| AGTGGG | 0.047% | −0.246 | no | 4 | −0.432 |
| AGTTAT | 0.033% | −0.091 | no | 4 | −1.026 |
| AGTTCT | 0.018% | 0.158 | no | 4 | −0.424 |
| AGTTGT | 0.024% | NA | no | 4 | 0.336 |
| AGTTTA | 0.040% | −0.216 | no | 4 | −2.315 |
| AGTTTC | 0.022% | 0.254 | no | 4 | −1.797 |
| AGTTTG | 0.026% | NA | no | 4 | 0.155 |
| ATAAAT | 0.052% | −0.446 | yes | 4 | −6.581 |
| ATAACT | 0.029% | −0.316 | yes | 4 | −3.848 |
| ATAAGT | 0.052% | NA | yes | 4 | −7.114 |
| ATAATA | 0.054% | −0.503 | yes | 4 | −5.864 |
| ATAATC | 0.032% | NA | yes | 4 | −5.629 |
| ATAATG | 0.049% | −0.335 | yes | 4 | −4.245 |
| ATACAT | 0.033% | NA | no | 4 | −3.054 |
| ATACCT | 0.018% | NA | no | 4 | −1.447 |
| ATACGT | 0.032% | NA | no | 4 | −0.910 |
| ATACTA | 0.037% | NA | no | 4 | −5.216 |
| ATACTC | 0.020% | NA | no | 4 | −1.292 |
| ATACTG | 0.033% | NA | no | 4 | −0.298 |
| ATAGAA | 0.053% | NA | yes | 4 | −8.271 |
| ATAGAC | 0.031% | NA | yes | 4 | −8.524 |
| ATAGAG | 0.049% | −0.265 | yes | 4 | −3.258 |
| ATAGCA | 0.040% | −0.383 | yes | 4 | −7.504 |
| ATAGCC | 0.018% | −0.279 | yes | 4 | −5.802 |
| ATAGCG | 0.040% | NA | yes | 4 | −4.412 |
| ATAGGA | 0.047% | NA | yes | 4 | −6.413 |
| ATAGGC | 0.021% | NA | yes | 4 | −5.793 |
| ATAGGG | 0.044% | −0.698 | yes | 4 | −8.103 |
| ATATAT | 0.034% | −0.374 | no | 4 | −3.377 |
| ATATCT | 0.027% | NA | no | 4 | −3.044 |
| ATATGT | 0.034% | NA | no | 4 | −3.775 |
| ATATTA | 0.045% | −0.360 | no | 4 | −3.798 |
| ATATTC | 0.024% | NA | no | 4 | −5.407 |
| ATATTG | 0.041% | −0.328 | no | 4 | −1.972 |
| ATCATT | 0.028% | NA | no | 4 | 0.861 |
| ATCCTT | 0.019% | NA | no | 4 | 0.264 |
| ATCGAT | 0.034% | 0.312 | no | 4 | −0.464 |
| ATCGCT | 0.021% | NA | no | 4 | 0.221 |
| ATCGGT | 0.032% | NA | no | 4 | 0.601 |
| ATCGTA | 0.032% | NA | no | 4 | 0.152 |
| ATCGTC | 0.020% | 0.652 | no | 4 | −0.624 |
| ATCGTG | 0.032% | 0.214 | no | 4 | −0.159 |
| ATCTTT | 0.018% | NA | no | 4 | −0.768 |
| ATGATT | 0.040% | NA | yes | 4 | −4.782 |
| ATGCTT | 0.025% | NA | no | 4 | −2.106 |
| ATGGAT | 0.042% | 0.299 | no | 4 | −0.973 |
| ATGGCT | 0.027% | NA | no | 4 | −0.920 |
| ATGGGT | 0.042% | −0.494 | no | 4 | −4.650 |
| ATGGTA | 0.043% | −0.484 | no | 4 | −3.345 |
| ATGGTC | 0.035% | NA | no | 4 | −3.585 |
| ATGGTG | 0.038% | −0.148 | no | 4 | −0.300 |
| ATGTTT | 0.030% | NA | no | 4 | −3.404 |
| ATTATT | 0.025% | −0.286 | no | 4 | −4.186 |
| ATTCTT | 0.018% | −0.283 | no | 4 | −3.950 |
| ATTGAT | 0.039% | NA | no | 4 | −2.368 |
| ATTGCT | 0.018% | −0.110 | no | 4 | −3.597 |
| ATTGGT | 0.033% | NA | no | 4 | −1.753 |
| ATTGTA | 0.037% | −0.367 | no | 4 | −3.645 |
| ATTGTC | 0.019% | NA | no | 4 | −4.034 |
| ATTGTG | 0.030% | −0.157 | no | 4 | −0.111 |
| ATTTTT | 0.020% | −0.599 | no | 4 | −6.439 |
| CAAAAT | 0.035% | −0.265 | no | 4 | −1.870 |
| CAAACT | 0.026% | NA | no | 4 | −1.080 |
| CAAAGT | 0.031% | NA | no | 4 | −1.396 |
| CAAATA | 0.026% | −0.390 | no | 4 | −2.759 |
| CAAATC | 0.020% | NA | no | 4 | −1.123 |
| CAAATG | 0.032% | NA | no | 4 | 0.408 |
| CAACAT | 0.027% | 0.196 | no | 4 | −1.648 |
| CAACCT | 0.013% | 0.189 | no | 4 | −0.682 |
| CAACGT | 0.017% | 0.414 | no | 4 | −0.326 |
| CAACTA | 0.018% | NA | no | 4 | −0.869 |
| CAACTC | 0.012% | 0.284 | no | 4 | −1.207 |
| CAACTG | 0.022% | 0.270 | no | 4 | 1.389 |
| CAAGAA | 0.041% | 0.922 | no | 4 | −0.995 |
| CAAGAC | 0.020% | 0.705 | no | 4 | −0.250 |
| CAAGAG | 0.031% | 0.411 | no | 4 | 0.703 |
| CAAGCA | 0.026% | NA | no | 4 | −1.164 |
| CAAGCC | 0.016% | NA | no | 4 | −0.329 |
| CAAGCG | 0.021% | 0.353 | no | 4 | 1.237 |
| CAAGGA | 0.038% | 0.620 | no | 4 | −0.511 |
| CAAGGC | 0.021% | 0.209 | no | 4 | −1.030 |
| CAAGGG | 0.034% | NA | no | 4 | −0.664 |
| CAATAT | 0.020% | −0.268 | no | 4 | −2.422 |
| CAATCT | 0.014% | NA | no | 4 | 1.219 |
| CAATGT | 0.019% | NA | no | 4 | −0.901 |
| CAATTA | 0.024% | −0.483 | no | 4 | −1.901 |
| CAATTG | 0.015% | NA | no | 4 | 0.420 |
| CACATT | 0.014% | NA | no | 4 | 3.185 |
| CACCTT | 0.009% | −0.286 | no | 4 | −1.341 |
| CACGAT | 0.023% | NA | no | 4 | −0.362 |
| CACGCT | 0.010% | NA | no | 4 | −0.082 |
| CACGGT | 0.019% | NA | no | 4 | −0.840 |
| CACGTA | 0.021% | −0.253 | no | 4 | −0.043 |
| CACGTC | 0.010% | 0.302 | no | 4 | 0.755 |
| CACGTG | 0.014% | NA | no | 4 | 0.005 |
| CACTTT | 0.013% | −0.551 | no | 4 | 0.538 |
| CAGATT | 0.024% | NA | no | 4 | −2.078 |
| CAGCTT | 0.017% | −0.379 | no | 4 | −0.713 |
| CAGGAT | 0.029% | NA | no | 4 | −0.515 |
| CAGGCT | 0.020% | −0.217 | no | 4 | −3.261 |
| CAGGGT | 0.031% | −0.438 | no | 4 | −5.036 |
| CAGGTA | 0.030% | −0.604 | no | 4 | −4.058 |
| CAGGTC | 0.016% | −0.283 | no | 4 | −3.073 |
| CAGGTG | 0.028% | −0.405 | no | 4 | −1.431 |
| CAGTTT | 0.017% | −0.231 | no | 4 | −1.048 |
| CATATT | 0.013% | −0.298 | no | 4 | −3.047 |
| CATCTT | 0.009% | −0.255 | no | 4 | −0.392 |
| CATGAT | 0.021% | NA | no | 4 | −1.500 |
| CATGCT | 0.015% | NA | no | 4 | 1.442 |
| CATGGT | 0.018% | NA | no | 4 | −1.403 |
| CATGTA | 0.021% | −0.228 | no | 4 | −1.773 |
| CATGTC | 0.011% | NA | no | 4 | −3.145 |
| CATGTG | 0.017% | NA | no | 4 | 1.066 |
| CATTTT | 0.013% | −0.488 | no | 4 | −5.269 |
| CCAAAT | 0.018% | −0.435 | no | 4 | −2.137 |
| CCAACT | 0.017% | NA | no | 4 | −1.663 |
| CCAAGT | 0.020% | NA | no | 4 | −1.211 |
| CCAATA | 0.020% | −0.660 | no | 4 | −2.066 |
| CCAATC | 0.016% | −0.206 | no | 4 | −2.827 |
| CCAATG | 0.019% | NA | no | 4 | −0.077 |
| CCACAT | 0.018% | NA | no | 4 | −1.707 |
| CCACCT | 0.013% | −0.247 | no | 4 | −1.130 |
| CCACGT | 0.013% | 0.193 | no | 4 | −0.068 |
| CCACTA | 0.015% | −0.448 | no | 4 | −1.820 |
| CCACTC | 0.011% | NA | no | 4 | −1.964 |
| CCACTG | 0.013% | NA | no | 4 | −0.784 |
| CCAGAA | 0.031% | NA | no | 4 | −1.968 |
| CCAGAC | 0.018% | −0.293 | no | 4 | 0.897 |
| CCAGAG | 0.023% | NA | no | 4 | −0.694 |
| CCAGCA | 0.020% | −0.894 | no | 4 | 0.249 |
| CCAGCC | 0.010% | −0.361 | no | 4 | −0.074 |
| CCAGCG | 0.016% | NA | no | 4 | 0.296 |
| CCAGGA | 0.023% | NA | no | 4 | −0.226 |
| CCAGGC | 0.014% | NA | no | 4 | 0.094 |
| CCAGGG | 0.023% | −0.081 | no | 4 | −0.517 |
| CCATAT | 0.014% | −0.454 | no | 4 | −4.374 |
| CCATCT | 0.011% | NA | no | 4 | −1.363 |
| CCATGT | 0.011% | NA | no | 4 | −0.157 |
| CCATTA | 0.017% | −0.770 | no | 4 | −1.677 |
| CCATTC | 0.009% | NA | no | 4 | −0.177 |
| CCATTG | 0.011% | NA | no | 4 | −1.246 |
| CCCATT | 0.007% | −0.354 | no | 4 | −1.982 |
| CCCCTT | 0.005% | −0.393 | no | 4 | −3.756 |
| CCCGAT | 0.013% | NA | no | 4 | −0.672 |
| CCCGCT | 0.012% | 0.270 | no | 4 | −1.878 |
| CCCGGT | 0.007% | 0.115 | no | 4 | −0.464 |
| CCCGTA | 0.010% | −0.151 | no | 4 | 0.711 |
| CCCGTC | 0.006% | 0.533 | no | 4 | −0.470 |
| CCCGTG | 0.009% | 0.323 | no | 4 | 2.700 |
| CCCTTT | 0.007% | −0.551 | no | 4 | −1.067 |
| CCGATT | 0.016% | NA | no | 4 | 0.036 |
| CCGCTT | 0.007% | NA | no | 4 | 1.319 |
| CCGGAT | 0.018% | 0.357 | no | 4 | −0.103 |
| CCGGCT | 0.012% | NA | no | 4 | 0.454 |
| CCGGGT | 0.015% | NA | no | 4 | −0.140 |
| CCGGTA | 0.015% | NA | no | 4 | −1.501 |
| CCGGTC | 0.008% | 0.221 | no | 4 | 0.045 |
| CCGGTG | 0.012% | 0.134 | no | 4 | −0.901 |
| CCGTTT | 0.008% | NA | no | 4 | −0.313 |
| CCTATT | 0.006% | −0.520 | no | 4 | −0.699 |
| CCTCTT | 0.007% | −0.371 | no | 4 | −1.879 |
| CCTGAT | 0.015% | NA | no | 4 | −0.602 |
| CCTGCT | 0.013% | 0.416 | no | 4 | −0.136 |
| CCTGGT | 0.011% | NA | no | 4 | 0.693 |
| CCTGTA | 0.007% | −0.279 | no | 4 | −0.935 |
| CCTGTC | 0.011% | NA | no | 4 | −0.597 |
| CCTGTG | 0.012% | 0.149 | no | 4 | −0.177 |
| CCTTTT | 0.005% | −0.777 | no | 4 | −3.580 |
| CGAAAA | 0.031% | NA | no | 4 | −0.325 |
| CGAAAC | 0.019% | 0.331 | no | 4 | 2.952 |
| CGAAAG | 0.030% | NA | no | 4 | −0.310 |
| CGAACA | 0.028% | NA | no | 4 | −0.344 |
| CGAACC | 0.018% | 0.405 | no | 4 | −0.518 |
| CGAACG | 0.028% | 0.614 | no | 4 | 0.655 |
| CGAAGA | 0.026% | 0.750 | no | 4 | −0.446 |
| CGAAGC | 0.016% | 0.640 | no | 4 | −1.093 |
| CGAAGG | 0.042% | 0.274 | no | 4 | −0.938 |
| CGACAA | 0.018% | NA | no | 4 | 1.181 |
| CGACAC | 0.020% | NA | no | 4 | −0.391 |
| CGACAG | 0.021% | NA | no | 4 | 0.596 |
| CGACCA | 0.018% | 0.529 | no | 4 | 0.429 |
| CGACCC | 0.011% | 0.219 | no | 4 | −2.014 |
| CGACCG | 0.016% | 0.578 | no | 4 | 0.463 |
| CGACGA | 0.015% | 0.805 | no | 4 | −0.028 |
| CGACGC | 0.011% | 0.740 | no | 4 | −0.538 |
| CGACGG | 0.013% | 0.825 | no | 4 | −0.848 |
| CGATAA | 0.020% | NA | no | 4 | 0.330 |
| CGATAC | 0.018% | NA | no | 4 | −1.775 |
| CGATAG | 0.018% | −0.372 | no | 4 | 0.689 |
| CGATCA | 0.018% | NA | no | 4 | −0.304 |
| CGATCC | 0.009% | 0.447 | no | 4 | 0.031 |
| CGATCG | 0.014% | 0.517 | no | 4 | 0.830 |
| CGATGA | 0.017% | 0.512 | no | 4 | −0.894 |
| CGATGC | 0.014% | 0.283 | no | 4 | −0.550 |
| CGATGG | 0.018% | 0.422 | no | 4 | 0.218 |
| CGCAAT | 0.007% | NA | no | 4 | −0.769 |
| CGCACT | 0.014% | NA | no | 4 | −0.908 |
| CGCAGT | 0.013% | NA | no | 4 | 0.009 |
| CGCATA | 0.013% | NA | no | 4 | 0.526 |
| CGCATC | 0.012% | 0.410 | no | 4 | −0.491 |
| CGCATG | 0.015% | NA | no | 4 | −0.422 |
| CGCCAT | 0.013% | NA | no | 4 | 0.443 |
| CGCCCT | 0.007% | 0.299 | no | 4 | 0.095 |
| CGCCGT | 0.010% | 0.479 | no | 4 | −1.365 |
| CGCCTA | 0.012% | NA | no | 4 | 0.513 |
| CGCCTC | 0.005% | NA | no | 4 | −1.467 |
| CGCCTG | 0.007% | 0.445 | no | 4 | −1.598 |
| CGCGAA | 0.021% | 0.542 | no | 4 | 0.075 |
| CGCGAC | 0.009% | 0.747 | no | 4 | 2.830 |
| CGCGAG | 0.014% | 0.503 | no | 4 | −0.598 |
| CGCGCA | 0.016% | 0.350 | no | 4 | −1.400 |
| CGCGCC | 0.006% | 0.622 | no | 4 | 0.969 |
| CGCGCG | 0.013% | 0.725 | no | 4 | −0.929 |
| CGCGGA | 0.017% | 0.784 | no | 4 | 0.208 |
| CGCGGC | 0.009% | NA | no | 4 | 1.168 |
| CGCGGG | 0.016% | 0.640 | no | 4 | 0.489 |
| CGCTAT | 0.010% | NA | no | 4 | 0.803 |
| CGCTCT | 0.009% | NA | no | 4 | −0.502 |
| CGCTGT | 0.008% | 0.248 | no | 4 | 0.701 |
| CGCTTA | 0.013% | NA | no | 4 | −0.500 |
| CGCTTC | 0.006% | 0.397 | no | 4 | −0.684 |
| CGCTTG | 0.010% | NA | no | 4 | 0.365 |
| CGGAAT | 0.022% | 0.487 | no | 4 | 0.037 |
| CGGACT | 0.018% | 0.444 | no | 4 | 1.943 |
| CGGAGT | 0.022% | 0.430 | no | 4 | −0.112 |
| CGGATA | 0.024% | NA | no | 4 | 0.093 |
| CGGATC | 0.010% | 0.439 | no | 4 | −0.003 |
| CGGATG | 0.023% | 0.573 | no | 4 | 0.003 |
| CGGCAT | 0.016% | NA | no | 4 | −0.147 |
| CGGCCT | 0.007% | NA | no | 4 | 0.935 |
| CGGCGT | 0.013% | 0.570 | no | 4 | −1.007 |
| CGGCTA | 0.014% | NA | no | 4 | 0.336 |
| CGGCTC | 0.012% | NA | no | 4 | 1.684 |
| CGGCTG | 0.013% | 0.249 | no | 4 | −0.227 |
| CGGGAA | 0.029% | 0.392 | no | 4 | 0.280 |
| CGGGAC | 0.020% | 0.486 | no | 4 | −0.590 |
| CGGGAG | 0.029% | 0.496 | no | 4 | −0.051 |
| CGGGCA | 0.025% | 0.387 | no | 4 | −0.248 |
| CGGGCC | 0.011% | 0.242 | no | 4 | 0.006 |
| CGGGCG | 0.021% | 0.335 | no | 4 | −1.589 |
| CGGGGA | 0.032% | 0.320 | no | 4 | −0.896 |
| CGGGGC | 0.015% | 0.291 | no | 4 | 0.341 |
| CGGGGG | 0.026% | 0.102 | no | 4 | 0.833 |
| CGGTAT | 0.015% | NA | no | 4 | 1.168 |
| CGGTCT | 0.009% | 0.261 | no | 4 | 0.198 |
| CGGTTA | 0.017% | NA | no | 4 | −0.187 |
| CGGTTC | 0.007% | 0.358 | no | 4 | −1.306 |
| CGGTTG | 0.013% | NA | no | 4 | −0.579 |
| CGTAAT | 0.017% | NA | no | 4 | 0.298 |
| CGTACT | 0.009% | NA | no | 4 | −1.035 |
| CGTAGT | 0.011% | NA | no | 4 | 1.330 |
| CGTATA | 0.017% | NA | no | 4 | −0.144 |
| CGTATC | 0.011% | NA | no | 4 | 0.476 |
| CGTATG | 0.011% | NA | no | 4 | 1.448 |
| CGTCAT | 0.008% | 0.367 | no | 4 | 0.543 |
| CGTCCT | 0.005% | 0.477 | no | 4 | −0.983 |
| CGTCGT | 0.006% | 0.685 | no | 4 | 0.655 |
| CGTCTA | 0.010% | NA | no | 4 | −0.340 |
| CGTCTC | 0.006% | 0.353 | no | 4 | −1.327 |
| CGTCTG | 0.012% | 0.574 | no | 4 | 0.286 |
| CGTGAA | 0.022% | 0.406 | no | 4 | −1.418 |
| CGTGAC | 0.011% | 0.488 | no | 4 | 2.362 |
| CGTGAG | 0.015% | NA | no | 4 | −0.829 |
| CGTGCA | 0.018% | 0.186 | no | 4 | 0.425 |
| CGTGCC | 0.011% | 0.437 | no | 4 | 0.169 |
| CGTGCG | 0.015% | 0.538 | no | 4 | −0.486 |
| CGTGGA | 0.014% | 0.693 | no | 4 | −1.367 |
| CGTGGC | 0.010% | 0.436 | no | 4 | 4.292 |
| CGTGGG | 0.018% | 0.366 | no | 4 | −0.381 |
| CGTTAT | 0.012% | NA | no | 4 | −2.301 |
| CGTTGT | 0.007% | NA | no | 4 | 1.554 |
| CGTTTA | 0.010% | NA | no | 4 | −0.902 |
| CGTTTC | 0.008% | NA | no | 4 | −1.057 |
| CGTTTG | 0.007% | NA | no | 4 | −0.386 |
| CTAAAT | 0.016% | −0.493 | yes | 4 | −4.226 |
| CTAACT | 0.011% | −0.371 | yes | 4 | −3.767 |
| CTAAGT | 0.015% | −0.515 | yes | 4 | −5.028 |
| CTAATA | 0.012% | −0.705 | yes | 4 | −5.432 |
| CTAATC | 0.010% | NA | yes | 4 | −3.637 |
| CTAATG | 0.015% | −0.382 | yes | 4 | −2.958 |
| CTACAT | 0.007% | NA | no | 4 | 1.999 |
| CTACCT | 0.004% | NA | no | 4 | −0.421 |
| CTACGT | 0.010% | 0.348 | no | 4 | 0.615 |
| CTACTA | 0.008% | NA | no | 4 | −1.895 |
| CTACTC | 0.009% | NA | no | 4 | −0.966 |
| CTACTG | 0.005% | 0.182 | no | 4 | −0.861 |
| CTAGAA | 0.015% | NA | yes | 4 | −5.617 |
| CTAGAC | 0.011% | −0.350 | yes | 4 | −4.679 |
| CTAGAG | 0.011% | −0.315 | yes | 4 | −3.370 |
| CTAGCA | 0.012% | −0.848 | yes | 4 | −6.462 |
| CTAGCC | 0.005% | −0.415 | yes | 4 | −2.428 |
| CTAGCG | 0.008% | NA | yes | 4 | −3.916 |
| CTAGGA | 0.015% | NA | yes | 4 | −3.358 |
| CTAGGC | 0.010% | −0.457 | yes | 4 | −5.406 |
| CTAGGG | 0.015% | −0.600 | yes | 4 | −7.655 |
| CTATAT | 0.008% | −0.636 | no | 4 | −2.937 |
| CTATCT | 0.005% | NA | no | 4 | −4.321 |
| CTATGT | 0.009% | −0.307 | no | 4 | −2.655 |
| CTATTA | 0.010% | −0.634 | no | 4 | −2.367 |
| CTATTC | 0.009% | −0.292 | no | 4 | −3.078 |
| CTATTG | 0.009% | −0.357 | no | 4 | −1.023 |
| CTCCTT | 0.006% | NA | no | 4 | 0.943 |
| CTCGAT | 0.008% | 0.285 | no | 4 | 0.153 |
| CTCGCT | 0.008% | NA | no | 4 | 1.062 |
| CTCGTA | 0.013% | NA | no | 4 | −1.622 |
| CTCGTC | 0.008% | 0.486 | no | 4 | 0.350 |
| CTCGTG | 0.009% | 0.250 | no | 4 | −0.417 |
| CTCTTT | 0.007% | −0.352 | no | 4 | 0.105 |
| CTGATT | 0.011% | NA | yes | 4 | −4.011 |
| CTGCTT | 0.010% | NA | no | 4 | 1.029 |
| CTGGAT | 0.013% | 0.434 | no | 4 | 1.593 |
| CTGGCT | 0.010% | NA | no | 4 | 1.129 |
| CTGGGT | 0.011% | NA | no | 4 | 0.004 |
| CTGGTA | 0.014% | −0.388 | no | 4 | 0.522 |
| CTGGTC | 0.007% | NA | no | 4 | 1.659 |
| CTGGTG | 0.009% | NA | no | 4 | −0.709 |
| CTGTTT | 0.008% | NA | no | 4 | −0.953 |
| CTTCTT | 0.006% | −0.279 | no | 4 | −1.950 |
| CTTGAT | 0.012% | NA | no | 4 | 1.198 |
| CTTGGT | 0.010% | NA | no | 4 | −0.218 |
| CTTGTA | 0.010% | −0.365 | no | 4 | −2.510 |
| CTTGTC | 0.005% | NA | no | 4 | −2.410 |
| CTTGTG | 0.008% | NA | no | 4 | 0.217 |
| CTTTTT | 0.006% | −0.780 | no | 4 | −5.717 |
| GAAAAT | 0.049% | NA | no | 4 | −1.270 |
| GAAACT | 0.048% | 0.383 | no | 4 | −1.006 |
| GAAAGT | 0.053% | NA | no | 4 | −2.007 |
| GAAATA | 0.036% | NA | no | 4 | −1.518 |
| GAAATC | 0.033% | 0.426 | no | 4 | −2.935 |
| GAAATG | 0.053% | NA | no | 4 | −0.642 |
| GAACAT | 0.044% | 0.361 | no | 4 | −1.180 |
| GAACCT | 0.024% | 0.566 | no | 4 | −0.178 |
| GAACGT | 0.038% | 0.508 | no | 4 | −0.118 |
| GAACTA | 0.038% | NA | no | 4 | −0.585 |
| GAACTC | 0.021% | 0.348 | no | 4 | −0.898 |
| GAACTG | 0.038% | 0.477 | no | 4 | 0.027 |
| GAAGAA | 0.068% | 0.845 | no | 4 | −0.634 |
| GAAGAC | 0.038% | 0.964 | no | 4 | −0.261 |
| GAAGAG | 0.073% | 0.490 | no | 4 | −0.123 |
| GAAGCA | 0.058% | 0.357 | no | 4 | −0.302 |
| GAAGCC | 0.032% | 0.598 | no | 4 | −0.252 |
| GAAGCG | 0.045% | 0.556 | no | 4 | −0.199 |
| GAAGGA | 0.066% | 0.565 | no | 4 | 0.084 |
| GAAGGC | 0.046% | 0.553 | no | 4 | −0.739 |
| GAAGGG | 0.069% | NA | no | 4 | −1.285 |
| GAATAT | 0.033% | NA | no | 4 | −2.215 |
| GAATCT | 0.027% | 0.354 | no | 4 | −1.541 |
| GAATGT | 0.045% | NA | no | 4 | −1.735 |
| GAATTA | 0.044% | NA | no | 4 | −1.820 |
| GAATTC | 0.020% | 0.350 | no | 4 | −2.628 |
| GAATTG | 0.039% | 0.142 | no | 4 | −0.239 |
| GACATT | 0.026% | 0.245 | no | 4 | −1.062 |
| GACCTT | 0.017% | 0.438 | no | 4 | −2.298 |
| GACGAT | 0.036% | 0.760 | no | 4 | −0.060 |
| GACGCT | 0.022% | 0.544 | no | 4 | −0.296 |
| GACGGT | 0.024% | 0.405 | no | 4 | 1.144 |
| GACGTA | 0.030% | 0.372 | no | 4 | −0.589 |
| GACGTC | 0.019% | 0.984 | no | 4 | 1.098 |
| GACGTG | 0.035% | 0.487 | no | 4 | 0.160 |
| GACTTT | 0.018% | NA | no | 4 | −0.754 |
| GAGATT | 0.031% | 0.237 | no | 4 | −1.633 |
| GAGCTT | 0.023% | NA | no | 4 | 0.449 |
| GAGGAT | 0.049% | 0.531 | no | 4 | −0.571 |
| GAGGCT | 0.031% | NA | no | 4 | 0.152 |
| GAGGGT | 0.032% | −0.340 | no | 4 | −6.092 |
| GAGGTA | 0.047% | −0.287 | no | 4 | −3.978 |
| GAGGTC | 0.022% | 0.262 | no | 4 | −1.787 |
| GAGGTG | 0.041% | NA | no | 4 | −0.762 |
| GAGTTT | 0.031% | NA | no | 4 | −1.716 |
| GATATT | 0.031% | NA | no | 4 | −2.944 |
| GATCTT | 0.015% | NA | no | 4 | −0.802 |
| GATGAT | 0.035% | 0.456 | no | 4 | −1.235 |
| GATGCT | 0.013% | 0.262 | no | 4 | −0.287 |
| GATGGT | 0.026% | NA | no | 4 | −1.077 |
| GATGTA | 0.028% | NA | no | 4 | −1.822 |
| GATGTC | 0.023% | 0.521 | no | 4 | −0.002 |
| GATGTG | 0.026% | NA | no | 4 | −0.720 |
| GATTTT | 0.023% | NA | no | 4 | −3.296 |
| GCAAAT | 0.035% | NA | no | 4 | −1.702 |
| GCAACT | 0.023% | 0.248 | no | 4 | −0.187 |
| GCAAGT | 0.036% | 0.245 | no | 4 | −0.425 |
| GCAATA | 0.032% | −0.178 | no | 4 | −1.745 |
| GCAATC | 0.019% | 0.247 | no | 4 | −1.903 |
| GCAATG | 0.036% | 0.107 | no | 4 | −0.278 |
| GCACAT | 0.026% | −0.166 | no | 4 | −1.646 |
| GCACCT | 0.020% | −0.246 | no | 4 | −0.666 |
| GCACGT | 0.027% | NA | no | 4 | −0.048 |
| GCACTA | 0.027% | −0.331 | no | 4 | −2.294 |
| GCACTC | 0.018% | −0.183 | no | 4 | −1.563 |
| GCACTG | 0.019% | NA | no | 4 | −0.601 |
| GCAGAA | 0.038% | 0.343 | no | 4 | −6.925 |
| GCAGAC | 0.035% | 0.271 | no | 4 | −2.639 |
| GCAGAG | 0.036% | NA | no | 4 | −1.011 |
| GCAGCA | 0.039% | 0.120 | no | 4 | −0.720 |
| GCAGCC | 0.018% | −0.320 | no | 4 | −1.494 |
| GCAGCG | 0.030% | 0.204 | no | 4 | −1.455 |
| GCAGGA | 0.044% | 0.120 | no | 4 | −2.136 |
| GCAGGC | 0.029% | NA | no | 4 | −1.642 |
| GCAGGG | 0.045% | −0.272 | no | 4 | −3.944 |
| GCATAT | 0.025% | NA | no | 4 | −1.368 |
| GCATCT | 0.015% | −0.139 | no | 4 | −0.114 |
| GCATGT | 0.026% | NA | no | 4 | −1.883 |
| GCATTA | 0.028% | −0.261 | no | 4 | −2.675 |
| GCATTC | 0.017% | NA | no | 4 | −2.250 |
| GCATTG | 0.022% | NA | no | 4 | −0.636 |
| GCCATT | 0.014% | −0.318 | no | 4 | −0.142 |
| GCCCTT | 0.011% | −0.247 | no | 4 | 4.529 |
| GCCGAT | 0.019% | NA | no | 4 | 0.211 |
| GCCGCT | 0.011% | NA | no | 4 | −0.760 |
| GCCGGT | 0.019% | NA | no | 4 | 0.710 |
| GCCGTA | 0.018% | NA | no | 4 | −1.313 |
| GCCGTC | 0.013% | 0.485 | no | 4 | 0.306 |
| GCCGTG | 0.017% | 0.215 | no | 4 | −0.949 |
| GCCTTT | 0.012% | −0.405 | no | 4 | 0.054 |
| GCGATT | 0.020% | NA | no | 4 | 0.051 |
| GCGCTT | 0.015% | NA | no | 4 | −0.932 |
| GCGGAT | 0.033% | 0.541 | no | 4 | −0.771 |
| GCGGCT | 0.022% | NA | no | 4 | 0.004 |
| GCGGGT | 0.022% | NA | no | 4 | −2.285 |
| GCGGTA | 0.029% | NA | no | 4 | −0.965 |
| GCGGTC | 0.016% | 0.359 | no | 4 | 1.092 |
| GCGGTG | 0.021% | NA | no | 4 | 0.297 |
| GCGTTT | 0.013% | NA | no | 4 | 1.112 |
| GCTATT | 0.009% | −0.350 | no | 4 | −0.895 |
| GCTCTT | 0.011% | −0.258 | no | 4 | −0.708 |
| GCTGAT | 0.018% | NA | no | 4 | −0.478 |
| GCTGCT | 0.011% | 0.337 | no | 4 | 1.276 |
| GCTGGT | 0.017% | NA | no | 4 | −0.001 |
| GCTGTA | 0.021% | NA | no | 4 | −0.812 |
| GCTGTC | 0.012% | 0.466 | no | 4 | 0.824 |
| GCTGTG | 0.022% | NA | no | 4 | −0.104 |
| GCTTTT | 0.019% | −0.329 | no | 4 | −3.786 |
| GGAAAA | 0.078% | NA | no | 4 | −0.519 |
| GGAAAC | 0.058% | 0.455 | no | 4 | −0.299 |
| GGAAAG | 0.073% | NA | no | 4 | −0.182 |
| GGAACA | 0.056% | 0.415 | no | 4 | −0.135 |
| GGAACC | 0.031% | 0.593 | no | 4 | 0.121 |
| GGAACG | 0.058% | 0.522 | no | 4 | 0.398 |
| GGAAGA | 0.070% | 0.651 | no | 4 | 0.217 |
| GGAAGC | 0.041% | 0.486 | no | 4 | 0.471 |
| GGAAGG | 0.063% | NA | no | 4 | −0.272 |
| GGACAA | 0.065% | 0.466 | no | 4 | −0.825 |
| GGACAC | 0.039% | 0.516 | no | 4 | 0.198 |
| GGACAG | 0.052% | NA | no | 4 | −0.415 |
| GGACCA | 0.034% | 0.364 | no | 4 | −0.509 |
| GGACCC | 0.022% | 0.372 | no | 4 | 0.013 |
| GGACCG | 0.029% | 0.636 | no | 4 | 0.962 |
| GGACGA | 0.041% | 0.697 | no | 4 | 0.739 |
| GGACGC | 0.034% | 0.761 | no | 4 | −0.271 |
| GGACGG | 0.034% | 0.396 | no | 4 | 0.562 |
| GGATAA | 0.063% | NA | no | 4 | 0.416 |
| GGATAC | 0.039% | 0.426 | no | 4 | 0.494 |
| GGATAG | 0.052% | −0.376 | no | 4 | −0.358 |
| GGATCA | 0.037% | 0.194 | no | 4 | 0.022 |
| GGATCC | 0.020% | 0.445 | no | 4 | −0.093 |
| GGATCG | 0.031% | 0.388 | no | 4 | 1.722 |
| GGATGA | 0.049% | 0.391 | no | 4 | −0.001 |
| GGATGC | 0.029% | 0.417 | no | 4 | 0.427 |
| GGATGG | 0.047% | NA | no | 4 | −0.240 |
| GGCAAT | 0.028% | 0.112 | no | 4 | −0.483 |
| GGCACT | 0.020% | −0.251 | no | 4 | 0.601 |
| GGCAGT | 0.050% | −0.107 | no | 4 | −0.032 |
| GGCATA | 0.027% | NA | no | 4 | −0.266 |
| GGCATC | 0.016% | NA | no | 4 | 0.824 |
| GGCATG | 0.020% | 0.074 | no | 4 | −0.492 |
| GGCCAT | 0.019% | NA | no | 4 | −0.309 |
| GGCCCT | 0.012% | NA | no | 4 | 2.149 |
| GGCCGT | 0.015% | NA | no | 4 | −0.527 |
| GGCCTA | 0.016% | −0.260 | no | 4 | 0.274 |
| GGCCTC | 0.011% | NA | no | 4 | 0.063 |
| GGCCTG | 0.018% | 0.128 | no | 4 | −0.511 |
| GGCGAA | 0.038% | 0.405 | no | 4 | 0.166 |
| GGCGAC | 0.024% | 0.608 | no | 4 | 0.471 |
| GGCGAG | 0.030% | 0.169 | no | 4 | 0.217 |
| GGCGCA | 0.027% | 0.299 | no | 4 | 1.041 |
| GGCGCC | 0.011% | 0.480 | no | 4 | 0.303 |
| GGCGCG | 0.023% | 0.455 | no | 4 | −0.923 |
| GGCGGA | 0.034% | 0.499 | no | 4 | −0.059 |
| GGCGGC | 0.021% | 0.302 | no | 4 | −1.131 |
| GGCGGG | 0.033% | 0.148 | no | 4 | 0.621 |
| GGCTAT | 0.021% | −0.223 | no | 4 | 0.232 |
| GGCTCT | 0.016% | −0.222 | no | 4 | 0.361 |
| GGCTGT | 0.021% | 0.169 | no | 4 | 1.237 |
| GGCTTA | 0.021% | −0.295 | no | 4 | 0.226 |
| GGCTTC | 0.009% | 0.251 | no | 4 | −0.796 |
| GGCTTG | 0.016% | −0.193 | no | 4 | 0.210 |
| GGGAAT | 0.063% | NA | no | 4 | −0.739 |
| GGGACT | 0.038% | NA | no | 4 | 0.251 |
| GGGAGT | 0.043% | NA | no | 4 | 0.533 |
| GGGATA | 0.049% | NA | no | 4 | −0.751 |
| GGGATC | 0.024% | NA | no | 4 | 1.275 |
| GGGATG | 0.052% | NA | no | 4 | −0.042 |
| GGGCAT | 0.041% | −0.190 | no | 4 | −0.624 |
| GGGCCT | 0.018% | NA | no | 4 | −0.153 |
| GGGCGT | 0.024% | NA | no | 4 | 0.901 |
| GGGCTA | 0.033% | −0.444 | no | 4 | 0.594 |
| GGGCTC | 0.018% | −0.236 | no | 4 | 0.395 |
| GGGCTG | 0.029% | NA | no | 4 | 0.530 |
| GGGGAA | 0.067% | −0.337 | no | 4 | −0.749 |
| GGGGAC | 0.034% | NA | no | 4 | 0.050 |
| GGGGAG | 0.053% | −0.073 | no | 4 | 0.293 |
| GGGGCA | 0.048% | −0.080 | no | 4 | −0.674 |
| GGGGCC | 0.019% | −0.206 | no | 4 | −1.051 |
| GGGGCG | 0.029% | −0.237 | no | 4 | −0.037 |
| GGGGGA | 0.059% | −0.232 | no | 4 | −1.399 |
| GGGGGC | 0.032% | −0.277 | no | 4 | −0.109 |
| GGGGGG | 0.043% | −0.358 | no | 4 | −4.336 |
| GGGTAT | 0.048% | −0.508 | no | 4 | −0.700 |
| GGGTCT | 0.019% | −0.347 | no | 4 | −0.111 |
| GGGTGT | 0.027% | −0.392 | no | 4 | −0.589 |
| GGGTTA | 0.031% | −0.737 | no | 4 | −1.798 |
| GGGTTC | 0.022% | NA | no | 4 | −0.140 |
| GGGTTG | 0.034% | −0.472 | no | 4 | −0.592 |
| GGTAAT | 0.043% | −0.489 | no | 4 | −0.439 |
| GGTACT | 0.023% | −0.215 | no | 4 | 0.632 |
| GGTAGT | 0.035% | −0.612 | no | 4 | −0.599 |
| GGTATA | 0.040% | −0.540 | no | 4 | 0.227 |
| GGTATC | 0.023% | NA | no | 4 | 0.044 |
| GGTATG | 0.036% | −0.317 | no | 4 | 0.528 |
| GGTCAT | 0.032% | NA | no | 4 | −0.896 |
| GGTCCT | 0.016% | NA | no | 4 | −1.892 |
| GGTCGT | 0.020% | NA | no | 4 | 0.208 |
| GGTCTA | 0.020% | −0.305 | no | 4 | −1.591 |
| GGTCTC | 0.015% | NA | no | 4 | 0.749 |
| GGTCTG | 0.027% | NA | no | 4 | −0.024 |
| GGTGAA | 0.050% | NA | no | 4 | 0.240 |
| GGTGAC | 0.027% | 0.200 | no | 4 | 0.088 |
| GGTGAG | 0.033% | −0.416 | no | 4 | −0.066 |
| GGTGCA | 0.030% | −0.150 | no | 4 | 0.432 |
| GGTGCC | 0.015% | NA | no | 4 | −1.141 |
| GGTGCG | 0.023% | NA | no | 4 | −0.078 |
| GGTGGA | 0.032% | NA | no | 4 | 0.676 |
| GGTGGC | 0.025% | −0.350 | no | 4 | −0.348 |
| GGTGGG | 0.033% | −0.359 | no | 4 | 0.100 |
| GGTTAT | 0.026% | −0.434 | no | 4 | −0.053 |
| GGTTGT | 0.019% | −0.427 | no | 4 | −0.566 |
| GGTTTA | 0.025% | −0.700 | no | 4 | −1.313 |
| GGTTTC | 0.015% | NA | no | 4 | −0.270 |
| GTAAAT | 0.039% | −0.450 | yes | 4 | −5.587 |
| GTAACT | 0.028% | −0.326 | yes | 4 | −8.508 |
| GTAAGT | 0.036% | −0.569 | yes | 4 | −8.380 |
| GTAATA | 0.034% | −0.538 | yes | 4 | −4.998 |
| GTAATC | 0.021% | −0.264 | yes | 4 | −4.319 |
| GTAATG | 0.041% | −0.313 | yes | 4 | −3.064 |
| GTACAT | 0.027% | −0.249 | no | 4 | −2.516 |
| GTACCT | 0.014% | NA | no | 4 | −1.269 |
| GTACGT | 0.025% | NA | no | 4 | −1.743 |
| GTACTA | 0.018% | −0.479 | no | 4 | −2.948 |
| GTACTC | 0.013% | NA | no | 4 | −2.897 |
| GTACTG | 0.020% | NA | no | 4 | −1.301 |
| GTAGAA | 0.046% | −0.357 | yes | 4 | −7.153 |
| GTAGAC | 0.020% | NA | yes | 4 | −7.542 |
| GTAGAG | 0.039% | −0.393 | yes | 4 | −3.410 |
| GTAGCA | 0.037% | −0.466 | yes | 4 | −4.915 |
| GTAGCC | 0.018% | −0.537 | yes | 4 | −7.972 |
| GTAGCG | 0.031% | NA | yes | 4 | −4.069 |
| GTAGGA | 0.033% | −0.351 | yes | 4 | −6.807 |
| GTAGGC | 0.022% | −0.566 | yes | 4 | −6.937 |
| GTAGGG | 0.035% | −0.733 | yes | 4 | −8.788 |
| GTATAT | 0.026% | −0.383 | no | 4 | −3.147 |
| GTATCT | 0.013% | −0.249 | no | 4 | −4.117 |
| GTATGT | 0.024% | −0.335 | no | 4 | −1.977 |
| GTATTA | 0.031% | −0.755 | no | 4 | −3.084 |
| GTATTC | 0.018% | NA | no | 4 | −4.031 |
| GTATTG | 0.030% | −0.271 | no | 4 | −0.604 |
| GTCATT | 0.014% | NA | no | 4 | −0.130 |
| GTCCTT | 0.008% | NA | no | 4 | −0.271 |
| GTCGAT | 0.018% | NA | no | 4 | 1.936 |
| GTCGCT | 0.016% | NA | no | 4 | −0.815 |
| GTCGGT | 0.020% | NA | no | 4 | −1.066 |
| GTCGTA | 0.023% | NA | no | 4 | −0.555 |
| GTCGTC | 0.012% | 0.774 | no | 4 | −1.239 |
| GTCGTG | 0.022% | 0.263 | no | 4 | −0.507 |
| GTCTTT | 0.012% | −0.243 | no | 4 | −0.916 |
| GTGATT | 0.025% | NA | yes | 4 | −3.033 |
| GTGCTT | 0.016% | −0.416 | no | 4 | −1.320 |
| GTGGAT | 0.034% | 0.389 | no | 4 | −0.576 |
| GTGGCT | 0.024% | NA | no | 4 | 0.805 |
| GTGGGT | 0.037% | −0.337 | no | 4 | −2.621 |
| GTGGTA | 0.033% | −0.223 | no | 4 | −0.949 |
| GTGGTC | 0.022% | NA | no | 4 | −0.247 |
| GTGGTG | 0.029% | NA | no | 4 | −1.036 |
| GTGTTT | 0.025% | −0.267 | no | 4 | −1.785 |
| GTTATT | 0.023% | −0.254 | no | 4 | −4.384 |
| GTTCTT | 0.011% | NA | no | 4 | −2.936 |
| GTTGAT | 0.031% | NA | no | 4 | −1.551 |
| GTTGCT | 0.013% | NA | no | 4 | −5.537 |
| GTTGGT | 0.019% | −0.158 | no | 4 | −0.195 |
| GTTGTA | 0.026% | −0.157 | no | 4 | −2.294 |
| GTTGTC | 0.010% | 0.267 | no | 4 | −0.361 |
| GTTGTG | 0.026% | −0.109 | no | 4 | −1.123 |
| GTTTTT | 0.019% | −0.256 | no | 4 | −4.352 |
| TAAAAA | 0.052% | −0.365 | no | 4 | −4.607 |
| TAAAAC | 0.044% | −0.380 | no | 4 | −5.540 |
| TAAAAG | 0.048% | −0.310 | no | 4 | −0.877 |
| TAAACA | 0.051% | NA | no | 4 | −5.134 |
| TAAACC | 0.026% | −0.306 | no | 4 | −3.324 |
| TAAACG | 0.038% | NA | no | 4 | −0.381 |
| TAAAGA | 0.048% | NA | no | 4 | −5.819 |
| TAAAGC | 0.031% | NA | no | 4 | −2.365 |
| TAAAGG | 0.048% | −0.407 | no | 4 | −2.467 |
| TAACAA | 0.044% | NA | no | 4 | −4.893 |
| TAACAC | 0.025% | NA | no | 4 | −3.223 |
| TAACAG | 0.038% | −0.436 | no | 4 | −2.106 |
| TAACCA | 0.022% | −0.233 | no | 4 | −3.482 |
| TAACCC | 0.018% | −0.442 | no | 4 | −5.544 |
| TAACCG | 0.026% | 0.286 | no | 4 | −0.202 |
| TAACGA | 0.030% | NA | no | 4 | −2.567 |
| TAACGC | 0.024% | NA | no | 4 | −2.384 |
| TAACGG | 0.032% | NA | no | 4 | −0.389 |
| TAATAA | 0.032% | −0.491 | no | 4 | −4.881 |
| TAATAC | 0.021% | −0.299 | no | 4 | −4.682 |
| TAATAG | 0.031% | −0.647 | no | 4 | −4.229 |
| TAATCA | 0.026% | NA | no | 4 | −4.280 |
| TAATCC | 0.010% | −0.266 | no | 4 | −2.751 |
| TAATCG | 0.027% | NA | no | 4 | −1.773 |
| TAATGA | 0.029% | NA | no | 4 | −4.909 |
| TAATGC | 0.025% | −0.398 | no | 4 | −4.292 |
| TAATGG | 0.026% | −0.300 | no | 4 | −2.506 |
| TACAAT | 0.022% | −0.257 | no | 4 | −1.284 |
| TACACT | 0.020% | NA | no | 4 | −1.208 |
| TACAGT | 0.022% | −0.265 | no | 4 | −0.749 |
| TACATA | 0.018% | NA | no | 4 | −1.980 |
| TACATC | 0.015% | 0.314 | no | 4 | −1.724 |
| TACATG | 0.014% | −0.190 | no | 4 | 1.238 |
| TACCAT | 0.014% | NA | no | 4 | −0.613 |
| TACCGT | 0.014% | NA | no | 4 | 0.179 |
| TACCTA | 0.007% | NA | no | 4 | −0.056 |
| TACCTC | 0.006% | NA | no | 4 | −1.598 |
| TACCTG | 0.010% | 0.337 | no | 4 | −2.466 |
| TACGAA | 0.028% | NA | no | 4 | −0.365 |
| TACGAC | 0.016% | 0.542 | no | 4 | −0.568 |
| TACGAG | 0.021% | NA | no | 4 | −0.315 |
| TACGCA | 0.021% | NA | no | 4 | 0.237 |
| TACGCC | 0.010% | 0.354 | no | 4 | 1.754 |
| TACGCG | 0.016% | 0.546 | no | 4 | −0.239 |
| TACGGA | 0.021% | 0.425 | no | 4 | −0.398 |
| TACGGC | 0.016% | 0.391 | no | 4 | 1.082 |
| TACGGG | 0.028% | 0.230 | no | 4 | −0.753 |
| TACTAT | 0.013% | −0.249 | no | 4 | −1.412 |
| TACTCT | 0.008% | NA | no | 4 | −2.017 |
| TACTGT | 0.013% | NA | no | 4 | −1.244 |
| TACTTA | 0.012% | −0.475 | no | 4 | −3.824 |
| TACTTC | 0.014% | NA | no | 4 | −1.406 |
| TACTTG | 0.008% | −0.255 | no | 4 | −1.427 |
| TAGAAT | 0.029% | −0.419 | no | 4 | −4.933 |
| TAGACT | 0.016% | NA | no | 4 | −2.457 |
| TAGAGT | 0.027% | −0.406 | no | 4 | −5.672 |
| TAGATA | 0.028% | −0.861 | no | 4 | −7.189 |
| TAGATC | 0.010% | NA | no | 4 | −7.528 |
| TAGATG | 0.030% | −0.328 | no | 4 | −3.495 |
| TAGCAT | 0.021% | −0.499 | no | 4 | −6.178 |
| TAGCCT | 0.010% | −0.266 | no | 4 | −5.320 |
| TAGCGT | 0.019% | −0.344 | no | 4 | −2.887 |
| TAGCTA | 0.016% | −0.812 | no | 4 | −7.930 |
| TAGCTC | 0.008% | −0.436 | no | 4 | −5.048 |
| TAGCTG | 0.016% | −0.306 | no | 4 | −2.301 |
| TAGGAA | 0.034% | −0.600 | no | 4 | −6.755 |
| TAGGAC | 0.021% | NA | no | 4 | −7.387 |
| TAGGAG | 0.028% | −0.464 | no | 4 | −3.571 |
| TAGGCA | 0.018% | −0.835 | no | 4 | −7.713 |
| TAGGCC | 0.011% | −0.517 | no | 4 | −5.906 |
| TAGGCG | 0.019% | −0.387 | no | 4 | −3.855 |
| TAGGGA | 0.031% | −0.838 | no | 4 | −9.429 |
| TAGGGC | 0.021% | −0.635 | no | 4 | −9.433 |
| TAGGGG | 0.030% | −0.659 | no | 4 | −6.441 |
| TAGTAT | 0.021% | −0.723 | no | 4 | −6.245 |
| TAGTCT | 0.013% | −0.369 | no | 4 | −7.485 |
| TAGTGT | 0.021% | −0.579 | no | 4 | −5.277 |
| TAGTTA | 0.019% | −0.893 | no | 4 | −7.370 |
| TAGTTC | 0.013% | −0.509 | no | 4 | −6.180 |
| TAGTTG | 0.016% | −0.692 | no | 4 | −5.193 |
| TATAAT | 0.033% | −0.595 | no | 4 | −5.470 |
| TATACT | 0.019% | NA | no | 4 | −2.988 |
| TATAGT | 0.023% | −0.551 | no | 4 | −5.083 |
| TATATA | 0.027% | −0.586 | no | 4 | −5.232 |
| TATATC | 0.019% | NA | no | 4 | −8.641 |
| TATATG | 0.021% | −0.443 | no | 4 | −1.371 |
| TATCAT | 0.021% | NA | no | 4 | −6.799 |
| TATCCT | 0.008% | NA | no | 4 | −1.638 |
| TATCGT | 0.015% | NA | no | 4 | −0.464 |
| TATCTA | 0.016% | NA | no | 4 | −2.487 |
| TATCTC | 0.013% | NA | no | 4 | −3.535 |
| TATCTG | 0.015% | NA | no | 4 | −0.043 |
| TATGAA | 0.035% | NA | no | 4 | −3.180 |
| TATGAC | 0.019% | NA | no | 4 | −3.071 |
| TATGAG | 0.033% | −0.294 | no | 4 | −0.513 |
| TATGCA | 0.027% | −0.299 | no | 4 | −3.824 |
| TATGCC | 0.010% | NA | no | 4 | −2.631 |
| TATGCG | 0.022% | NA | no | 4 | −0.008 |
| TATGGA | 0.032% | NA | no | 4 | −0.571 |
| TATGGC | 0.027% | NA | no | 4 | −2.052 |
| TATGGG | 0.017% | −0.217 | no | 4 | −2.241 |
| TATTAT | 0.024% | −0.434 | no | 4 | −5.274 |
| TATTCT | 0.010% | NA | no | 4 | −3.611 |
| TATTGT | 0.024% | −0.361 | no | 4 | −2.956 |
| TATTTA | 0.022% | −0.674 | no | 4 | −7.627 |
| TATTTC | 0.011% | −0.407 | no | 4 | −3.981 |
| TATTTG | 0.020% | −0.421 | no | 4 | −2.248 |
| TCAAAA | 0.040% | NA | no | 4 | −3.057 |
| TCAAAC | 0.027% | NA | no | 4 | −1.499 |
| TCAAAG | 0.041% | NA | no | 4 | −0.991 |
| TCAACA | 0.034% | NA | no | 4 | −1.340 |
| TCAACC | 0.017% | 0.222 | no | 4 | −0.613 |
| TCAACG | 0.021% | 0.643 | no | 4 | −0.347 |
| TCAAGA | 0.038% | 0.464 | no | 4 | −0.818 |
| TCAAGC | 0.027% | 0.356 | no | 4 | −1.126 |
| TCAAGG | 0.036% | NA | no | 4 | −1.564 |
| TCACAA | 0.028% | NA | no | 4 | −2.351 |
| TCACAC | 0.019% | NA | no | 4 | −1.523 |
| TCACAG | 0.024% | −0.253 | no | 4 | −1.167 |
| TCACCA | 0.017% | −0.146 | no | 4 | 0.899 |
| TCACCC | 0.012% | NA | no | 4 | 0.396 |
| TCACCG | 0.011% | 0.651 | no | 4 | 1.213 |
| TCACGA | 0.024% | NA | no | 4 | −1.241 |
| TCACGC | 0.013% | 0.449 | no | 4 | −0.583 |
| TCACGG | 0.021% | 0.253 | no | 4 | −0.521 |
| TCATAA | 0.024% | NA | no | 4 | −2.852 |
| TCATAC | 0.019% | NA | no | 4 | −2.260 |
| TCATAG | 0.018% | −0.517 | no | 4 | −1.141 |
| TCATCA | 0.022% | NA | no | 4 | −1.249 |
| TCATCC | 0.012% | 0.274 | no | 4 | −0.772 |
| TCATCG | 0.020% | 0.752 | no | 4 | −1.097 |
| TCATGA | 0.024% | NA | no | 4 | −2.227 |
| TCATGC | 0.018% | NA | no | 4 | −0.559 |
| TCATGG | 0.023% | NA | no | 4 | −0.187 |
| TCCAAT | 0.016% | NA | no | 4 | −1.686 |
| TCCACT | 0.012% | NA | no | 4 | −2.259 |
| TCCAGT | 0.013% | NA | no | 4 | −0.435 |
| TCCATA | 0.011% | NA | no | 4 | −1.884 |
| TCCATC | 0.013% | NA | no | 4 | −2.238 |
| TCCATG | 0.014% | NA | no | 4 | −0.464 |
| TCCCCT | 0.006% | NA | no | 4 | −2.834 |
| TCCCGT | 0.010% | NA | no | 4 | −1.561 |
| TCCCTA | 0.010% | −0.566 | no | 4 | −1.649 |
| TCCCTC | 0.007% | NA | no | 4 | −1.868 |
| TCCCTG | 0.011% | 0.247 | no | 4 | −1.229 |
| TCCGAA | 0.018% | NA | no | 4 | −0.603 |
| TCCGAC | 0.013% | 0.526 | no | 4 | −0.994 |
| TCCGAG | 0.014% | 0.308 | no | 4 | 0.708 |
| TCCGCA | 0.017% | NA | no | 4 | 3.924 |
| TCCGCC | 0.006% | 0.192 | no | 4 | −0.356 |
| TCCGCG | 0.014% | 0.675 | no | 4 | −0.386 |
| TCCGGA | 0.014% | 0.636 | no | 4 | 0.382 |
| TCCGGC | 0.008% | 0.480 | no | 4 | 0.198 |
| TCCGGG | 0.011% | 0.514 | no | 4 | −0.296 |
| TCCTAT | 0.008% | −0.293 | no | 4 | −1.800 |
| TCCTCT | 0.006% | NA | no | 4 | 1.476 |
| TCCTGT | 0.008% | NA | no | 4 | −0.731 |
| TCCTTA | 0.008% | −0.527 | no | 4 | −1.399 |
| TCCTTC | 0.006% | NA | no | 4 | −2.387 |
| TCCTTG | 0.009% | NA | no | 4 | −0.389 |
| TCGAAT | 0.023% | NA | no | 4 | −0.659 |
| TCGACT | 0.016% | 0.542 | no | 4 | −0.567 |
| TCGAGT | 0.024% | NA | no | 4 | −0.308 |
| TCGATA | 0.017% | NA | no | 4 | −1.960 |
| TCGATC | 0.014% | 0.582 | no | 4 | −1.568 |
| TCGATG | 0.023% | 0.337 | no | 4 | 0.044 |
| TCGCAT | 0.016% | NA | no | 4 | −0.904 |
| TCGCCT | 0.009% | 0.492 | no | 4 | −2.184 |
| TCGCGT | 0.011% | 0.436 | no | 4 | −0.065 |
| TCGCTA | 0.016% | NA | no | 4 | −1.448 |
| TCGCTC | 0.008% | 0.356 | no | 4 | −1.002 |
| TCGCTG | 0.013% | 0.311 | no | 4 | −1.082 |
| TCGGAA | 0.035% | 0.447 | no | 4 | −0.690 |
| TCGGAC | 0.018% | 0.559 | no | 4 | −0.018 |
| TCGGAG | 0.021% | 0.315 | no | 4 | −0.928 |
| TCGGCA | 0.023% | NA | no | 4 | −0.004 |
| TCGGCC | 0.009% | 0.441 | no | 4 | −0.727 |
| TCGGCG | 0.018% | 0.863 | no | 4 | −0.307 |
| TCGGGA | 0.025% | 0.421 | no | 4 | 0.414 |
| TCGGGC | 0.008% | 0.350 | no | 4 | 3.317 |
| TCGGGG | 0.024% | 0.237 | no | 4 | 0.171 |
| TCGTAT | 0.011% | NA | no | 4 | −0.103 |
| TCGTGT | 0.015% | 0.255 | no | 4 | 1.091 |
| TCGTTA | 0.018% | NA | no | 4 | −1.875 |
| TCGTTC | 0.010% | 0.473 | no | 4 | 0.488 |
| TCGTTG | 0.017% | 0.189 | no | 4 | 0.022 |
| TCTAAT | 0.016% | −0.402 | no | 4 | −1.638 |
| TCTACT | 0.012% | NA | no | 4 | −1.419 |
| TCTAGT | 0.008% | −0.450 | no | 4 | −6.596 |
| TCTATA | 0.012% | −0.491 | no | 4 | −5.035 |
| TCTATC | 0.008% | NA | no | 4 | −2.815 |
| TCTATG | 0.014% | −0.240 | no | 4 | −0.022 |
| TCTCAT | 0.009% | NA | no | 4 | −1.436 |
| TCTCCT | 0.007% | 0.362 | no | 4 | −2.722 |
| TCTCGT | 0.008% | NA | no | 4 | 2.443 |
| TCTCTA | 0.012% | NA | no | 4 | −2.895 |
| TCTCTC | 0.009% | NA | no | 4 | −1.950 |
| TCTCTG | 0.010% | 0.164 | no | 4 | −0.454 |
| TCTGAA | 0.025% | NA | no | 4 | −1.857 |
| TCTGAC | 0.014% | 0.417 | no | 4 | −0.726 |
| TCTGAG | 0.019% | NA | no | 4 | −0.435 |
| TCTGCA | 0.013% | 0.322 | no | 4 | −6.092 |
| TCTGCC | 0.011% | 0.248 | no | 4 | −2.157 |
| TCTGCG | 0.015% | 0.698 | no | 4 | −0.979 |
| TCTGGA | 0.021% | NA | no | 4 | −0.788 |
| TCTGGC | 0.014% | NA | no | 4 | 0.062 |
| TCTGGG | 0.020% | NA | no | 4 | −0.821 |
| TCTTAT | 0.010% | −0.317 | no | 4 | −4.585 |
| TCTTGT | 0.008% | NA | no | 4 | −3.419 |
| TCTTTA | 0.013% | −0.477 | no | 4 | −5.750 |
| TCTTTC | 0.011% | NA | no | 4 | −4.034 |
| TCTTTG | 0.016% | −0.218 | no | 4 | −0.563 |
| TGCAAA | 0.037% | −0.487 | no | 4 | 0.628 |
| TGCAAC | 0.021% | NA | no | 4 | −0.765 |
| TGCAAG | 0.033% | NA | no | 4 | −0.646 |
| TGCACA | 0.023% | −0.335 | no | 4 | −0.123 |
| TGCACC | 0.012% | NA | no | 4 | 1.585 |
| TGCACG | 0.025% | NA | no | 4 | −0.571 |
| TGCAGA | 0.021% | NA | no | 4 | −2.629 |
| TGCAGC | 0.015% | −0.365 | no | 4 | −0.747 |
| TGCAGG | 0.020% | −0.190 | no | 4 | −0.828 |
| TGCCAA | 0.023% | −0.257 | no | 4 | −0.822 |
| TGCCAC | 0.014% | −0.258 | no | 4 | 0.707 |
| TGCCAG | 0.018% | NA | no | 4 | −0.233 |
| TGCCCC | 0.010% | −0.255 | no | 4 | 1.354 |
| TGCCCG | 0.013% | NA | no | 4 | 0.875 |
| TGCCGA | 0.016% | NA | no | 4 | −0.401 |
| TGCCGC | 0.015% | NA | no | 4 | −0.379 |
| TGCCGG | 0.018% | 0.357 | no | 4 | −0.880 |
| TGCTAA | 0.030% | −0.319 | no | 4 | 0.980 |
| TGCTAC | 0.010% | NA | no | 4 | 0.541 |
| TGCTAG | 0.021% | −0.680 | no | 4 | −1.668 |
| TGCTCA | 0.013% | −0.284 | no | 4 | 1.841 |
| TGCTCC | 0.007% | NA | no | 4 | 0.367 |
| TGCTCG | 0.013% | NA | no | 4 | −0.775 |
| TGCTGA | 0.017% | NA | no | 4 | −0.005 |
| TGCTGC | 0.009% | 0.229 | no | 4 | −0.943 |
| TGCTGG | 0.017% | −0.210 | no | 4 | 1.781 |
| TGGAAA | 0.037% | 0.333 | no | 4 | −0.668 |
| TGGAAC | 0.022% | 0.612 | no | 4 | −0.753 |
| TGGAAG | 0.036% | 0.395 | no | 4 | −0.299 |
| TGGACA | 0.029% | 0.482 | no | 4 | −0.524 |
| TGGACC | 0.014% | 0.608 | no | 4 | −1.530 |
| TGGACG | 0.026% | 0.686 | no | 4 | −0.755 |
| TGGAGA | 0.041% | 0.427 | no | 4 | −1.040 |
| TGGAGC | 0.022% | 0.299 | no | 4 | −0.481 |
| TGGAGG | 0.034% | 0.101 | no | 4 | 0.826 |
| TGGCAA | 0.029% | 0.259 | no | 4 | −0.639 |
| TGGCAC | 0.022% | −0.133 | no | 4 | −0.046 |
| TGGCAG | 0.032% | NA | no | 4 | −0.242 |
| TGGCCA | 0.018% | NA | no | 4 | −0.328 |
| TGGCCC | 0.012% | NA | no | 4 | 0.218 |
| TGGCCG | 0.011% | 0.195 | no | 4 | −0.952 |
| TGGCGA | 0.023% | 0.443 | no | 4 | −0.868 |
| TGGCGC | 0.008% | 0.422 | no | 4 | −0.003 |
| TGGCGG | 0.021% | 0.301 | no | 4 | 0.063 |
| TGGTAA | 0.030% | −0.538 | no | 4 | −0.010 |
| TGGTAC | 0.019% | −0.144 | no | 4 | 0.413 |
| TGGTAG | 0.023% | −0.626 | no | 4 | −0.138 |
| TGGTCA | 0.017% | NA | no | 4 | −0.950 |
| TGGTCC | 0.009% | 0.136 | no | 4 | −0.215 |
| TGGTCG | 0.013% | 0.156 | no | 4 | 1.450 |
| TGGTGA | 0.023% | NA | no | 4 | −0.493 |
| TGGTGC | 0.018% | NA | no | 4 | −0.289 |
| TGGTGG | 0.025% | −0.308 | no | 4 | 0.415 |
| TGTAAA | 0.043% | −0.304 | no | 4 | −0.358 |
| TGTAAC | 0.026% | NA | no | 4 | −0.018 |
| TGTAAG | 0.035% | −0.362 | no | 4 | 0.147 |
| TGTACA | 0.024% | −0.205 | no | 4 | −0.191 |
| TGTACC | 0.013% | NA | no | 4 | −0.029 |
| TGTACG | 0.021% | 0.188 | no | 4 | −0.778 |
| TGTAGA | 0.037% | −0.303 | no | 4 | −1.079 |
| TGTAGC | 0.020% | −0.359 | no | 4 | −0.526 |
| TGTAGG | 0.030% | −0.688 | no | 4 | −0.622 |
| TGTCAA | 0.027% | NA | no | 4 | 0.482 |
| TGTCAC | 0.013% | NA | no | 4 | −0.717 |
| TGTCAG | 0.021% | −0.433 | no | 4 | 0.164 |
| TGTCCA | 0.015% | NA | no | 4 | −0.235 |
| TGTCCC | 0.007% | NA | no | 4 | 1.386 |
| TGTCCG | 0.007% | 0.234 | no | 4 | 0.057 |
| TGTCGA | 0.021% | 0.362 | no | 4 | −0.882 |
| TGTCGC | 0.014% | 0.219 | no | 4 | −0.680 |
| TGTCGG | 0.015% | 0.312 | no | 4 | −0.819 |
| TGTTAA | 0.028% | −0.439 | no | 4 | −0.240 |
| TGTTAC | 0.016% | NA | no | 4 | 0.656 |
| TGTTAG | 0.017% | −0.758 | no | 4 | −1.458 |
| TGTTCA | 0.019% | NA | no | 4 | 0.643 |
| TGTTCG | 0.013% | 0.424 | no | 4 | 1.367 |
| TGTTGA | 0.019% | NA | no | 4 | 0.146 |
| TGTTGC | 0.012% | NA | no | 4 | 0.101 |
| TGTTGG | 0.022% | NA | no | 4 | 0.046 |
| TTAAAA | 0.041% | −0.349 | yes | 4 | −6.257 |
| TTAAAC | 0.019% | NA | yes | 4 | −5.625 |
| TTAAAG | 0.037% | −0.242 | yes | 4 | −3.276 |
| TTAACA | 0.035% | NA | yes | 4 | −4.812 |
| TTAACC | 0.023% | NA | yes | 4 | −4.758 |
| TTAACG | 0.029% | NA | yes | 4 | −3.931 |
| TTAAGA | 0.033% | −0.217 | yes | 4 | −7.483 |
| TTAAGC | 0.029% | −0.260 | yes | 4 | −5.977 |
| TTAAGG | 0.035% | −0.489 | yes | 4 | −5.448 |
| TTACAA | 0.027% | NA | no | 4 | −4.111 |
| TTACAC | 0.023% | NA | no | 4 | −4.213 |
| TTACAG | 0.018% | −0.207 | no | 4 | −2.331 |
| TTACCA | 0.014% | NA | no | 4 | −4.483 |
| TTACCC | 0.010% | NA | no | 4 | −2.309 |
| TTACCG | 0.011% | 0.416 | no | 4 | −1.287 |
| TTACGA | 0.014% | 0.307 | no | 4 | −1.468 |
| TTACGC | 0.014% | 0.331 | no | 4 | −1.441 |
| TTACGG | 0.018% | NA | no | 4 | −1.053 |
| TTATAA | 0.029% | −0.441 | no | 4 | −4.000 |
| TTATAC | 0.019% | NA | no | 4 | −5.871 |
| TTATAG | 0.026% | −0.594 | no | 4 | −3.787 |
| TTATCA | 0.026% | NA | no | 4 | −3.704 |
| TTATCC | 0.008% | NA | no | 4 | −2.232 |
| TTATCG | 0.019% | 0.238 | no | 4 | −0.251 |
| TTATGA | 0.026% | NA | no | 4 | −5.339 |
| TTATGC | 0.018% | −0.300 | no | 4 | −0.623 |
| TTATGG | 0.022% | −0.263 | no | 4 | −0.347 |
| TTCAAT | 0.018% | NA | no | 4 | −2.208 |
| TTCACT | 0.010% | NA | no | 4 | −1.187 |
| TTCAGT | 0.019% | −0.197 | no | 4 | −0.135 |
| TTCATA | 0.016% | NA | no | 4 | −2.081 |
| TTCATC | 0.015% | 0.597 | no | 4 | −0.541 |
| TTCATG | 0.016% | NA | no | 4 | −0.760 |
| TTCCAT | 0.008% | NA | no | 4 | 1.476 |
| TTCCGT | 0.007% | NA | no | 4 | −0.918 |
| TTCCTC | 0.006% | NA | no | 4 | −0.978 |
| TTCCTG | 0.011% | 0.388 | no | 4 | −0.489 |
| TTCGAA | 0.022% | 0.421 | no | 4 | −0.274 |
| TTCGAC | 0.013% | 0.749 | no | 4 | 1.026 |
| TTCGAG | 0.015% | 0.397 | no | 4 | 0.701 |
| TTCGCA | 0.016% | 0.460 | no | 4 | −0.065 |
| TTCGCC | 0.010% | 0.637 | no | 4 | −0.549 |
| TTCGCG | 0.013% | 0.720 | no | 4 | −0.507 |
| TTCGGA | 0.017% | 0.487 | no | 4 | −0.120 |
| TTCGGC | 0.012% | 0.615 | no | 4 | −0.684 |
| TTCGGG | 0.022% | 0.326 | no | 4 | −0.467 |
| TTCTAT | 0.009% | −0.185 | no | 4 | −3.859 |
| TTCTCT | 0.010% | NA | no | 4 | 0.032 |
| TTCTGT | 0.013% | NA | no | 4 | −0.825 |
| TTCTTA | 0.012% | −0.207 | no | 4 | −4.174 |
| TTCTTC | 0.009% | 0.446 | no | 4 | −2.850 |
| TTCTTG | 0.012% | −0.217 | no | 4 | −0.295 |
| TTGAAT | 0.029% | NA | yes | 4 | −3.343 |
| TTGACT | 0.018% | NA | yes | 4 | −3.981 |
| TTGAGT | 0.021% | −0.224 | yes | 4 | −3.268 |
| TTGATA | 0.026% | −0.226 | yes | 4 | −3.154 |
| TTGATC | 0.014% | 0.330 | yes | 4 | −3.707 |
| TTGATG | 0.024% | NA | yes | 4 | −2.991 |
| TTGCAT | 0.020% | NA | no | 4 | −1.414 |
| TTGCCT | 0.009% | 0.262 | no | 4 | 0.493 |
| TTGCGT | 0.014% | 0.240 | no | 4 | −1.217 |
| TTGCTA | 0.010% | NA | no | 4 | −0.981 |
| TTGCTC | 0.011% | −0.151 | no | 4 | −1.593 |
| TTGCTG | 0.010% | 0.184 | no | 4 | −1.199 |
| TTGGAA | 0.032% | 0.206 | no | 4 | −0.962 |
| TTGGAC | 0.021% | 0.315 | no | 4 | 0.463 |
| TTGGAG | 0.031% | NA | no | 4 | −0.221 |
| TTGGCA | 0.021% | NA | no | 4 | −1.925 |
| TTGGCC | 0.008% | NA | no | 4 | −1.264 |
| TTGGCG | 0.018% | 0.355 | no | 4 | −0.284 |
| TTGGGA | 0.024% | NA | no | 4 | −2.123 |
| TTGGGC | 0.011% | −0.138 | no | 4 | −2.203 |
| TTGGGG | 0.023% | −0.218 | no | 4 | −2.416 |
| TTGTAT | 0.017% | NA | no | 4 | −2.518 |
| TTGTCT | 0.014% | NA | no | 4 | −2.588 |
| TTGTGT | 0.013% | NA | no | 4 | −2.719 |
| TTGTTA | 0.025% | −0.337 | no | 4 | −4.186 |
| TTGTTC | 0.011% | 0.230 | no | 4 | −3.284 |
| TTGTTG | 0.014% | −0.150 | no | 4 | −1.180 |
| TTTAAT | 0.020% | −0.494 | no | 4 | −6.052 |
| TTTACT | 0.015% | NA | no | 4 | −5.274 |
| TTTAGT | 0.018% | −0.635 | no | 4 | −9.622 |
| TTTATA | 0.019% | −0.421 | no | 4 | −5.136 |
| TTTATC | 0.014% | NA | no | 4 | −5.023 |
| TTTATG | 0.015% | −0.370 | no | 4 | −1.536 |
| TTTCAT | 0.014% | NA | no | 4 | −2.833 |
| TTTCCT | 0.008% | NA | no | 4 | −3.520 |
| TTTCGT | 0.013% | 0.291 | no | 4 | −1.753 |
| TTTCTA | 0.019% | NA | no | 4 | −3.406 |
| TTTCTC | 0.014% | NA | no | 4 | −5.454 |
| TTTCTG | 0.012% | NA | no | 4 | −3.087 |
| TTTGAA | 0.031% | NA | no | 4 | −2.719 |
| TTTGAC | 0.017% | NA | no | 4 | −2.842 |
| TTTGAG | 0.021% | −0.225 | no | 4 | 0.059 |
| TTTGCA | 0.018% | −0.243 | no | 4 | −3.287 |
| TTTGCC | 0.013% | NA | no | 4 | −1.957 |
| TTTGCG | 0.016% | 0.274 | no | 4 | 1.190 |
| TTTGGA | 0.031% | NA | no | 4 | −0.740 |
| TTTGGC | 0.018% | NA | no | 4 | −1.142 |
| TTTGGG | 0.021% | −0.200 | no | 4 | −2.438 |
| TTTTAT | 0.016% | −0.433 | no | 4 | −3.129 |
| TTTTCT | 0.012% | NA | no | 4 | −5.242 |
| TTTTGT | 0.015% | −0.281 | no | 4 | −3.951 |
| TTTTTA | 0.017% | −0.436 | no | 4 | −5.801 |
| TTTTTC | 0.009% | NA | no | 4 | −4.067 |
| TTTTTG | 0.015% | −0.451 | no | 4 | −4.376 |
| AAAAAA | 0.116% | NA | no | 5 | −4.298 |
| AAAAAC | 0.083% | NA | no | 5 | −3.426 |
| AAAAAG | 0.096% | NA | no | 5 | −0.607 |
| AAAACA | 0.070% | NA | no | 5 | −2.840 |
| AAAACC | 0.036% | NA | no | 5 | −2.847 |
| AAAACG | 0.066% | NA | no | 5 | −0.718 |
| AAAAGA | 0.099% | NA | no | 5 | −3.161 |
| AAAAGC | 0.070% | NA | no | 5 | −2.806 |
| AAAAGG | 0.101% | −0.210 | no | 5 | −2.186 |
| AAACAA | 0.076% | NA | no | 5 | −2.675 |
| AAACAC | 0.040% | NA | no | 5 | −4.391 |
| AAACAG | 0.063% | NA | no | 5 | −1.710 |
| AAACCA | 0.044% | NA | no | 5 | −1.597 |
| AAACCC | 0.023% | −0.292 | no | 5 | −2.155 |
| AAACCG | 0.040% | 0.295 | no | 5 | 0.265 |
| AAACGA | 0.061% | 0.408 | no | 5 | −1.551 |
| AAACGC | 0.035% | 0.399 | no | 5 | −1.063 |
| AAACGG | 0.056% | 0.177 | no | 5 | −0.487 |
| AAATAA | 0.064% | NA | no | 5 | −4.264 |
| AAATAC | 0.038% | NA | no | 5 | −3.313 |
| AAATAG | 0.047% | −0.518 | no | 5 | −2.333 |
| AAATCA | 0.044% | NA | no | 5 | −3.412 |
| AAATCC | 0.014% | NA | no | 5 | −3.685 |
| AAATCG | 0.035% | 0.271 | no | 5 | −0.576 |
| AAATGA | 0.058% | NA | no | 5 | −2.896 |
| AAATGC | 0.042% | −0.223 | no | 5 | −2.509 |
| AAATGG | 0.055% | NA | no | 5 | −1.576 |
| AACAAT | 0.046% | NA | no | 5 | −1.641 |
| AACACT | 0.031% | 0.256 | no | 5 | −1.171 |
| AACAGT | 0.044% | NA | no | 5 | −0.666 |
| AACATA | 0.049% | NA | no | 5 | −1.138 |
| AACATC | 0.033% | 0.351 | no | 5 | −0.742 |
| AACATG | 0.042% | NA | no | 5 | −0.489 |
| AACCAT | 0.029% | NA | no | 5 | −0.757 |
| AACCCT | 0.018% | 0.324 | no | 5 | −1.525 |
| AACCGT | 0.021% | NA | no | 5 | 0.533 |
| AACCTA | 0.023% | NA | no | 5 | −0.741 |
| AACCTC | 0.019% | 0.286 | no | 5 | −1.607 |
| AACCTG | 0.022% | 0.458 | no | 5 | 0.184 |
| AACGAA | 0.059% | NA | no | 5 | −0.009 |
| AACGAC | 0.036% | 0.481 | no | 5 | −0.297 |
| AACGAG | 0.051% | NA | no | 5 | −0.840 |
| AACGCA | 0.041% | NA | no | 5 | −1.264 |
| AACGCC | 0.022% | 0.516 | no | 5 | 0.729 |
| AACGCG | 0.042% | 0.692 | no | 5 | 0.372 |
| AACGGA | 0.061% | 0.780 | no | 5 | −0.270 |
| AACGGC | 0.034% | 0.523 | no | 5 | 0.480 |
| AACGGG | 0.048% | 0.288 | no | 5 | −0.238 |
| AACTAT | 0.019% | NA | no | 5 | −1.804 |
| AACTCT | 0.017% | NA | no | 5 | −0.369 |
| AACTGT | 0.021% | NA | no | 5 | 0.684 |
| AACTTA | 0.024% | NA | no | 5 | −2.119 |
| AACTTC | 0.015% | 0.414 | no | 5 | −0.767 |
| AACTTG | 0.032% | NA | no | 5 | 0.526 |
| AAGAAT | 0.068% | 0.613 | no | 5 | −1.100 |
| AAGACT | 0.032% | 0.778 | no | 5 | −0.590 |
| AAGAGT | 0.058% | 0.316 | no | 5 | −1.370 |
| AAGATA | 0.063% | 0.391 | no | 5 | −1.436 |
| AAGATC | 0.036% | 0.637 | no | 5 | −1.025 |
| AAGATG | 0.058% | 0.465 | no | 5 | −0.342 |
| AAGCAT | 0.041% | NA | no | 5 | −1.973 |
| AAGCCT | 0.024% | NA | no | 5 | −1.669 |
| AAGCGT | 0.027% | NA | no | 5 | −1.179 |
| AAGCTA | 0.035% | NA | no | 5 | −1.865 |
| AAGCTC | 0.020% | NA | no | 5 | −0.488 |
| AAGCTG | 0.040% | 0.235 | no | 5 | 0.107 |
| AAGGAA | 0.083% | 0.545 | no | 5 | −2.097 |
| AAGGAC | 0.047% | 0.571 | no | 5 | −1.555 |
| AAGGAG | 0.058% | NA | no | 5 | −0.136 |
| AAGGCA | 0.053% | NA | no | 5 | −2.465 |
| AAGGCC | 0.023% | NA | no | 5 | −1.155 |
| AAGGCG | 0.035% | 0.306 | no | 5 | −0.214 |
| AAGGGA | 0.074% | −0.207 | no | 5 | −3.974 |
| AAGGGC | 0.038% | −0.146 | no | 5 | −4.913 |
| AAGGGG | 0.069% | −0.193 | no | 5 | −2.523 |
| AAGTAT | 0.039% | NA | no | 5 | −2.379 |
| AAGTCT | 0.024% | 0.381 | no | 5 | −1.489 |
| AAGTGT | 0.046% | NA | no | 5 | −1.648 |
| AAGTTA | 0.043% | NA | no | 5 | −2.458 |
| AAGTTC | 0.026% | 0.292 | no | 5 | −3.454 |
| AAGTTG | 0.034% | NA | no | 5 | −1.179 |
| AATAAT | 0.040% | −0.289 | no | 5 | −3.529 |
| AATACT | 0.022% | NA | no | 5 | −3.995 |
| AATAGT | 0.035% | −0.463 | no | 5 | −7.162 |
| AATATA | 0.042% | −0.477 | no | 5 | −3.683 |
| AATATC | 0.021% | NA | no | 5 | −2.339 |
| AATATG | 0.040% | −0.245 | no | 5 | −0.688 |
| AATCAT | 0.033% | NA | no | 5 | −2.366 |
| AATCCT | 0.018% | 0.417 | no | 5 | −1.551 |
| AATCGT | 0.027% | 0.314 | no | 5 | −1.588 |
| AATCTA | 0.024% | NA | no | 5 | −3.222 |
| AATCTC | 0.010% | NA | no | 5 | −2.828 |
| AATCTG | 0.030% | 0.228 | no | 5 | −0.659 |
| AATGAA | 0.056% | NA | no | 5 | −2.047 |
| AATGAC | 0.018% | 0.349 | no | 5 | −1.261 |
| AATGAG | 0.029% | NA | no | 5 | −0.549 |
| AATGCA | 0.035% | −0.225 | no | 5 | −3.043 |
| AATGCC | 0.009% | NA | no | 5 | −1.035 |
| AATGCG | 0.025% | 0.295 | no | 5 | −0.655 |
| AATGGA | 0.043% | 0.386 | no | 5 | −0.864 |
| AATGGC | 0.024% | 0.119 | no | 5 | −0.700 |
| AATGGG | 0.040% | −0.266 | no | 5 | −2.138 |
| AATTAT | 0.024% | −0.392 | no | 5 | −4.140 |
| AATTCT | 0.006% | NA | no | 5 | −3.905 |
| AATTGT | 0.020% | NA | no | 5 | −3.972 |
| AATTTA | 0.026% | NA | no | 5 | −3.956 |
| AATTTC | 0.013% | NA | no | 5 | −5.056 |
| AATTTG | 0.021% | −0.175 | no | 5 | −2.374 |
| ACAAAA | 0.082% | NA | no | 5 | −2.854 |
| ACAAAC | 0.049% | NA | no | 5 | −2.644 |
| ACAAAG | 0.070% | NA | no | 5 | −0.187 |
| ACAACA | 0.053% | NA | no | 5 | −1.154 |
| ACAACC | 0.027% | NA | no | 5 | −0.819 |
| ACAACG | 0.043% | 0.587 | no | 5 | 0.377 |
| ACAAGA | 0.065% | 0.488 | no | 5 | −0.926 |
| ACAAGC | 0.048% | 0.353 | no | 5 | −1.421 |
| ACAAGG | 0.065% | NA | no | 5 | −0.420 |
| ACACAA | 0.048% | NA | no | 5 | −2.059 |
| ACACAC | 0.028% | NA | no | 5 | −3.690 |
| ACACAG | 0.048% | NA | no | 5 | −0.472 |
| ACACCA | 0.030% | −0.127 | no | 5 | −1.609 |
| ACACCC | 0.017% | NA | no | 5 | −1.031 |
| ACACCG | 0.025% | 0.536 | no | 5 | −0.067 |
| ACACGA | 0.044% | NA | no | 5 | −0.517 |
| ACACGC | 0.027% | 0.497 | no | 5 | −1.236 |
| ACACGG | 0.044% | 0.350 | no | 5 | −0.332 |
| ACATAA | 0.047% | NA | no | 5 | −3.398 |
| ACATAC | 0.032% | NA | no | 5 | −3.445 |
| ACATAG | 0.045% | −0.435 | no | 5 | −1.259 |
| ACATCA | 0.034% | NA | no | 5 | −2.158 |
| ACATCC | 0.015% | 0.231 | no | 5 | −0.653 |
| ACATCG | 0.030% | 0.571 | no | 5 | −0.659 |
| ACATGA | 0.037% | NA | no | 5 | −2.797 |
| ACATGC | 0.026% | NA | no | 5 | −0.300 |
| ACATGG | 0.049% | NA | no | 5 | −0.971 |
| ACCAAT | 0.029% | NA | no | 5 | 0.074 |
| ACCACT | 0.015% | NA | no | 5 | −0.829 |
| ACCAGT | 0.030% | NA | no | 5 | −0.548 |
| ACCATA | 0.028% | −0.569 | no | 5 | −1.492 |
| ACCATC | 0.017% | NA | no | 5 | 0.452 |
| ACCATG | 0.029% | NA | no | 5 | −0.179 |
| ACCCAT | 0.019% | −0.188 | no | 5 | −0.502 |
| ACCCCT | 0.013% | −0.152 | no | 5 | −1.971 |
| ACCCGT | 0.016% | 0.279 | no | 5 | −0.472 |
| ACCCTA | 0.012% | −0.298 | no | 5 | −3.091 |
| ACCCTC | 0.010% | 0.137 | no | 5 | −1.155 |
| ACCCTG | 0.016% | 0.155 | no | 5 | 0.953 |
| ACCGAA | 0.035% | NA | no | 5 | −0.100 |
| ACCGAC | 0.025% | NA | no | 5 | 0.296 |
| ACCGAG | 0.032% | NA | no | 5 | 0.018 |
| ACCGCA | 0.021% | NA | no | 5 | −0.489 |
| ACCGCC | 0.017% | NA | no | 5 | 1.759 |
| ACCGCG | 0.022% | 0.487 | no | 5 | −0.743 |
| ACCGGA | 0.034% | NA | no | 5 | −0.278 |
| ACCGGC | 0.019% | NA | no | 5 | 0.949 |
| ACCGGG | 0.030% | 0.550 | no | 5 | −0.455 |
| ACCTAT | 0.016% | −0.268 | no | 5 | −1.735 |
| ACCTCT | 0.012% | NA | no | 5 | −1.503 |
| ACCTGT | 0.020% | NA | no | 5 | 0.204 |
| ACCTTA | 0.021% | NA | no | 5 | −1.504 |
| ACCTTC | 0.012% | NA | no | 5 | −1.703 |
| ACCTTG | 0.016% | NA | no | 5 | −0.361 |
| ACGAAT | 0.047% | NA | no | 5 | −0.211 |
| ACGACT | 0.021% | 0.496 | no | 5 | −0.097 |
| ACGAGT | 0.037% | NA | no | 5 | −0.119 |
| ACGATA | 0.033% | NA | no | 5 | −0.708 |
| ACGATC | 0.025% | 0.631 | no | 5 | −0.942 |
| ACGATG | 0.035% | 0.361 | no | 5 | 0.446 |
| ACGCAT | 0.025% | NA | no | 5 | −1.115 |
| ACGCCT | 0.020% | 0.525 | no | 5 | −0.441 |
| ACGCGT | 0.022% | 0.363 | no | 5 | −0.420 |
| ACGCTA | 0.023% | NA | no | 5 | −0.307 |
| ACGCTC | 0.017% | 0.387 | no | 5 | 0.271 |
| ACGCTG | 0.023% | 0.380 | no | 5 | −0.317 |
| ACGGAA | 0.061% | 0.504 | no | 5 | −0.653 |
| ACGGAC | 0.036% | 0.676 | no | 5 | −0.672 |
| ACGGAG | 0.041% | 0.381 | no | 5 | −0.388 |
| ACGGCA | 0.031% | NA | no | 5 | −0.083 |
| ACGGCC | 0.021% | 0.363 | no | 5 | 0.717 |
| ACGGCG | 0.024% | 0.709 | no | 5 | 0.796 |
| ACGGGA | 0.044% | 0.425 | no | 5 | 0.084 |
| ACGGGC | 0.035% | 0.359 | no | 5 | 0.315 |
| ACGGGG | 0.043% | 0.204 | no | 5 | −0.443 |
| ACGTAT | 0.030% | NA | no | 5 | −1.543 |
| ACGTCT | 0.021% | 0.513 | no | 5 | −0.717 |
| ACGTGT | 0.028% | NA | no | 5 | −0.255 |
| ACGTTA | 0.033% | NA | no | 5 | −1.795 |
| ACGTTC | 0.018% | 0.520 | no | 5 | −0.743 |
| ACGTTG | 0.032% | 0.252 | no | 5 | −0.021 |
| ACTAAT | 0.033% | −0.334 | no | 5 | −3.642 |
| ACTACT | 0.016% | 0.308 | no | 5 | −2.192 |
| ACTAGT | 0.025% | −0.409 | no | 5 | −7.390 |
| ACTATA | 0.024% | −0.391 | no | 5 | −4.008 |
| ACTATC | 0.021% | NA | no | 5 | −3.883 |
| ACTATG | 0.021% | NA | no | 5 | −0.814 |
| ACTCAT | 0.023% | NA | no | 5 | −3.471 |
| ACTCCT | 0.017% | 0.416 | no | 5 | −2.373 |
| ACTCGT | 0.018% | 0.266 | no | 5 | −1.264 |
| ACTCTA | 0.022% | −0.394 | no | 5 | −3.332 |
| ACTCTC | 0.014% | NA | no | 5 | −2.268 |
| ACTCTG | 0.021% | 0.194 | no | 5 | −0.477 |
| ACTGAA | 0.040% | NA | no | 5 | −0.118 |
| ACTGAC | 0.023% | 0.335 | no | 5 | −1.737 |
| ACTGAG | 0.025% | NA | no | 5 | 0.822 |
| ACTGCA | 0.025% | 0.203 | no | 5 | −2.160 |
| ACTGCC | 0.018% | NA | no | 5 | 0.120 |
| ACTGCG | 0.024% | 0.510 | no | 5 | 0.118 |
| ACTGGA | 0.037% | NA | no | 5 | −0.286 |
| ACTGGC | 0.025% | NA | no | 5 | −0.588 |
| ACTGGG | 0.037% | NA | no | 5 | −1.048 |
| ACTTAT | 0.023% | NA | no | 5 | −3.348 |
| ACTTCT | 0.012% | 0.263 | no | 5 | −2.599 |
| ACTTGT | 0.018% | NA | no | 5 | −2.260 |
| ACTTTA | 0.025% | −0.551 | no | 5 | −6.474 |
| ACTTTC | 0.012% | NA | no | 5 | −2.527 |
| ACTTTG | 0.018% | −0.155 | no | 5 | −0.536 |
| AGCAAA | 0.066% | NA | no | 5 | −0.231 |
| AGCAAC | 0.040% | NA | no | 5 | −0.478 |
| AGCAAG | 0.052% | NA | no | 5 | −0.850 |
| AGCACA | 0.035% | NA | no | 5 | −0.568 |
| AGCACC | 0.020% | −0.302 | no | 5 | −1.046 |
| AGCACG | 0.038% | NA | no | 5 | −0.230 |
| AGCAGA | 0.048% | NA | no | 5 | −0.701 |
| AGCAGC | 0.027% | −0.295 | no | 5 | 0.466 |
| AGCAGG | 0.039% | −0.304 | no | 5 | −0.610 |
| AGCCAA | 0.033% | NA | no | 5 | 0.048 |
| AGCCAC | 0.021% | NA | no | 5 | −0.708 |
| AGCCAG | 0.035% | −0.193 | no | 5 | −0.625 |
| AGCCCA | 0.024% | −0.292 | no | 5 | −1.079 |
| AGCCCC | 0.018% | −0.233 | no | 5 | 0.248 |
| AGCCCG | 0.022% | NA | no | 5 | 0.030 |
| AGCCGA | 0.031% | NA | no | 5 | 0.240 |
| AGCCGC | 0.018% | NA | no | 5 | −0.319 |
| AGCCGG | 0.029% | 0.257 | no | 5 | −0.016 |
| AGCTAA | 0.033% | −0.436 | no | 5 | −0.386 |
| AGCTAC | 0.025% | NA | no | 5 | −0.221 |
| AGCTAG | 0.041% | −0.772 | no | 5 | −0.344 |
| AGCTCA | 0.034% | NA | no | 5 | −0.220 |
| AGCTCC | 0.018% | NA | no | 5 | −0.968 |
| AGCTCG | 0.023% | NA | no | 5 | 1.106 |
| AGCTGA | 0.032% | NA | no | 5 | −0.143 |
| AGCTGC | 0.017% | 0.152 | no | 5 | −0.427 |
| AGCTGG | 0.029% | −0.227 | no | 5 | −0.206 |
| AGGAAA | 0.092% | NA | no | 5 | −3.561 |
| AGGAAC | 0.064% | 0.338 | no | 5 | −2.293 |
| AGGAAG | 0.066% | NA | no | 5 | −1.064 |
| AGGACA | 0.066% | NA | no | 5 | −3.251 |
| AGGACC | 0.034% | 0.246 | no | 5 | −1.549 |
| AGGACG | 0.047% | 0.394 | no | 5 | −0.096 |
| AGGAGA | 0.078% | NA | no | 5 | −1.908 |
| AGGAGC | 0.040% | 0.336 | no | 5 | −1.938 |
| AGGAGG | 0.065% | −0.147 | no | 5 | −1.028 |
| AGGCAA | 0.055% | NA | no | 5 | −2.114 |
| AGGCAC | 0.039% | NA | no | 5 | −3.296 |
| AGGCAG | 0.052% | −0.490 | no | 5 | −1.343 |
| AGGCCA | 0.035% | NA | no | 5 | −1.280 |
| AGGCCC | 0.029% | NA | no | 5 | −0.420 |
| AGGCCG | 0.031% | NA | no | 5 | −0.324 |
| AGGCGA | 0.040% | NA | no | 5 | −0.846 |
| AGGCGC | 0.025% | NA | no | 5 | 1.091 |
| AGGCGG | 0.035% | NA | no | 5 | −0.488 |
| AGGTAA | 0.065% | −0.555 | no | 5 | −5.154 |
| AGGTAC | 0.038% | NA | no | 5 | −4.296 |
| AGGTAG | 0.042% | −0.871 | no | 5 | −3.122 |
| AGGTCA | 0.043% | NA | no | 5 | −2.539 |
| AGGTCC | 0.025% | −0.226 | no | 5 | −1.406 |
| AGGTCG | 0.032% | NA | no | 5 | −0.550 |
| AGGTGA | 0.039% | −0.429 | no | 5 | −2.689 |
| AGGTGC | 0.033% | −0.262 | no | 5 | −1.279 |
| AGGTGG | 0.044% | −0.423 | no | 5 | −0.072 |
| AGTAAA | 0.063% | NA | no | 5 | −1.632 |
| AGTAAC | 0.050% | NA | no | 5 | −0.695 |
| AGTAAG | 0.062% | −0.505 | no | 5 | −0.778 |
| AGTACA | 0.044% | NA | no | 5 | −1.271 |
| AGTACC | 0.029% | NA | no | 5 | −1.082 |
| AGTACG | 0.046% | NA | no | 5 | 0.024 |
| AGTAGA | 0.052% | NA | no | 5 | −3.087 |
| AGTAGC | 0.032% | −0.483 | no | 5 | −2.096 |
| AGTAGG | 0.052% | −0.703 | no | 5 | −2.394 |
| AGTCAA | 0.049% | NA | no | 5 | −0.576 |
| AGTCAC | 0.024% | NA | no | 5 | −0.941 |
| AGTCAG | 0.039% | −0.684 | no | 5 | −1.114 |
| AGTCCA | 0.024% | NA | no | 5 | −0.521 |
| AGTCCC | 0.015% | −0.237 | no | 5 | −0.021 |
| AGTCCG | 0.018% | 0.277 | no | 5 | 0.367 |
| AGTCGA | 0.034% | NA | no | 5 | 0.016 |
| AGTCGC | 0.023% | 0.365 | no | 5 | −0.564 |
| AGTCGG | 0.031% | 0.160 | no | 5 | −0.096 |
| AGTTAA | 0.054% | −0.172 | no | 5 | −1.972 |
| AGTTAC | 0.020% | 0.192 | no | 5 | −0.533 |
| AGTTAG | 0.040% | −0.645 | no | 5 | −0.840 |
| AGTTCA | 0.031% | 0.204 | no | 5 | −1.079 |
| AGTTCC | 0.013% | −0.098 | no | 5 | 1.774 |
| AGTTCG | 0.021% | 0.505 | no | 5 | 2.317 |
| AGTTGA | 0.039% | 0.119 | no | 5 | 0.377 |
| AGTTGC | 0.021% | 0.176 | no | 5 | 1.008 |
| AGTTGG | 0.034% | NA | no | 5 | −0.493 |
| ATAAAA | 0.080% | −0.542 | yes | 5 | −5.299 |
| ATAAAC | 0.059% | NA | yes | 5 | −4.890 |
| ATAAAG | 0.058% | −0.271 | yes | 5 | −3.286 |
| ATAACA | 0.058% | −0.364 | yes | 5 | −6.056 |
| ATAACC | 0.031% | −0.274 | yes | 5 | −3.913 |
| ATAACG | 0.047% | NA | yes | 5 | −3.211 |
| ATAAGA | 0.056% | NA | yes | 5 | −5.331 |
| ATAAGC | 0.040% | NA | yes | 5 | −5.799 |
| ATAAGG | 0.061% | −0.365 | yes | 5 | −5.184 |
| ATACAA | 0.047% | NA | no | 5 | −2.992 |
| ATACAC | 0.032% | NA | no | 5 | −3.030 |
| ATACAG | 0.040% | NA | no | 5 | −1.862 |
| ATACCA | 0.029% | NA | no | 5 | −1.355 |
| ATACCC | 0.017% | −0.229 | no | 5 | −1.258 |
| ATACCG | 0.019% | 0.319 | no | 5 | −0.173 |
| ATACGA | 0.043% | NA | no | 5 | −2.295 |
| ATACGC | 0.022% | 0.358 | no | 5 | −1.628 |
| ATACGG | 0.038% | 0.193 | no | 5 | −0.746 |
| ATATAA | 0.056% | −0.361 | no | 5 | −5.270 |
| ATATAC | 0.037% | NA | no | 5 | −4.232 |
| ATATAG | 0.041% | −0.674 | no | 5 | −2.537 |
| ATATCA | 0.036% | NA | no | 5 | −4.023 |
| ATATCC | 0.016% | NA | no | 5 | −0.528 |
| ATATCG | 0.032% | NA | no | 5 | 0.356 |
| ATATGA | 0.043% | NA | no | 5 | −1.698 |
| ATATGC | 0.030% | −0.243 | no | 5 | −2.881 |
| ATATGG | 0.043% | NA | no | 5 | −0.719 |
| ATCAAT | 0.036% | NA | no | 5 | −0.510 |
| ATCACT | 0.021% | NA | no | 5 | 0.192 |
| ATCAGT | 0.029% | −0.409 | no | 5 | −1.810 |
| ATCATA | 0.034% | NA | no | 5 | −0.557 |
| ATCATC | 0.021% | NA | no | 5 | 0.534 |
| ATCATG | 0.028% | NA | no | 5 | −0.848 |
| ATCCAT | 0.026% | NA | no | 5 | −0.850 |
| ATCCCT | 0.014% | NA | no | 5 | −1.684 |
| ATCCGT | 0.015% | NA | no | 5 | −1.359 |
| ATCCTA | 0.017% | NA | no | 5 | −0.827 |
| ATCCTC | 0.014% | NA | no | 5 | −0.712 |
| ATCCTG | 0.016% | 0.447 | no | 5 | 0.126 |
| ATCGAA | 0.041% | NA | no | 5 | −0.076 |
| ATCGAC | 0.020% | 0.539 | no | 5 | −0.078 |
| ATCGAG | 0.037% | NA | no | 5 | −0.805 |
| ATCGCA | 0.030% | NA | no | 5 | 0.443 |
| ATCGCC | 0.022% | 0.290 | no | 5 | 0.958 |
| ATCGCG | 0.025% | 0.541 | no | 5 | −0.167 |
| ATCGGA | 0.039% | 0.342 | no | 5 | −0.115 |
| ATCGGC | 0.025% | 0.334 | no | 5 | 0.218 |
| ATCGGG | 0.035% | 0.390 | no | 5 | −0.354 |
| ATCTAT | 0.021% | −0.315 | no | 5 | −0.895 |
| ATCTCT | 0.015% | NA | no | 5 | −1.080 |
| ATCTGT | 0.023% | NA | no | 5 | 0.256 |
| ATCTTA | 0.022% | −0.350 | no | 5 | −0.819 |
| ATCTTC | 0.016% | NA | no | 5 | −1.648 |
| ATCTTG | 0.017% | −0.191 | no | 5 | −0.218 |
| ATGAAT | 0.047% | 0.279 | yes | 5 | −3.010 |
| ATGACT | 0.032% | 0.367 | yes | 5 | −1.853 |
| ATGAGT | 0.043% | NA | yes | 5 | −4.072 |
| ATGATA | 0.041% | NA | yes | 5 | −5.173 |
| ATGATC | 0.029% | 0.442 | yes | 5 | −4.543 |
| ATGATG | 0.035% | 0.187 | yes | 5 | −2.675 |
| ATGCAT | 0.031% | NA | no | 5 | −2.752 |
| ATGCCT | 0.012% | 0.331 | no | 5 | 0.031 |
| ATGCGT | 0.024% | NA | no | 5 | −1.629 |
| ATGCTA | 0.030% | NA | no | 5 | −2.856 |
| ATGCTC | 0.020% | NA | no | 5 | −4.102 |
| ATGCTG | 0.021% | 0.208 | no | 5 | 0.015 |
| ATGGAA | 0.052% | 0.393 | no | 5 | −0.996 |
| ATGGAC | 0.039% | 0.589 | no | 5 | −0.534 |
| ATGGAG | 0.047% | 0.229 | no | 5 | −0.418 |
| ATGGCA | 0.044% | NA | no | 5 | −1.948 |
| ATGGCC | 0.022% | NA | no | 5 | 0.689 |
| ATGGCG | 0.034% | 0.388 | no | 5 | −0.119 |
| ATGGGA | 0.064% | 0.054 | no | 5 | −2.200 |
| ATGGGC | 0.028% | −0.106 | no | 5 | −2.348 |
| ATGGGG | 0.052% | −0.331 | no | 5 | −2.867 |
| ATGTAT | 0.034% | NA | no | 5 | −3.231 |
| ATGTCT | 0.019% | NA | no | 5 | −3.313 |
| ATGTGT | 0.030% | NA | no | 5 | −2.322 |
| ATGTTA | 0.041% | NA | no | 5 | −3.521 |
| ATGTTC | 0.024% | 0.354 | no | 5 | −4.463 |
| ATGTTG | 0.030% | NA | no | 5 | −1.595 |
| ATTAAT | 0.042% | −0.360 | no | 5 | −4.518 |
| ATTACT | 0.016% | NA | no | 5 | −1.163 |
| ATTAGT | 0.034% | −0.437 | no | 5 | −9.337 |
| ATTATA | 0.038% | −0.813 | no | 5 | −4.559 |
| ATTATC | 0.025% | NA | no | 5 | −5.888 |
| ATTATG | 0.030% | −0.355 | no | 5 | −2.309 |
| ATTCAT | 0.019% | NA | no | 5 | −1.547 |
| ATTCCT | 0.007% | NA | no | 5 | −0.611 |
| ATTCGT | 0.017% | NA | no | 5 | 1.588 |
| ATTCTA | 0.020% | NA | no | 5 | −3.930 |
| ATTCTC | 0.015% | NA | no | 5 | −2.827 |
| ATTCTG | 0.020% | 0.188 | no | 5 | −1.028 |
| ATTGAA | 0.049% | NA | no | 5 | −2.491 |
| ATTGAC | 0.029% | NA | no | 5 | −2.342 |
| ATTGAG | 0.034% | NA | no | 5 | −0.692 |
| ATTGCA | 0.027% | −0.178 | no | 5 | −3.833 |
| ATTGCC | 0.015% | NA | no | 5 | −2.134 |
| ATTGCG | 0.026% | 0.287 | no | 5 | −0.600 |
| ATTGGA | 0.042% | 0.347 | no | 5 | −0.875 |
| ATTGGC | 0.028% | NA | no | 5 | −1.255 |
| ATTGGG | 0.040% | −0.103 | no | 5 | −2.010 |
| ATTTAT | 0.023% | −0.260 | no | 5 | −3.982 |
| ATTTCT | 0.017% | NA | no | 5 | −6.887 |
| ATTTGT | 0.023% | NA | no | 5 | −4.055 |
| ATTTTA | 0.027% | −0.725 | no | 5 | −5.043 |
| ATTTTC | 0.017% | NA | no | 5 | −3.757 |
| ATTTTG | 0.029% | −0.257 | no | 5 | −2.146 |
| CAAAAA | 0.047% | −0.255 | no | 5 | −2.163 |
| CAAAAC | 0.042% | NA | no | 5 | −2.136 |
| CAAAAG | 0.037% | NA | no | 5 | −0.198 |
| CAAACA | 0.040% | NA | no | 5 | −1.402 |
| CAAACC | 0.025% | NA | no | 5 | −1.664 |
| CAAACG | 0.035% | 0.292 | no | 5 | 0.022 |
| CAAAGA | 0.034% | 0.230 | no | 5 | −0.798 |
| CAAAGC | 0.025% | NA | no | 5 | −1.331 |
| CAAAGG | 0.043% | NA | no | 5 | −0.013 |
| CAACAA | 0.035% | 0.269 | no | 5 | −0.491 |
| CAACAC | 0.019% | 0.282 | no | 5 | −1.086 |
| CAACAG | 0.026% | NA | no | 5 | −0.336 |
| CAACCA | 0.021% | NA | no | 5 | −0.273 |
| CAACCC | 0.012% | NA | no | 5 | −0.916 |
| CAACCG | 0.021% | NA | no | 5 | −0.342 |
| CAACGA | 0.027% | 0.224 | no | 5 | −0.189 |
| CAACGC | 0.020% | 0.542 | no | 5 | −0.395 |
| CAACGG | 0.031% | 0.503 | no | 5 | −0.182 |
| CAATAA | 0.032% | −0.286 | no | 5 | −2.882 |
| CAATAC | 0.018% | NA | no | 5 | −0.569 |
| CAATAG | 0.029% | −0.465 | no | 5 | −1.951 |
| CAATCA | 0.026% | NA | no | 5 | −1.283 |
| CAATCC | 0.014% | NA | no | 5 | 0.622 |
| CAATCG | 0.020% | 0.297 | no | 5 | −0.238 |
| CAATGA | 0.024% | 0.266 | no | 5 | −1.142 |
| CAATGC | 0.017% | NA | no | 5 | −0.317 |
| CAATGG | 0.028% | NA | no | 5 | −0.403 |
| CACAAT | 0.029% | −0.217 | no | 5 | −0.731 |
| CACACT | 0.017% | NA | no | 5 | −1.344 |
| CACAGT | 0.019% | −0.197 | no | 5 | −1.502 |
| CACATA | 0.023% | −0.538 | no | 5 | −1.630 |
| CACATC | 0.012% | NA | no | 5 | 0.617 |
| CACATG | 0.019% | NA | no | 5 | 0.305 |
| CACCAT | 0.016% | −0.373 | no | 5 | −0.703 |
| CACCCT | 0.012% | NA | no | 5 | −3.190 |
| CACCGT | 0.012% | NA | no | 5 | 0.124 |
| CACCTA | 0.015% | −0.737 | no | 5 | 0.587 |
| CACCTC | 0.011% | NA | no | 5 | −1.006 |
| CACCTG | 0.010% | 0.154 | no | 5 | −0.859 |
| CACGAA | 0.034% | NA | no | 5 | −1.082 |
| CACGAC | 0.016% | NA | no | 5 | 0.161 |
| CACGAG | 0.027% | NA | no | 5 | 0.053 |
| CACGCA | 0.017% | NA | no | 5 | 0.202 |
| CACGCC | 0.014% | 0.268 | no | 5 | −0.845 |
| CACGCG | 0.020% | 0.289 | no | 5 | −0.662 |
| CACGGA | 0.021% | 0.304 | no | 5 | −0.142 |
| CACGGC | 0.019% | NA | no | 5 | −0.459 |
| CACGGG | 0.024% | 0.344 | no | 5 | 0.662 |
| CACTAT | 0.013% | −0.324 | no | 5 | −1.230 |
| CACTCT | 0.016% | −0.268 | no | 5 | −2.049 |
| CACTGT | 0.011% | NA | no | 5 | −1.174 |
| CACTTA | 0.015% | −0.562 | no | 5 | −1.130 |
| CACTTC | 0.011% | NA | no | 5 | −1.667 |
| CACTTG | 0.015% | −0.275 | no | 5 | −1.849 |
| CAGAAT | 0.029% | 0.207 | no | 5 | −1.732 |
| CAGACT | 0.027% | NA | no | 5 | −1.070 |
| CAGAGT | 0.031% | NA | no | 5 | −0.940 |
| CAGATA | 0.032% | −0.309 | no | 5 | −1.291 |
| CAGATC | 0.014% | NA | no | 5 | −0.205 |
| CAGATG | 0.031% | 0.190 | no | 5 | 0.162 |
| CAGCAT | 0.025% | −0.194 | no | 5 | 0.369 |
| CAGCCT | 0.016% | −0.303 | no | 5 | −0.481 |
| CAGCGT | 0.015% | NA | no | 5 | 0.330 |
| CAGCTA | 0.018% | −0.349 | no | 5 | −1.378 |
| CAGCTC | 0.010% | NA | no | 5 | −0.268 |
| CAGCTG | 0.015% | NA | no | 5 | 0.199 |
| CAGGAA | 0.039% | NA | no | 5 | −0.800 |
| CAGGAC | 0.025% | 0.250 | no | 5 | −0.298 |
| CAGGAG | 0.033% | NA | no | 5 | 0.020 |
| CAGGCA | 0.029% | −0.223 | no | 5 | −3.182 |
| CAGGCC | 0.019% | NA | no | 5 | −0.707 |
| CAGGCG | 0.023% | NA | no | 5 | −0.949 |
| CAGGGA | 0.045% | −0.328 | no | 5 | −4.328 |
| CAGGGC | 0.016% | −0.181 | no | 5 | −3.990 |
| CAGGGG | 0.033% | −0.227 | no | 5 | −2.819 |
| CAGTAT | 0.024% | −0.363 | no | 5 | −0.732 |
| CAGTCT | 0.013% | NA | no | 5 | −1.272 |
| CAGTGT | 0.020% | −0.196 | no | 5 | −0.107 |
| CAGTTA | 0.023% | −0.341 | no | 5 | −1.461 |
| CAGTTC | 0.011% | NA | no | 5 | −1.817 |
| CAGTTG | 0.016% | NA | no | 5 | −0.664 |
| CATAAT | 0.025% | −0.348 | no | 5 | −1.706 |
| CATACT | 0.012% | NA | no | 5 | −2.389 |
| CATAGT | 0.016% | −0.342 | no | 5 | −4.310 |
| CATATA | 0.015% | −0.414 | no | 5 | −1.444 |
| CATATC | 0.013% | NA | no | 5 | −1.787 |
| CATATG | 0.020% | −0.169 | no | 5 | 0.027 |
| CATCAT | 0.019% | NA | no | 5 | −0.887 |
| CATCCT | 0.008% | NA | no | 5 | −0.945 |
| CATCGT | 0.014% | NA | no | 5 | 0.552 |
| CATCTA | 0.016% | −0.335 | no | 5 | −0.187 |
| CATCTC | 0.009% | NA | no | 5 | −0.670 |
| CATCTG | 0.020% | 0.215 | no | 5 | 0.405 |
| CATGAA | 0.033% | 0.373 | no | 5 | −1.218 |
| CATGAC | 0.009% | 0.274 | no | 5 | 0.391 |
| CATGAG | 0.016% | NA | no | 5 | 0.562 |
| CATGCA | 0.026% | −0.095 | no | 5 | −1.129 |
| CATGCC | 0.009% | NA | no | 5 | 0.172 |
| CATGCG | 0.020% | NA | no | 5 | −0.372 |
| CATGGA | 0.027% | 0.524 | no | 5 | 0.007 |
| CATGGC | 0.016% | NA | no | 5 | −0.052 |
| CATGGG | 0.026% | 0.101 | no | 5 | −0.444 |
| CATTAT | 0.015% | −0.435 | no | 5 | −4.230 |
| CATTCT | 0.009% | NA | no | 5 | −1.799 |
| CATTGT | 0.011% | NA | no | 5 | −2.348 |
| CATTTA | 0.014% | −0.404 | no | 5 | −1.953 |
| CATTTC | 0.008% | NA | no | 5 | −3.017 |
| CATTTG | 0.017% | −0.160 | no | 5 | −2.055 |
| CCAAAA | 0.027% | −0.592 | no | 5 | −2.109 |
| CCAAAC | 0.022% | −0.360 | no | 5 | −1.518 |
| CCAAAG | 0.025% | −0.218 | no | 5 | −0.565 |
| CCAACA | 0.019% | NA | no | 5 | −0.150 |
| CCAACC | 0.011% | NA | no | 5 | −0.488 |
| CCAACG | 0.020% | 0.408 | no | 5 | 0.002 |
| CCAAGA | 0.019% | NA | no | 5 | −0.890 |
| CCAAGC | 0.016% | NA | no | 5 | −0.437 |
| CCAAGG | 0.022% | NA | no | 5 | −0.256 |
| CCACAA | 0.027% | NA | no | 5 | −1.804 |
| CCACAC | 0.015% | NA | no | 5 | −1.872 |
| CCACAG | 0.015% | −0.283 | no | 5 | −0.639 |
| CCACCA | 0.014% | −0.597 | no | 5 | −1.636 |
| CCACCC | 0.010% | −0.303 | no | 5 | 1.119 |
| CCACCG | 0.012% | NA | no | 5 | −0.229 |
| CCACGA | 0.018% | 0.317 | no | 5 | −0.224 |
| CCACGC | 0.011% | NA | no | 5 | −0.187 |
| CCACGG | 0.018% | 0.426 | no | 5 | −0.510 |
| CCATAA | 0.018% | −0.566 | no | 5 | −2.321 |
| CCATAC | 0.013% | −0.342 | no | 5 | −1.822 |
| CCATAG | 0.015% | −0.497 | no | 5 | −1.244 |
| CCATCA | 0.014% | −0.216 | no | 5 | −1.072 |
| CCATCC | 0.009% | NA | no | 5 | 0.526 |
| CCATCG | 0.013% | 0.391 | no | 5 | −0.053 |
| CCATGA | 0.014% | NA | no | 5 | −0.894 |
| CCATGC | 0.011% | NA | no | 5 | −0.891 |
| CCATGG | 0.014% | 0.183 | no | 5 | −1.268 |
| CCCAAT | 0.014% | −0.365 | no | 5 | −1.469 |
| CCCACT | 0.011% | −0.255 | no | 5 | −1.277 |
| CCCAGT | 0.010% | −0.388 | no | 5 | −2.238 |
| CCCATA | 0.012% | −0.475 | no | 5 | −3.772 |
| CCCATC | 0.010% | NA | no | 5 | −1.096 |
| CCCATG | 0.012% | NA | no | 5 | 0.331 |
| CCCCCT | 0.006% | −0.217 | no | 5 | −3.983 |
| CCCCGT | 0.010% | NA | no | 5 | −1.571 |
| CCCCTA | 0.007% | −0.434 | no | 5 | −4.929 |
| CCCCTG | 0.007% | 0.114 | no | 5 | 0.211 |
| CCCGAA | 0.015% | 0.281 | no | 5 | −0.873 |
| CCCGAC | 0.014% | 0.264 | no | 5 | 0.265 |
| CCCGAG | 0.016% | 0.320 | no | 5 | 0.805 |
| CCCGCA | 0.014% | NA | no | 5 | −1.168 |
| CCCGCC | 0.008% | NA | no | 5 | 0.112 |
| CCCGCG | 0.008% | 0.571 | no | 5 | −0.368 |
| CCCGGA | 0.013% | 0.454 | no | 5 | 0.068 |
| CCCGGC | 0.010% | NA | no | 5 | −0.045 |
| CCCGGG | 0.010% | 0.386 | no | 5 | 0.233 |
| CCCTAT | 0.006% | −0.386 | no | 5 | −4.151 |
| CCCTCT | 0.006% | NA | no | 5 | −0.306 |
| CCCTGT | 0.007% | NA | no | 5 | −0.619 |
| CCCTTA | 0.013% | −0.622 | no | 5 | −5.544 |
| CCCTTC | 0.006% | NA | no | 5 | 0.005 |
| CCCTTG | 0.006% | −0.092 | no | 5 | −1.191 |
| CCGAAT | 0.017% | NA | no | 5 | −0.405 |
| CCGACT | 0.009% | NA | no | 5 | −0.619 |
| CCGAGT | 0.018% | 0.211 | no | 5 | −0.240 |
| CCGATA | 0.016% | NA | no | 5 | −0.167 |
| CCGATC | 0.008% | NA | no | 5 | 0.961 |
| CCGATG | 0.018% | 0.275 | no | 5 | 0.722 |
| CCGCAT | 0.014% | NA | no | 5 | −0.323 |
| CCGCCT | 0.010% | NA | no | 5 | −0.453 |
| CCGCGT | 0.007% | 0.411 | no | 5 | 0.802 |
| CCGCTA | 0.017% | NA | no | 5 | −0.641 |
| CCGCTC | 0.008% | 0.224 | no | 5 | −1.731 |
| CCGCTG | 0.006% | 0.337 | no | 5 | 1.427 |
| CCGGAA | 0.028% | 0.524 | no | 5 | 0.192 |
| CCGGAC | 0.012% | 0.288 | no | 5 | −0.652 |
| CCGGAG | 0.020% | 0.622 | no | 5 | −0.362 |
| CCGGCA | 0.017% | −0.276 | no | 5 | 1.306 |
| CCGGCC | 0.010% | NA | no | 5 | 0.175 |
| CCGGCG | 0.017% | 0.621 | no | 5 | −1.036 |
| CCGGGA | 0.020% | 0.590 | no | 5 | 0.206 |
| CCGGGC | 0.012% | 0.454 | no | 5 | −0.736 |
| CCGGGG | 0.019% | 0.358 | no | 5 | −0.436 |
| CCGTAT | 0.009% | NA | no | 5 | 0.872 |
| CCGTCT | 0.006% | 0.337 | no | 5 | −0.172 |
| CCGTTA | 0.013% | −0.283 | no | 5 | −3.315 |
| CCGTTC | 0.009% | 0.196 | no | 5 | 0.261 |
| CCGTTG | 0.010% | 0.241 | no | 5 | −0.661 |
| CCTAAT | 0.010% | −0.628 | no | 5 | −3.190 |
| CCTACT | 0.008% | NA | no | 5 | 0.297 |
| CCTAGT | 0.006% | −0.517 | no | 5 | −5.949 |
| CCTATA | 0.010% | −0.599 | no | 5 | −2.207 |
| CCTATC | 0.007% | −0.285 | no | 5 | −6.011 |
| CCTATG | 0.008% | −0.201 | no | 5 | −1.218 |
| CCTCAT | 0.009% | NA | no | 5 | −0.277 |
| CCTCCT | 0.006% | NA | no | 5 | −1.459 |
| CCTCGT | 0.009% | 0.284 | no | 5 | −0.414 |
| CCTCTA | 0.011% | −0.215 | no | 5 | −2.105 |
| CCTCTC | 0.005% | NA | no | 5 | 1.159 |
| CCTCTG | 0.008% | 0.206 | no | 5 | 0.809 |
| CCTGAA | 0.021% | NA | no | 5 | −0.274 |
| CCTGAC | 0.010% | NA | no | 5 | −0.072 |
| CCTGAG | 0.011% | NA | no | 5 | 0.683 |
| CCTGCA | 0.013% | 0.332 | no | 5 | −1.474 |
| CCTGCC | 0.007% | 0.151 | no | 5 | −0.210 |
| CCTGCG | 0.015% | 0.584 | no | 5 | 1.012 |
| CCTGGA | 0.011% | 0.407 | no | 5 | 0.176 |
| CCTGGC | 0.012% | NA | no | 5 | −0.365 |
| CCTGGG | 0.013% | 0.277 | no | 5 | 0.803 |
| CCTTAT | 0.007% | −0.537 | no | 5 | −1.326 |
| CCTTCT | 0.005% | NA | no | 5 | −2.992 |
| CCTTGT | 0.009% | −0.207 | no | 5 | −1.603 |
| CCTTTA | 0.005% | −0.881 | no | 5 | −4.138 |
| CCTTTG | 0.006% | −0.241 | no | 5 | −1.149 |
| CGCAAA | 0.026% | NA | no | 5 | 0.561 |
| CGCAAC | 0.021% | 0.386 | no | 5 | 0.278 |
| CGCAAG | 0.020% | NA | no | 5 | 0.669 |
| CGCACA | 0.020% | NA | no | 5 | −2.160 |
| CGCACC | 0.009% | 0.280 | no | 5 | 1.388 |
| CGCACG | 0.013% | 0.604 | no | 5 | 0.400 |
| CGCAGA | 0.023% | NA | no | 5 | −0.597 |
| CGCAGC | 0.012% | −0.402 | no | 5 | −0.575 |
| CGCAGG | 0.022% | NA | no | 5 | 0.050 |
| CGCCAA | 0.014% | NA | no | 5 | 0.443 |
| CGCCAC | 0.011% | NA | no | 5 | 0.179 |
| CGCCAG | 0.010% | 0.298 | no | 5 | −1.002 |
| CGCCCA | 0.007% | 0.225 | no | 5 | −0.574 |
| CGCCCC | 0.008% | NA | no | 5 | −1.953 |
| CGCCCG | 0.005% | 0.636 | no | 5 | 0.220 |
| CGCCGA | 0.009% | 0.450 | no | 5 | −1.108 |
| CGCCGC | 0.006% | 0.353 | no | 5 | 2.704 |
| CGCCGG | 0.010% | 0.746 | no | 5 | 0.998 |
| CGCTAA | 0.014% | NA | no | 5 | 0.149 |
| CGCTAC | 0.008% | 0.302 | no | 5 | 0.629 |
| CGCTAG | 0.012% | −0.399 | no | 5 | −0.124 |
| CGCTCA | 0.009% | NA | no | 5 | 0.549 |
| CGCTCC | 0.008% | 0.260 | no | 5 | 0.173 |
| CGCTCG | 0.009% | 0.487 | no | 5 | −0.835 |
| CGCTGA | 0.013% | 0.545 | no | 5 | −1.332 |
| CGCTGC | 0.009% | 0.418 | no | 5 | −1.093 |
| CGCTGG | 0.011% | NA | no | 5 | −0.925 |
| CGGAAA | 0.035% | NA | no | 5 | 0.338 |
| CGGAAC | 0.023% | 0.618 | no | 5 | 0.258 |
| CGGAAG | 0.026% | 0.528 | no | 5 | 0.224 |
| CGGACA | 0.026% | NA | no | 5 | −0.284 |
| CGGACC | 0.016% | 0.447 | no | 5 | 0.568 |
| CGGACG | 0.026% | 0.841 | no | 5 | 0.639 |
| CGGAGA | 0.026% | 0.599 | no | 5 | −1.101 |
| CGGAGC | 0.017% | 0.618 | no | 5 | 0.538 |
| CGGAGG | 0.029% | 0.385 | no | 5 | −0.032 |
| CGGCAA | 0.025% | NA | no | 5 | −0.210 |
| CGGCAC | 0.015% | NA | no | 5 | 0.170 |
| CGGCAG | 0.021% | NA | no | 5 | 0.374 |
| CGGCCA | 0.012% | 0.142 | no | 5 | −0.568 |
| CGGCCC | 0.011% | NA | no | 5 | 0.288 |
| CGGCCG | 0.009% | 0.460 | no | 5 | 2.910 |
| CGGCGA | 0.018% | 0.686 | no | 5 | 1.084 |
| CGGCGC | 0.009% | 0.595 | no | 5 | −0.606 |
| CGGCGG | 0.018% | 0.721 | no | 5 | −0.511 |
| CGGTAA | 0.024% | NA | no | 5 | −0.222 |
| CGGTAC | 0.015% | 0.364 | no | 5 | 0.310 |
| CGGTAG | 0.015% | −0.530 | no | 5 | −0.516 |
| CGGTCA | 0.014% | NA | no | 5 | −0.746 |
| CGGTCC | 0.007% | 0.431 | no | 5 | −0.815 |
| CGGTCG | 0.013% | 0.483 | no | 5 | 0.514 |
| CGGTGA | 0.018% | NA | no | 5 | 0.579 |
| CGGTGC | 0.011% | NA | no | 5 | −0.800 |
| CGGTGG | 0.015% | NA | no | 5 | 1.378 |
| CGTAAA | 0.026% | NA | no | 5 | 0.878 |
| CGTAAC | 0.016% | NA | no | 5 | 0.417 |
| CGTAAG | 0.018% | NA | no | 5 | 0.358 |
| CGTACA | 0.016% | NA | no | 5 | 0.019 |
| CGTACC | 0.010% | 0.277 | no | 5 | 0.658 |
| CGTACG | 0.016% | 0.408 | no | 5 | 1.020 |
| CGTAGA | 0.020% | NA | no | 5 | −0.415 |
| CGTAGC | 0.011% | −0.294 | no | 5 | 0.979 |
| CGTAGG | 0.015% | −0.484 | no | 5 | −0.882 |
| CGTCAA | 0.019% | 0.515 | no | 5 | 0.678 |
| CGTCAC | 0.011% | 0.401 | no | 5 | 0.156 |
| CGTCAG | 0.014% | NA | no | 5 | 0.073 |
| CGTCCA | 0.008% | 0.640 | no | 5 | 0.170 |
| CGTCCC | 0.007% | 0.218 | no | 5 | 0.041 |
| CGTCCG | 0.010% | 0.753 | no | 5 | −0.220 |
| CGTCGA | 0.015% | 0.885 | no | 5 | 0.287 |
| CGTCGC | 0.010% | 0.885 | no | 5 | −0.034 |
| CGTCGG | 0.006% | 0.844 | no | 5 | −0.670 |
| CGTTAA | 0.021% | NA | no | 5 | 0.155 |
| CGTTAC | 0.009% | NA | no | 5 | −1.673 |
| CGTTAG | 0.012% | −0.626 | no | 5 | −2.079 |
| CGTTCA | 0.012% | NA | no | 5 | 0.168 |
| CGTTCC | 0.006% | 0.395 | no | 5 | 1.633 |
| CGTTCG | 0.010% | 0.601 | no | 5 | 0.885 |
| CGTTGA | 0.013% | NA | no | 5 | −1.480 |
| CGTTGC | 0.010% | 0.191 | no | 5 | −0.171 |
| CGTTGG | 0.012% | 0.290 | no | 5 | −0.722 |
| CTAAAA | 0.020% | −0.424 | yes | 5 | −5.083 |
| CTAAAC | 0.012% | −0.456 | yes | 5 | −3.039 |
| CTAAAG | 0.020% | −0.364 | yes | 5 | −3.512 |
| CTAACA | 0.013% | NA | yes | 5 | −4.892 |
| CTAACC | 0.007% | −0.217 | yes | 5 | −2.610 |
| CTAACG | 0.008% | NA | yes | 5 | −2.195 |
| CTAAGA | 0.019% | NA | yes | 5 | −4.433 |
| CTAAGC | 0.012% | −0.283 | yes | 5 | −4.345 |
| CTAAGG | 0.018% | −0.377 | yes | 5 | −4.084 |
| CTACAA | 0.013% | NA | no | 5 | 0.791 |
| CTACAC | 0.007% | NA | no | 5 | −0.163 |
| CTACAG | 0.012% | NA | no | 5 | −0.758 |
| CTACCA | 0.006% | −0.188 | no | 5 | 0.181 |
| CTACCC | 0.004% | −0.211 | no | 5 | −0.343 |
| CTACCG | 0.010% | 0.356 | no | 5 | −0.436 |
| CTACGA | 0.012% | 0.366 | no | 5 | −0.213 |
| CTACGC | 0.006% | 0.446 | no | 5 | 0.518 |
| CTACGG | 0.010% | 0.503 | no | 5 | 0.285 |
| CTATAA | 0.019% | −0.566 | no | 5 | −3.209 |
| CTATAC | 0.006% | NA | no | 5 | −2.641 |
| CTATAG | 0.015% | −0.604 | no | 5 | −0.860 |
| CTATCA | 0.009% | −0.456 | no | 5 | −4.031 |
| CTATCC | 0.005% | NA | no | 5 | −5.014 |
| CTATCG | 0.010% | NA | no | 5 | 0.587 |
| CTATGA | 0.011% | NA | no | 5 | −2.579 |
| CTATGC | 0.006% | −0.228 | no | 5 | −1.462 |
| CTATGG | 0.011% | NA | no | 5 | −1.357 |
| CTCAAT | 0.016% | NA | no | 5 | −2.638 |
| CTCACT | 0.007% | NA | no | 5 | −3.336 |
| CTCAGT | 0.014% | NA | no | 5 | −2.816 |
| CTCATA | 0.011% | −0.431 | no | 5 | −1.054 |
| CTCATC | 0.011% | NA | no | 5 | −0.961 |
| CTCATG | 0.010% | NA | no | 5 | 0.531 |
| CTCCAT | 0.009% | NA | no | 5 | 0.155 |
| CTCCGT | 0.007% | 0.481 | no | 5 | −1.212 |
| CTCCTA | 0.005% | NA | no | 5 | −3.446 |
| CTCCTC | 0.005% | NA | no | 5 | −1.099 |
| CTCCTG | 0.007% | 0.444 | no | 5 | 0.418 |
| CTCGAA | 0.015% | NA | no | 5 | −0.108 |
| CTCGAC | 0.010% | 0.491 | no | 5 | −1.782 |
| CTCGAG | 0.012% | 0.308 | no | 5 | 1.826 |
| CTCGCA | 0.008% | NA | no | 5 | −1.403 |
| CTCGCG | 0.015% | 0.536 | no | 5 | −0.761 |
| CTCGGA | 0.013% | 0.520 | no | 5 | 0.278 |
| CTCGGC | 0.009% | 0.432 | no | 5 | 1.334 |
| CTCGGG | 0.012% | 0.323 | no | 5 | 1.920 |
| CTCTAT | 0.009% | −0.374 | no | 5 | −4.034 |
| CTCTCT | 0.007% | NA | no | 5 | −0.997 |
| CTCTGT | 0.006% | NA | no | 5 | −3.627 |
| CTCTTA | 0.012% | −0.642 | no | 5 | −2.883 |
| CTCTTC | 0.004% | NA | no | 5 | 0.471 |
| CTCTTG | 0.008% | −0.257 | no | 5 | −0.439 |
| CTGAAT | 0.023% | NA | yes | 5 | −1.424 |
| CTGACT | 0.012% | NA | yes | 5 | −2.077 |
| CTGAGT | 0.015% | NA | yes | 5 | −3.901 |
| CTGATA | 0.014% | NA | yes | 5 | −5.101 |
| CTGATC | 0.012% | NA | yes | 5 | −2.539 |
| CTGATG | 0.016% | 0.235 | yes | 5 | −2.001 |
| CTGCAT | 0.019% | NA | no | 5 | −1.930 |
| CTGCCT | 0.008% | 0.310 | no | 5 | −1.990 |
| CTGCGT | 0.011% | 0.423 | no | 5 | −0.420 |
| CTGCTA | 0.010% | NA | no | 5 | −1.068 |
| CTGCTC | 0.008% | 0.334 | no | 5 | −0.467 |
| CTGCTG | 0.013% | 0.333 | no | 5 | −1.002 |
| CTGGAA | 0.016% | NA | no | 5 | 1.036 |
| CTGGAC | 0.009% | NA | no | 5 | −0.724 |
| CTGGAG | 0.017% | NA | no | 5 | −0.675 |
| CTGGCA | 0.016% | NA | no | 5 | 0.172 |
| CTGGCC | 0.007% | NA | no | 5 | 0.854 |
| CTGGCG | 0.013% | 0.317 | no | 5 | −0.298 |
| CTGGGA | 0.015% | 0.115 | no | 5 | −0.526 |
| CTGGGC | 0.010% | NA | no | 5 | −0.027 |
| CTGGGG | 0.015% | NA | no | 5 | 0.165 |
| CTGTAT | 0.012% | NA | no | 5 | −0.148 |
| CTGTCT | 0.008% | NA | no | 5 | 0.684 |
| CTGTGT | 0.013% | −0.177 | no | 5 | 2.444 |
| CTGTTA | 0.011% | NA | no | 5 | 1.526 |
| CTGTTC | 0.008% | NA | no | 5 | −0.152 |
| CTGTTG | 0.013% | NA | no | 5 | 0.424 |
| CTTAAT | 0.011% | −0.598 | no | 5 | −1.921 |
| CTTACT | 0.010% | NA | no | 5 | −3.914 |
| CTTAGT | 0.010% | −0.774 | no | 5 | −7.156 |
| CTTATA | 0.011% | −0.791 | no | 5 | −1.113 |
| CTTATC | 0.009% | NA | no | 5 | −3.039 |
| CTTATG | 0.012% | −0.307 | no | 5 | −1.075 |
| CTTCAT | 0.008% | NA | no | 5 | 0.389 |
| CTTCCT | 0.005% | 0.298 | no | 5 | 1.368 |
| CTTCGT | 0.007% | 0.413 | no | 5 | 1.632 |
| CTTCTA | 0.008% | −0.363 | no | 5 | −0.565 |
| CTTCTC | 0.006% | NA | no | 5 | −0.968 |
| CTTCTG | 0.006% | 0.309 | no | 5 | −0.153 |
| CTTGAA | 0.013% | NA | no | 5 | 0.006 |
| CTTGAC | 0.008% | NA | no | 5 | 2.668 |
| CTTGAG | 0.012% | NA | no | 5 | −0.635 |
| CTTGCA | 0.011% | −0.191 | no | 5 | −0.758 |
| CTTGCG | 0.013% | NA | no | 5 | −1.131 |
| CTTGGA | 0.019% | NA | no | 5 | −0.476 |
| CTTGGC | 0.011% | NA | no | 5 | −0.152 |
| CTTGGG | 0.013% | NA | no | 5 | −0.037 |
| CTTTAT | 0.008% | −0.753 | no | 5 | −1.800 |
| CTTTCT | 0.004% | NA | no | 5 | −3.554 |
| CTTTGT | 0.010% | −0.303 | no | 5 | −0.930 |
| CTTTTA | 0.011% | −1.061 | no | 5 | −2.689 |
| CTTTTG | 0.007% | −0.322 | no | 5 | 1.020 |
| GAAAAA | 0.073% | NA | no | 5 | −1.912 |
| GAAAAC | 0.063% | 0.303 | no | 5 | −0.762 |
| GAAAAG | 0.070% | NA | no | 5 | −0.456 |
| GAAACA | 0.067% | NA | no | 5 | −1.225 |
| GAAACC | 0.039% | 0.340 | no | 5 | −0.658 |
| GAAACG | 0.049% | 0.468 | no | 5 | −0.535 |
| GAAAGA | 0.067% | 0.346 | no | 5 | −1.562 |
| GAAAGC | 0.048% | 0.298 | no | 5 | −0.957 |
| GAAAGG | 0.074% | −0.141 | no | 5 | −0.732 |
| GAACAA | 0.071% | 0.467 | no | 5 | −1.161 |
| GAACAC | 0.043% | 0.417 | no | 5 | −0.564 |
| GAACAG | 0.054% | NA | no | 5 | −1.772 |
| GAACCA | 0.036% | 0.363 | no | 5 | −0.524 |
| GAACCC | 0.022% | NA | no | 5 | −0.257 |
| GAACCG | 0.032% | 0.585 | no | 5 | 0.157 |
| GAACGA | 0.046% | 0.642 | no | 5 | −0.098 |
| GAACGC | 0.032% | 0.703 | no | 5 | −0.930 |
| GAACGG | 0.056% | 0.441 | no | 5 | −0.213 |
| GAATAA | 0.067% | NA | no | 5 | −2.770 |
| GAATAC | 0.037% | 0.406 | no | 5 | −1.138 |
| GAATAG | 0.066% | −0.335 | no | 5 | −1.653 |
| GAATCA | 0.043% | NA | no | 5 | −0.566 |
| GAATCC | 0.023% | 0.470 | no | 5 | −0.292 |
| GAATCG | 0.041% | 0.586 | no | 5 | 0.067 |
| GAATGA | 0.061% | NA | no | 5 | −2.111 |
| GAATGC | 0.039% | 0.197 | no | 5 | −0.256 |
| GAATGG | 0.060% | NA | no | 5 | −0.599 |
| GACAAT | 0.030% | NA | no | 5 | 0.539 |
| GACACT | 0.021% | 0.281 | no | 5 | −0.605 |
| GACAGT | 0.040% | NA | no | 5 | −1.244 |
| GACATA | 0.032% | NA | no | 5 | −0.663 |
| GACATC | 0.023% | 0.682 | no | 5 | 0.415 |
| GACATG | 0.029% | 0.262 | no | 5 | 0.258 |
| GACCAT | 0.024% | 0.380 | no | 5 | −0.090 |
| GACCCT | 0.018% | 0.545 | no | 5 | −0.032 |
| GACCGT | 0.017% | 0.536 | no | 5 | 0.344 |
| GACCTA | 0.020% | NA | no | 5 | 1.043 |
| GACCTC | 0.014% | 0.384 | no | 5 | −0.229 |
| GACCTG | 0.016% | 0.664 | no | 5 | 0.902 |
| GACGAA | 0.034% | 0.718 | no | 5 | 0.028 |
| GACGAC | 0.027% | 0.846 | no | 5 | −0.223 |
| GACGAG | 0.038% | 0.529 | no | 5 | −0.271 |
| GACGCA | 0.032% | 0.514 | no | 5 | −1.035 |
| GACGCC | 0.011% | 0.663 | no | 5 | −0.620 |
| GACGCG | 0.024% | 0.821 | no | 5 | 0.142 |
| GACGGA | 0.041% | 0.873 | no | 5 | −0.316 |
| GACGGC | 0.021% | 0.724 | no | 5 | 0.421 |
| GACGGG | 0.032% | 0.470 | no | 5 | 0.153 |
| GACTAT | 0.022% | NA | no | 5 | −0.196 |
| GACTCT | 0.014% | 0.397 | no | 5 | −1.331 |
| GACTGT | 0.021% | 0.323 | no | 5 | 0.052 |
| GACTTA | 0.027% | NA | no | 5 | −0.558 |
| GACTTC | 0.016% | 0.445 | no | 5 | −0.267 |
| GACTTG | 0.021% | 0.168 | no | 5 | 0.013 |
| GAGAAT | 0.062% | 0.309 | no | 5 | −1.153 |
| GAGACT | 0.033% | 0.490 | no | 5 | −0.030 |
| GAGAGT | 0.050% | 0.247 | no | 5 | −1.343 |
| GAGATA | 0.052% | NA | no | 5 | −0.848 |
| GAGATC | 0.023% | 0.477 | no | 5 | −1.372 |
| GAGATG | 0.050% | 0.327 | no | 5 | −0.289 |
| GAGCAT | 0.034% | NA | no | 5 | −0.396 |
| GAGCCT | 0.014% | NA | no | 5 | −0.557 |
| GAGCGT | 0.028% | 0.305 | no | 5 | −0.085 |
| GAGCTA | 0.031% | −0.294 | no | 5 | −0.698 |
| GAGCTC | 0.016% | NA | no | 5 | −0.583 |
| GAGCTG | 0.024% | 0.270 | no | 5 | 0.367 |
| GAGGAA | 0.070% | 0.410 | no | 5 | −0.457 |
| GAGGAC | 0.032% | 0.626 | no | 5 | −0.912 |
| GAGGAG | 0.056% | 0.247 | no | 5 | −0.609 |
| GAGGCA | 0.043% | NA | no | 5 | −1.224 |
| GAGGCC | 0.025% | 0.380 | no | 5 | −0.061 |
| GAGGCG | 0.031% | 0.332 | no | 5 | −0.424 |
| GAGGGA | 0.058% | NA | no | 5 | −4.266 |
| GAGGGC | 0.029% | −0.084 | no | 5 | −2.555 |
| GAGGGG | 0.053% | −0.144 | no | 5 | −3.081 |
| GAGTAT | 0.039% | NA | no | 5 | −1.700 |
| GAGTCT | 0.019% | 0.271 | no | 5 | −0.853 |
| GAGTGT | 0.030% | NA | no | 5 | −0.801 |
| GAGTTA | 0.033% | −0.179 | no | 5 | −2.132 |
| GAGTTC | 0.023% | 0.210 | no | 5 | −0.912 |
| GAGTTG | 0.028% | 0.132 | no | 5 | 0.633 |
| GATAAT | 0.036% | NA | no | 5 | −2.443 |
| GATACT | 0.018% | 0.225 | no | 5 | −1.391 |
| GATAGT | 0.026% | −0.235 | no | 5 | −6.217 |
| GATATA | 0.037% | NA | no | 5 | −2.538 |
| GATATC | 0.030% | NA | no | 5 | −2.461 |
| GATATG | 0.035% | NA | no | 5 | −0.826 |
| GATCAT | 0.020% | 0.298 | no | 5 | −1.305 |
| GATCCT | 0.013% | 0.275 | no | 5 | −0.810 |
| GATCGT | 0.019% | 0.458 | no | 5 | −0.584 |
| GATCTA | 0.021% | NA | no | 5 | −1.813 |
| GATCTC | 0.012% | NA | no | 5 | −1.894 |
| GATCTG | 0.025% | 0.385 | no | 5 | 0.068 |
| GATGAA | 0.052% | 0.510 | no | 5 | −0.891 |
| GATGAC | 0.018% | 0.643 | no | 5 | 0.550 |
| GATGAG | 0.024% | NA | no | 5 | −0.869 |
| GATGCA | 0.036% | 0.205 | no | 5 | −1.369 |
| GATGCC | 0.013% | 0.546 | no | 5 | 0.690 |
| GATGCG | 0.021% | 0.540 | no | 5 | −0.126 |
| GATGGA | 0.033% | 0.664 | no | 5 | 0.483 |
| GATGGC | 0.018% | 0.576 | no | 5 | 0.448 |
| GATGGG | 0.034% | 0.131 | no | 5 | −0.136 |
| GATTAT | 0.020% | NA | no | 5 | −3.377 |
| GATTCT | 0.013% | 0.273 | no | 5 | −4.732 |
| GATTGT | 0.022% | NA | no | 5 | −2.355 |
| GATTTA | 0.033% | NA | no | 5 | −3.989 |
| GATTTC | 0.021% | NA | no | 5 | −3.935 |
| GATTTG | 0.023% | NA | no | 5 | −0.041 |
| GCAAAA | 0.051% | NA | no | 5 | −1.794 |
| GCAAAC | 0.033% | 0.251 | no | 5 | −1.201 |
| GCAAAG | 0.054% | NA | no | 5 | −0.704 |
| GCAACA | 0.042% | 0.306 | no | 5 | −0.302 |
| GCAACC | 0.019% | 0.106 | no | 5 | −0.899 |
| GCAACG | 0.035% | 0.392 | no | 5 | 0.324 |
| GCAAGA | 0.047% | 0.891 | no | 5 | −0.150 |
| GCAAGC | 0.038% | 0.195 | no | 5 | −0.964 |
| GCAAGG | 0.039% | NA | no | 5 | −1.281 |
| GCACAA | 0.033% | NA | no | 5 | −1.175 |
| GCACAC | 0.027% | NA | no | 5 | −0.308 |
| GCACAG | 0.028% | NA | no | 5 | −0.687 |
| GCACCA | 0.029% | −0.116 | no | 5 | −0.412 |
| GCACCC | 0.012% | −0.117 | no | 5 | −1.725 |
| GCACCG | 0.020% | NA | no | 5 | 0.718 |
| GCACGA | 0.033% | NA | no | 5 | −0.733 |
| GCACGC | 0.022% | NA | no | 5 | −0.224 |
| GCACGG | 0.028% | 0.164 | no | 5 | −0.404 |
| GCATAA | 0.037% | −0.153 | no | 5 | −1.497 |
| GCATAC | 0.022% | NA | no | 5 | −1.032 |
| GCATAG | 0.036% | −0.161 | no | 5 | −0.258 |
| GCATCA | 0.021% | NA | no | 5 | 0.441 |
| GCATCC | 0.012% | NA | no | 5 | 0.670 |
| GCATCG | 0.022% | NA | no | 5 | −0.098 |
| GCATGA | 0.035% | 0.253 | no | 5 | −0.535 |
| GCATGC | 0.018% | NA | no | 5 | −0.325 |
| GCATGG | 0.035% | 0.097 | no | 5 | −0.014 |
| GCCAAT | 0.023% | NA | no | 5 | −0.619 |
| GCCACT | 0.017% | NA | no | 5 | −0.735 |
| GCCAGT | 0.022% | −0.227 | no | 5 | 1.500 |
| GCCATA | 0.019% | −0.395 | no | 5 | −1.324 |
| GCCATC | 0.015% | NA | no | 5 | −0.140 |
| GCCATG | 0.021% | NA | no | 5 | 0.377 |
| GCCCAT | 0.017% | NA | no | 5 | −0.072 |
| GCCCGT | 0.012% | NA | no | 5 | −0.015 |
| GCCCTA | 0.009% | −0.425 | no | 5 | −0.434 |
| GCCCTC | 0.009% | NA | no | 5 | −0.362 |
| GCCCTG | 0.011% | NA | no | 5 | 0.271 |
| GCCGAA | 0.027% | NA | no | 5 | −0.995 |
| GCCGAC | 0.014% | 0.435 | no | 5 | −0.378 |
| GCCGAG | 0.022% | NA | no | 5 | 0.343 |
| GCCGCA | 0.018% | NA | no | 5 | −0.175 |
| GCCGCC | 0.016% | NA | no | 5 | −1.112 |
| GCCGCG | 0.017% | 0.487 | no | 5 | 1.475 |
| GCCGGA | 0.023% | 0.428 | no | 5 | 0.006 |
| GCCGGC | 0.013% | NA | no | 5 | −0.237 |
| GCCGGG | 0.022% | 0.409 | no | 5 | 0.540 |
| GCCTAT | 0.016% | −0.310 | no | 5 | −0.958 |
| GCCTCT | 0.009% | NA | no | 5 | −0.415 |
| GCCTGT | 0.014% | NA | no | 5 | 0.090 |
| GCCTTA | 0.016% | −0.481 | no | 5 | −1.227 |
| GCCTTC | 0.010% | NA | no | 5 | −0.560 |
| GCCTTG | 0.015% | NA | no | 5 | 0.114 |
| GCGAAT | 0.037% | NA | no | 5 | 0.140 |
| GCGACT | 0.023% | 0.434 | no | 5 | 0.664 |
| GCGAGT | 0.026% | NA | no | 5 | 0.903 |
| GCGATA | 0.032% | NA | no | 5 | −0.079 |
| GCGATC | 0.015% | 0.474 | no | 5 | −0.188 |
| GCGATG | 0.023% | 0.320 | no | 5 | 0.666 |
| GCGCAT | 0.020% | NA | no | 5 | 0.153 |
| GCGCCT | 0.011% | 0.298 | no | 5 | −0.807 |
| GCGCGT | 0.016% | 0.285 | no | 5 | −1.638 |
| GCGCTA | 0.019% | NA | no | 5 | −0.341 |
| GCGCTC | 0.014% | NA | no | 5 | 0.385 |
| GCGCTG | 0.020% | 0.251 | no | 5 | −0.476 |
| GCGGAA | 0.040% | 0.461 | no | 5 | −0.051 |
| GCGGAC | 0.021% | 0.725 | no | 5 | −0.390 |
| GCGGAG | 0.036% | 0.346 | no | 5 | −0.355 |
| GCGGCA | 0.025% | NA | no | 5 | 0.120 |
| GCGGCC | 0.015% | NA | no | 5 | 0.790 |
| GCGGCG | 0.027% | 0.555 | no | 5 | 1.289 |
| GCGGGA | 0.042% | 0.461 | no | 5 | −0.986 |
| GCGGGC | 0.020% | 0.289 | no | 5 | −1.162 |
| GCGGGG | 0.030% | 0.178 | no | 5 | −0.662 |
| GCGTAT | 0.019% | NA | no | 5 | −1.712 |
| GCGTCT | 0.011% | 0.302 | no | 5 | −1.378 |
| GCGTGT | 0.017% | NA | no | 5 | −1.271 |
| GCGTTA | 0.016% | NA | no | 5 | −0.524 |
| GCGTTC | 0.012% | 0.447 | no | 5 | −0.686 |
| GCGTTG | 0.014% | 0.149 | no | 5 | −0.045 |
| GCTAAT | 0.019% | −0.321 | no | 5 | −4.088 |
| GCTACT | 0.013% | NA | no | 5 | −1.692 |
| GCTAGT | 0.017% | −0.474 | no | 5 | −7.760 |
| GCTATA | 0.024% | −0.534 | no | 5 | −1.898 |
| GCTATC | 0.011% | NA | no | 5 | −2.675 |
| GCTATG | 0.015% | −0.251 | no | 5 | 1.611 |
| GCTCAT | 0.017% | NA | no | 5 | −1.340 |
| GCTCCT | 0.011% | NA | no | 5 | −0.085 |
| GCTCGT | 0.014% | NA | no | 5 | 0.090 |
| GCTCTA | 0.020% | −0.349 | no | 5 | −3.023 |
| GCTCTC | 0.013% | NA | no | 5 | −0.873 |
| GCTCTG | 0.016% | NA | no | 5 | 0.288 |
| GCTGAA | 0.034% | NA | no | 5 | 0.186 |
| GCTGAC | 0.017% | 0.488 | no | 5 | 0.663 |
| GCTGAG | 0.024% | NA | no | 5 | −0.329 |
| GCTGCA | 0.021% | 0.115 | no | 5 | −0.911 |
| GCTGCC | 0.008% | 0.179 | no | 5 | 2.076 |
| GCTGCG | 0.021% | 0.446 | no | 5 | −0.124 |
| GCTGGA | 0.028% | NA | no | 5 | −0.633 |
| GCTGGC | 0.013% | NA | no | 5 | 0.510 |
| GCTGGG | 0.025% | −0.216 | no | 5 | 0.086 |
| GCTTAT | 0.018% | −0.369 | no | 5 | −2.526 |
| GCTTCT | 0.008% | NA | no | 5 | −1.708 |
| GCTTGT | 0.022% | NA | no | 5 | 0.587 |
| GCTTTA | 0.017% | −0.795 | no | 5 | −2.826 |
| GCTTTC | 0.011% | NA | no | 5 | −1.505 |
| GCTTTG | 0.016% | −0.241 | no | 5 | −0.412 |
| GGCAAA | 0.047% | 0.128 | no | 5 | −0.459 |
| GGCAAC | 0.026% | 0.283 | no | 5 | −0.188 |
| GGCAAG | 0.036% | 0.398 | no | 5 | 0.412 |
| GGCACA | 0.030% | −0.086 | no | 5 | −0.271 |
| GGCACC | 0.015% | −0.171 | no | 5 | 0.260 |
| GGCACG | 0.024% | NA | no | 5 | −0.495 |
| GGCAGA | 0.041% | 0.210 | no | 5 | 0.887 |
| GGCAGC | 0.029% | NA | no | 5 | −0.243 |
| GGCAGG | 0.043% | −0.229 | no | 5 | 0.010 |
| GGCCAA | 0.028% | NA | no | 5 | 0.253 |
| GGCCAC | 0.019% | NA | no | 5 | 0.005 |
| GGCCAG | 0.025% | NA | no | 5 | −0.011 |
| GGCCCA | 0.015% | NA | no | 5 | −0.133 |
| GGCCCC | 0.012% | NA | no | 5 | −0.295 |
| GGCCCG | 0.014% | NA | no | 5 | 0.293 |
| GGCCGA | 0.026% | 0.204 | no | 5 | 0.231 |
| GGCCGC | 0.009% | 0.202 | no | 5 | 2.060 |
| GGCCGG | 0.019% | 0.129 | no | 5 | −0.641 |
| GGCTAA | 0.027% | −0.257 | no | 5 | 0.240 |
| GGCTAC | 0.019% | NA | no | 5 | −0.109 |
| GGCTAG | 0.022% | −0.519 | no | 5 | 0.275 |
| GGCTCA | 0.016% | −0.282 | no | 5 | 0.232 |
| GGCTCC | 0.009% | NA | no | 5 | 3.465 |
| GGCTCG | 0.019% | NA | no | 5 | 1.424 |
| GGCTGA | 0.022% | 0.192 | no | 5 | 0.203 |
| GGCTGC | 0.015% | 0.151 | no | 5 | 0.186 |
| GGCTGG | 0.028% | −0.221 | no | 5 | 0.177 |
| GGGAAA | 0.073% | −0.218 | no | 5 | −0.163 |
| GGGAAC | 0.046% | 0.211 | no | 5 | 0.186 |
| GGGAAG | 0.065% | NA | no | 5 | −0.359 |
| GGGACA | 0.050% | NA | no | 5 | 0.256 |
| GGGACC | 0.027% | 0.292 | no | 5 | 0.047 |
| GGGACG | 0.039% | 0.266 | no | 5 | −0.327 |
| GGGAGA | 0.053% | NA | no | 5 | 0.171 |
| GGGAGC | 0.030% | 0.194 | no | 5 | 0.400 |
| GGGAGG | 0.045% | −0.461 | no | 5 | −0.705 |
| GGGCAA | 0.044% | −0.271 | no | 5 | −0.283 |
| GGGCAC | 0.031% | NA | no | 5 | 0.330 |
| GGGCAG | 0.037% | −0.367 | no | 5 | −0.572 |
| GGGCCA | 0.028% | NA | no | 5 | 0.059 |
| GGGCCC | 0.016% | NA | no | 5 | 0.440 |
| GGGCCG | 0.023% | NA | no | 5 | −0.113 |
| GGGCGA | 0.036% | NA | no | 5 | −0.193 |
| GGGCGC | 0.023% | 0.189 | no | 5 | −0.169 |
| GGGCGG | 0.028% | −0.213 | no | 5 | 0.023 |
| GGGTAA | 0.063% | −0.710 | no | 5 | −1.042 |
| GGGTAC | 0.031% | −0.297 | no | 5 | −0.338 |
| GGGTAG | 0.042% | −0.786 | no | 5 | 0.223 |
| GGGTCA | 0.031% | −0.432 | no | 5 | 0.385 |
| GGGTCC | 0.019% | NA | no | 5 | 0.232 |
| GGGTCG | 0.024% | NA | no | 5 | −0.370 |
| GGGTGA | 0.031% | −0.361 | no | 5 | 0.084 |
| GGGTGC | 0.027% | −0.248 | no | 5 | −0.622 |
| GGGTGG | 0.039% | −0.643 | no | 5 | −0.238 |
| GGTAAA | 0.067% | −0.588 | no | 5 | 0.380 |
| GGTAAC | 0.035% | −0.330 | no | 5 | 0.469 |
| GGTAAG | 0.047% | −0.770 | no | 5 | −1.643 |
| GGTACA | 0.041% | −0.359 | no | 5 | 0.125 |
| GGTACG | 0.029% | NA | no | 5 | −0.411 |
| GGTAGA | 0.040% | −0.530 | no | 5 | −0.432 |
| GGTAGC | 0.028% | −0.532 | no | 5 | −0.268 |
| GGTAGG | 0.039% | −0.818 | no | 5 | −3.067 |
| GGTCAA | 0.038% | NA | no | 5 | 0.106 |
| GGTCAC | 0.026% | NA | no | 5 | 0.375 |
| GGTCAG | 0.032% | −0.351 | no | 5 | −0.019 |
| GGTCCA | 0.017% | NA | no | 5 | −0.107 |
| GGTCCC | 0.009% | NA | no | 5 | 0.610 |
| GGTCCG | 0.010% | NA | no | 5 | −0.116 |
| GGTCGA | 0.031% | NA | no | 5 | 0.781 |
| GGTCGC | 0.017% | 0.211 | no | 5 | −0.479 |
| GGTCGG | 0.021% | NA | no | 5 | −0.400 |
| GGTTAA | 0.042% | −0.569 | no | 5 | 0.857 |
| GGTTAC | 0.021% | −0.225 | no | 5 | 0.462 |
| GGTTAG | 0.038% | −0.781 | no | 5 | −0.483 |
| GGTTCA | 0.025% | −0.261 | no | 5 | 0.137 |
| GGTTCC | 0.006% | NA | no | 5 | −1.212 |
| GGTTCG | 0.019% | NA | no | 5 | −1.113 |
| GGTTGA | 0.031% | −0.204 | no | 5 | 0.084 |
| GGTTGC | 0.016% | −0.236 | no | 5 | 0.246 |
| GGTTGG | 0.027% | −0.324 | no | 5 | −0.233 |
| GTAAAA | 0.060% | −0.504 | yes | 5 | −5.354 |
| GTAAAC | 0.044% | −0.319 | yes | 5 | −4.406 |
| GTAAAG | 0.047% | −0.374 | yes | 5 | −3.346 |
| GTAACA | 0.040% | −0.466 | yes | 5 | −4.312 |
| GTAACC | 0.027% | NA | yes | 5 | −3.350 |
| GTAACG | 0.040% | NA | yes | 5 | −2.749 |
| GTAAGA | 0.047% | −0.374 | yes | 5 | −4.868 |
| GTAAGC | 0.033% | −0.425 | yes | 5 | −5.240 |
| GTAAGG | 0.046% | −0.549 | yes | 5 | −5.149 |
| GTACAA | 0.033% | −0.338 | no | 5 | −2.376 |
| GTACAC | 0.027% | NA | no | 5 | −1.926 |
| GTACAG | 0.028% | −0.323 | no | 5 | −0.605 |
| GTACCA | 0.022% | NA | no | 5 | −0.815 |
| GTACCC | 0.011% | NA | no | 5 | −0.631 |
| GTACCG | 0.018% | 0.241 | no | 5 | −0.081 |
| GTACGA | 0.023% | NA | no | 5 | −0.940 |
| GTACGC | 0.017% | NA | no | 5 | 0.275 |
| GTACGG | 0.026% | 0.162 | no | 5 | −0.333 |
| GTATAA | 0.040% | −0.642 | no | 5 | −4.297 |
| GTATAC | 0.025% | NA | no | 5 | −2.689 |
| GTATAG | 0.026% | −0.715 | no | 5 | −1.271 |
| GTATCA | 0.029% | −0.292 | no | 5 | −1.926 |
| GTATCC | 0.017% | NA | no | 5 | 0.022 |
| GTATCG | 0.021% | NA | no | 5 | −0.770 |
| GTATGA | 0.037% | NA | no | 5 | −2.042 |
| GTATGC | 0.025% | −0.144 | no | 5 | −0.688 |
| GTATGG | 0.030% | −0.214 | no | 5 | −0.812 |
| GTCAAT | 0.025% | NA | no | 5 | −1.344 |
| GTCACT | 0.018% | NA | no | 5 | 0.785 |
| GTCAGT | 0.032% | −0.308 | no | 5 | −1.632 |
| GTCATA | 0.028% | NA | no | 5 | −0.727 |
| GTCATC | 0.018% | 0.352 | no | 5 | −0.151 |
| GTCATG | 0.025% | NA | no | 5 | −0.051 |
| GTCCAT | 0.018% | NA | no | 5 | −0.880 |
| GTCCCT | 0.008% | NA | no | 5 | −0.675 |
| GTCCTA | 0.013% | NA | no | 5 | −1.202 |
| GTCCTC | 0.007% | NA | no | 5 | −5.098 |
| GTCCTG | 0.010% | 0.400 | no | 5 | −2.620 |
| GTCGAA | 0.039% | NA | no | 5 | −1.055 |
| GTCGAC | 0.020% | 0.686 | no | 5 | 0.900 |
| GTCGAG | 0.026% | NA | no | 5 | −0.778 |
| GTCGCA | 0.019% | NA | no | 5 | −1.538 |
| GTCGCC | 0.010% | 0.441 | no | 5 | −0.453 |
| GTCGCG | 0.022% | 0.497 | no | 5 | −0.592 |
| GTCGGA | 0.024% | 0.394 | no | 5 | −0.690 |
| GTCGGC | 0.020% | 0.440 | no | 5 | 1.670 |
| GTCGGG | 0.025% | 0.312 | no | 5 | 0.431 |
| GTCTAT | 0.020% | NA | no | 5 | −0.762 |
| GTCTCT | 0.011% | NA | no | 5 | −0.765 |
| GTCTGT | 0.016% | NA | no | 5 | −1.155 |
| GTCTTA | 0.013% | −0.386 | no | 5 | −1.347 |
| GTCTTC | 0.008% | 0.325 | no | 5 | −2.003 |
| GTCTTG | 0.019% | NA | no | 5 | −0.336 |
| GTGAAT | 0.040% | 0.199 | yes | 5 | −2.115 |
| GTGACT | 0.020% | 0.221 | yes | 5 | −2.125 |
| GTGAGT | 0.039% | NA | yes | 5 | −3.111 |
| GTGATA | 0.035% | −0.165 | yes | 5 | −2.428 |
| GTGATC | 0.023% | 0.249 | yes | 5 | −3.472 |
| GTGATG | 0.033% | 0.235 | yes | 5 | −3.399 |
| GTGCAT | 0.025% | −0.441 | no | 5 | −0.232 |
| GTGCCT | 0.011% | −0.422 | no | 5 | −0.446 |
| GTGCGT | 0.022% | −0.172 | no | 5 | −0.824 |
| GTGCTA | 0.020% | −0.525 | no | 5 | −0.885 |
| GTGCTC | 0.013% | −0.092 | no | 5 | −0.179 |
| GTGCTG | 0.020% | NA | no | 5 | −1.182 |
| GTGGAA | 0.048% | 0.587 | no | 5 | −0.533 |
| GTGGAC | 0.031% | 0.622 | no | 5 | −0.439 |
| GTGGAG | 0.031% | 0.512 | no | 5 | 0.284 |
| GTGGCA | 0.039% | NA | no | 5 | −0.129 |
| GTGGCC | 0.017% | NA | no | 5 | 1.182 |
| GTGGCG | 0.024% | 0.308 | no | 5 | 0.828 |
| GTGGGA | 0.043% | 0.153 | no | 5 | −1.383 |
| GTGGGC | 0.025% | −0.140 | no | 5 | −0.349 |
| GTGGGG | 0.034% | −0.214 | no | 5 | −0.841 |
| GTGTAT | 0.019% | −0.295 | no | 5 | −0.725 |
| GTGTCT | 0.019% | NA | no | 5 | −1.105 |
| GTGTGT | 0.023% | −0.159 | no | 5 | −0.949 |
| GTGTTA | 0.024% | −0.436 | no | 5 | −0.211 |
| GTGTTC | 0.017% | NA | no | 5 | −0.341 |
| GTGTTG | 0.021% | −0.111 | no | 5 | 0.886 |
| GTTAAT | 0.026% | −0.287 | no | 5 | −3.781 |
| GTTACT | 0.017% | NA | no | 5 | −2.622 |
| GTTAGT | 0.027% | −0.707 | no | 5 | −8.956 |
| GTTATA | 0.021% | −0.314 | no | 5 | −3.026 |
| GTTATC | 0.023% | 0.149 | no | 5 | −3.730 |
| GTTATG | 0.021% | −0.147 | no | 5 | −1.779 |
| GTTCAT | 0.024% | 0.162 | no | 5 | −0.873 |
| GTTCCT | 0.005% | NA | no | 5 | 0.484 |
| GTTCGT | 0.016% | 0.428 | no | 5 | −0.689 |
| GTTCTA | 0.017% | NA | no | 5 | −2.578 |
| GTTCTC | 0.011% | 0.246 | no | 5 | −2.189 |
| GTTCTG | 0.017% | 0.165 | no | 5 | 0.789 |
| GTTGAA | 0.037% | 0.175 | no | 5 | −1.037 |
| GTTGAC | 0.014% | 0.362 | no | 5 | −1.330 |
| GTTGAG | 0.026% | −0.218 | no | 5 | 0.387 |
| GTTGCA | 0.025% | NA | no | 5 | −1.266 |
| GTTGCC | 0.014% | 0.338 | no | 5 | −0.182 |
| GTTGCG | 0.018% | 0.366 | no | 5 | 0.269 |
| GTTGGA | 0.032% | 0.175 | no | 5 | 0.049 |
| GTTGGC | 0.017% | 0.180 | no | 5 | −0.486 |
| GTTGGG | 0.024% | −0.182 | no | 5 | −0.890 |
| GTTTAT | 0.017% | −0.244 | no | 5 | −2.759 |
| GTTTCT | 0.015% | NA | no | 5 | −3.512 |
| GTTTGT | 0.022% | NA | no | 5 | −2.699 |
| GTTTTA | 0.020% | −0.312 | no | 5 | −3.897 |
| GTTTTC | 0.012% | 0.142 | no | 5 | −6.195 |
| GTTTTG | 0.020% | −0.196 | no | 5 | −0.776 |
| TACAAA | 0.034% | NA | no | 5 | −1.744 |
| TACAAC | 0.019% | NA | no | 5 | −0.751 |
| TACAAG | 0.035% | NA | no | 5 | −0.198 |
| TACACA | 0.026% | NA | no | 5 | −0.939 |
| TACACC | 0.014% | NA | no | 5 | −1.353 |
| TACACG | 0.023% | 0.299 | no | 5 | −0.284 |
| TACAGA | 0.029% | NA | no | 5 | −0.020 |
| TACAGC | 0.020% | NA | no | 5 | 0.419 |
| TACAGG | 0.024% | −0.303 | no | 5 | −0.484 |
| TACCAA | 0.020% | NA | no | 5 | −0.661 |
| TACCAC | 0.010% | NA | no | 5 | −1.428 |
| TACCAG | 0.018% | −0.081 | no | 5 | −0.955 |
| TACCCA | 0.012% | −0.192 | no | 5 | −0.595 |
| TACCCC | 0.010% | −0.148 | no | 5 | −1.060 |
| TACCCG | 0.006% | 0.302 | no | 5 | −1.404 |
| TACCGA | 0.013% | 0.439 | no | 5 | −0.416 |
| TACCGC | 0.015% | NA | no | 5 | −0.105 |
| TACCGG | 0.014% | 0.450 | no | 5 | −0.806 |
| TACTAA | 0.014% | NA | no | 5 | −2.034 |
| TACTAC | 0.010% | NA | no | 5 | −1.549 |
| TACTAG | 0.017% | −0.521 | no | 5 | −1.505 |
| TACTCA | 0.020% | NA | no | 5 | −2.617 |
| TACTCC | 0.007% | NA | no | 5 | −0.371 |
| TACTCG | 0.013% | 0.268 | no | 5 | −0.441 |
| TACTGA | 0.018% | NA | no | 5 | −1.449 |
| TACTGC | 0.015% | 0.131 | no | 5 | −0.786 |
| TACTGG | 0.015% | NA | no | 5 | −0.002 |
| TAGAAA | 0.039% | −0.457 | no | 5 | −6.589 |
| TAGAAC | 0.016% | NA | no | 5 | −3.798 |
| TAGAAG | 0.041% | −0.334 | no | 5 | −2.207 |
| TAGACA | 0.022% | −0.586 | no | 5 | −5.210 |
| TAGACC | 0.014% | −0.252 | no | 5 | −3.376 |
| TAGACG | 0.023% | NA | no | 5 | −2.794 |
| TAGAGA | 0.026% | −0.424 | no | 5 | −4.534 |
| TAGAGC | 0.010% | NA | no | 5 | −3.536 |
| TAGAGG | 0.033% | −0.452 | no | 5 | −4.480 |
| TAGCAA | 0.025% | −0.545 | no | 5 | −6.255 |
| TAGCAC | 0.016% | −0.451 | no | 5 | −7.027 |
| TAGCAG | 0.020% | −0.644 | no | 5 | −1.294 |
| TAGCCA | 0.015% | −0.561 | no | 5 | −5.679 |
| TAGCCC | 0.009% | −0.599 | no | 5 | −4.306 |
| TAGCCG | 0.021% | NA | no | 5 | −0.532 |
| TAGCGA | 0.019% | NA | no | 5 | −4.953 |
| TAGCGC | 0.008% | NA | no | 5 | −2.377 |
| TAGCGG | 0.025% | −0.179 | no | 5 | −0.275 |
| TAGTAA | 0.026% | −0.812 | no | 5 | −8.863 |
| TAGTAC | 0.017% | −0.557 | no | 5 | −7.798 |
| TAGTAG | 0.025% | −0.938 | no | 5 | −4.603 |
| TAGTCA | 0.015% | −0.684 | no | 5 | −4.130 |
| TAGTCC | 0.007% | −0.447 | no | 5 | −4.568 |
| TAGTCG | 0.015% | NA | no | 5 | −5.048 |
| TAGTGA | 0.021% | −0.578 | no | 5 | −8.614 |
| TAGTGC | 0.014% | −0.352 | no | 5 | −4.905 |
| TAGTGG | 0.021% | −0.589 | no | 5 | −4.232 |
| TATAAA | 0.035% | −0.504 | no | 5 | −4.694 |
| TATAAC | 0.025% | −0.328 | no | 5 | −4.439 |
| TATAAG | 0.037% | −0.518 | no | 5 | −1.982 |
| TATACA | 0.024% | NA | no | 5 | −6.137 |
| TATACC | 0.010% | −0.224 | no | 5 | −3.607 |
| TATACG | 0.020% | NA | no | 5 | −1.105 |
| TATAGA | 0.025% | −0.447 | no | 5 | −5.487 |
| TATAGC | 0.023% | −0.401 | no | 5 | −9.389 |
| TATAGG | 0.025% | −0.771 | no | 5 | −3.740 |
| TATCAA | 0.019% | NA | no | 5 | −3.486 |
| TATCAC | 0.018% | NA | no | 5 | −3.211 |
| TATCAG | 0.020% | −0.379 | no | 5 | −2.726 |
| TATCCA | 0.016% | NA | no | 5 | −2.892 |
| TATCCC | 0.008% | −0.329 | no | 5 | −1.310 |
| TATCCG | 0.009% | 0.426 | no | 5 | −0.178 |
| TATCGA | 0.019% | NA | no | 5 | −0.977 |
| TATCGC | 0.017% | NA | no | 5 | −1.008 |
| TATCGG | 0.014% | 0.215 | no | 5 | −1.506 |
| TATTAA | 0.037% | −0.602 | no | 5 | −5.478 |
| TATTAC | 0.023% | −0.356 | no | 5 | −3.997 |
| TATTAG | 0.024% | −0.781 | no | 5 | −3.825 |
| TATTCA | 0.025% | NA | no | 5 | −4.573 |
| TATTCC | 0.005% | −0.329 | no | 5 | −5.515 |
| TATTCG | 0.011% | NA | no | 5 | −1.459 |
| TATTGA | 0.028% | NA | no | 5 | −4.403 |
| TATTGC | 0.021% | −0.358 | no | 5 | −4.090 |
| TATTGG | 0.025% | −0.256 | no | 5 | −1.227 |
| TCCAAA | 0.025% | NA | no | 5 | −0.289 |
| TCCAAC | 0.012% | NA | no | 5 | 0.190 |
| TCCAAG | 0.021% | NA | no | 5 | −0.417 |
| TCCACA | 0.016% | NA | no | 5 | 0.579 |
| TCCACC | 0.011% | −0.225 | no | 5 | −0.574 |
| TCCACG | 0.013% | 0.537 | no | 5 | −0.677 |
| TCCAGA | 0.025% | 0.053 | no | 5 | 0.050 |
| TCCAGC | 0.013% | NA | no | 5 | 0.101 |
| TCCAGG | 0.017% | NA | no | 5 | −0.781 |
| TCCCAA | 0.013% | −0.444 | no | 5 | −0.474 |
| TCCCAC | 0.010% | NA | no | 5 | −2.680 |
| TCCCAG | 0.014% | −0.292 | no | 5 | −1.114 |
| TCCCCA | 0.012% | −0.476 | no | 5 | 0.763 |
| TCCCCC | 0.005% | −0.393 | no | 5 | −0.635 |
| TCCCCG | 0.009% | 0.350 | no | 5 | 0.046 |
| TCCCGA | 0.011% | NA | no | 5 | −0.342 |
| TCCCGC | 0.009% | NA | no | 5 | −0.775 |
| TCCCGG | 0.012% | 0.401 | no | 5 | 1.328 |
| TCCTAA | 0.010% | NA | no | 5 | −1.571 |
| TCCTAC | 0.004% | NA | no | 5 | −1.401 |
| TCCTAG | 0.005% | −0.437 | no | 5 | −0.513 |
| TCCTCA | 0.010% | NA | no | 5 | −0.458 |
| TCCTCC | 0.009% | NA | no | 5 | −0.145 |
| TCCTCG | 0.010% | 0.623 | no | 5 | 0.593 |
| TCCTGA | 0.010% | NA | no | 5 | 0.509 |
| TCCTGC | 0.010% | 0.484 | no | 5 | 0.942 |
| TCCTGG | 0.010% | 0.335 | no | 5 | −0.442 |
| TCGAAA | 0.034% | NA | no | 5 | −0.872 |
| TCGAAC | 0.021% | 0.477 | no | 5 | −0.143 |
| TCGAAG | 0.035% | 0.297 | no | 5 | −0.951 |
| TCGACA | 0.018% | 0.428 | no | 5 | −1.033 |
| TCGACC | 0.013% | 0.650 | no | 5 | 0.297 |
| TCGACG | 0.015% | 0.875 | no | 5 | −1.137 |
| TCGAGA | 0.025% | 0.467 | no | 5 | 1.436 |
| TCGAGC | 0.022% | 0.449 | no | 5 | −0.432 |
| TCGAGG | 0.027% | 0.256 | no | 5 | −0.282 |
| TCGCAA | 0.026% | NA | no | 5 | 0.831 |
| TCGCAC | 0.013% | 0.502 | no | 5 | −0.998 |
| TCGCAG | 0.016% | NA | no | 5 | −0.069 |
| TCGCCA | 0.016% | 0.422 | no | 5 | −0.507 |
| TCGCCC | 0.005% | 0.352 | no | 5 | −0.672 |
| TCGCCG | 0.007% | 0.911 | no | 5 | 0.036 |
| TCGCGA | 0.014% | 0.661 | no | 5 | −0.336 |
| TCGCGC | 0.011% | 0.674 | no | 5 | −0.179 |
| TCGCGG | 0.022% | 0.621 | no | 5 | −0.504 |
| TCGTAA | 0.027% | NA | no | 5 | −2.058 |
| TCGTAC | 0.013% | 0.472 | no | 5 | 0.648 |
| TCGTAG | 0.013% | −0.318 | no | 5 | −1.878 |
| TCGTCA | 0.012% | 0.502 | no | 5 | −0.100 |
| TCGTCC | 0.008% | 0.650 | no | 5 | 0.701 |
| TCGTCG | 0.012% | 0.952 | no | 5 | 0.033 |
| TCGTGA | 0.018% | 0.435 | no | 5 | −0.519 |
| TCGTGC | 0.010% | 0.216 | no | 5 | −0.335 |
| TCGTGG | 0.017% | 0.404 | no | 5 | 0.453 |
| TCTAAA | 0.022% | NA | no | 5 | −3.197 |
| TCTAAC | 0.015% | NA | no | 5 | −3.877 |
| TCTAAG | 0.017% | −0.346 | no | 5 | −1.726 |
| TCTACA | 0.015% | NA | no | 5 | −1.131 |
| TCTACC | 0.006% | NA | no | 5 | −0.777 |
| TCTACG | 0.011% | 0.646 | no | 5 | 0.472 |
| TCTAGA | 0.018% | NA | no | 5 | −4.489 |
| TCTAGC | 0.012% | NA | no | 5 | −4.016 |
| TCTAGG | 0.012% | −0.561 | no | 5 | −4.521 |
| TCTCAA | 0.019% | NA | no | 5 | −2.451 |
| TCTCAC | 0.006% | NA | no | 5 | −0.333 |
| TCTCAG | 0.021% | NA | no | 5 | −1.602 |
| TCTCCA | 0.013% | −0.144 | no | 5 | −0.625 |
| TCTCCC | 0.008% | NA | no | 5 | −1.035 |
| TCTCCG | 0.007% | 0.647 | no | 5 | 0.334 |
| TCTCGA | 0.013% | 0.385 | no | 5 | −0.680 |
| TCTCGC | 0.015% | 0.475 | no | 5 | −0.125 |
| TCTCGG | 0.012% | 0.283 | no | 5 | 1.031 |
| TCTTAA | 0.017% | NA | no | 5 | −4.390 |
| TCTTAC | 0.010% | NA | no | 5 | −2.830 |
| TCTTAG | 0.015% | −0.622 | no | 5 | −2.510 |
| TCTTCA | 0.013% | 0.348 | no | 5 | −0.233 |
| TCTTCC | 0.004% | NA | no | 5 | −2.014 |
| TCTTCG | 0.008% | 0.741 | no | 5 | 3.396 |
| TCTTGA | 0.018% | NA | no | 5 | −2.890 |
| TCTTGC | 0.007% | −0.251 | no | 5 | −1.897 |
| TCTTGG | 0.017% | NA | no | 5 | 0.655 |
| TTCAAA | 0.031% | NA | no | 5 | −0.805 |
| TTCAAC | 0.019% | 0.504 | no | 5 | −0.794 |
| TTCAAG | 0.024% | 0.257 | no | 5 | 0.367 |
| TTCACA | 0.017% | NA | no | 5 | −1.522 |
| TTCACC | 0.010% | 0.229 | no | 5 | 0.986 |
| TTCACG | 0.014% | 0.388 | no | 5 | −0.429 |
| TTCAGA | 0.028% | NA | no | 5 | −0.116 |
| TTCAGC | 0.013% | 0.200 | no | 5 | 0.379 |
| TTCAGG | 0.022% | −0.322 | no | 5 | −0.183 |
| TTCCAA | 0.016% | NA | no | 5 | −0.030 |
| TTCCAG | 0.013% | −0.066 | no | 5 | −0.407 |
| TTCCCA | 0.011% | −0.468 | no | 5 | −2.337 |
| TTCCCG | 0.007% | NA | no | 5 | −1.343 |
| TTCCGA | 0.011% | 0.249 | no | 5 | −0.972 |
| TTCCGC | 0.007% | 0.275 | no | 5 | −0.268 |
| TTCCGG | 0.005% | 0.489 | no | 5 | 1.161 |
| TTCTAA | 0.014% | NA | no | 5 | −2.612 |
| TTCTAC | 0.011% | 0.436 | no | 5 | −0.142 |
| TTCTAG | 0.013% | −0.396 | no | 5 | −1.354 |
| TTCTCA | 0.011% | NA | no | 5 | −2.262 |
| TTCTCC | 0.007% | 0.170 | no | 5 | 0.765 |
| TTCTCG | 0.009% | 0.383 | no | 5 | −1.025 |
| TTCTGA | 0.015% | 0.215 | no | 5 | −0.870 |
| TTCTGC | 0.007% | 0.286 | no | 5 | −2.414 |
| TTCTGG | 0.013% | NA | no | 5 | 0.018 |
| TTGAAA | 0.033% | NA | yes | 5 | −4.030 |
| TTGAAC | 0.018% | 0.310 | yes | 5 | −3.608 |
| TTGAAG | 0.025% | NA | yes | 5 | −1.958 |
| TTGACA | 0.025% | NA | yes | 5 | −2.711 |
| TTGACC | 0.015% | 0.275 | yes | 5 | −0.405 |
| TTGACG | 0.015% | 0.454 | yes | 5 | −2.523 |
| TTGAGA | 0.034% | NA | yes | 5 | −4.157 |
| TTGAGC | 0.018% | NA | yes | 5 | −4.963 |
| TTGAGG | 0.028% | −0.328 | yes | 5 | −3.720 |
| TTGCAA | 0.022% | NA | no | 5 | −2.424 |
| TTGCAC | 0.015% | −0.183 | no | 5 | −1.474 |
| TTGCAG | 0.021% | NA | no | 5 | −0.307 |
| TTGCCA | 0.017% | NA | no | 5 | −0.548 |
| TTGCCC | 0.009% | NA | no | 5 | 0.299 |
| TTGCCG | 0.009% | 0.568 | no | 5 | 1.067 |
| TTGCGA | 0.022% | 0.421 | no | 5 | −0.172 |
| TTGCGC | 0.013% | 0.383 | no | 5 | −1.171 |
| TTGCGG | 0.017% | 0.281 | no | 5 | −0.356 |
| TTGTAA | 0.030% | −0.266 | no | 5 | −4.571 |
| TTGTAC | 0.018% | NA | no | 5 | −3.284 |
| TTGTAG | 0.024% | −0.529 | no | 5 | −2.313 |
| TTGTCA | 0.019% | NA | no | 5 | −2.718 |
| TTGTCC | 0.008% | NA | no | 5 | −1.316 |
| TTGTCG | 0.011% | 0.393 | no | 5 | −2.406 |
| TTGTGA | 0.017% | NA | no | 5 | −1.559 |
| TTGTGC | 0.016% | −0.151 | no | 5 | −1.858 |
| TTGTGG | 0.018% | −0.200 | no | 5 | −1.023 |
| TTTAAA | 0.024% | −0.374 | no | 5 | −5.352 |
| TTTAAC | 0.020% | −0.205 | no | 5 | −4.093 |
| TTTAAG | 0.027% | −0.444 | no | 5 | −1.160 |
| TTTACA | 0.019% | NA | no | 5 | −3.337 |
| TTTACC | 0.011% | NA | no | 5 | −1.276 |
| TTTACG | 0.015% | NA | no | 5 | −1.019 |
| TTTAGA | 0.022% | −0.485 | no | 5 | −6.601 |
| TTTAGC | 0.017% | −0.551 | no | 5 | −6.999 |
| TTTAGG | 0.024% | −0.785 | no | 5 | −5.226 |
| TTTCAA | 0.021% | NA | no | 5 | −2.008 |
| TTTCAC | 0.012% | NA | no | 5 | −2.971 |
| TTTCAG | 0.018% | −0.189 | no | 5 | −4.757 |
| TTTCCA | 0.018% | −0.218 | no | 5 | −1.259 |
| TTTCCC | 0.005% | −0.179 | no | 5 | −2.008 |
| TTTCCG | 0.009% | 0.475 | no | 5 | −0.553 |
| TTTCGA | 0.014% | 0.442 | no | 5 | −1.799 |
| TTTCGC | 0.012% | 0.453 | no | 5 | −1.907 |
| TTTCGG | 0.019% | 0.214 | no | 5 | −2.131 |
| TTTTAA | 0.021% | −0.387 | no | 5 | −4.022 |
| TTTTAC | 0.015% | −0.193 | no | 5 | −5.053 |
| TTTTAG | 0.025% | −0.648 | no | 5 | −6.442 |
| TTTTCA | 0.017% | NA | no | 5 | −9.618 |
| TTTTCC | 0.005% | −0.165 | no | 5 | −3.637 |
| TTTTCG | 0.012% | 0.251 | no | 5 | −2.527 |
| TTTTGA | 0.022% | NA | no | 5 | −5.532 |
| TTTTGC | 0.009% | −0.331 | no | 5 | −3.122 |
| TTTTGG | 0.015% | −0.268 | no | 5 | −1.089 |
| AACAAA | 0.079% | NA | no | 6 | −1.223 |
| AACAAC | 0.045% | 0.551 | no | 6 | −0.435 |
| AACAAG | 0.066% | NA | no | 6 | −0.152 |
| AACACA | 0.041% | NA | no | 6 | −0.450 |
| AACACC | 0.020% | NA | no | 6 | 0.583 |
| AACACG | 0.039% | 0.474 | no | 6 | −0.449 |
| AACAGA | 0.064% | 0.537 | no | 6 | −0.863 |
| AACAGC | 0.038% | NA | no | 6 | −0.176 |
| AACAGG | 0.050% | −0.274 | no | 6 | −0.552 |
| AACCAA | 0.044% | 0.403 | no | 6 | −0.664 |
| AACCAC | 0.025% | NA | no | 6 | −0.872 |
| AACCAG | 0.026% | NA | no | 6 | −0.484 |
| AACCCA | 0.027% | −0.352 | no | 6 | −0.786 |
| AACCCC | 0.017% | −0.255 | no | 6 | −0.008 |
| AACCCG | 0.020% | 0.470 | no | 6 | 0.317 |
| AACCGA | 0.027% | NA | no | 6 | −0.091 |
| AACCGC | 0.022% | NA | no | 6 | 0.099 |
| AACCGG | 0.027% | 0.439 | no | 6 | −0.504 |
| AACTAA | 0.032% | NA | no | 6 | −2.020 |
| AACTAC | 0.022% | 0.312 | no | 6 | −1.103 |
| AACTAG | 0.029% | −0.317 | no | 6 | −0.749 |
| AACTCA | 0.028% | NA | no | 6 | −1.181 |
| AACTCC | 0.012% | NA | no | 6 | −1.754 |
| AACTCG | 0.027% | 0.416 | no | 6 | −0.043 |
| AACTGA | 0.038% | NA | no | 6 | −0.866 |
| AACTGC | 0.024% | 0.209 | no | 6 | −1.788 |
| AACTGG | 0.033% | NA | no | 6 | −0.353 |
| AAGAAA | 0.100% | 0.795 | no | 6 | −1.070 |
| AAGAAC | 0.051% | 0.630 | no | 6 | −0.752 |
| AAGAAG | 0.091% | 0.591 | no | 6 | −0.870 |
| AAGACA | 0.066% | 0.424 | no | 6 | −1.011 |
| AAGACC | 0.032% | 0.397 | no | 6 | −0.096 |
| AAGACG | 0.043% | 0.725 | no | 6 | −0.365 |
| AAGAGA | 0.071% | 0.675 | no | 6 | −2.059 |
| AAGAGC | 0.045% | 0.394 | no | 6 | −1.261 |
| AAGAGG | 0.074% | NA | no | 6 | −0.907 |
| AAGCAA | 0.051% | NA | no | 6 | −2.326 |
| AAGCAC | 0.040% | NA | no | 6 | −1.268 |
| AAGCAG | 0.045% | NA | no | 6 | −0.556 |
| AAGCCA | 0.031% | NA | no | 6 | −1.117 |
| AAGCCC | 0.015% | NA | no | 6 | −0.267 |
| AAGCCG | 0.022% | 0.355 | no | 6 | 0.761 |
| AAGCGA | 0.039% | 0.418 | no | 6 | 0.113 |
| AAGCGC | 0.028% | 0.435 | no | 6 | −0.954 |
| AAGCGG | 0.043% | 0.286 | no | 6 | −0.174 |
| AAGTAA | 0.070% | NA | no | 6 | −3.365 |
| AAGTAC | 0.030% | NA | no | 6 | −2.618 |
| AAGTAG | 0.058% | −0.478 | no | 6 | −2.525 |
| AAGTCA | 0.033% | NA | no | 6 | −2.071 |
| AAGTCC | 0.009% | NA | no | 6 | −0.638 |
| AAGTCG | 0.026% | 0.427 | no | 6 | 0.228 |
| AAGTGA | 0.053% | NA | no | 6 | −2.274 |
| AAGTGC | 0.029% | NA | no | 6 | −1.434 |
| AAGTGG | 0.049% | −0.168 | no | 6 | −0.181 |
| AATAAA | 0.066% | −0.344 | no | 6 | −3.830 |
| AATAAC | 0.026% | NA | no | 6 | −3.783 |
| AATAAG | 0.056% | −0.292 | no | 6 | −1.313 |
| AATACA | 0.046% | NA | no | 6 | −4.253 |
| AATACC | 0.012% | NA | no | 6 | −2.592 |
| AATACG | 0.034% | NA | no | 6 | −0.468 |
| AATAGA | 0.048% | NA | no | 6 | −6.561 |
| AATAGC | 0.025% | NA | no | 6 | −4.772 |
| AATAGG | 0.047% | −0.629 | no | 6 | −6.146 |
| AATCAA | 0.042% | NA | no | 6 | −2.974 |
| AATCAC | 0.033% | NA | no | 6 | −3.043 |
| AATCAG | 0.044% | NA | no | 6 | −2.719 |
| AATCCA | 0.027% | NA | no | 6 | −1.675 |
| AATCCC | 0.008% | −0.213 | no | 6 | −3.348 |
| AATCCG | 0.018% | 0.457 | no | 6 | 0.042 |
| AATCGA | 0.030% | 0.468 | no | 6 | −2.530 |
| AATCGC | 0.021% | 0.396 | no | 6 | 0.006 |
| AATCGG | 0.034% | 0.266 | no | 6 | −0.083 |
| AATTAA | 0.031% | NA | no | 6 | −4.301 |
| AATTAC | 0.010% | NA | no | 6 | −3.265 |
| AATTAG | 0.026% | −0.609 | no | 6 | −3.921 |
| AATTCA | 0.012% | NA | no | 6 | −6.142 |
| AATTCC | 0.002% | −0.220 | no | 6 | −4.851 |
| AATTCG | 0.010% | 0.321 | no | 6 | −1.960 |
| AATTGA | 0.037% | NA | no | 6 | −3.507 |
| AATTGC | 0.012% | −0.205 | no | 6 | −1.931 |
| AATTGG | 0.026% | NA | no | 6 | −0.842 |
| ACCAAA | 0.041% | NA | no | 6 | −1.640 |
| ACCAAC | 0.026% | NA | no | 6 | 0.173 |
| ACCAAG | 0.039% | NA | no | 6 | −0.064 |
| ACCACA | 0.025% | NA | no | 6 | −1.649 |
| ACCACC | 0.015% | −0.424 | no | 6 | −0.772 |
| ACCACG | 0.030% | 0.371 | no | 6 | −0.266 |
| ACCAGA | 0.032% | NA | no | 6 | 0.760 |
| ACCAGC | 0.018% | NA | no | 6 | 0.644 |
| ACCAGG | 0.038% | NA | no | 6 | 0.447 |
| ACCCAA | 0.031% | −0.146 | no | 6 | −1.064 |
| ACCCAC | 0.015% | −0.138 | no | 6 | −1.499 |
| ACCCAG | 0.023% | −0.227 | no | 6 | −0.827 |
| ACCCCA | 0.016% | −0.328 | no | 6 | −1.261 |
| ACCCCC | 0.011% | −0.181 | no | 6 | −1.060 |
| ACCCCG | 0.016% | 0.122 | no | 6 | 0.043 |
| ACCCGA | 0.022% | 0.318 | no | 6 | 0.129 |
| ACCCGC | 0.015% | 0.273 | no | 6 | −0.661 |
| ACCCGG | 0.022% | 0.317 | no | 6 | −0.080 |
| ACCTAA | 0.021% | −0.587 | no | 6 | −1.676 |
| ACCTAC | 0.016% | NA | no | 6 | 0.459 |
| ACCTAG | 0.020% | −0.327 | no | 6 | −0.439 |
| ACCTCA | 0.018% | NA | no | 6 | −0.718 |
| ACCTCC | 0.011% | NA | no | 6 | 0.266 |
| ACCTCG | 0.017% | 0.548 | no | 6 | −0.647 |
| ACCTGA | 0.027% | NA | no | 6 | −0.620 |
| ACCTGC | 0.016% | 0.442 | no | 6 | 0.784 |
| ACCTGG | 0.022% | 0.352 | no | 6 | −0.530 |
| ACGAAA | 0.059% | NA | no | 6 | −0.509 |
| ACGAAC | 0.034% | 0.473 | no | 6 | −0.601 |
| ACGAAG | 0.054% | 0.405 | no | 6 | 0.044 |
| ACGACA | 0.033% | NA | no | 6 | −0.435 |
| ACGACC | 0.019% | 0.415 | no | 6 | −0.709 |
| ACGACG | 0.035% | 0.724 | no | 6 | −0.168 |
| ACGAGA | 0.054% | NA | no | 6 | 0.255 |
| ACGAGC | 0.032% | 0.325 | no | 6 | −1.146 |
| ACGAGG | 0.046% | 0.215 | no | 6 | −0.521 |
| ACGCAA | 0.033% | NA | no | 6 | −0.561 |
| ACGCAC | 0.021% | NA | no | 6 | −1.187 |
| ACGCAG | 0.028% | NA | no | 6 | 0.578 |
| ACGCCA | 0.030% | 0.372 | no | 6 | −0.027 |
| ACGCCC | 0.014% | 0.323 | no | 6 | −0.528 |
| ACGCCG | 0.019% | 0.809 | no | 6 | 0.736 |
| ACGCGA | 0.023% | 0.485 | no | 6 | 0.189 |
| ACGCGC | 0.026% | 0.608 | no | 6 | −0.105 |
| ACGCGG | 0.029% | 0.600 | no | 6 | 0.058 |
| ACGTAA | 0.033% | NA | no | 6 | −2.273 |
| ACGTAC | 0.021% | 0.286 | no | 6 | −1.357 |
| ACGTAG | 0.032% | −0.390 | no | 6 | −0.423 |
| ACGTCA | 0.026% | NA | no | 6 | −0.306 |
| ACGTCC | 0.016% | 0.553 | no | 6 | −0.992 |
| ACGTCG | 0.019% | 0.810 | no | 6 | 1.044 |
| ACGTGA | 0.031% | NA | no | 6 | 0.058 |
| ACGTGC | 0.019% | 0.195 | no | 6 | 0.168 |
| ACGTGG | 0.028% | 0.352 | no | 6 | −0.177 |
| ACTAAA | 0.040% | −0.583 | no | 6 | −2.421 |
| ACTAAC | 0.028% | NA | no | 6 | −4.117 |
| ACTAAG | 0.040% | −0.359 | no | 6 | −0.554 |
| ACTACA | 0.026% | NA | no | 6 | −1.668 |
| ACTACC | 0.016% | −0.209 | no | 6 | −1.170 |
| ACTACG | 0.026% | 0.510 | no | 6 | −0.436 |
| ACTAGA | 0.031% | NA | no | 6 | −4.202 |
| ACTAGC | 0.016% | NA | no | 6 | −5.326 |
| ACTAGG | 0.027% | −0.542 | no | 6 | −3.660 |
| ACTCAA | 0.033% | NA | no | 6 | −5.224 |
| ACTCAC | 0.020% | NA | no | 6 | −4.276 |
| ACTCAG | 0.030% | NA | no | 6 | −3.063 |
| ACTCCA | 0.021% | −0.139 | no | 6 | −1.530 |
| ACTCCC | 0.013% | NA | no | 6 | −2.385 |
| ACTCCG | 0.017% | 0.602 | no | 6 | −0.524 |
| ACTCGA | 0.030% | NA | no | 6 | −2.188 |
| ACTCGC | 0.017% | 0.389 | no | 6 | −2.331 |
| ACTCGG | 0.025% | 0.290 | no | 6 | −0.663 |
| ACTTAA | 0.032% | −0.672 | no | 6 | −2.935 |
| ACTTAC | 0.018% | NA | no | 6 | −2.808 |
| ACTTAG | 0.028% | −0.656 | no | 6 | −2.578 |
| ACTTCA | 0.023% | NA | no | 6 | −2.397 |
| ACTTCC | 0.005% | NA | no | 6 | −1.028 |
| ACTTCG | 0.026% | 0.621 | no | 6 | 0.417 |
| ACTTGA | 0.024% | NA | no | 6 | −1.997 |
| ACTTGC | 0.010% | −0.139 | no | 6 | −1.414 |
| ACTTGG | 0.024% | NA | no | 6 | −0.685 |
| ATCAAA | 0.056% | NA | no | 6 | −0.835 |
| ATCAAC | 0.029% | NA | no | 6 | −0.314 |
| ATCAAG | 0.045% | NA | no | 6 | 0.147 |
| ATCACA | 0.037% | NA | no | 6 | −0.680 |
| ATCACC | 0.019% | −0.206 | no | 6 | −0.633 |
| ATCACG | 0.029% | 0.259 | no | 6 | 0.279 |
| ATCAGA | 0.046% | NA | no | 6 | −0.826 |
| ATCAGC | 0.030% | NA | no | 6 | −2.575 |
| ATCAGG | 0.037% | −0.262 | no | 6 | −0.642 |
| ATCCAA | 0.034% | NA | no | 6 | −0.925 |
| ATCCAC | 0.021% | NA | no | 6 | −0.263 |
| ATCCAG | 0.028% | 0.145 | no | 6 | 0.100 |
| ATCCCA | 0.022% | −0.279 | no | 6 | −0.105 |
| ATCCCC | 0.014% | −0.303 | no | 6 | −1.336 |
| ATCCCG | 0.017% | 0.506 | no | 6 | 0.680 |
| ATCCGA | 0.027% | NA | no | 6 | 0.176 |
| ATCCGC | 0.014% | NA | no | 6 | 0.323 |
| ATCCGG | 0.020% | 0.511 | no | 6 | −1.276 |
| ATCTAA | 0.034% | NA | no | 6 | −1.364 |
| ATCTAC | 0.021% | NA | no | 6 | −0.528 |
| ATCTAG | 0.021% | −0.547 | no | 6 | −0.354 |
| ATCTCA | 0.023% | NA | no | 6 | −1.512 |
| ATCTCC | 0.010% | NA | no | 6 | 0.658 |
| ATCTCG | 0.022% | 0.322 | no | 6 | 0.927 |
| ATCTGA | 0.027% | NA | no | 6 | −0.341 |
| ATCTGC | 0.016% | 0.286 | no | 6 | −1.701 |
| ATCTGG | 0.031% | NA | no | 6 | −0.188 |
| ATGAAA | 0.066% | NA | yes | 6 | −3.745 |
| ATGAAC | 0.046% | 0.449 | yes | 6 | −3.231 |
| ATGAAG | 0.064% | 0.338 | yes | 6 | −2.507 |
| ATGACA | 0.041% | NA | yes | 6 | −5.400 |
| ATGACC | 0.022% | 0.325 | yes | 6 | −3.099 |
| ATGACG | 0.026% | 0.544 | yes | 6 | −2.841 |
| ATGAGA | 0.058% | NA | yes | 6 | −4.763 |
| ATGAGC | 0.032% | NA | yes | 6 | −4.554 |
| ATGAGG | 0.061% | −0.233 | yes | 6 | −3.655 |
| ATGCAA | 0.043% | NA | no | 6 | −3.871 |
| ATGCAC | 0.028% | −0.109 | no | 6 | −4.321 |
| ATGCAG | 0.032% | NA | no | 6 | −0.954 |
| ATGCCA | 0.029% | 0.121 | no | 6 | −2.194 |
| ATGCCC | 0.017% | NA | no | 6 | −1.692 |
| ATGCCG | 0.017% | 0.422 | no | 6 | −0.635 |
| ATGCGA | 0.040% | NA | no | 6 | −1.188 |
| ATGCGC | 0.024% | 0.408 | no | 6 | −1.314 |
| ATGCGG | 0.032% | 0.291 | no | 6 | −0.769 |
| ATGTAA | 0.050% | NA | no | 6 | −3.819 |
| ATGTAC | 0.028% | NA | no | 6 | −2.984 |
| ATGTAG | 0.044% | −0.544 | no | 6 | −2.805 |
| ATGTCA | 0.032% | NA | no | 6 | −2.740 |
| ATGTCC | 0.014% | NA | no | 6 | −1.365 |
| ATGTCG | 0.024% | 0.397 | no | 6 | −0.366 |
| ATGTGA | 0.040% | NA | no | 6 | −2.236 |
| ATGTGC | 0.026% | NA | no | 6 | −2.607 |
| ATGTGG | 0.040% | NA | no | 6 | −0.587 |
| ATTAAA | 0.059% | −0.520 | no | 6 | −3.838 |
| ATTAAC | 0.034% | NA | no | 6 | −3.640 |
| ATTAAG | 0.047% | −0.359 | no | 6 | −1.145 |
| ATTACA | 0.046% | NA | no | 6 | −3.134 |
| ATTACC | 0.019% | −0.226 | no | 6 | −0.545 |
| ATTACG | 0.020% | NA | no | 6 | −0.823 |
| ATTAGA | 0.045% | NA | no | 6 | −6.264 |
| ATTAGC | 0.025% | −0.441 | no | 6 | −4.786 |
| ATTAGG | 0.036% | −0.727 | no | 6 | −6.758 |
| ATTCAA | 0.035% | NA | no | 6 | −3.119 |
| ATTCAC | 0.021% | NA | no | 6 | −2.825 |
| ATTCAG | 0.031% | NA | no | 6 | −4.356 |
| ATTCCA | 0.016% | −0.152 | no | 6 | −2.110 |
| ATTCCG | 0.009% | 0.453 | no | 6 | 0.015 |
| ATTCGA | 0.026% | NA | no | 6 | −1.285 |
| ATTCGC | 0.017% | 0.453 | no | 6 | −0.274 |
| ATTCGG | 0.024% | 0.274 | no | 6 | −0.292 |
| ATTTAA | 0.035% | −0.490 | no | 6 | −6.043 |
| ATTTAC | 0.021% | NA | no | 6 | −4.089 |
| ATTTAG | 0.034% | −0.668 | no | 6 | −2.827 |
| ATTTCA | 0.030% | NA | no | 6 | −5.418 |
| ATTTCC | 0.009% | −0.330 | no | 6 | −4.799 |
| ATTTCG | 0.024% | 0.301 | no | 6 | −1.362 |
| ATTTGA | 0.033% | NA | no | 6 | −3.536 |
| ATTTGC | 0.023% | −0.177 | no | 6 | −3.379 |
| ATTTGG | 0.031% | NA | no | 6 | −1.002 |
| CACAAA | 0.030% | −0.211 | no | 6 | −0.771 |
| CACAAC | 0.019% | NA | no | 6 | 0.539 |
| CACAAG | 0.026% | NA | no | 6 | −0.359 |
| CACACA | 0.024% | NA | no | 6 | −2.081 |
| CACACC | 0.016% | NA | no | 6 | 0.247 |
| CACACG | 0.020% | NA | no | 6 | 0.003 |
| CACAGA | 0.022% | NA | no | 6 | −0.253 |
| CACAGC | 0.019% | −0.222 | no | 6 | −0.260 |
| CACAGG | 0.026% | −0.235 | no | 6 | −0.779 |
| CACCAA | 0.020% | −0.265 | no | 6 | −0.763 |
| CACCAC | 0.013% | NA | no | 6 | −0.990 |
| CACCAG | 0.017% | −0.233 | no | 6 | 0.104 |
| CACCCA | 0.013% | −0.268 | no | 6 | −1.539 |
| CACCCC | 0.012% | −0.179 | no | 6 | −1.296 |
| CACCCG | 0.012% | 0.193 | no | 6 | 0.915 |
| CACCGA | 0.022% | NA | no | 6 | −0.286 |
| CACCGC | 0.013% | NA | no | 6 | 0.030 |
| CACCGG | 0.019% | 0.390 | no | 6 | −0.746 |
| CACTAA | 0.024% | −0.481 | no | 6 | −2.068 |
| CACTAC | 0.011% | NA | no | 6 | −0.250 |
| CACTAG | 0.016% | −0.509 | no | 6 | −0.299 |
| CACTCA | 0.019% | −0.208 | no | 6 | −0.887 |
| CACTCC | 0.008% | NA | no | 6 | −1.706 |
| CACTCG | 0.014% | NA | no | 6 | −0.577 |
| CACTGA | 0.013% | NA | no | 6 | −1.397 |
| CACTGC | 0.010% | NA | no | 6 | 0.823 |
| CACTGG | 0.019% | NA | no | 6 | 0.426 |
| CAGAAA | 0.044% | NA | no | 6 | −1.080 |
| CAGAAC | 0.030% | 0.200 | no | 6 | −0.460 |
| CAGAAG | 0.044% | 0.390 | no | 6 | 0.587 |
| CAGACA | 0.033% | NA | no | 6 | −1.182 |
| CAGACC | 0.014% | NA | no | 6 | −0.740 |
| CAGACG | 0.026% | 0.466 | no | 6 | −0.530 |
| CAGAGA | 0.038% | 0.349 | no | 6 | −0.391 |
| CAGAGC | 0.022% | NA | no | 6 | −0.552 |
| CAGAGG | 0.039% | NA | no | 6 | 0.289 |
| CAGCAA | 0.026% | NA | no | 6 | −0.836 |
| CAGCAC | 0.016% | NA | no | 6 | −0.794 |
| CAGCAG | 0.022% | NA | no | 6 | −0.735 |
| CAGCCA | 0.018% | −0.445 | no | 6 | −1.254 |
| CAGCCC | 0.011% | −0.242 | no | 6 | 3.943 |
| CAGCCG | 0.014% | NA | no | 6 | −0.381 |
| CAGCGA | 0.021% | NA | no | 6 | −0.777 |
| CAGCGC | 0.017% | NA | no | 6 | 0.405 |
| CAGCGG | 0.025% | 0.262 | no | 6 | 0.134 |
| CAGTAA | 0.036% | −0.456 | no | 6 | −1.590 |
| CAGTAC | 0.019% | NA | no | 6 | 1.194 |
| CAGTAG | 0.027% | −0.530 | no | 6 | −0.331 |
| CAGTCA | 0.020% | −0.297 | no | 6 | −0.655 |
| CAGTCC | 0.013% | NA | no | 6 | −0.147 |
| CAGTCG | 0.020% | 0.213 | no | 6 | −0.761 |
| CAGTGA | 0.025% | NA | no | 6 | −0.735 |
| CAGTGC | 0.012% | −0.163 | no | 6 | −0.562 |
| CAGTGG | 0.029% | NA | no | 6 | −0.249 |
| CATAAA | 0.029% | −0.324 | no | 6 | −2.097 |
| CATAAC | 0.021% | −0.238 | no | 6 | −2.125 |
| CATAAG | 0.035% | NA | no | 6 | −0.479 |
| CATACA | 0.021% | NA | no | 6 | −1.533 |
| CATACC | 0.010% | NA | no | 6 | −1.021 |
| CATACG | 0.020% | NA | no | 6 | 1.296 |
| CATAGA | 0.024% | NA | no | 6 | −2.030 |
| CATAGC | 0.015% | NA | no | 6 | −3.406 |
| CATAGG | 0.023% | −0.456 | no | 6 | −2.964 |
| CATCAA | 0.021% | NA | no | 6 | 0.271 |
| CATCAC | 0.018% | NA | no | 6 | −0.493 |
| CATCAG | 0.016% | NA | no | 6 | −0.703 |
| CATCCA | 0.012% | 0.125 | no | 6 | −0.239 |
| CATCCC | 0.008% | NA | no | 6 | −0.847 |
| CATCCG | 0.010% | 0.184 | no | 6 | 0.198 |
| CATCGA | 0.023% | NA | no | 6 | −0.618 |
| CATCGC | 0.016% | 0.232 | no | 6 | 0.067 |
| CATCGG | 0.020% | 0.420 | no | 6 | −0.054 |
| CATTAA | 0.018% | −0.355 | no | 6 | −2.843 |
| CATTAC | 0.015% | NA | no | 6 | −2.805 |
| CATTAG | 0.017% | −0.514 | no | 6 | −3.177 |
| CATTCA | 0.015% | NA | no | 6 | −2.604 |
| CATTCC | 0.006% | NA | no | 6 | −1.749 |
| CATTCG | 0.011% | 0.186 | no | 6 | 1.072 |
| CATTGA | 0.016% | NA | no | 6 | −0.844 |
| CATTGC | 0.012% | NA | no | 6 | 1.900 |
| CATTGG | 0.020% | NA | no | 6 | −1.491 |
| CCCAAA | 0.018% | −0.434 | no | 6 | −2.557 |
| CCCAAC | 0.012% | NA | no | 6 | −0.519 |
| CCCAAG | 0.017% | NA | no | 6 | −0.776 |
| CCCACA | 0.012% | −0.150 | no | 6 | −3.281 |
| CCCACC | 0.005% | −0.429 | no | 6 | 0.806 |
| CCCACG | 0.008% | 0.302 | no | 6 | −0.005 |
| CCCAGA | 0.010% | −0.243 | no | 6 | 0.265 |
| CCCAGC | 0.012% | −0.541 | no | 6 | 0.198 |
| CCCAGG | 0.010% | −0.091 | no | 6 | −0.919 |
| CCCCAA | 0.010% | −0.359 | no | 6 | −3.543 |
| CCCCAC | 0.005% | −0.155 | no | 6 | −4.110 |
| CCCCAG | 0.011% | −0.423 | no | 6 | −0.160 |
| CCCCCA | 0.010% | −0.396 | no | 6 | −1.610 |
| CCCCCC | 0.004% | −0.137 | no | 6 | −3.894 |
| CCCCCG | 0.004% | 0.128 | no | 6 | 0.494 |
| CCCCGA | 0.010% | NA | no | 6 | −1.287 |
| CCCCGC | 0.008% | 0.197 | no | 6 | −0.294 |
| CCCCGG | 0.007% | 0.192 | no | 6 | −1.772 |
| CCCTAA | 0.012% | −0.467 | no | 6 | −2.684 |
| CCCTAC | 0.003% | NA | no | 6 | −2.166 |
| CCCTAG | 0.005% | −0.531 | no | 6 | −1.334 |
| CCCTCA | 0.010% | NA | no | 6 | −1.825 |
| CCCTCC | 0.005% | 0.115 | no | 6 | −1.343 |
| CCCTCG | 0.008% | 0.365 | no | 6 | −0.699 |
| CCCTGA | 0.012% | NA | no | 6 | 0.587 |
| CCCTGC | 0.008% | 0.317 | no | 6 | 0.904 |
| CCCTGG | 0.012% | 0.134 | no | 6 | 0.020 |
| CCGAAA | 0.022% | NA | no | 6 | 0.254 |
| CCGAAC | 0.016% | 0.309 | no | 6 | −0.653 |
| CCGAAG | 0.017% | 0.455 | no | 6 | −1.615 |
| CCGACA | 0.016% | NA | no | 6 | −0.232 |
| CCGACC | 0.008% | 0.193 | no | 6 | 0.208 |
| CCGACG | 0.010% | 0.786 | no | 6 | −0.263 |
| CCGAGA | 0.020% | 0.263 | no | 6 | 0.081 |
| CCGAGC | 0.012% | 0.216 | no | 6 | −0.027 |
| CCGAGG | 0.019% | 0.358 | no | 6 | 0.033 |
| CCGCAA | 0.003% | NA | no | 6 | −2.683 |
| CCGCAC | 0.014% | NA | no | 6 | −1.423 |
| CCGCAG | 0.013% | −0.179 | no | 6 | 0.287 |
| CCGCCA | 0.011% | −0.119 | no | 6 | −1.825 |
| CCGCCC | 0.006% | NA | no | 6 | −1.404 |
| CCGCCG | 0.007% | 0.387 | no | 6 | 0.270 |
| CCGCGA | 0.014% | 0.583 | no | 6 | −0.361 |
| CCGCGC | 0.008% | 0.485 | no | 6 | −1.506 |
| CCGCGG | 0.008% | 0.662 | no | 6 | −0.293 |
| CCGTAA | 0.012% | NA | no | 6 | −0.303 |
| CCGTAC | 0.009% | NA | no | 6 | 1.249 |
| CCGTAG | 0.011% | −0.348 | no | 6 | −0.076 |
| CCGTCA | 0.010% | 0.347 | no | 6 | −0.858 |
| CCGTCC | 0.009% | 0.395 | no | 6 | 0.551 |
| CCGTCG | 0.008% | 0.857 | no | 6 | −0.255 |
| CCGTGA | 0.015% | 0.356 | no | 6 | −0.091 |
| CCGTGC | 0.008% | 0.205 | no | 6 | 0.378 |
| CCGTGG | 0.014% | 0.447 | no | 6 | 0.216 |
| CCTAAA | 0.013% | −0.562 | no | 6 | −2.307 |
| CCTAAC | 0.006% | −0.367 | no | 6 | −1.831 |
| CCTAAG | 0.011% | −0.380 | no | 6 | 1.499 |
| CCTACA | 0.011% | NA | no | 6 | 0.212 |
| CCTACC | 0.006% | NA | no | 6 | −0.373 |
| CCTACG | 0.012% | 0.382 | no | 6 | 0.262 |
| CCTAGA | 0.010% | −0.469 | no | 6 | −5.503 |
| CCTAGC | 0.007% | −0.608 | no | 6 | −2.402 |
| CCTAGG | 0.010% | −0.465 | no | 6 | −3.453 |
| CCTCAA | 0.010% | NA | no | 6 | −1.196 |
| CCTCAC | 0.008% | NA | no | 6 | −1.720 |
| CCTCAG | 0.012% | NA | no | 6 | −0.872 |
| CCTCCA | 0.011% | NA | no | 6 | −1.175 |
| CCTCCC | 0.005% | NA | no | 6 | −1.838 |
| CCTCCG | 0.007% | 0.532 | no | 6 | 0.464 |
| CCTCGA | 0.009% | 0.378 | no | 6 | −0.270 |
| CCTCGC | 0.010% | 0.374 | no | 6 | −1.018 |
| CCTCGG | 0.011% | 0.483 | no | 6 | 0.379 |
| CCTTAA | 0.013% | −0.629 | no | 6 | −3.250 |
| CCTTAC | 0.005% | −0.322 | no | 6 | −2.738 |
| CCTTAG | 0.008% | −0.773 | no | 6 | −2.026 |
| CCTTCA | 0.008% | NA | no | 6 | −1.677 |
| CCTTCC | 0.006% | NA | no | 6 | −0.883 |
| CCTTCG | 0.006% | 0.414 | no | 6 | 0.015 |
| CCTTGA | 0.008% | NA | no | 6 | −0.905 |
| CCTTGC | 0.005% | NA | no | 6 | 0.222 |
| CCTTGG | 0.007% | 0.141 | no | 6 | −0.611 |
| CTCAAA | 0.023% | −0.488 | no | 6 | 0.380 |
| CTCAAC | 0.008% | NA | no | 6 | −1.472 |
| CTCAAG | 0.014% | NA | no | 6 | 0.634 |
| CTCACA | 0.014% | NA | no | 6 | −0.348 |
| CTCACC | 0.008% | NA | no | 6 | 0.519 |
| CTCACG | 0.009% | NA | no | 6 | −0.693 |
| CTCAGA | 0.022% | NA | no | 6 | −1.547 |
| CTCAGC | 0.008% | NA | no | 6 | −0.792 |
| CTCAGG | 0.016% | −0.316 | no | 6 | −0.412 |
| CTCCAA | 0.012% | NA | no | 6 | 0.477 |
| CTCCAC | 0.006% | NA | no | 6 | −0.726 |
| CTCCAG | 0.011% | −0.112 | no | 6 | −0.419 |
| CTCCCA | 0.007% | −0.225 | no | 6 | −3.150 |
| CTCCCC | 0.006% | −0.260 | no | 6 | −3.218 |
| CTCCGA | 0.008% | 0.507 | no | 6 | −0.550 |
| CTCCGC | 0.007% | 0.458 | no | 6 | −2.501 |
| CTCCGG | 0.009% | 0.532 | no | 6 | −1.221 |
| CTCTAA | 0.012% | −0.382 | no | 6 | −2.334 |
| CTCTAC | 0.009% | NA | no | 6 | −0.627 |
| CTCTAG | 0.009% | −0.387 | no | 6 | −1.260 |
| CTCTCA | 0.006% | NA | no | 6 | −0.599 |
| CTCTCG | 0.009% | 0.264 | no | 6 | −1.408 |
| CTCTGA | 0.014% | NA | no | 6 | 0.324 |
| CTCTGC | 0.013% | 0.263 | no | 6 | −1.298 |
| CTCTGG | 0.012% | NA | no | 6 | −0.087 |
| CTGAAA | 0.022% | NA | yes | 6 | −2.700 |
| CTGAAC | 0.011% | NA | yes | 6 | −4.087 |
| CTGAAG | 0.023% | 0.266 | yes | 6 | −1.983 |
| CTGACA | 0.013% | NA | yes | 6 | −2.374 |
| CTGACC | 0.009% | 0.264 | yes | 6 | −1.931 |
| CTGACG | 0.014% | 0.522 | yes | 6 | −2.443 |
| CTGAGA | 0.017% | NA | yes | 6 | −2.198 |
| CTGAGG | 0.014% | NA | yes | 6 | −3.700 |
| CTGCAA | 0.020% | NA | no | 6 | −0.143 |
| CTGCAC | 0.015% | 0.260 | no | 6 | 0.900 |
| CTGCAG | 0.012% | NA | no | 6 | −0.984 |
| CTGCCA | 0.006% | NA | no | 6 | 2.754 |
| CTGCCC | 0.010% | NA | no | 6 | −0.749 |
| CTGCCG | 0.011% | 0.619 | no | 6 | −0.021 |
| CTGCGA | 0.019% | 0.659 | no | 6 | 0.152 |
| CTGCGC | 0.010% | 0.585 | no | 6 | 1.570 |
| CTGCGG | 0.019% | 0.581 | no | 6 | 0.797 |
| CTGTAA | 0.023% | NA | no | 6 | −0.148 |
| CTGTAC | 0.007% | NA | no | 6 | 1.658 |
| CTGTAG | 0.013% | −0.556 | no | 6 | 1.942 |
| CTGTCA | 0.010% | NA | no | 6 | −0.657 |
| CTGTCC | 0.008% | 0.214 | no | 6 | 1.759 |
| CTGTCG | 0.008% | 0.540 | no | 6 | 1.654 |
| CTGTGA | 0.014% | 0.272 | no | 6 | −0.346 |
| CTGTGC | 0.008% | −0.312 | no | 6 | −0.669 |
| CTGTGG | 0.014% | 0.169 | no | 6 | −0.424 |
| CTTAAA | 0.017% | −0.879 | no | 6 | −1.900 |
| CTTAAC | 0.012% | −0.432 | no | 6 | −1.462 |
| CTTAAG | 0.019% | −0.427 | no | 6 | −0.539 |
| CTTACA | 0.012% | −0.564 | no | 6 | −2.129 |
| CTTACC | 0.010% | NA | no | 6 | −1.288 |
| CTTACG | 0.009% | NA | no | 6 | −0.300 |
| CTTAGA | 0.014% | −0.503 | no | 6 | −3.987 |
| CTTAGG | 0.017% | −0.727 | no | 6 | −4.087 |
| CTTCAA | 0.014% | NA | no | 6 | −0.449 |
| CTTCAC | 0.007% | NA | no | 6 | −1.351 |
| CTTCAG | 0.009% | NA | no | 6 | −1.926 |
| CTTCCA | 0.008% | NA | no | 6 | 1.026 |
| CTTCCC | 0.009% | −0.231 | no | 6 | −1.730 |
| CTTCGA | 0.009% | 0.509 | no | 6 | −0.601 |
| CTTCGC | 0.009% | 0.571 | no | 6 | −0.222 |
| CTTCGG | 0.006% | 0.540 | no | 6 | −0.297 |
| CTTTAA | 0.014% | −1.050 | no | 6 | −4.048 |
| CTTTAC | 0.006% | −0.372 | no | 6 | −2.111 |
| CTTTAG | 0.011% | −0.855 | no | 6 | −4.136 |
| CTTTCA | 0.009% | −0.561 | no | 6 | −3.027 |
| CTTTCC | 0.005% | −0.238 | no | 6 | −2.440 |
| CTTTCG | 0.007% | NA | no | 6 | −0.773 |
| CTTTGA | 0.010% | NA | no | 6 | −1.354 |
| CTTTGC | 0.005% | −0.256 | no | 6 | −0.927 |
| CTTTGG | 0.011% | NA | no | 6 | 0.375 |
| GACAAA | 0.048% | NA | no | 6 | −1.019 |
| GACAAC | 0.036% | 0.513 | no | 6 | 0.299 |
| GACAAG | 0.047% | 0.288 | no | 6 | −0.317 |
| GACACA | 0.040% | NA | no | 6 | −0.164 |
| GACACC | 0.025% | NA | no | 6 | −0.184 |
| GACACG | 0.033% | 0.493 | no | 6 | −0.023 |
| GACAGA | 0.048% | 0.414 | no | 6 | −0.131 |
| GACAGC | 0.036% | NA | no | 6 | 0.088 |
| GACAGG | 0.042% | −0.224 | no | 6 | −0.521 |
| GACCAA | 0.033% | 0.412 | no | 6 | −0.634 |
| GACCAC | 0.020% | NA | no | 6 | −0.639 |
| GACCAG | 0.027% | 0.338 | no | 6 | −0.990 |
| GACCCA | 0.021% | NA | no | 6 | −0.026 |
| GACCCC | 0.014% | NA | no | 6 | 0.042 |
| GACCCG | 0.015% | 0.625 | no | 6 | −0.042 |
| GACCGA | 0.022% | 0.598 | no | 6 | −0.695 |
| GACCGC | 0.017% | 0.342 | no | 6 | 1.073 |
| GACCGG | 0.026% | 0.647 | no | 6 | 0.166 |
| GACTAA | 0.030% | NA | no | 6 | −0.387 |
| GACTAC | 0.018% | 0.414 | no | 6 | −0.665 |
| GACTAG | 0.025% | −0.304 | no | 6 | 0.221 |
| GACTCA | 0.027% | NA | no | 6 | −1.031 |
| GACTCC | 0.012% | 0.198 | no | 6 | 1.074 |
| GACTCG | 0.021% | 0.502 | no | 6 | 0.492 |
| GACTGA | 0.025% | 0.472 | no | 6 | −0.563 |
| GACTGC | 0.018% | 0.515 | no | 6 | 0.507 |
| GACTGG | 0.025% | 0.199 | no | 6 | −0.051 |
| GAGAAA | 0.075% | 0.302 | no | 6 | −0.608 |
| GAGAAC | 0.046% | 0.504 | no | 6 | −0.072 |
| GAGAAG | 0.064% | 0.245 | no | 6 | −0.118 |
| GAGACA | 0.042% | NA | no | 6 | −0.990 |
| GAGACC | 0.025% | 0.480 | no | 6 | −0.090 |
| GAGACG | 0.036% | 0.528 | no | 6 | 0.029 |
| GAGAGA | 0.055% | 0.355 | no | 6 | −0.401 |
| GAGAGC | 0.035% | 0.462 | no | 6 | 0.030 |
| GAGAGG | 0.056% | NA | no | 6 | −0.680 |
| GAGCAA | 0.050% | NA | no | 6 | −0.602 |
| GAGCAC | 0.021% | NA | no | 6 | −0.633 |
| GAGCAG | 0.030% | NA | no | 6 | −0.687 |
| GAGCCA | 0.028% | 0.299 | no | 6 | −0.128 |
| GAGCCC | 0.012% | NA | no | 6 | −0.182 |
| GAGCCG | 0.021% | 0.367 | no | 6 | 0.563 |
| GAGCGA | 0.033% | 0.483 | no | 6 | −0.104 |
| GAGCGC | 0.023% | 0.535 | no | 6 | 0.993 |
| GAGCGG | 0.025% | 0.252 | no | 6 | 0.596 |
| GAGTAA | 0.059% | NA | no | 6 | −1.639 |
| GAGTAC | 0.030% | 0.276 | no | 6 | −0.956 |
| GAGTAG | 0.040% | −0.344 | no | 6 | −0.575 |
| GAGTCA | 0.036% | NA | no | 6 | −1.241 |
| GAGTCC | 0.016% | 0.454 | no | 6 | 2.359 |
| GAGTCG | 0.023% | 0.400 | no | 6 | −1.485 |
| GAGTGA | 0.049% | NA | no | 6 | −0.933 |
| GAGTGC | 0.023% | NA | no | 6 | −0.697 |
| GAGTGG | 0.046% | NA | no | 6 | 0.097 |
| GATAAA | 0.046% | NA | no | 6 | −2.806 |
| GATAAC | 0.025% | NA | no | 6 | −2.792 |
| GATAAG | 0.039% | NA | no | 6 | −0.480 |
| GATACA | 0.037% | NA | no | 6 | −1.321 |
| GATACC | 0.015% | 0.232 | no | 6 | −0.654 |
| GATACG | 0.028% | 0.389 | no | 6 | 0.151 |
| GATAGA | 0.036% | NA | no | 6 | −4.398 |
| GATAGC | 0.031% | NA | no | 6 | −4.817 |
| GATAGG | 0.033% | −0.688 | no | 6 | −4.666 |
| GATCAA | 0.035% | NA | no | 6 | −1.017 |
| GATCAC | 0.018% | NA | no | 6 | −1.087 |
| GATCAG | 0.030% | −0.223 | no | 6 | −0.915 |
| GATCCA | 0.021% | 0.400 | no | 6 | −0.191 |
| GATCCC | 0.010% | NA | no | 6 | −0.396 |
| GATCCG | 0.013% | 0.488 | no | 6 | −0.810 |
| GATCGA | 0.025% | 0.518 | no | 6 | 0.807 |
| GATCGC | 0.018% | 0.392 | no | 6 | 0.474 |
| GATCGG | 0.024% | 0.443 | no | 6 | −0.459 |
| GATTAA | 0.032% | −0.297 | no | 6 | −2.387 |
| GATTAC | 0.016% | NA | no | 6 | −2.926 |
| GATTAG | 0.028% | −0.496 | no | 6 | −2.360 |
| GATTCA | 0.017% | NA | no | 6 | −3.109 |
| GATTCC | 0.010% | 0.223 | no | 6 | −2.216 |
| GATTCG | 0.015% | 0.518 | no | 6 | −1.842 |
| GATTGA | 0.035% | 0.284 | no | 6 | −1.102 |
| GATTGC | 0.017% | 0.123 | no | 6 | 0.027 |
| GATTGG | 0.031% | NA | no | 6 | 0.014 |
| GCCAAA | 0.039% | −0.438 | no | 6 | −0.555 |
| GCCAAC | 0.018% | NA | no | 6 | −1.562 |
| GCCAAG | 0.018% | NA | no | 6 | 0.564 |
| GCCACA | 0.023% | NA | no | 6 | −1.006 |
| GCCACC | 0.012% | −0.311 | no | 6 | 0.422 |
| GCCACG | 0.023% | NA | no | 6 | −0.096 |
| GCCAGA | 0.032% | 0.072 | no | 6 | −0.378 |
| GCCAGC | 0.021% | −0.294 | no | 6 | −0.658 |
| GCCAGG | 0.026% | −0.320 | no | 6 | −0.140 |
| GCCCAA | 0.020% | −0.363 | no | 6 | −0.822 |
| GCCCAG | 0.016% | NA | no | 6 | −0.433 |
| GCCCCA | 0.011% | −0.238 | no | 6 | −1.599 |
| GCCCCC | 0.007% | −0.212 | no | 6 | −1.362 |
| GCCCCG | 0.010% | NA | no | 6 | 0.358 |
| GCCCGA | 0.016% | NA | no | 6 | 0.050 |
| GCCCGC | 0.010% | NA | no | 6 | −0.827 |
| GCCCGG | 0.012% | 0.337 | no | 6 | −0.658 |
| GCCTAA | 0.021% | −0.647 | no | 6 | −1.440 |
| GCCTAC | 0.012% | NA | no | 6 | −0.634 |
| GCCTAG | 0.014% | −0.583 | no | 6 | −0.571 |
| GCCTCA | 0.017% | NA | no | 6 | −1.086 |
| GCCTCC | 0.009% | NA | no | 6 | −0.243 |
| GCCTCG | 0.015% | 0.277 | no | 6 | −0.446 |
| GCCTGA | 0.018% | NA | no | 6 | −0.038 |
| GCCTGC | 0.012% | 0.260 | no | 6 | −0.509 |
| GCCTGG | 0.017% | 0.174 | no | 6 | −0.392 |
| GCGAAA | 0.045% | NA | no | 6 | −0.470 |
| GCGAAC | 0.028% | 0.478 | no | 6 | −0.177 |
| GCGAAG | 0.043% | 0.358 | no | 6 | 0.338 |
| GCGACA | 0.028% | NA | no | 6 | −0.690 |
| GCGACC | 0.016% | 0.595 | no | 6 | 0.321 |
| GCGACG | 0.023% | 0.718 | no | 6 | 0.170 |
| GCGAGA | 0.034% | 0.353 | no | 6 | −0.578 |
| GCGAGC | 0.023% | 0.331 | no | 6 | −0.297 |
| GCGAGG | 0.031% | NA | no | 6 | −0.506 |
| GCGCAA | 0.035% | NA | no | 6 | −0.479 |
| GCGCAC | 0.016% | 0.375 | no | 6 | −0.288 |
| GCGCAG | 0.021% | NA | no | 6 | −0.199 |
| GCGCCA | 0.022% | 0.434 | no | 6 | −0.253 |
| GCGCCC | 0.010% | 0.289 | no | 6 | −0.851 |
| GCGCCG | 0.010% | 0.536 | no | 6 | 1.066 |
| GCGCGA | 0.029% | 0.506 | no | 6 | −0.952 |
| GCGCGC | 0.010% | 0.556 | no | 6 | −0.880 |
| GCGCGG | 0.017% | 0.462 | no | 6 | −0.572 |
| GCGTAA | 0.034% | NA | no | 6 | −0.710 |
| GCGTAC | 0.017% | NA | no | 6 | −0.423 |
| GCGTAG | 0.022% | −0.428 | no | 6 | 0.175 |
| GCGTCA | 0.020% | NA | no | 6 | −0.702 |
| GCGTCC | 0.012% | 0.618 | no | 6 | −1.056 |
| GCGTCG | 0.014% | 0.676 | no | 6 | 0.218 |
| GCGTGA | 0.024% | NA | no | 6 | 0.073 |
| GCGTGC | 0.021% | 0.128 | no | 6 | 0.219 |
| GCGTGG | 0.023% | 0.287 | no | 6 | 0.105 |
| GCTAAA | 0.032% | NA | no | 6 | −1.867 |
| GCTAAC | 0.017% | NA | no | 6 | −1.272 |
| GCTAAG | 0.027% | −0.416 | no | 6 | 0.145 |
| GCTACA | 0.019% | NA | no | 6 | −0.911 |
| GCTACC | 0.014% | NA | no | 6 | −0.258 |
| GCTACG | 0.023% | 0.351 | no | 6 | −0.409 |
| GCTAGA | 0.024% | NA | no | 6 | −3.703 |
| GCTAGC | 0.014% | −0.448 | no | 6 | −4.537 |
| GCTAGG | 0.029% | −0.764 | no | 6 | −5.587 |
| GCTCAA | 0.027% | NA | no | 6 | −0.588 |
| GCTCAC | 0.018% | NA | no | 6 | 0.296 |
| GCTCAG | 0.021% | −0.343 | no | 6 | −1.498 |
| GCTCCA | 0.021% | NA | no | 6 | −0.338 |
| GCTCCC | 0.010% | NA | no | 6 | 0.651 |
| GCTCCG | 0.010% | 0.283 | no | 6 | 0.669 |
| GCTCGA | 0.018% | NA | no | 6 | −1.157 |
| GCTCGC | 0.014% | NA | no | 6 | −0.417 |
| GCTCGG | 0.017% | 0.150 | no | 6 | −0.412 |
| GCTTAA | 0.026% | −0.508 | no | 6 | −2.096 |
| GCTTAC | 0.014% | NA | no | 6 | −1.977 |
| GCTTAG | 0.019% | −0.606 | no | 6 | −2.010 |
| GCTTCA | 0.014% | NA | no | 6 | −0.754 |
| GCTTCG | 0.014% | 0.412 | no | 6 | −0.136 |
| GCTTGA | 0.019% | NA | no | 6 | −1.411 |
| GCTTGC | 0.012% | −0.236 | no | 6 | −0.179 |
| GCTTGG | 0.012% | NA | no | 6 | 1.196 |
| GTCAAA | 0.045% | NA | no | 6 | −0.412 |
| GTCAAC | 0.020% | NA | no | 6 | 0.672 |
| GTCAAG | 0.032% | NA | no | 6 | −0.189 |
| GTCACA | 0.030% | NA | no | 6 | −1.449 |
| GTCACC | 0.020% | NA | no | 6 | −0.815 |
| GTCACG | 0.024% | NA | no | 6 | −0.600 |
| GTCAGA | 0.039% | NA | no | 6 | −0.765 |
| GTCAGC | 0.020% | −0.251 | no | 6 | −1.363 |
| GTCAGG | 0.044% | −0.467 | no | 6 | −1.558 |
| GTCCAA | 0.023% | NA | no | 6 | −1.184 |
| GTCCAC | 0.015% | NA | no | 6 | −0.609 |
| GTCCAG | 0.023% | 0.249 | no | 6 | −0.943 |
| GTCCCA | 0.014% | −0.124 | no | 6 | −1.573 |
| GTCCCC | 0.013% | −0.215 | no | 6 | −0.382 |
| GTCCCG | 0.011% | 0.260 | no | 6 | 0.504 |
| GTCCGA | 0.014% | NA | no | 6 | −0.097 |
| GTCCGC | 0.010% | NA | no | 6 | 1.157 |
| GTCCGG | 0.015% | 0.422 | no | 6 | 0.354 |
| GTCTAA | 0.020% | −0.418 | no | 6 | 0.162 |
| GTCTAC | 0.013% | NA | no | 6 | −1.077 |
| GTCTAG | 0.019% | −0.552 | no | 6 | −0.533 |
| GTCTCA | 0.016% | NA | no | 6 | −1.110 |
| GTCTCC | 0.012% | NA | no | 6 | −0.588 |
| GTCTCG | 0.019% | 0.226 | no | 6 | 0.787 |
| GTCTGA | 0.023% | NA | no | 6 | 0.604 |
| GTCTGC | 0.018% | 0.308 | no | 6 | −4.146 |
| GTCTGG | 0.024% | NA | no | 6 | 0.134 |
| GTGAAA | 0.050% | 0.237 | yes | 6 | −3.116 |
| GTGAAC | 0.035% | 0.362 | yes | 6 | −2.820 |
| GTGAAG | 0.047% | 0.561 | yes | 6 | −2.380 |
| GTGACA | 0.037% | 0.119 | yes | 6 | −2.181 |
| GTGACC | 0.017% | 0.335 | yes | 6 | −1.395 |
| GTGACG | 0.023% | 0.584 | yes | 6 | −3.743 |
| GTGAGA | 0.046% | 0.363 | yes | 6 | −3.601 |
| GTGAGC | 0.031% | NA | yes | 6 | −3.323 |
| GTGAGG | 0.047% | NA | yes | 6 | −1.848 |
| GTGCAA | 0.034% | −0.448 | no | 6 | −1.188 |
| GTGCAC | 0.027% | −0.066 | no | 6 | −0.335 |
| GTGCAG | 0.024% | −0.378 | no | 6 | −0.583 |
| GTGCCA | 0.019% | −0.185 | no | 6 | 0.193 |
| GTGCCC | 0.013% | −0.378 | no | 6 | 1.213 |
| GTGCCG | 0.013% | NA | no | 6 | −0.739 |
| GTGCGA | 0.023% | NA | no | 6 | 0.420 |
| GTGCGC | 0.013% | NA | no | 6 | −0.636 |
| GTGCGG | 0.030% | NA | no | 6 | 0.075 |
| GTGTAA | 0.037% | −0.335 | no | 6 | −1.019 |
| GTGTAC | 0.025% | NA | no | 6 | −0.619 |
| GTGTAG | 0.024% | −0.504 | no | 6 | −0.551 |
| GTGTCA | 0.029% | −0.246 | no | 6 | −1.512 |
| GTGTCC | 0.013% | NA | no | 6 | 0.463 |
| GTGTCG | 0.020% | 0.188 | no | 6 | −0.356 |
| GTGTGA | 0.036% | 0.125 | no | 6 | −0.522 |
| GTGTGC | 0.022% | −0.174 | no | 6 | 0.097 |
| GTGTGG | 0.024% | NA | no | 6 | 0.418 |
| GTTAAA | 0.041% | −0.232 | no | 6 | −3.467 |
| GTTAAC | 0.024% | NA | no | 6 | −3.293 |
| GTTAAG | 0.036% | −0.390 | no | 6 | −0.661 |
| GTTACA | 0.032% | NA | no | 6 | −3.654 |
| GTTACC | 0.016% | 0.258 | no | 6 | −2.418 |
| GTTACG | 0.017% | 0.226 | no | 6 | −0.631 |
| GTTAGA | 0.031% | −0.551 | no | 6 | −6.895 |
| GTTAGC | 0.026% | −0.483 | no | 6 | −3.657 |
| GTTAGG | 0.028% | −0.831 | no | 6 | −9.046 |
| GTTCAA | 0.025% | 0.224 | no | 6 | −1.432 |
| GTTCAC | 0.015% | 0.275 | no | 6 | −0.576 |
| GTTCAG | 0.022% | NA | no | 6 | −3.395 |
| GTTCCA | 0.019% | 0.146 | no | 6 | −0.906 |
| GTTCCC | 0.009% | −0.385 | no | 6 | −1.074 |
| GTTCCG | 0.008% | 0.212 | no | 6 | 1.510 |
| GTTCGA | 0.026% | 0.508 | no | 6 | −1.121 |
| GTTCGC | 0.015% | 0.589 | no | 6 | −1.492 |
| GTTCGG | 0.016% | 0.354 | no | 6 | −0.379 |
| GTTTAA | 0.035% | −0.296 | no | 6 | −3.228 |
| GTTTAC | 0.015% | NA | no | 6 | −2.741 |
| GTTTAG | 0.026% | −0.622 | no | 6 | −2.072 |
| GTTTCA | 0.019% | NA | no | 6 | −4.684 |
| GTTTCC | 0.008% | 0.180 | no | 6 | −1.709 |
| GTTTCG | 0.015% | 0.367 | no | 6 | 0.269 |
| GTTTGA | 0.025% | NA | no | 6 | −1.573 |
| GTTTGC | 0.015% | NA | no | 6 | −1.237 |
| GTTTGG | 0.019% | −0.200 | no | 6 | 0.065 |

TABLE 3
Effect sizes for the effects of each individual SNV in exon 18 of BRCA1 on exon splicing/stability

| | | | | | | Library | Library | Library | Library | Library | Library |
|---|---|---|---|---|---|---|---|---|---|---|---|
| | | Average | | | | R1 | R1 | R | R | L | L |
| | | effect | | | | Repli- | Repli- | Repli- | Repli- | Repli- | Repli- |
| | | size (both | | | | cate 1 | cate 2 | cate 1 | cate 2 | cate 1 | cate 2 |
| Exon | | reps of | MutPredSplice | | Mutation | effect | effect | effect | effect | effect | effect |
| Position | Variant | L and R) | score | MutPredSplice output | Type | size | size | size | size | size | size |
| 1 | C | −0.476 | 0.82 | Splice Affecting Variant (SAV) | sense | NA | NA | NA | NA | −0.577 | −0.376 |
| | | | | Loss of natural 3′ SS | | | | | | | |
| | | | | (P < 0.000001) | | | | | | | |
| 1 | G | −0.493 | 0.9 | Splice Affecting Variant (SAV) | sense | NA | NA | NA | NA | −0.839 | −0.147 |
| 1 | T | −0.661 | 0.85 | Splice Affecting Variant (SAV) | sense | NA | NA | NA | NA | −1.048 | −0.274 |
| | | | | Loss of natural 3′ SS | | | | | | | |
| | | | | (P < 0.000001) | | | | | | | |
| 2 | A | −0.504 | 0.83 | Splice Affecting Variant (SAV) | sense | NA | NA | NA | NA | −0.529 | −0.478 |
| | | | | Loss of natural 3′ SS | | | | | | | |
| | | | | (P < 0.000001) | | | | | | | |
| 2 | C | −0.630 | 0.85 | Splice Affecting Variant (SAV) | sense | NA | NA | NA | NA | −0.746 | −0.513 |
| | | | | Loss of natural 3′ SS | | | | | | | |
| | | | | (P < 0.000001) | | | | | | | |
| 2 | G | −0.535 | 0.85 | Splice Affecting Variant (SAV) | sense | NA | NA | NA | NA | −0.791 | −0.278 |
| | | | | Loss of natural 3′ SS | | | | | | | |
| | | | | (P < 0.000001) | | | | | | | |
| 3 | A | −0.494 | 0.77 | Splice Affecting Variant (SAV) | sense | NA | NA | NA | NA | −0.528 | −0.460 |
| 3 | C | −0.300 | 0.65 | Splice Affecting Variant (SAV) | sense | NA | NA | NA | NA | −0.022 | −0.579 |
| 3 | T | −0.466 | 0.8 | Splice Affecting Variant (SAV) | sense | NA | NA | NA | NA | −0.563 | −0.369 |
| 4 | A | −0.119 | 0.52 | Splice Neutral Variant (SNV) | sense | NA | NA | NA | NA | −0.232 | −0.007 |
| 4 | G | −0.432 | 0.95 | Splice Affecting Variant (SAV) | sense | NA | NA | NA | NA | −0.369 | −0.495 |
| | | | | Cryptic 5′ SS (P = 0.001003) | | | | | | | |
| 4 | T | −0.547 | 0.62 | Splice Affecting Variant (SAV) | sense | NA | NA | NA | NA | −0.300 | −0.794 |
| 5 | A | −0.399 | 0.34 | Splice Neutral Variant (SNV) | sense | NA | NA | NA | NA | −0.599 | −0.198 |
| 5 | C | −0.841 | 0.39 | Splice Neutral Variant (SNV) | sense | NA | NA | NA | NA | −0.824 | −0.859 |
| 5 | G | −0.387 | 0.4 | Splice Neutral Variant (SNV) | sense | NA | NA | NA | NA | −0.437 | −0.338 |
| 6 | A | −0.436 | 0.79 | Splice Affecting Variant (SAV) | sense | NA | NA | NA | NA | −0.330 | −0.543 |
| 6 | C | −0.192 | 0.53 | Splice Neutral Variant (SNV) | sense | NA | NA | NA | NA | −0.326 | −0.058 |
| 6 | T | −2.084 | 0.88 | Splice Affecting Variant (SAV) | nonsense | NA | NA | NA | NA | −2.001 | −2.167 |
| | | | | ESS Gain (P < 0.000001) | | | | | | | |
| 7 | C | 0.044 | 0.39 | Splice Neutral Variant (SNV) | sense | NA | NA | NA | NA | 0.023 | 0.065 |
| 7 | G | −0.192 | 0.74 | Splice Affecting Variant (SAV) | sense | NA | NA | NA | NA | −0.293 | −0.091 |
| 7 | T | −0.246 | 0.53 | Splice Neutral Variant (SNV) | sense | NA | NA | NA | NA | −0.440 | −0.052 |
| 8 | A | −0.039 | 0.28 | Splice Neutral Variant (SNV) | sense | NA | NA | NA | NA | 0.173 | −0.251 |
| 8 | C | −0.675 | 0.28 | Splice Neutral Variant (SNV) | sense | NA | NA | NA | NA | −1.104 | −0.246 |
| 8 | T | 0.092 | 0.35 | Splice Neutral Variant (SNV) | sense | NA | NA | NA | NA | 0.084 | 0.100 |
| 9 | A | −0.342 | 0.3 | Splice Neutral Variant (SNV) | sense | NA | NA | NA | NA | −0.551 | −0.134 |
| 9 | C | −0.269 | 0.29 | Splice Neutral Variant (SNV) | sense | NA | NA | NA | NA | −0.063 | −0.475 |
| 9 | G | −0.294 | 0.48 | Splice Neutral Variant (SNV) | sense | NA | NA | NA | NA | −0.288 | −0.301 |
| 10 | A | −0.211 | 0.74 | Splice Affecting Variant (SAV) | sense | NA | NA | NA | NA | −0.566 | 0.143 |
| 10 | C | −0.027 | 0.35 | Splice Neutral Variant (SNV) | sense | NA | NA | NA | NA | −0.457 | 0.403 |
| 10 | G | −0.138 | 0.39 | Splice Neutral Variant (SNV) | sense | NA | NA | NA | NA | −0.234 | −0.043 |
| 11 | A | −0.735 | 0.66 | Splice Affecting Variant (SAV) | sense | NA | NA | NA | NA | −0.644 | −0.827 |
| 11 | C | −0.391 | 0.35 | Splice Neutral Variant (SNV) | sense | NA | NA | NA | NA | −0.277 | −0.504 |
| 11 | G | −0.171 | 0.72 | Splice Affecting Variant (SAV) | sense | NA | NA | NA | NA | −0.169 | −0.172 |
| 12 | A | −0.191 | 0.56 | Splice Neutral Variant (SNV) | sense | NA | NA | NA | NA | −0.355 | −0.027 |
| 12 | C | −0.152 | 0.43 | Splice Neutral Variant (SNV) | sense | NA | NA | NA | NA | −0.301 | −0.004 |
| 12 | T | −0.321 | 0.53 | Splice Neutral Variant (SNV) | sense | NA | NA | NA | NA | −0.439 | −0.203 |
| 13 | A | −0.088 | 0.44 | Splice Neutral Variant (SNV) | sense | NA | NA | NA | NA | 0.001 | −0.178 |
| 13 | C | −0.036 | 0.31 | Splice Neutral Variant (SNV) | sense | NA | NA | NA | NA | −0.073 | 0.002 |
| 13 | G | −0.052 | 0.66 | Splice Affecting Variant (SAV) | sense | NA | NA | NA | NA | −0.160 | 0.057 |
| 14 | A | −0.074 | 0.76 | Splice Affecting Variant (SAV) | sense | NA | NA | NA | NA | −0.009 | −0.139 |
| 14 | C | −0.034 | 0.36 | Splice Neutral Variant (SNV) | sense | NA | NA | NA | NA | −0.153 | 0.085 |
| 14 | T | −0.068 | 0.55 | Splice Neutral Variant (SNV) | sense | NA | NA | NA | NA | −0.007 | −0.130 |
| 15 | A | 0.195 | 0.77 | Splice Affecting Variant (SAV) | sense | NA | NA | NA | NA | 0.058 | 0.332 |
| | | | | Cryptic 5′ SS (P = 0.015766) | | | | | | | |
| 15 | C | −0.047 | 0.34 | Splice Neutral Variant (SNV) | sense | NA | NA | NA | NA | −0.009 | −0.084 |
| 15 | G | −0.111 | 0.56 | Splice Neutral Variant (SNV) | sense | NA | NA | NA | NA | −0.185 | −0.038 |
| 16 | A | 0.148 | 0.6 | Splice Affecting Variant (SAV) | sense | NA | NA | NA | NA | 0.103 | 0.192 |
| 16 | C | −0.020 | 0.33 | Splice Neutral Variant (SNV) | sense | NA | NA | NA | NA | −0.081 | 0.040 |
| 16 | T | −0.047 | 0.59 | Splice Neutral Variant (SNV) | sense | NA | NA | NA | NA | −0.123 | 0.028 |
| 17 | A | −1.387 | 0.4 | Splice Neutral Variant (SNV) | nonsense | NA | NA | NA | NA | −1.483 | −1.292 |
| 17 | C | −0.058 | 0.35 | Splice Neutral Variant (SNV) | sense | NA | NA | NA | NA | −0.038 | −0.078 |
| 17 | G | −0.074 | 0.36 | Splice Neutral Variant (SNV) | sense | NA | NA | NA | NA | −0.085 | −0.063 |
| 18 | A | −0.028 | 0.77 | Splice Affecting Variant (SAV) | sense | NA | NA | NA | NA | 0.101 | −0.156 |
| 18 | C | −0.194 | 0.36 | Splice Neutral Variant (SNV) | sense | NA | NA | NA | NA | −0.368 | −0.021 |
| 18 | T | −1.699 | 0.84 | Splice Affecting Variant (SAV) | nonsense | NA | NA | NA | NA | −1.657 | −1.740 |
| | | | | ESE Loss (P = 0.046524) | | | | | | | |
| | | | | ESS Gain (P = 0.000732) | | | | | | | |
| 19 | C | −0.018 | 0.4 | Splice Neutral Variant (SNV) | sense | NA | NA | NA | NA | −0.034 | −0.003 |
| 19 | G | −0.069 | 0.39 | Splice Neutral Variant (SNV) | sense | NA | NA | NA | NA | −0.117 | −0.021 |
| 19 | T | 0.226 | 0.77 | Splice Affecting Variant (SAV) | sense | NA | NA | NA | NA | 0.238 | 0.214 |
| | | | | ESE Loss (P = 0.004064) | | | | | | | |
| 20 | C | 0.054 | 0.33 | Splice Neutral Variant (SNV) | sense | NA | NA | NA | NA | 0.047 | 0.060 |
| 20 | G | 0.049 | 0.49 | Splice Neutral Variant (SNV) | sense | NA | NA | NA | NA | −0.096 | 0.193 |
| 20 | T | 0.131 | 0.49 | Splice Neutral Variant (SNV) | sense | NA | NA | NA | NA | 0.059 | 0.204 |
| 21 | A | 0.194 | 0.42 | Splice Neutral Variant (SNV) | sense | NA | NA | NA | NA | 0.000 | 0.389 |
| 21 | G | 0.005 | 0.58 | Splice Neutral Variant (SNV) | sense | NA | NA | NA | NA | 0.005 | 0.005 |
| 21 | T | −0.529 | 0.53 | Splice Neutral Variant (SNV) | sense | NA | NA | NA | NA | −1.231 | 0.174 |
| 22 | A | 0.169 | 0.41 | Splice Neutral Variant (SNV) | sense | NA | NA | NA | NA | 0.225 | 0.114 |
| 22 | C | 0.064 | 0.33 | Splice Neutral Variant (SNV) | sense | NA | NA | NA | NA | 0.113 | 0.015 |
| 22 | T | −0.232 | 0.37 | Splice Neutral Variant (SNV) | sense | NA | NA | NA | NA | −0.272 | −0.193 |
| 23 | A | 0.134 | 0.39 | Splice Neutral Variant (SNV) | sense | NA | NA | NA | NA | 0.047 | 0.221 |
| 23 | C | 0.270 | 0.58 | Splice Neutral Variant (SNV) | sense | NA | NA | NA | NA | 0.419 | 0.121 |
| 23 | T | 0.164 | 0.58 | Splice Neutral Variant (SNV) | sense | NA | NA | NA | NA | 0.254 | 0.075 |
| 24 | C | 0.187 | 0.59 | Splice Neutral Variant (SNV) | sense | NA | NA | NA | NA | 0.040 | 0.335 |
| 24 | G | 0.161 | 0.54 | Splice Neutral Variant (SNV) | sense | NA | NA | NA | NA | 0.132 | 0.190 |
| 24 | T | 0.071 | 0.67 | Splice Affecting Variant (SAV) | sense | NA | NA | NA | NA | 0.062 | 0.081 |
| | | | | ESE Loss (P = 0.000173) | | | | | | | |
| 25 | A | 0.098 | 0.35 | Splice Neutral Variant (SNV) | sense | NA | NA | NA | NA | 0.241 | −0.044 |
| 25 | G | 0.046 | 0.35 | Splice Neutral Variant (SNV) | sense | NA | NA | NA | NA | −0.041 | 0.133 |
| 25 | T | −0.084 | 0.57 | Splice Neutral Variant (SNV) | sense | NA | NA | NA | NA | −0.145 | −0.023 |
| 26 | C | 0.031 | 0.29 | Splice Neutral Variant (SNV) | sense | NA | NA | NA | NA | 0.167 | −0.105 |
| 26 | G | −0.155 | 0.39 | Splice Neutral Variant (SNV) | sense | NA | NA | NA | NA | −0.177 | −0.134 |
| 26 | T | −0.088 | 0.34 | Splice Neutral Variant (SNV) | sense | NA | NA | NA | NA | −0.041 | −0.134 |
| 27 | A | −0.154 | 0.44 | Splice Neutral Variant (SNV) | sense | NA | NA | NA | NA | −0.216 | −0.091 |
| 27 | G | 0.235 | 0.5 | Splice Neutral Variant (SNV) | sense | NA | NA | NA | NA | 0.132 | 0.337 |
| 27 | T | −0.144 | 0.36 | Splice Neutral Variant (SNV) | sense | NA | NA | NA | NA | −0.173 | −0.116 |
| 28 | A | −0.071 | 0.36 | Splice Neutral Variant (SNV) | sense | NA | NA | NA | NA | −0.017 | −0.125 |
| 28 | C | −0.334 | 0.34 | Splice Neutral Variant (SNV) | sense | NA | NA | NA | NA | −0.646 | −0.022 |
| 28 | G | −0.181 | 0.37 | Splice Neutral Variant (SNV) | sense | NA | NA | NA | NA | −0.363 | 0.002 |
| 29 | A | 0.000 | 0.76 | Splice Affecting Variant (SAV) | sense | NA | NA | NA | NA | 0.082 | −0.081 |
| | | | | ESE Loss (P = 0.046524) | | | | | | | |
| | | | | ESS Gain (P = 0.034846) | | | | | | | |
| 29 | C | 0.010 | 0.48 | Splice Neutral Variant (SNV) | sense | NA | NA | NA | NA | 0.073 | −0.053 |
| 29 | T | −0.537 | 0.8 | Splice Affecting Variant (SAV) | sense | NA | NA | NA | NA | −0.637 | −0.436 |
| | | | | ESE Loss (P = 0.046524) | | | | | | | |
| | | | | ESS Gain (P = 0.034846) | | | | | | | |
| 30 | C | −0.039 | 0.43 | Splice Neutral Variant (SNV) | sense | NA | NA | NA | NA | −0.279 | 0.201 |
| 30 | G | 0.109 | 0.31 | Splice Neutral Variant (SNV) | sense | NA | NA | NA | NA | 0.284 | −0.065 |
| 30 | T | −1.353 | 0.82 | Splice Affecting Variant (SAV) | nonsense | NA | NA | NA | NA | −1.626 | −1.081 |
| | | | | Cryptic 3′ SS (P = 0.03882) | | | | | | | |
| 31 | C | −0.205 | 0.37 | Splice Neutral Variant (SNV) | sense | NA | NA | NA | NA | −0.211 | −0.199 |
| 31 | G | 0.029 | 0.33 | Splice Neutral Variant (SNV) | sense | NA | NA | NA | NA | −0.102 | 0.160 |
| 31 | T | −0.006 | 0.49 | Splice Neutral Variant (SNV) | sense | NA | NA | NA | NA | −0.074 | 0.062 |
| 32 | C | 0.228 | 0.28 | Splice Neutral Variant (SNV) | sense | NA | NA | NA | NA | 0.367 | 0.088 |
| 32 | G | −0.067 | 0.45 | Splice Neutral Variant (SNV) | sense | NA | NA | NA | NA | −0.015 | −0.118 |
| 32 | T | −0.097 | 0.38 | Splice Neutral Variant (SNV) | sense | NA | NA | NA | NA | −0.006 | −0.188 |
| 33 | A | 0.189 | 0.28 | Splice Neutral Variant (SNV) | sense | NA | NA | NA | NA | 0.023 | 0.355 |
| 33 | C | −0.057 | 0.26 | Splice Neutral Variant (SNV) | sense | NA | NA | NA | NA | 0.055 | −0.169 |
| 33 | G | −0.078 | 0.28 | Splice Neutral Variant (SNV) | sense | NA | NA | NA | NA | −0.683 | 0.527 |
| 34 | C | 0.085 | 0.31 | Splice Neutral Variant (SNV) | sense | NA | NA | NA | NA | 0.066 | 0.104 |
| 34 | G | 0.207 | 0.45 | Splice Neutral Variant (SNV) | sense | NA | NA | NA | NA | 0.163 | 0.252 |
| 34 | T | −0.180 | 0.37 | Splice Neutral Variant (SNV) | sense | NA | NA | NA | NA | −0.300 | −0.061 |
| 35 | A | −1.130 | 0.36 | Splice Neutral Variant (SNV) | nonsense | NA | NA | NA | NA | −0.842 | −1.417 |
| 35 | C | 0.041 | 0.29 | Splice Neutral Variant (SNV) | sense | NA | NA | NA | NA | 0.087 | −0.004 |
| 35 | G | −1.279 | 0.51 | Splice Neutral Variant (SNV) | nonsense | NA | NA | NA | NA | −1.364 | −1.195 |
| 36 | A | −0.023 | 0.36 | Splice Neutral Variant (SNV) | sense | NA | NA | NA | NA | −0.065 | 0.018 |
| 36 | C | 0.039 | 0.32 | Splice Neutral Variant (SNV) | sense | NA | NA | NA | NA | 0.243 | −0.165 |
| 36 | G | 0.024 | 0.37 | Splice Neutral Variant (SNV) | sense | NA | NA | NA | NA | −0.028 | 0.077 |
| 37 | A | 0.111 | 0.37 | Splice Neutral Variant (SNV) | sense | NA | NA | NA | NA | 0.170 | 0.052 |
| 37 | C | −0.137 | 0.33 | Splice Neutral Variant (SNV) | sense | NA | NA | NA | NA | −0.455 | 0.180 |
| 37 | G | −0.309 | 0.33 | Splice Neutral Variant (SNV) | sense | NA | NA | NA | NA | −0.270 | −0.347 |
| 38 | A | −0.010 | 0.42 | Splice Neutral Variant (SNV) | sense | NA | NA | NA | NA | −0.295 | 0.276 |
| 38 | C | 0.100 | 0.34 | Splice Neutral Variant (SNV) | sense | NA | NA | NA | NA | 0.050 | 0.149 |
| 38 | G | −0.146 | 0.36 | Splice Neutral Variant (SNV) | sense | NA | NA | NA | NA | −0.382 | 0.090 |
| 39 | A | −0.034 | 0.44 | Splice Neutral Variant (SNV) | sense | NA | NA | NA | NA | −0.106 | 0.038 |
| 39 | G | −0.073 | 0.47 | Splice Neutral Variant (SNV) | sense | NA | NA | NA | NA | −0.053 | −0.093 |
| 39 | T | −0.130 | 0.49 | Splice Neutral Variant (SNV) | sense | NA | NA | NA | NA | 0.055 | −0.315 |
| 40 | A | 0.273 | 0.31 | Splice Neutral Variant (SNV) | sense | 0.857 | −0.291 | 0.164 | 1.299 | −0.084 | −0.285 |
| 40 | C | 0.157 | 0.35 | Splice Neutral Variant (SNV) | sense | 1.050 | 0.107 | 0.496 | 0.146 | −0.075 | 0.063 |
| 40 | G | 0.031 | 0.32 | Splice Neutral Variant (SNV) | sense | 1.239 | 0.694 | −0.122 | 0.185 | −0.102 | 0.164 |
| 41 | C | −0.008 | 0.32 | Splice Neutral Variant (SNV) | sense | 0.475 | 0.340 | 0.578 | −0.178 | −0.212 | −0.219 |
| 41 | G | 0.057 | 0.4 | Splice Neutral Variant (SNV) | sense | 0.042 | 0.078 | 0.515 | 0.253 | −0.030 | −0.509 |
| 41 | T | 0.094 | 0.32 | Splice Neutral Variant (SNV) | nonsense | −0.027 | 0.287 | 0.170 | 0.267 | −0.101 | 0.042 |
| 42 | A | −0.166 | 0.42 | Splice Neutral Variant (SNV) | sense | −0.084 | 0.671 | −0.288 | 0.034 | −0.413 | 0.004 |
| 42 | C | 0.102 | 0.36 | Splice Neutral Variant (SNV) | sense | 0.308 | 0.090 | 0.109 | 0.139 | −0.154 | 0.312 |
| 42 | T | −1.417 | 0.48 | Splice Neutral Variant (SNV) | sense | −1.140 | −0.760 | −1.491 | −1.723 | −1.501 | −0.952 |
| 43 | A | 0.117 | 0.34 | Splice Neutral Variant (SNV) | sense | 0.145 | 0.495 | −0.119 | 0.195 | 0.065 | 0.326 |
| 43 | C | 0.151 | 0.42 | Splice Neutral Variant (SNV) | sense | 0.046 | 0.608 | 0.110 | 0.431 | −0.057 | 0.119 |
| 43 | T | −0.377 | 0.8 | Splice Affecting Variant (SAV) | sense | −0.071 | −0.219 | −1.125 | −0.950 | 0.162 | 0.404 |
| 44 | C | −0.047 | 0.48 | Splice Neutral Variant (SNV) | sense | 0.044 | 0.120 | −0.235 | 0.157 | 0.293 | −0.402 |
| 44 | G | −0.824 | 0.53 | Splice Neutral Variant (SNV) | sense | 0.041 | 0.049 | −1.455 | −0.786 | −0.884 | −0.171 |
| 44 | T | −0.616 | 0.96 | Splice Affecting Variant (SAV) | sense | −0.162 | 0.335 | −1.208 | −0.547 | −0.569 | −0.141 |
| | | | | ESS Gain (P = 0.000003) | | | | | | | |
| | | | | Cryptic 5′ SS (P = 0.018361) | | | | | | | |
| 45 | C | 0.114 | 0.37 | Splice Neutral Variant (SNV) | sense | 0.079 | 0.109 | 0.087 | 0.141 | NA | NA |
| 45 | G | −0.257 | 0.42 | Splice Neutral Variant (SNV) | sense | 0.178 | 0.241 | −0.316 | −0.198 | NA | NA |
| 45 | T | 0.120 | 0.46 | Splice Neutral Variant (SNV) | sense | 0.046 | −0.011 | 0.029 | 0.211 | NA | NA |
| 46 | A | 0.055 | 0.43 | Splice Neutral Variant (SNV) | sense | 0.352 | 0.488 | −0.328 | 0.437 | NA | NA |
| 46 | C | 0.966 | 0.24 | Splice Neutral Variant (SNV) | sense | 0.244 | 0.316 | 0.550 | 1.382 | NA | NA |
| 46 | G | 0.071 | 0.36 | Splice Neutral Variant (SNV) | sense | 0.207 | 0.183 | −0.030 | 0.173 | NA | NA |
| 47 | A | 0.149 | 0.46 | Splice Neutral Variant (SNV) | sense | 0.064 | −0.220 | 0.395 | −0.097 | NA | NA |
| 47 | C | 0.234 | 0.26 | Splice Neutral Variant (SNV) | sense | 0.252 | 0.660 | −0.031 | 0.499 | NA | NA |
| 47 | G | −0.494 | 0.35 | Splice Neutral Variant (SNV) | sense | −0.272 | 0.232 | −0.588 | −0.400 | NA | NA |
| 48 | A | 0.211 | 0.42 | Splice Neutral Variant (SNV) | sense | −0.183 | 0.321 | 0.127 | 0.294 | NA | NA |
| 48 | C | 0.222 | 0.3 | Splice Neutral Variant (SNV) | sense | 0.099 | 0.208 | 0.031 | 0.414 | NA | NA |
| 48 | T | 0.138 | 0.37 | Splice Neutral Variant (SNV) | sense | 0.072 | 0.146 | 0.153 | 0.124 | NA | NA |
| 49 | A | −0.759 | 0.36 | Splice Neutral Variant (SNV) | sense | −0.332 | 0.288 | −0.610 | −0.909 | NA | NA |
| 49 | G | −2.574 | 0.61 | Splice Affecting Variant (SAV) | sense | −1.447 | −1.282 | −2.905 | −2.243 | NA | NA |
| 49 | T | −0.784 | 0.48 | Splice Neutral Variant (SNV) | sense | −0.264 | −0.202 | −0.877 | −0.692 | NA | NA |
| 50 | A | 0.450 | 0.36 | Splice Neutral Variant (SNV) | sense | 0.532 | 0.158 | 0.310 | 0.590 | NA | NA |
| 50 | C | 0.106 | 0.25 | Splice Neutral Variant (SNV) | sense | 0.158 | 0.629 | 0.075 | 0.137 | NA | NA |
| 50 | T | 0.255 | 0.25 | Splice Neutral Variant (SNV) | sense | 0.152 | 0.198 | 0.382 | 0.127 | NA | NA |
| 51 | A | 0.408 | 0.29 | Splice Neutral Variant (SNV) | sense | 0.194 | −0.245 | 0.265 | 0.551 | NA | NA |
| 51 | C | 0.567 | 0.33 | Splice Neutral Variant (SNV) | sense | 0.337 | −0.034 | 0.767 | 0.367 | NA | NA |
| 51 | T | −1.076 | 0.54 | Splice Neutral Variant (SNV) | nonsense | −1.157 | −0.704 | −0.905 | −1.248 | NA | NA |
| 52 | A | 0.039 | 0.34 | Splice Neutral Variant (SNV) | sense | −0.027 | 0.076 | 0.100 | −0.022 | NA | NA |
| 52 | C | 0.476 | 0.43 | Splice Neutral Variant (SNV) | sense | 0.079 | −0.061 | 0.484 | 0.468 | NA | NA |
| 52 | T | −0.334 | 0.92 | Splice Affecting Variant (SAV) | sense | −0.340 | −0.502 | −0.268 | −0.400 | NA | NA |
| | | | | Cryptic 5′ SS (P = 0.009018) | | | | | | | |
| 53 | C | −0.048 | 0.4 | Splice Neutral Variant (SNV) | sense | 0.298 | 0.836 | −0.067 | −0.028 | NA | NA |
| 53 | G | −1.757 | 0.69 | Splice Affecting Variant (SAV) | sense | −0.949 | −0.312 | −1.933 | −1.580 | NA | NA |
| | | | | ESE Loss (P = 0.046524) | | | | | | | |
| | | | | ESS Gain (P = 0.034846) | | | | | | | |
| 53 | T | −0.070 | 0.46 | Splice Neutral Variant (SNV) | sense | 0.303 | 0.246 | −0.077 | −0.062 | NA | NA |
| 54 | A | 0.470 | 0.43 | Splice Neutral Variant (SNV) | sense | −0.303 | −0.264 | 0.035 | 0.905 | NA | NA |
| 54 | C | 0.222 | 0.4 | Splice Neutral Variant (SNV) | sense | 0.186 | 0.551 | 0.448 | −0.004 | NA | NA |
| 54 | T | −0.673 | 0.47 | Splice Neutral Variant (SNV) | nonsense | −1.089 | −0.743 | −0.796 | −0.549 | NA | NA |
| 55 | A | 0.796 | 0.5 | Splice Neutral Variant (SNV) | sense | 0.193 | 0.075 | 0.508 | 1.083 | NA | NA |
| 55 | C | 0.140 | 0.43 | Splice Neutral Variant (SNV) | sense | 0.171 | −0.046 | −0.201 | 0.481 | NA | NA |
| 55 | T | 0.073 | 0.84 | Splice Affecting Variant (SAV) | sense | 0.062 | 0.357 | 0.219 | −0.073 | NA | NA |
| | | | | ESE Loss (P = 0.004064) | | | | | | | |
| | | | | ESS Gain (P = 0.034846) | | | | | | | |
| 56 | C | −0.464 | 0.57 | Splice Neutral Variant (SNV) | sense | 0.380 | 0.579 | −0.466 | −0.463 | NA | NA |
| 56 | G | −1.586 | 0.75 | Splice Affecting Variant (SAV) | sense | −1.392 | −0.978 | −1.516 | −1.656 | NA | NA |
| | | | | ESE Loss (P = 0.004064) | | | | | | | |
| 56 | T | −0.926 | 0.98 | Splice Affecting Variant (SAV) | sense | −0.836 | −1.042 | −0.923 | −0.930 | NA | NA |
| | | | | ESE Loss (P = 0.004064) | | | | | | | |
| | | | | Cryptic 5′ SS (P = 0.003403) | | | | | | | |
| 57 | C | 0.275 | 0.31 | Splice Neutral Variant (SNV) | sense | 0.617 | 0.091 | 0.063 | 0.488 | NA | NA |
| 57 | G | −0.159 | 0.31 | Splice Neutral Variant (SNV) | sense | 0.028 | 0.147 | −0.594 | 0.276 | NA | NA |
| 57 | T | −1.998 | 0.62 | Splice Affecting Variant (SAV) | nonsense | −1.675 | −1.613 | −1.949 | −2.048 | NA | NA |
| 58 | C | −0.181 | 0.29 | Splice Neutral Variant (SNV) | sense | 0.206 | −0.031 | −0.209 | −0.154 | NA | NA |
| 58 | G | −0.068 | 0.35 | Splice Neutral Variant (SNV) | sense | 0.657 | 0.338 | 0.009 | −0.145 | NA | NA |
| 58 | T | 0.344 | 0.44 | Splice Neutral Variant (SNV) | sense | 0.149 | 0.321 | 0.246 | 0.441 | NA | NA |
| 59 | C | −0.037 | 0.31 | Splice Neutral Variant (SNV) | sense | −0.110 | 0.042 | 0.427 | −0.500 | NA | NA |
| 59 | G | −0.192 | 0.56 | Splice Neutral Variant (SNV) | sense | −0.067 | −0.130 | −0.366 | −0.017 | NA | NA |
| 59 | T | 0.137 | 0.41 | Splice Neutral Variant (SNV) | sense | −0.332 | −0.015 | 0.283 | −0.010 | NA | NA |
| 60 | A | 0.046 | 0.58 | Splice Neutral Variant (SNV) | sense | 0.350 | 0.033 | −0.102 | 0.193 | NA | NA |
| 60 | C | 0.325 | 0.58 | Splice Neutral Variant (SNV) | sense | 0.079 | −0.308 | 0.517 | 0.133 | NA | NA |
| 60 | G | −0.473 | 0.59 | Splice Neutral Variant (SNV) | sense | −0.622 | −0.343 | −0.440 | −0.505 | NA | NA |
| 61 | A | −2.485 | 0.84 | Splice Affecting Variant (SAV) | nonsense | −1.871 | −1.798 | −2.150 | −2.820 | NA | NA |
| 61 | C | 0.504 | 0.6 | Splice Affecting Variant (SAV) | sense | −0.015 | 0.149 | 0.396 | 0.612 | NA | NA |
| | | | | ESS Loss (P < 0.000001) | | | | | | | |
| 61 | T | 0.316 | 0.56 | Splice Neutral Variant (SNV) | sense | 0.278 | 0.295 | 0.448 | 0.184 | NA | NA |
| 62 | A | −0.827 | 0.61 | Splice Affecting Variant (SAV) | nonsense | −0.638 | −0.412 | −0.674 | −0.980 | NA | NA |
| | | | | ESS Loss (P < 0.000001) | | | | | | | |
| 62 | C | 0.112 | 0.52 | Splice Neutral Variant (SNV) | sense | 0.367 | 0.571 | 0.120 | 0.105 | NA | NA |
| 62 | T | 0.737 | 0.55 | Splice Neutral Variant (SNV) | sense | 0.396 | −0.456 | 0.824 | 0.651 | NA | NA |
| 63 | A | 0.133 | 0.39 | Splice Neutral Variant (SNV) | sense | 0.570 | 0.316 | 0.311 | −0.045 | NA | NA |
| 63 | C | −0.144 | 0.44 | Splice Neutral Variant (SNV) | sense | 0.897 | 0.597 | 0.087 | −0.374 | NA | NA |
| 63 | T | −2.229 | 0.82 | Splice Affecting Variant (SAV) | sense | −2.707 | −2.655 | −2.037 | −2.421 | NA | NA |
| | | | | Cryptic 5′ SS (P = 0.007933) | | | | | | | |
| 64 | A | 0.247 | 0.53 | Splice Neutral Variant (SNV) | sense | 0.153 | −0.203 | 0.031 | 0.463 | NA | NA |
| 64 | C | 0.260 | 0.47 | Splice Neutral Variant (SNV) | sense | 0.505 | 1.164 | −0.026 | 0.546 | NA | NA |
| 64 | G | −0.056 | 0.55 | Splice Neutral Variant (SNV) | sense | −0.095 | 0.672 | −0.656 | 0.544 | NA | NA |
| 65 | C | 0.535 | 0.44 | Splice Neutral Variant (SNV) | sense | 0.917 | 0.447 | 0.258 | 0.813 | NA | NA |
| 65 | G | 0.291 | 0.54 | Splice Neutral Variant (SNV) | sense | 0.271 | 0.442 | 0.517 | 0.064 | NA | NA |
| 65 | T | 0.362 | 0.5 | Splice Neutral Variant (SNV) | sense | 0.152 | 0.415 | 0.377 | 0.348 | NA | NA |
| 66 | A | −0.386 | 0.87 | Splice Affecting Variant (SAV) | sense | −0.490 | −0.570 | −0.088 | −0.684 | NA | NA |
| | | | | Cryptic 5′ SS (P = 0.013422) | | | | | | | |
| 66 | C | 0.301 | 0.7 | Splice Affecting Variant (SAV) | sense | 0.393 | 0.115 | 0.104 | 0.499 | NA | NA |
| 66 | T | 0.217 | 0.75 | Splice Affecting Variant (SAV) | sense | 0.202 | 0.131 | 0.149 | 0.285 | NA | NA |
| 67 | A | −0.154 | 0.59 | Splice Neutral Variant (SNV) | sense | 0.125 | 0.081 | −0.431 | 0.123 | NA | NA |
| 67 | C | 0.117 | 0.55 | Splice Neutral Variant (SNV) | sense | 0.012 | 0.052 | 0.138 | 0.096 | NA | NA |
| 67 | G | −4.136 | 0.93 | Splice Affecting Variant (SAV) | sense | −4.512 | −3.529 | −3.967 | −4.306 | NA | NA |
| | | | | Cryptic 5′ SS (P = 0.009018) | | | | | | | |
| 68 | A | −0.097 | 0.75 | Splice Affecting Variant (SAV) | sense | 0.011 | 0.173 | 0.289 | −0.483 | NA | NA |
| 68 | C | 0.054 | 0.37 | Splice Neutral Variant (SNV) | sense | 0.367 | 0.025 | 0.248 | −0.140 | NA | NA |
| 68 | G | 0.102 | 0.48 | Splice Neutral Variant (SNV) | sense | 0.211 | 0.111 | 0.297 | −0.093 | NA | NA |
| 69 | C | 0.001 | 0.4 | Splice Neutral Variant (SNV) | sense | 0.300 | −0.419 | 0.124 | −0.122 | NA | NA |
| 69 | G | −0.131 | 0.38 | Splice Neutral Variant (SNV) | sense | 0.061 | 0.140 | −0.251 | −0.011 | NA | NA |
| 69 | T | −0.030 | 0.57 | Splice Neutral Variant (SNV) | sense | −0.008 | −0.170 | −0.137 | 0.076 | NA | NA |
| 70 | A | −0.212 | 0.58 | Splice Neutral Variant (SNV) | sense | 0.019 | −0.386 | −0.182 | −0.242 | NA | NA |
| 70 | C | −0.191 | 0.38 | Splice Neutral Variant (SNV) | sense | −0.334 | −0.909 | −0.001 | −0.381 | NA | NA |
| 70 | T | −0.159 | 0.56 | Splice Neutral Variant (SNV) | sense | −0.217 | 0.183 | 0.087 | −0.405 | NA | NA |
| 71 | A | −0.020 | 0.59 | Splice Neutral Variant (SNV) | sense | 0.433 | 0.021 | −0.712 | 0.671 | NA | NA |
| 71 | G | −0.547 | 0.97 | Splice Affecting Variant (SAV) | sense | −0.203 | −0.771 | −0.761 | −0.332 | NA | NA |
| | | | | ESS Gain (P < 0.000001) | | | | | | | |
| | | | | Cryptic 5′ SS (P = 0.021088) | | | | | | | |
| 71 | T | −0.325 | 0.76 | Splice Affecting Variant (SAV) | sense | 0.060 | 0.257 | −0.317 | −0.332 | NA | NA |
| | | | | ESS Gain (P < 0.000001) | | | | | | | |
| 72 | A | −0.320 | 0.32 | Splice Neutral Variant (SNV) | sense | 0.623 | 0.348 | −0.168 | −0.473 | NA | NA |
| 72 | C | 0.106 | 0.34 | Splice Neutral Variant (SNV) | sense | −0.167 | 0.024 | −0.017 | 0.229 | NA | NA |
| 72 | G | −0.044 | 0.37 | Splice Neutral Variant (SNV) | sense | 0.627 | 0.199 | −0.086 | −0.002 | NA | NA |
| 73 | C | 0.234 | 0.35 | Splice Neutral Variant (SNV) | sense | 0.181 | 0.119 | 0.491 | −0.024 | NA | NA |
| 73 | G | 0.089 | 0.34 | Splice Neutral Variant (SNV) | sense | 0.352 | 0.113 | −0.248 | 0.427 | NA | NA |
| 73 | T | −0.229 | 0.4 | Splice Neutral Variant (SNV) | sense | 0.103 | 0.196 | 0.013 | −0.471 | NA | NA |
| 74 | A | −1.758 | 0.45 | Splice Neutral Variant (SNV) | nonsense | −0.942 | −1.421 | −1.427 | −2.090 | NA | NA |
| 74 | C | −0.153 | 0.37 | Splice Neutral Variant (SNV) | sense | −0.153 | −0.065 | −0.100 | −0.206 | NA | NA |
| 74 | G | −2.168 | 0.59 | Splice Neutral Variant (SNV) | nonsense | −1.380 | −1.661 | −2.321 | −2.016 | NA | NA |
| 75 | A | 0.422 | 0.51 | Splice Neutral Variant (SNV) | sense | 0.847 | 0.405 | 0.340 | 0.504 | NA | NA |
| 75 | C | 0.019 | 0.45 | Splice Neutral Variant (SNV) | sense | 0.624 | 0.503 | −0.035 | 0.073 | NA | NA |
| 75 | G | 0.415 | 0.54 | Splice Neutral Variant (SNV) | sense | 0.206 | 0.384 | 0.577 | 0.253 | NA | NA |
| 76 | A | −0.002 | 0.69 | Splice Affecting Variant (SAV) | sense | −0.103 | 0.311 | −0.328 | 0.323 | NA | NA |
| 76 | C | 0.100 | 0.75 | Splice Affecting Variant (SAV) | sense | −0.023 | −0.306 | −0.132 | 0.332 | NA | NA |
| 76 | G | −0.031 | 0.68 | Splice Affecting Variant (SAV) | sense | 0.441 | 0.298 | −0.362 | 0.301 | NA | NA |
| 77 | A | −0.095 | 0.85 | Splice Affecting Variant (SAV) | sense | −0.386 | −0.936 | 0.220 | −0.409 | NA | NA |
| | | | | Loss of natural 5′ SS | | | | | | | |
| | | | | (P < 0.000001) | | | | | | | |
| 77 | G | −0.005 | 0.85 | Splice Affecting Variant (SAV) | sense | 0.318 | 0.769 | −0.223 | 0.213 | NA | NA |
| 77 | T | −0.564 | 0.85 | Splice Affecting Variant (SAV) | sense | 0.094 | −0.340 | −0.385 | −0.743 | NA | NA |
| 78 | A | −0.277 | 0.82 | Splice Affecting Variant (SAV) | sense | −0.094 | −0.418 | 0.054 | −0.608 | NA | NA |
| 78 | C | −0.024 | 0.82 | Splice Affecting Variant (SAV) | sense | −0.269 | 0.327 | 0.170 | −0.219 | NA | NA |
| 78 | G | 0.686 | 0.83 | Splice Affecting Variant (SAV) | sense | −0.004 | 0.512 | 0.753 | 0.620 | NA | NA |

TABLE 4
Emprical measurement of the effects of genome edits to exon 2 of DBR1 on cellular growth

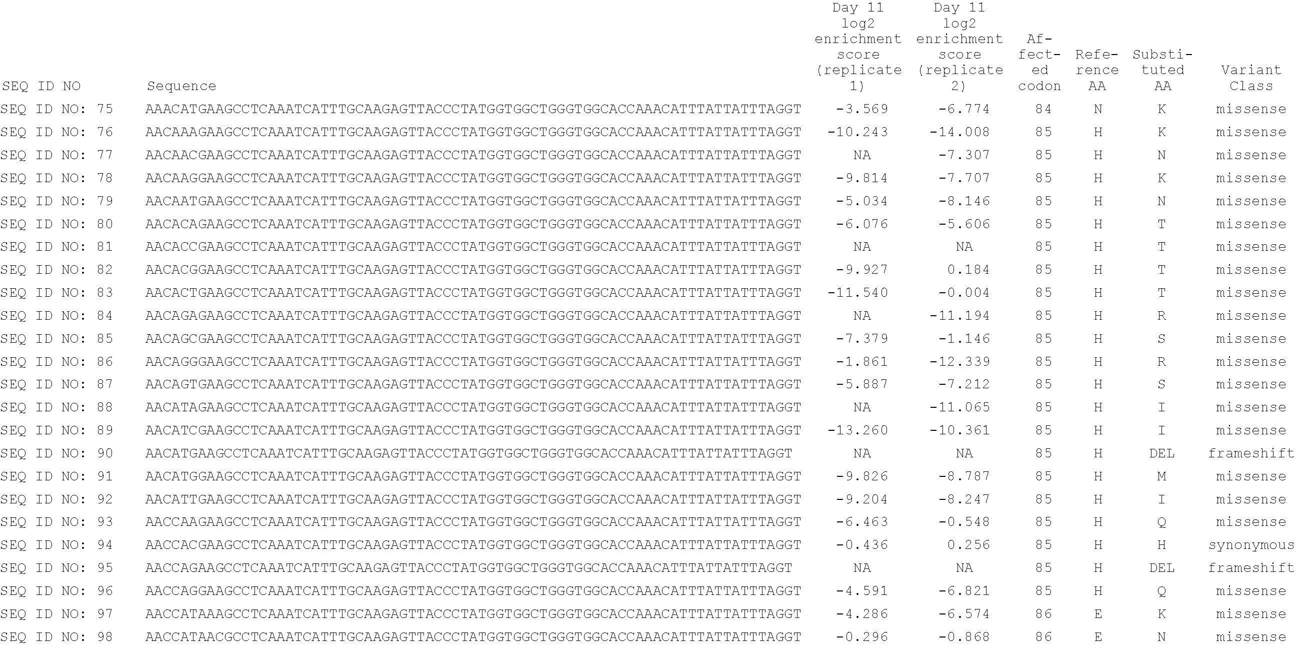

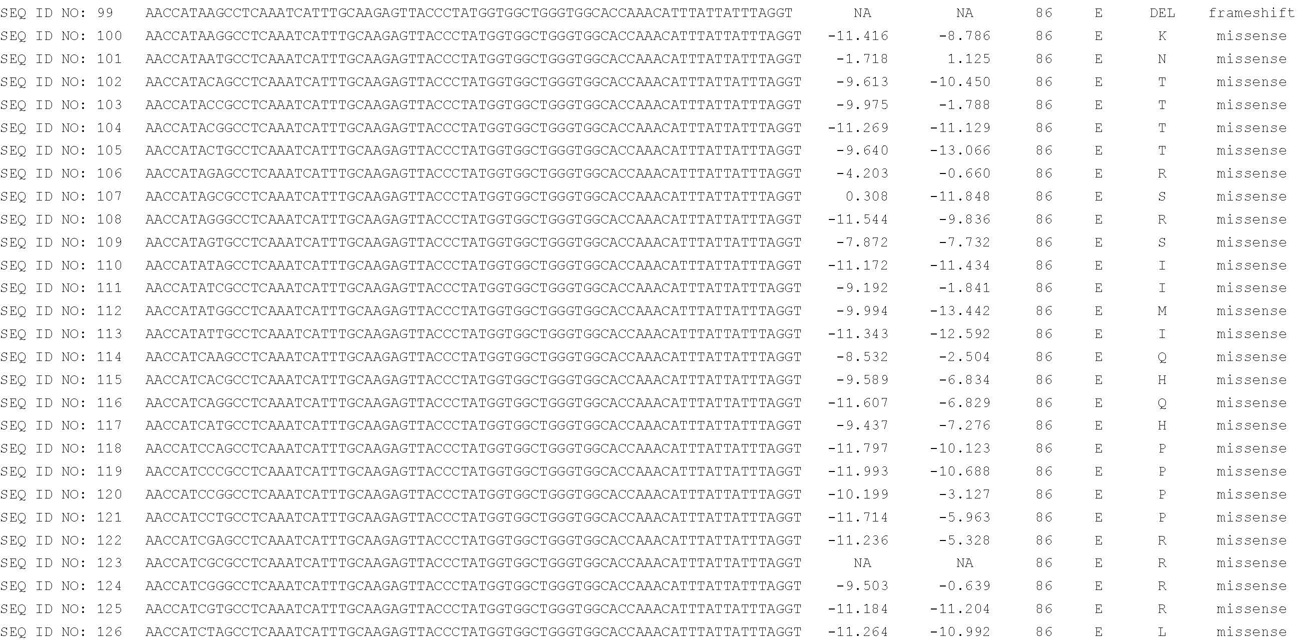

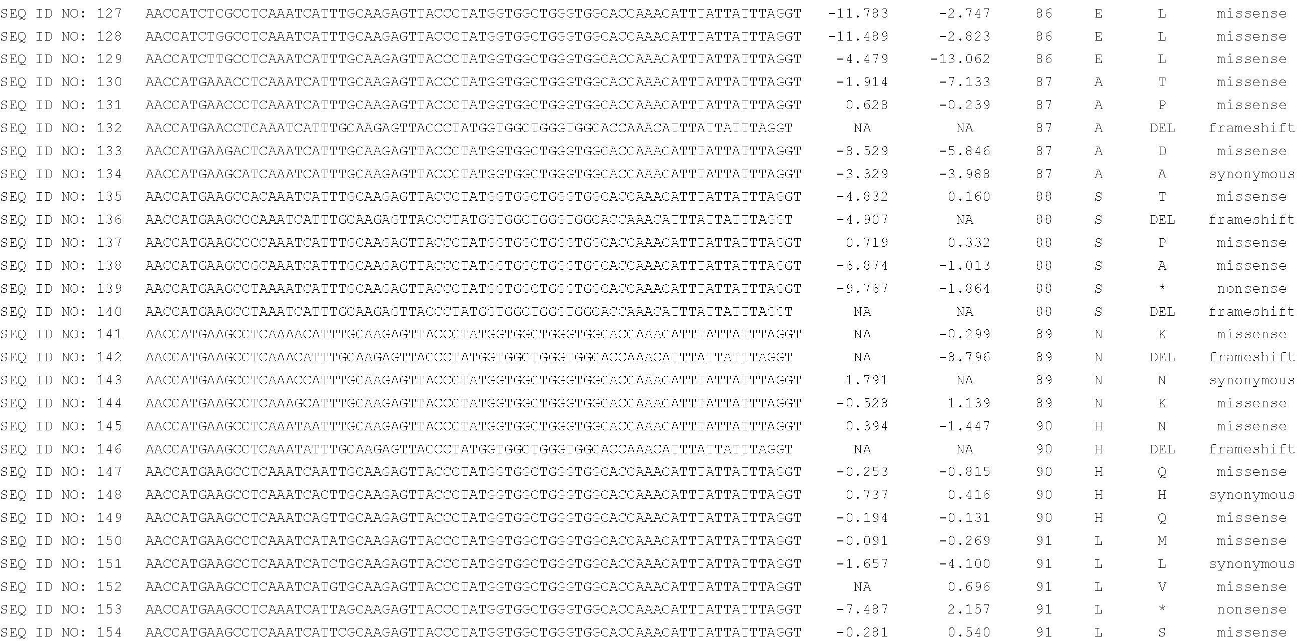

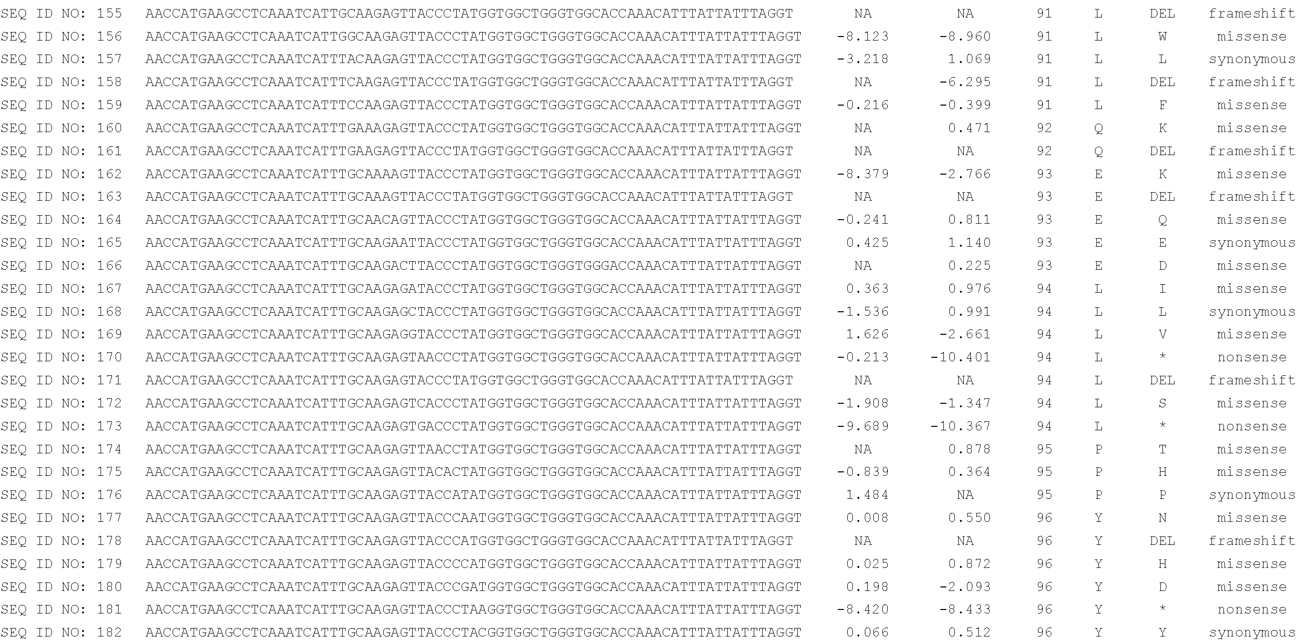

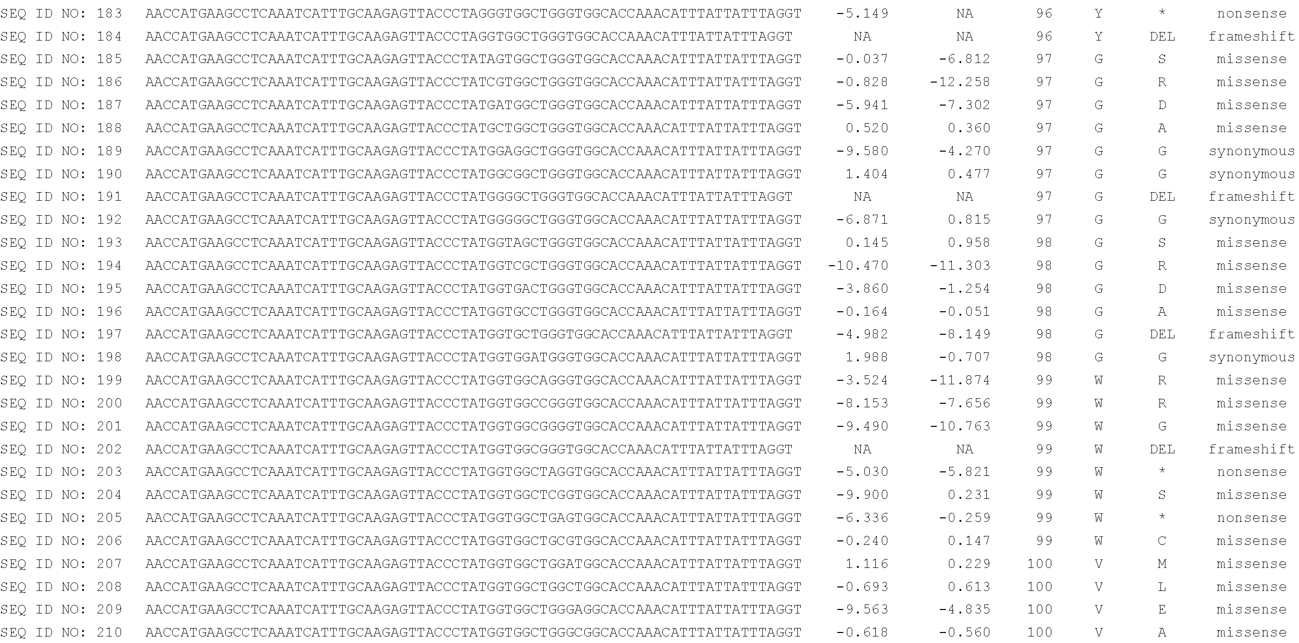

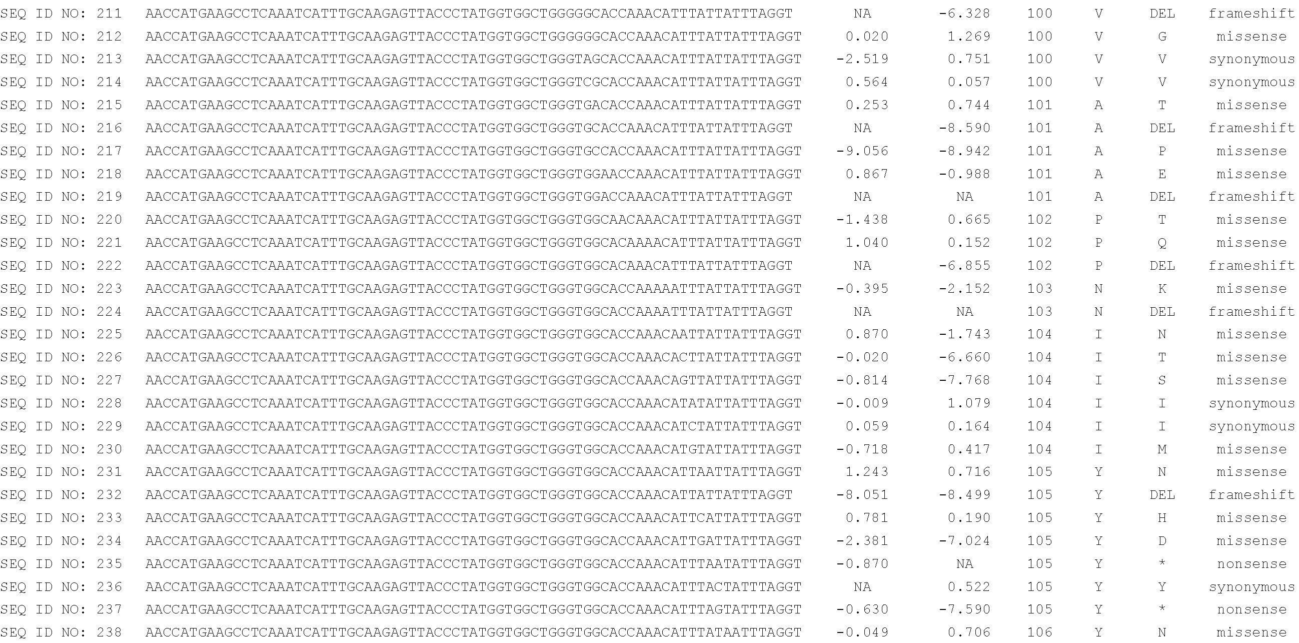

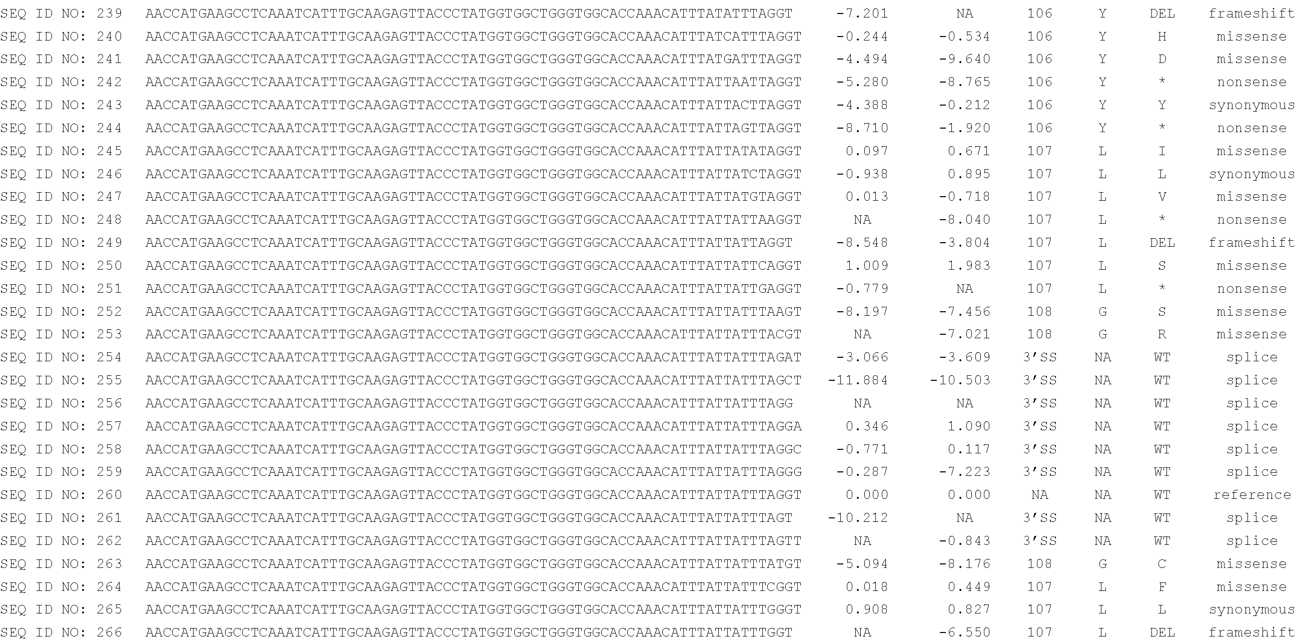

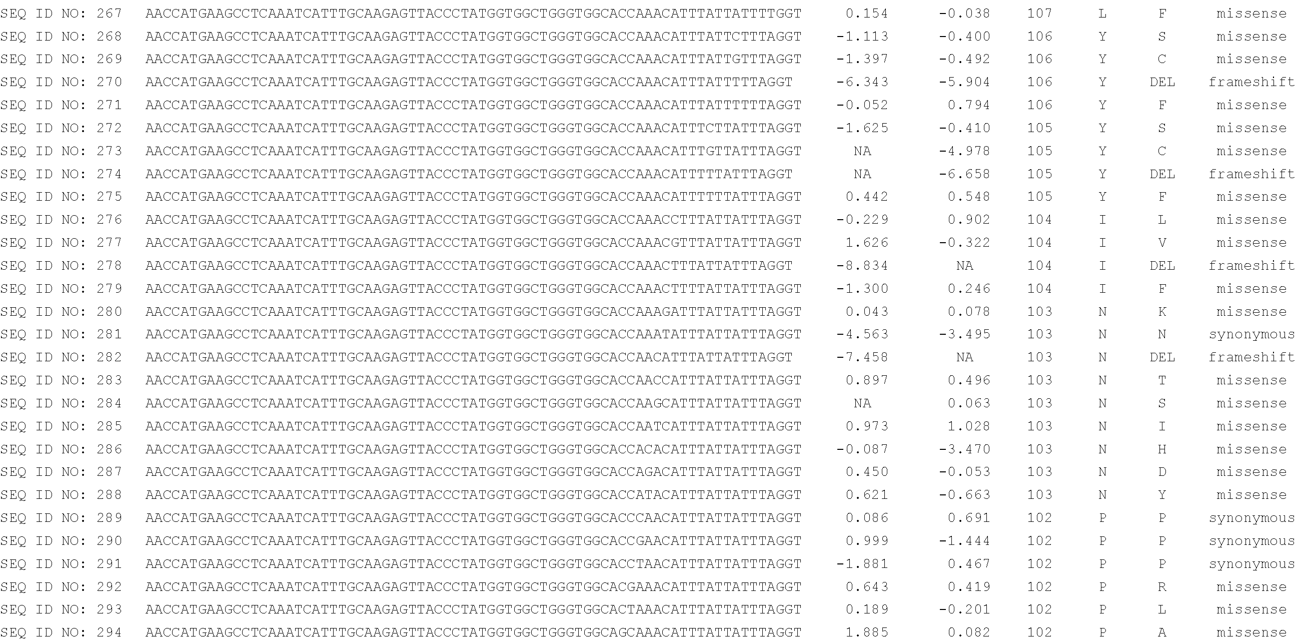

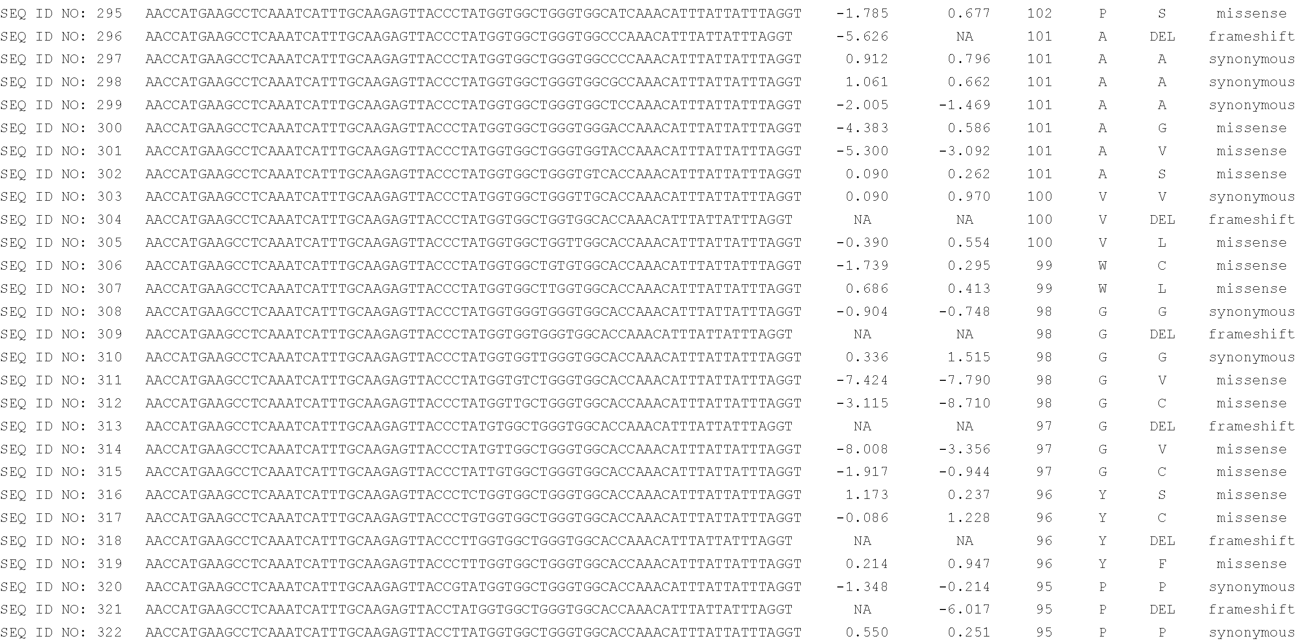

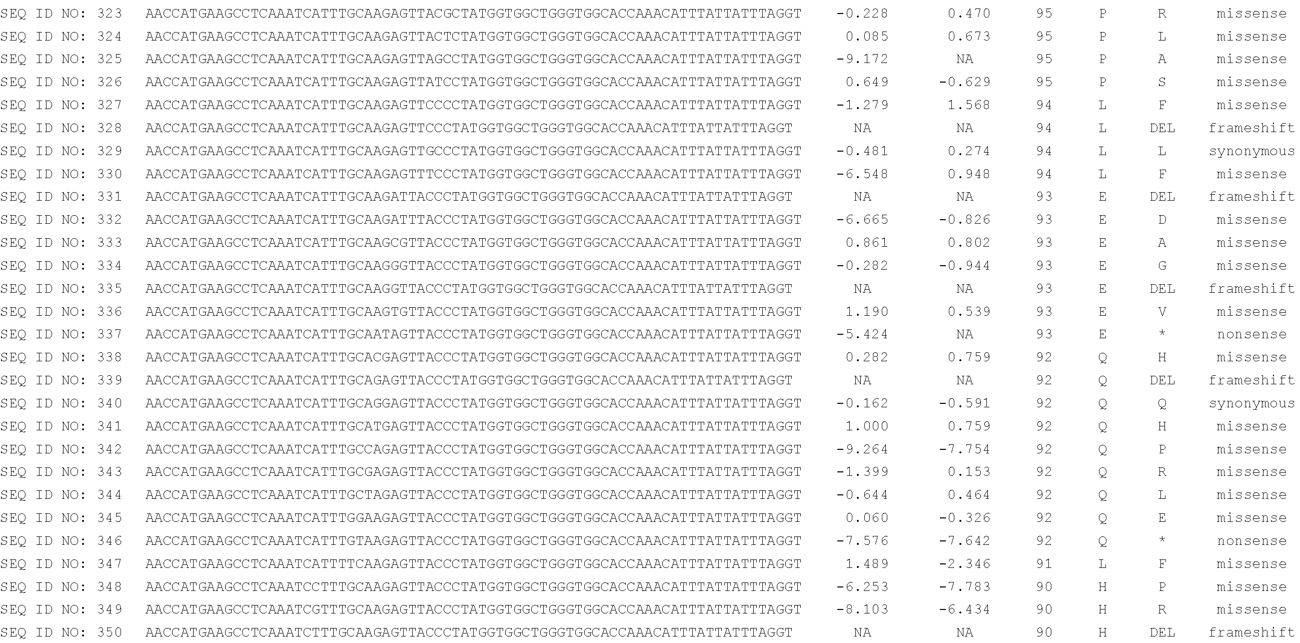

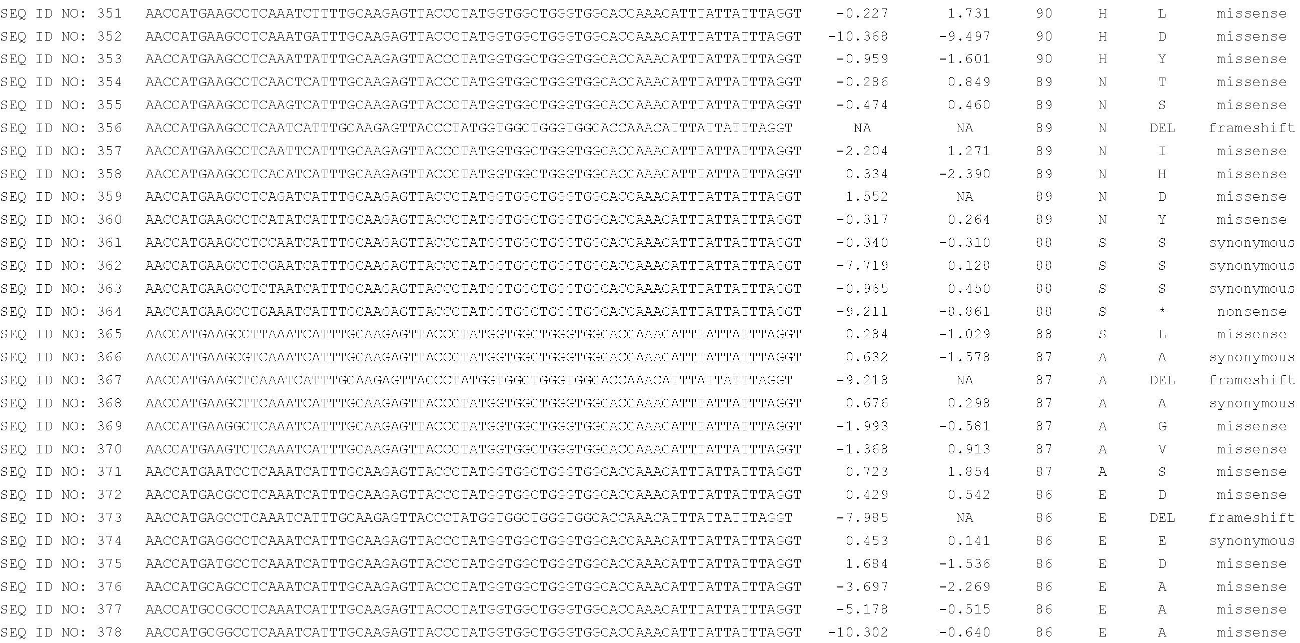

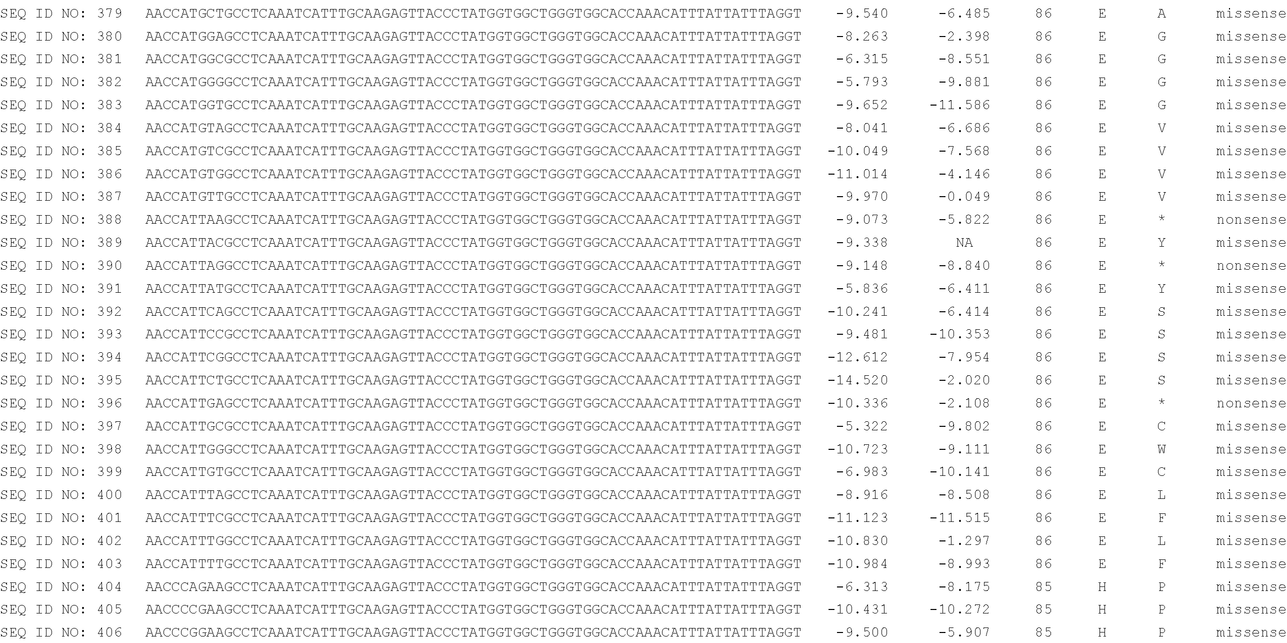

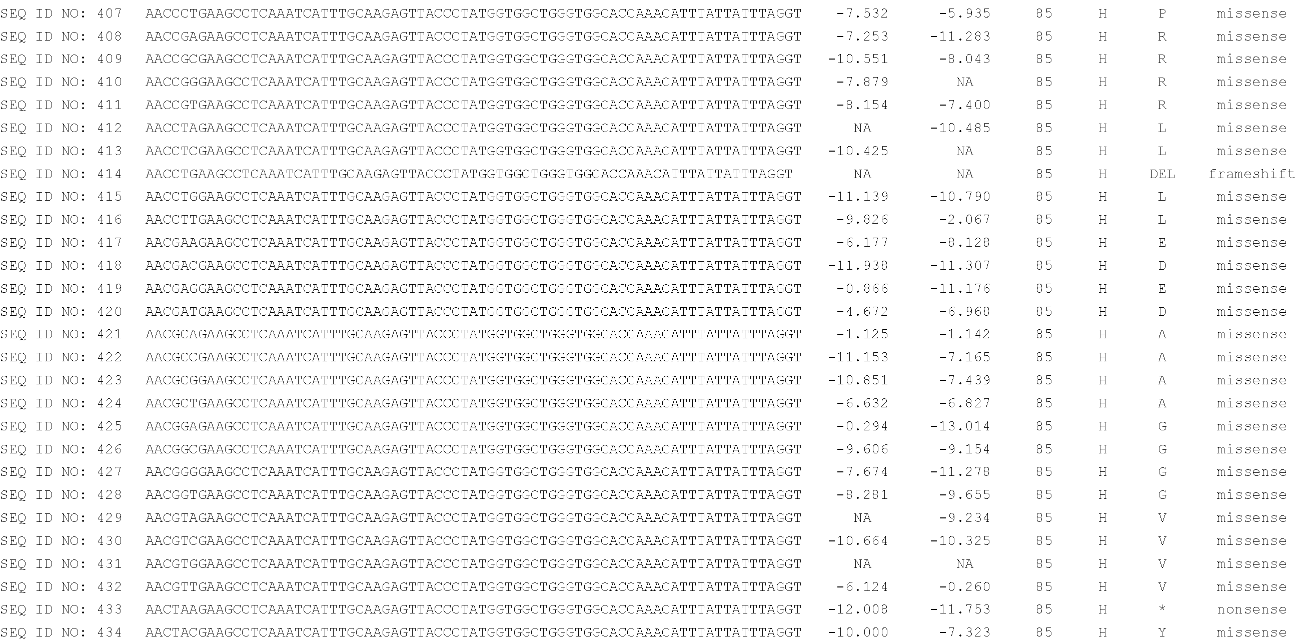

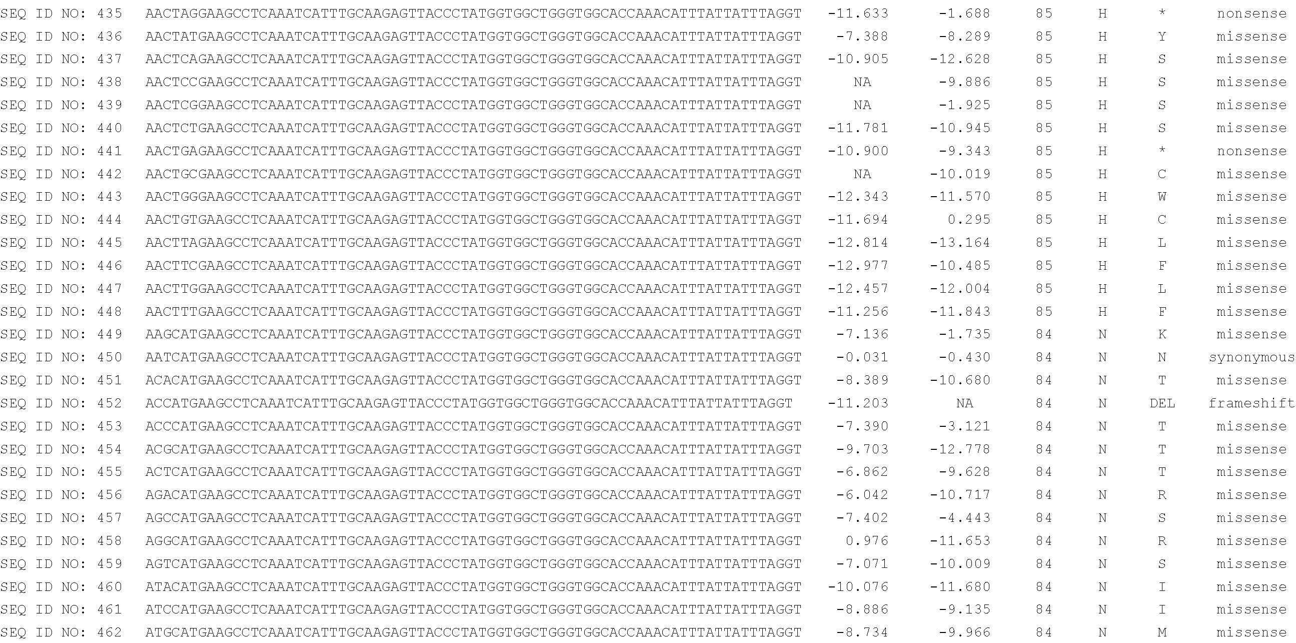

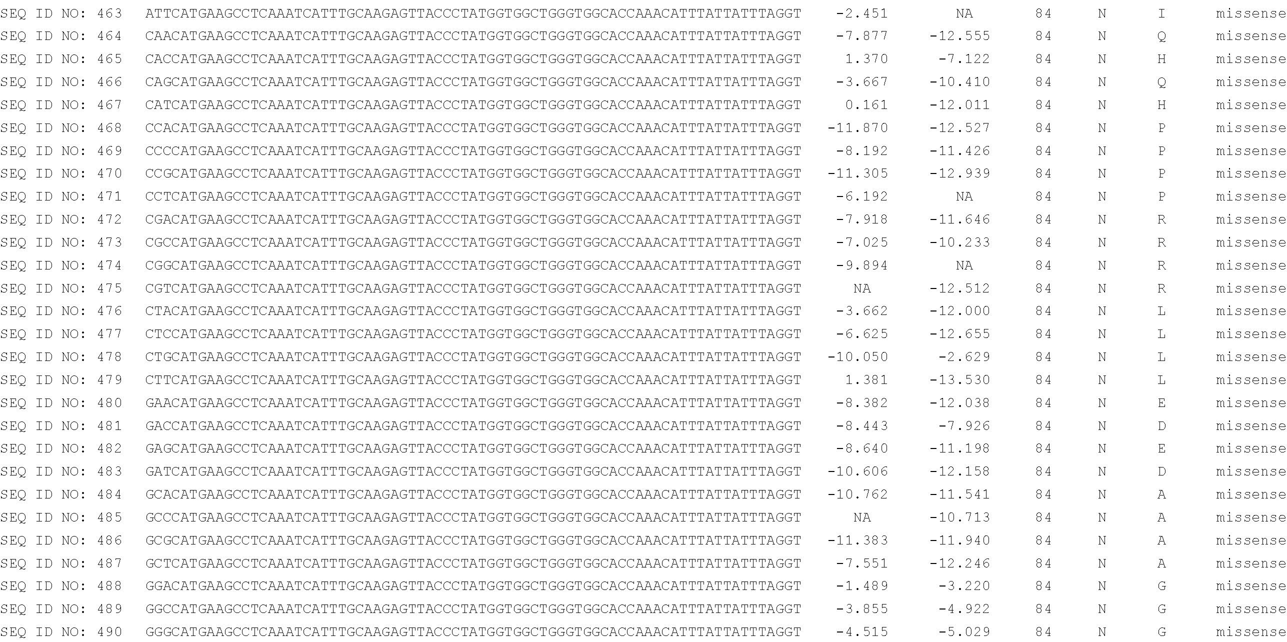

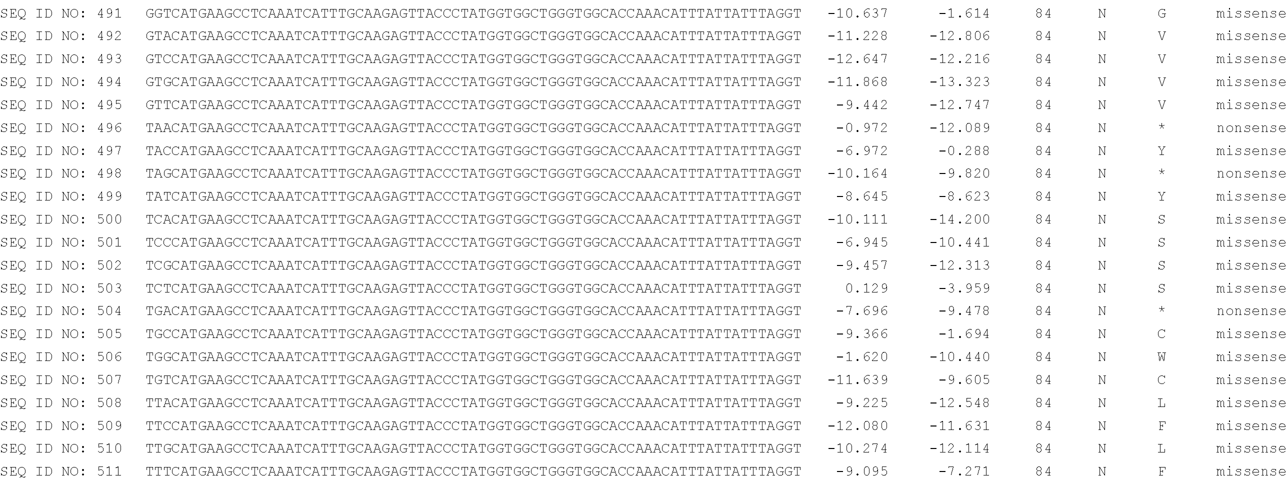

Gray rows indicate missing data across both replicates

What is claimed is:

1. A method for introducing a plurality of programmed nucleotide modifications into a single locus of a desired genomic DNA sequence, comprising:
(a) synthesizing a homology-directed repair (HDR) library comprising a plurality of oligonucleotides, wherein each oligonucleotide comprises a programmed nucleotide modification in the locus of the desired genome; and
(b) co-transfecting a population of cells with (i) an expression system capable of expressing Cas9 and a single guide RNA (sgRNA), and (ii) the HDR library, wherein the expression system is capable of introducing the plurality of oligonucleotides having the programmed nucleotide modifications to the locus of the desired genomic DNA sequence in one or more cells of the population;
wherein the method is carried out in a single experiment.

2. The method of claim 1, wherein each programmed nucleotide modification is a single nucleotide variant.

3. The method of claim 1, wherein the HDR library is constructed using an oligonucleotide comprising a degenerate sequence.

4. The method of claim 3, wherein the degenerate sequence is between 1 and 100 nucleotides in length.

5. The method of claim 4, wherein the HDR library comprises a set of oligonucleotides having at least 100 unique programmed nucleotide modifications, a least 200 unique programmed nucleotide modifications, at least 300 unique programmed nucleotide modifications, at least 400 unique programmed nucleotide modifications, at least 500 unique programmed nucleotide modifications, at least 600 unique programmed nucleotide modifications, at least 700 unique programmed nucleotide modifications, at least 800 unique programmed nucleotide modifications, at least 900 unique programmed nucleotide modifications, at least 1,000 unique programmed nucleotide modifications, at least 3,000 unique programmed nucleotide modifications, at least 4,000 unique programmed nucleotide modifications, at least 5,000 unique programmed nucleotide modifications, at least 6,000 unique programmed nucleotide modifications, at least 7,000 unique programmed nucleotide modifications, at least 8,000 unique programmed nucleotide modifications, at least 9,000 unique programmed nucleotide modifications, at least 10,000 unique programmed nucleotide modifications, at least 12,000 unique programmed nucleotide modifications, at least 14,000 unique programmed nucleotide modifications, at least 16,000 unique programmed nucleotide modifications, at least 18,000 unique programmed nucleotide modifications, at least 20,000 unique programmed nucleotide modifications, at least 25,000 unique programmed nucleotide modifications, at least 30,000 unique programmed nucleotide modifications, at least 40,000 unique programmed nucleotide modifications, or at least 50,000 unique programmed nucleotide modifications.

6. The method of claim 1, wherein the plurality of programmed nucleotide modifications that are introduced to the locus of the desired genomic DNA sequence results in a saturating set of programmed nucleotide modifications.

7. The method of claim 1, wherein the plurality of oligonucleotides are synthesized on a microarray or in column-based synthesis.

8. The method of claim 1, wherein the expression system comprises a plasmid which comprises (i) a Cas9 expression cassette that includes a nucleotide sequence which encodes a Cas9 nuclease, (ii) an sgRNA expression cassette, and (iii) a species-specific promoter that is specific to the population of cells.

9. The method of claim 1, further comprising cloning the HDR library prior to the step of co-transfecting the population of cells.

10. The method of claim 1, wherein each oligonucleotide of the HDR library further comprises a pair of homology arms.

11. The method of claim 1, further comprising
(c) harvesting the population of cells,
(d) selectively amplifying a genomic DNA and RNA sample, wherein the edited sequences are amplified and the non-edited sequence are not amplified, and
(e) sequencing the genomic DNA and RNA sample that has been selectively amplified, resulting in a set of genomic transcripts which include the plurality of programmed nucleotide modifications.

12. The method of claim 11, further comprising functionally analyzing the set of genomic transcripts using a functional assay.

13. The method of claim 12, wherein the functional assay is selected from the group consisting of targeted RNA sequencing to measure transcript abundance, targeted DNA sequencing to measure reduced cellular fitness, targeted chromatin immunoprecipitation-sequencing (CHiP-seq) of co-activators to assay enhancers, increased cellular growth rate to assay cancer drivers or drug resistance, and FACS-based phenotypic sorting for cellular assays.

14. A method for analyzing the functional consequence of a genomic mutation comprising:
(a) synthesizing a homology-directed repair (HDR) library comprising a plurality of oligonucleotides, wherein each oligonucleotide comprises a programmed nucleotide modification in the locus of the desired genome;
(b) co-transfecting a population of cells with (i) an expression system capable of expressing Cas9 and a guide RNA (sgRNA) and (ii) the HDR library, wherein the expression system is capable of introducing the plurality of oligonucleotides having the programmed nucleotide modifications to the locus of the desired genomic DNA sequence in one or more cells of the population;
(c) harvesting the population of cells;
(d) selectively amplifying a genomic DNA and RNA sample, wherein the edited sequences are amplified and the non-edited sequence are not amplified;
(e) sequencing the genomic DNA and RNA sample that has been selectively amplified, resulting in a set of genomic transcripts which include the plurality of programmed nucleotide modifications; and
(f) functionally analyzing the set of genomic transcripts using a functional assay;
wherein the method is carried out in a single experiment.

15. The method of claim 14, wherein the HDR library is constructed using an oligonucleotide comprising a degenerate sequence, wherein the degenerate sequence is between 1 and 100 nucleotides in length.

16. The method of claim 14, wherein the plurality of oligonucleotides are synthesized on a microarray or in column-based synthesis.

17. The method of claim 14, wherein the expression system comprises a plasmid which comprises (i) a Cas9 expression cassette that includes a nucleotide sequence which encodes a Cas9 nuclease, (ii) an sgRNA expression cassette, and (iii) a species-specific promoter that is specific to the population of cells.

18. The method of claim 14, wherein the functional assay is selected from the group consisting of targeted RNA sequencing to measure transcript abundance, targeted DNA sequencing to measure reduced cellular fitness, targeted chromatin immunoprecipitation-sequencing (CHiP-seq) of co-activators to assay enhancers, increased cellular growth rate to assay cancer drivers or drug resistance, and FACS-based phenotypic sorting for cellular assays.

19. A method for genomic screening, comprising:
(a) introducing a plurality of programmed nucleotide modifications to a single genomic locus in a single experiment, wherein step (a) comprises the method of claim 1;
(b) sequencing the genomic DNA or cDNA of the edited locus; and
(c) quantifying the transcript abundance of each mutation.

20. The method of claim 19, wherein step (c) comprises calculating an enrichment score for each mutation.
